SEQUENCE LISTING (528 sequences)

SEQ ID NO: 1 - protein, 11 aa, Artificial Sequence
  binding polypeptide
XGXNXXELXTP

SEQ ID NO: 2 - protein, 11 aa, Artificial Sequence
  binding polypeptide
XERNGRXLXTP

SEQ ID NO: 3 - protein, 10 aa, Artificial Sequence
  binding polypeptide
XGXNXRXXIP

SEQ ID NO: 4 - protein, 10 aa, Artificial Sequence
  binding polypeptide
XGXNXRXXIP

SEQ ID NO: 5 - protein, 94 aa, Homo sapiens
VSDVPRDLEVVAATPTSLLISWDAPAVTVRYYRITYGETGGNSPVQEFTVPGSKSTATISGLKPGVDYTITGYAVTGRGDSPASSKPISINYRT

SEQ ID NO: 6 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMGLYGHELLTPISINYRT

SEQ ID NO: 7 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDGENGQFLLVPISINYRT

SEQ ID NO: 8 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMGPNDNELLTPISINYRT

SEQ ID NO: 9 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTAGWDDHELFIPISINYRT

SEQ ID NO: 10 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTSGHNDHMLMIPISINYRT

SEQ ID NO: 11 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTAGYNDQILMTPISINYRT

SEQ ID NO: 12 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTFGLYGKELLIPISINYRT

SEQ ID NO: 13 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTTGPNDRLLFVPISINYRT

SEQ ID NO: 14 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDVYNDHEIKTPISINYRT

SEQ ID NO: 15 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDGKDGRVLLTPISINYRT

SEQ ID NO: 16 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEVHHDREIKTPISINYRT

SEQ ID NO: 17 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTQAPNDRVLYTPISINYRT

SEQ ID NO: 18 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTREENDHELLIPISINYRT

SEQ ID NO: 19 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVTHNGHPLMTPISINYRT

SEQ ID NO: 20 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLALKGHELLTPISINYRT

SEQ ID NO: 21 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTVAQNDHELITPISINYRT

SEQ ID NO: 22 - protein, 95 aa, Artificial Sequence
  binding polypeptide
VSDVPRDLQEVVAATPTSLLISWRHPHFPTRYYRITYGETGGNSPVQEFTVPLQPPAATISGLKPGVDYTITGYAVTMAQSGHELFTPISINYRT

SEQ ID NO: 23 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRKLMTPISINYRT

SEQ ID NO: 24 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRVLMTPISINYRT

SEQ ID NO: 25 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRHLMTPISINYRT

SEQ ID NO: 26 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRMLMTPISINYRT

SEQ ID NO: 27 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLERNGRVLMTPISINYRT

SEQ ID NO: 28 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLERNGRVLNTPISINYRT

SEQ ID NO: 29 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRQLMTPISINYRT

SEQ ID NO: 30 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRTLFTPISINYRT

SEQ ID NO: 31 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLERNGRELMTPISINYRT

SEQ ID NO: 32 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLERNGRLLNTPISINYRT

SEQ ID NO: 33 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTHERNGRVLMTPISINYRT

SEQ ID NO: 34 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEERNGRVLFTPISINYRT

SEQ ID NO: 35 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRQLYTPISINYRT

SEQ ID NO: 36 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRALMTPISINYRT

SEQ ID NO: 37 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRNLMTPISINYRT

SEQ ID NO: 38 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLERNGRVLITPISINYRT

SEQ ID NO: 39 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRVLNTPISINYRT

SEQ ID NO: 40 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGKVLMTPISINYRT

SEQ ID NO: 41 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRTLMMPISINYRT

SEQ ID NO: 42 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMERNGRELMTPISINYRT

SEQ ID NO: 43 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEERNGRTLRTPISINYRT

SEQ ID NO: 44 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGKTLMTPISINYRT

SEQ ID NO: 45 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLERNDRVLLTPISINYRT

SEQ ID NO: 46 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLERNGRKLMTPISINYRT

SEQ ID NO: 47 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVEPNGRVLNTPISINYRT

SEQ ID NO: 48 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNDRVLFTPISINYRT

SEQ ID NO: 49 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRELKTPISINYRT

SEQ ID NO: 50 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRELRTPISINYRT

SEQ ID NO: 51 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTQERNGRELMTPISINYRT

SEQ ID NO: 52 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRELMTPISINYRT

SEQ ID NO: 53 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRVLSVPISINYRT

SEQ ID NO: 54 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERDGRTLRTPISINYRT

SEQ ID NO: 55 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRELNTPISINYRT

SEQ ID NO: 56 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRVLIVPISINYRT

SEQ ID NO: 57 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLERNGRELMVPISINYRT

SEQ ID NO: 58 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDGRNDRKLMVPISINYRT

SEQ ID NO: 59 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVEHNGRTSFTPISINYRT

SEQ ID NO: 60 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERDGRKLYTPISINYRT

SEQ ID NO: 61 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLERNGRELNTPISINYRT

SEQ ID NO: 62 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDGWNGRLLSIPISINYRT

SEQ ID NO: 63 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDGQNGRLLNVPISINYRT

SEQ ID NO: 64 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTIEKNGRHLNIPISINYRT

SEQ ID NO: 65 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDGWNGKMLSVPISINYRT

SEQ ID NO: 66 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDGYNDRLLFIPISINYRT

SEQ ID NO: 67 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDGPNDRLLNIPISINYRT

SEQ ID NO: 68 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDGPNNRELIVPISINYRT

SEQ ID NO: 69 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDGLNGKYLFVPISINYRT

SEQ ID NO: 70 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEGWNDRELFVPISINYRT

SEQ ID NO: 71 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTFGWNGRELLTPISINYRT

SEQ ID NO: 72 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTFGWNDRELLIPISINYRT

SEQ ID NO: 73 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLEWNNRVLMTPISINYRT

SEQ ID NO: 74 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVEWNGRVLMTPISINYRT

SEQ ID NO: 75 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTNERNGRELMTPISINYRT

SEQ ID NO: 76 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLERNGKELMTPISINYRT

SEQ ID NO: 77 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRELLTPISINYRT

SEQ ID NO: 78 - protein, 86 aa, Artificial Sequence; identical to SEQ ID NO: 49
  binding polypeptide

SEQ ID NO: 79 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTQERNGRELRTPISINYRT

SEQ ID NO: 80 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRELLWPISINYRT

SEQ ID NO: 81 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLERNGRELMIPISINYRT

SEQ ID NO: 82 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGLVLMTPISINYRT

SEQ ID NO: 83 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGRVLIIPISINYRT

SEQ ID NO: 84 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGHKLFTPISINYRT

SEQ ID NO: 85 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNERVLMTPISINYRT

SEQ ID NO: 86 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTFGPNDRELLTPISINYRT

SEQ ID NO: 87 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMGPNDRELLTPISINYRT

SEQ ID NO: 88 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMGKNDRELLTPISINYRT

SEQ ID NO: 89 - protein, 87 aa, Artificial Sequence
  binding polypeptide
EVVAATPTSLLISWRHHPHFPTRYYRITYGETGGNSPVQEFTVPLQPPTATISGLKPGVDYTITGYAVTVHWNGRELMTPISINYRT

SEQ ID NO: 90 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEEWNGRVLMTPISINYRT

SEQ ID NO: 91 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGHTLMTPISINYRT

SEQ ID NO: 92 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVEENGRQLMTPISINYRT

SEQ ID NO: 93 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLERNGQVLFTPISINYRT

SEQ ID NO: 94 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERNGQVLYTPISINYRT

SEQ ID NO: 95 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTWGYKDHELLIPISINYRT

SEQ ID NO: 96 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGRNDRELLTPISINYRT

SEQ ID NO: 97 - protein, 86 aa, Artificial Sequence; identical to SEQ ID NO: 67
  binding polypeptide

SEQ ID NO: 98 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTFARDGHEILTPISINYRT

SEQ ID NO: 99 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLEQNGRELMTPISINYRT

SEQ ID NO: 100 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVEENGRVLNTPISINYRT

SEQ ID NO: 101 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLEPNGRYLMVPISINYRT

SEQ ID NO: 102 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEGRNGRELFIPISINYRT

SEQ ID NO: 103 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTSGRNDRELLVPISINYRT

SEQ ID NO: 104 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVERDGRELNIPISINYRT

SEQ ID NO: 105 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVEQNGRVLMTPISINYRT

SEQ ID NO: 106 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVEHNGRVLNIPISINYRT

SEQ ID NO: 107 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMAPNGRELLTPISINYRT

SEQ ID NO: 108 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVEQNGRVLNTPISINYRT

SEQ ID NO: 109 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDGRNGHELMTPISINYRT

SEQ ID NO: 110 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEGRNGRELMVPISINYRT

SEQ ID NO: 111 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLERNNRELLTPISINYRT

SEQ ID NO: 112 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMERSGRELMTPISINYRT

SEQ ID NO: 113 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTRALLSIELFTPISINYRT

SEQ ID NO: 114 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTFARKGTELFTPISINYRT

SEQ ID NO: 115 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTLERCGRELFTPISINYRT

SEQ ID NO: 116 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTRERNGRELFTPISINYRT

SEQ ID NO: 117 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTKERNGRELFTPISINYRT

SEQ ID NO: 118 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTCERNGRELFTPISINYRT

SEQ ID NO: 119 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTLERTGRELFTPISINYRT

SEQ ID NO: 120 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTWERTGKELFTPISINYRT

SEQ ID NO: 121 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTIERTCRELFTPISINYRT

SEQ ID NO: 122 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTGGMIVRELFTPISINYRT

SEQ ID NO: 123 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTFGRSSRELFTPISINYRT

SEQ ID NO: 124 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTRHKSRGELFTPISINYRT

SEQ ID NO: 125 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTRHRDKRELFTPISINYRT

SEQ ID NO: 126 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTYHRGRGELFTPISINYRT

SEQ ID NO: 127 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTRHRGCRELFTPISINYRT

SEQ ID NO: 128 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTSHRLRKELFTPISINYRT

SEQ ID NO: 129 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTMHRQRGELFTPISINYRT

SEQ ID NO: 130 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTFHRRRGELFTPISINYRT

SEQ ID NO: 131 - protein, 94 aa, Artificial Sequence; identical to SEQ ID NO: 130
  binding polypeptide

SEQ ID NO: 132 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTSHRRRNELFTPISINYRT

SEQ ID NO: 133 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTLHRRVRELFTPISINYRT

SEQ ID NO: 134 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTRHRRRGELFTPISINYRT

SEQ ID NO: 135 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTWHRSRKELFTPISINYRT

SEQ ID NO: 136 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTRHRSRGELFTPISINYRT

SEQ ID NO: 137 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTVHRTGRELFTPISINYRT

SEQ ID NO: 138 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTWHRVRGELFTPISINYRT

SEQ ID NO: 139 - protein, 94 aa, Artificial Sequence; identical to SEQ ID NO: 138
  binding polypeptide

SEQ ID NO: 140 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTWHRWRGELFTPISINYRT

SEQ ID NO: 141 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTWKRSGGELFTPISINYRT

SEQ ID NO: 142 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTRLXNXVELFTPISINYRT

SEQ ID NO: 143 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTWRTPHAELFTPISINYRT

SEQ ID NO: 144 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTLSPHSVELFTPISINYRT

SEQ ID NO: 145 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTVSRQKAELFTPISINYRT

SEQ ID NO: 146 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTSSYSKLELFTPISINYRT

SEQ ID NO: 147 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTLTDRGSELFTPISINYRT

SEQ ID NO: 148 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTGTRTRSELFTPISINYRT

SEQ ID NO: 149 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTPVAGCSELFTPISINYRT

SEQ ID NO: 150 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTWWQTPRELFTPISINYRT

SEQ ID NO: 151 - protein, 94 aa, Artificial Sequence; identical to SEQ ID NO: 150
  binding polypeptide

SEQ ID NO: 152 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTWERNGRELFTPISINYRT

SEQ ID NO: 153 - protein, 94 aa, Artificial Sequence
  binding polypeptide
VSDVPRDLEVVAATPTSLLISWRHPHFPTRYYRITYGETGGNSPVQEFTVPLQPPAATISGLKPGVDYTITGYAVTWEWNGRELFTPISINYRT

SEQ ID NO: 154 - protein, 94 aa, Artificial Sequence; identical to SEQ ID NO: 117
  binding polypeptide

SEQ ID NO: 155 - protein, 94 aa, Artificial Sequence
  binding polypeptide
VSDVPRDLEVVAATPTSLLISWLHPHFPTHYYRITYGETGGNSPVQEFTVPLQPPAATISGLKPGVDYTITGYAVTGALNTSELFTPISINYRT

SEQ ID NO: 156 - protein, 94 aa, Artificial Sequence
  binding polypeptide
VSDVPRDLEVVAATPTSLLISWRHPHFPTHYYRITYGETGGNSPVQEFTVPLQPPAATISGLKPGVDYTITGYAVTFGRERRELFTPISINYRT

SEQ ID NO: 157 - protein, 94 aa, Artificial Sequence
  binding polypeptide
VSDVPRDLEVVAATPTSLLISWLHPHFPTHYYRITYGETGGNSPVQEFTVPLQPPAATISGLKPGVDYTITGYAVTSGRVSFELFTPISINYRT

SEQ ID NO: 158 - protein, 94 aa, Artificial Sequence
  binding polypeptide
VSDVPRDLEVVAATPTSLLISWRHPHFPTHYYRITYGETGGNSPVQEFTVPLQPPAATISGLKPGVDYTITGYAVTFHRRRGELFTPISINYRT

SEQ ID NO: 159 - protein, 94 aa, Artificial Sequence
  binding polypeptide
VSDVPRDLEVVAATPTSLLISWLHPHFPTHYYRITYGETGGNSPVQEFTVPLQPPAATISGLKPGVDYTITGYAVTLIRMNTELFTPISINYRT

SEQ ID NO: 160 - protein, 94 aa, Artificial Sequence
  binding polypeptide
VSDVPRDLEVVAATPTSLLISWLHPHFPTHYYRITYGETGGNSPVQEFTVPLQPPAATISGLKPGVDYTITGYAVTCLHLITELFTPISINYRT

SEQ ID NO: 161 - protein, 94 aa, Artificial Sequence
  binding polypeptide
VSDVPRDLEVVAATPTSLLISWLHPHFPTHYYRITYGETGGNSPVQEFTVPLQPPAATISGLKPGVDYTITGYAVTVLKLTLELFTPISINYRT

SEQ ID NO: 162 - protein, 94 aa, Artificial Sequence; identical to SEQ ID NO: 161
  binding polypeptide

SEQ ID NO: 163 - protein, 94 aa, Artificial Sequence
  binding polypeptide
VSDVPRDLEVVAATPTSLLISWFHPHFPTHYYRITYGETGGNSPVQEFTVPLQPPAATISGLKPGVDYTITGYAVTVLKLTLELFTPISINYRT

SEQ ID NO: 164 - protein, 94 aa, Artificial Sequence
  binding polypeptide
VSDVPRDLEVVAATPTSLLISWLHPHFPTHYYRITYGETGGNSPVQEFTVPLQPPAATISGLKPGVDYTITGYAVTALMASGELFTPISINYRT

SEQ ID NO: 165 - protein, 94 aa, Artificial Sequence
  binding polypeptide
VSDVPRDLEVVAATPTSLLISWLHPHFPTHYYRITYGETGGNSPVQEFTVPLQPPAATISGLKPGVDYTITGYAVTSMKNRLELFTPISINYRT

SEQ ID NO: 166 - protein, 94 aa, Artificial Sequence
  binding polypeptide
VSDVPRDLEVVAATPTSLLISWFHPHFPTHYYRITYGETGGNSPVQEFTVPLQPPAATISGLKPGVDYTITGYAVTLRCLIPELFTPISINYRT

SEQ ID NO: 167 - protein, 94 aa, Artificial Sequence
  binding polypeptide
VSDVPRDLEVVAATPTSLLISWLHPHFPTHYYRITYGETGGNSPVQEFTVPLQPPAATISGLKPGVDYTITGYAVTVSRQKAELFTPISINYRT

SEQ ID NO: 168 - protein, 94 aa, Artificial Sequence
  binding polypeptide
VSDVPRDLEVVAATPTSLLISWRHPHFPTHYYRITYGETGGNSPVQEFTVPLQPPAATISGLKPGVDYTITGYAVTWSRTGRELFTPISINYRT

SEQ ID NO: 169 - protein, 94 aa, Artificial Sequence
  binding polypeptide
VSDVPRDLEVVAATPTSLLISWRHPHFPTHYYRITYGETGGNSPVQEFTVPLQPPAATISGLKPGVDYTITGYAVTVWRTGRELFTPISINYRT

SEQ ID NO: 170 - protein, 94 aa, Artificial Sequence
  binding polypeptide
VSDVPRDLEVVAATPTSLLISWRHPHFPTHYYRITYGETGGNSPVQEFTVPLQPPAATISGLKPGVDYTITGYAVTTERTGRELFTPISINYRT

SEQ ID NO: 171 - protein, 94 aa, Artificial Sequence
  binding polypeptide
VSDVPRDLEVVAATPTSLLISWRHPHFPTHYYRITYGETGGNSPVQEFTVPLQPPAATISGLKPGVDYTITGYAVTKERSGRELFTPISINYRT

SEQ ID NO: 172 - protein, 94 aa, Artificial Sequence
  binding polypeptide
VSDVPRDLEVVAATPTSLLISWRHPHFPTHYYRITYGETGGNSPVQEFTVPLQPPAATISGLKPGVDYTITGYAVTLERNDRELFTPISINYRT

SEQ ID NO: 173 - protein, 94 aa, Artificial Sequence
  binding polypeptide
VSDVPRDLEVVAATPTSLLISWRHPHFPTHYYRITYGETGGNSPVQEFTVPLQPPAATISGLKPGVDYTITGYAVTLERDGRELFTPISINYRT

SEQ ID NO: 174 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTQGRHKRELFTPISINYRT

SEQ ID NO: 175 - protein, 94 aa, Artificial Sequence
  binding polypeptide
VSDVPRDLEVVAATPTSLLISWRHPHFPTRYYRITYGETGGNSPVQEFTVPLQPPLATISGLKPGVDYTITGYAVTKERNGRELFTPISINYRT

SEQ ID NO: 176 - protein, 94 aa, Artificial Sequence
  binding polypeptide
VSDVPRDLEVVAATPTSLLISWRHPHFPTRYYRITYGETGGNSPVQEFTVPLQPPIATISGLKPGVDYTITGYAVTMAQNDHELITPISINYRT

SEQ ID NO: 177 - protein, 94 aa, Artificial Sequence; identical to SEQ ID NO: 176
  binding polypeptide

SEQ ID NO: 178 - protein, 94 aa, Artificial Sequence
  binding polypeptide
VSDVPRDLEVVAATPTSLLISWRHPHFPTRYYRITYGETGGNSPVQEFTVPLQPTTATISGLKPGVDYTITGYAVTWERNGRELFTPISINYRT

SEQ ID NO: 179 - protein, 94 aa, Artificial Sequence
  binding polypeptide
VSDVPRDLEVVAATPTSLLISWRHPHFPTRYYRITYGETGGNSPVQEFTVPLQPTVATISGLKPGVDYTITGYAVTLERNDRELFTPISINYRT

SEQ ID NO: 180 - protein, 94 aa, Artificial Sequence
  binding polypeptide
VSDVPRDLEVVAATPTSLLISWRPPHFPTRYYRITYGETGGNSPVQEFTVPLQPTVATISGLKPGVDYTITGYAVTLERNDRELFTPISINYRT

SEQ ID NO: 181 - protein, 94 aa, Artificial Sequence
  binding polypeptide
VSDVPRDLEVVAATPTSLLISWRHPHFPTRYYRITYGETGGNSPVQEFTVPPQPPAATISGLKPGVDYTITGYAVTKERSGRELFTPISINYRT

SEQ ID NO: 182 - protein, 94 aa, Artificial Sequence
  binding polypeptide
VSDVPRDLEVVAATPTSLLISWRHPHFPTRYYRITYGETGGNSPVQEFTVPPQPPAATISGLKPGVDYTITGYAVTLERNDRELFTPISINYRT

SEQ ID NO: 183 - protein, 94 aa, Artificial Sequence
  binding polypeptide
VSDVPRDLEVVAATPTSLLISWCHPHFPTRYYRITYGETGGNSPVQEFTVPLQPPIATISGLKPGVDYTITGYAVTMAQNDHELITPISINYRT

SEQ ID NO: 184 - DNA, 285 bp, Artificial Sequence
  binding polypeptide
atgggcgaagttgttgctgcgacccccaccagcctactgatcagctggcgccacccgcacttcccgactagatattacaggatcacttacggagaaacaggaggaaatagccctgtccaggagttcactgtgcctctgcagccccccacagctaccatcagcggccttaaacctggagttgattataccatcactgtgtatgctgtcactgacggccggaacgggcgcctcctgagcatcccaatttccattaattaccgcacagaaattgacaaaccatgccag

SEQ ID NO: 185 - DNA, 264 bp, Artificial Sequence
  binding polypeptide
atgggcgaagttgttgctgcgacccccaccagcctactgatcagctggcgccacccgcacttcccgactagatattacaggatcacttacggagaaacaggaggaaatagccctgtccaggagttcactgtgcctctgcagccccccacagctaccatcagcggccttaaacctggagttgattataccatcactgtgtatgctgtcactgacggccggaacgggcgcctcctgagcatcccaatttccattaattaccgcaca

SEQ ID NO: 186 - protein, 95 aa, Artificial Sequence
  binding polypeptide
MGEVVAATPTSLLISWRHPHFPTRYYRITYGETGGNSPVQEFTVPLQPPTATISGLKPGVDYTITVYAVTDGRNGRLLSIPISINYRTEIDKPSQ

SEQ ID NO: 187 - protein, 95 aa, Artificial Sequence
  binding polypeptide
MGEVVAATPTSLLISWRHPHFPTRYYRITYGETGGNSPVQEFTVPLQPPTATISGLKPGVDYTITVYAVTDGRNGRLLSIPISINYRTEIDKPCQ

SEQ ID NO: 188 - protein, 102 aa, Artificial Sequence
  binding polypeptide
MVSDVPRDLEVVAATPTSLLISWRHPHFPTRYYRITYGETGGNSPVQEFTVPLQPPTATISGLKPGVDYTITVYAVTDGRNGRLLSIPISINYRTEIDKPSQ

SEQ ID NO: 189 - protein, 88 aa, Artificial Sequence
  binding polypeptide
MGEVVAATPTSLLISWRHPHFPTRYYRITYGETGGNSPVQEFTVPLQPPTATISGLKPGVDYTITVYAVTDGWNGRLLSIPISINYRT

SEQ ID NO: 190 - protein, 88 aa, Artificial Sequence
  binding polypeptide
MGEVVAATPTSLLISWRHPHFPTRYYRITYGETGGNSPVQEFTVPLQPPTATISGLKPGVDYTITVYAVTEGPNERSLFIPISINYRT

SEQ ID NO: 191 - protein, 95 aa, Artificial Sequence
  binding polypeptide
MVSDVPRDLEVVAATPTSLLISWRHPHFPTRYYRITYGETGGNSPVQEFTVPLQPPTATISGLKPGVDYTITVYAVTEGPNERSLFIPISINYRT

SEQ ID NO: 192 - protein, 86 aa, Artificial Sequence
  binding polypeptide
EVVAATPTSLLISWRHPHFPTRYYRITYGETGGNSPVQEFTVPLQPPTATISGLKPGVDYTITVYAVTDGRNGRLLSIPISINYRT

SEQ ID NO: 193 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITVYAVTDGRNGRLLSIPISINYRT

SEQ ID NO: 194 - protein, 94 aa, Artificial Sequence
  binding polypeptide
GEVVAATPTSLLISWRHPHFPTRYYRITYGETGGNSPVQEFTVPLQPPTATISGLKPGVDYTITVYAVTDGRNGRLLSIPISINYRTEIDKPCQ

SEQ ID NO: 195 - protein, 94 aa, Artificial Sequence
  binding polypeptide
GEVVAATPTSLLISWRHPHFPTRYYRITYGETGGNSPVQEFTVPLQPPTATISGLKPGVDYTITVYAVTDGRNGRLLSIPISINYRTEIDKPSQ

SEQ ID NO: 196 - protein, 86 aa, Artificial Sequence
  binding polypeptide
EVVAATPTSLLISWRHPHFPTRYYRITYGETGGNSPVQEFTVPLQPPTATISGLKPGVDYTITVYAVTEGPNERSLFIPISINYRT

SEQ ID NO: 197 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITVYAVTEGPNERSLFIPISINYRT

SEQ ID NO: 198 - protein, 10 aa, Artificial Sequence
  binding polypeptide
VAQNDHELIT

SEQ ID NO: 199 - protein, 109 aa, Artificial Sequence
  binding polypeptide
MGVSDVPRDLEVVAATPTSLLISWRHPHFPTRYYRITYGETGGNSPVQEFTVPLQPPLATISGLKPGVDYTITVYAVTKERNGRELFTPISINYRTEIDKPCQHHHHHH

SEQ ID NO: 200 - protein, 87 aa, Artificial Sequence
  binding polypeptide
GEVVAATPTSLLISWRHPHFPTRYYRITYGETGGNSPVQEFTVPLQPPTATISGLKPGVDYTITVYAVTDGWNGRLLSIPISINYRT

SEQ ID NO: 201 - DNA, 66 bp, Artificial Sequence
  binding polypeptide
gcgtaatacgactcactatagggacaattactatttacaattacaatggtttctgatgttccgagg

SEQ ID NO: 202 - DNA, 78 bp, Artificial Sequence
  binding polypeptide
gcgtaatacgactcactatagggacaattactatttacaattacaatggaagttgttgctgcgacccccaccagccta

SEQ ID NO: 203 - DNA, 57 bp, Artificial Sequence
  binding polypeptide
ttttttttttttttttttttaaatagcggatgccttgtcgtcgtcgtccttgtagtc

SEQ ID NO: 204 - DNA, 69 bp, Artificial Sequence
  binding polypeptide
atggtttctgatgttccgagggacctggaagttgttgctgcgacccccaccagcctactgatcagctgg

SEQ ID NO: 205 - DNA, 66 bp, Artificial Sequence
  binding polypeptide
aggcacagtgaactcctggacagggctatttcctcctgtttctccgtaagtgatcctgtaatatct

SEQ ID NO: 206 - DNA, 66 bp, Artificial Sequence
  binding polypeptide
agatattacaggatcacttacggagaaacaggaggaaatagccctgtccaggagttcactgtgcct

SEQ ID NO: 207 - DNA, 63 bp, Artificial Sequence
  binding polypeptide
agtgacagcatacacagtgatggtataatcaactccaggtttaaggccgctgatggtagctgt

SEQ ID NO: 208 - DNA, 63 bp, Artificial Sequence
  binding polypeptide
acagctaccatcagcggccttaaacctggagttgattataccatcactgtgtatgctgtcact

SEQ ID NO: 209 - DNA, 77 bp, Artificial Sequence
  binding polypeptide
tttttttttttttttttttaaatagcggatgccttgtcgtcgtcgtccttgtagtctgttcggtaattaatggaaat

SEQ ID NO: 210 - DNA, 58 bp, Artificial Sequence
  binding polypeptide
ttttaaatagcggatgccttgtcgtcgtcgtccttgtagtctgttcggtaattaatgg

SEQ ID NO: 211 - DNA, 30 bp, Artificial Sequence
  binding polypeptide
gtgtatgctgtcactatttccattaattac

SEQ ID NO: 212 - DNA, 68 bp, Artificial Sequence
  binding polypeptide
taatacgactcactatagggacaattactatttacaattctatcaatacaatggtgtctgatgtgccg

SEQ ID NO: 213 - DNA, 72 bp, Artificial Sequence
  binding polypeptide
ccaggagatcagcagggaggtcggggtggcagccaccacttccaggtcgcgcggcacatcagacaccattgt

SEQ ID NO: 214 - DNA, 64 bp, Artificial Sequence
  binding polypeptide
acctccctgctgatctcctggcgccatccgcattttccgacccgctattaccgcatcacttacg

SEQ ID NO: 215 - DNA, 63 bp, Artificial Sequence
  binding polypeptide
cacagtgaactcctggaccgggctattgcctcctgtttcgccgtaagtgatgcggtaatagcg

SEQ ID NO: 216 - DNA, 61 bp, Artificial Sequence
  binding polypeptide
cggtccaggagttcactgtgccgctgcagccgccggcggctaccatcagcggccttaaacc

SEQ ID NO: 217 - DNA, 61 bp, Artificial Sequence
  binding polypeptide
cggtccaggagttcactgtgccgnnsnnsnnsnnsnnsgctaccatcagcggccttaaacc

SEQ ID NO: 218 - DNA, 59 bp, Artificial Sequence
  binding polypeptide
agtgacagcatacacagtgatggtataatcaacgccaggtttaaggccgctgatggtag

SEQ ID NO: 219 - DNA, 87 bp, Artificial Sequence
  binding polypeptide
accatcactgtgtatgctgtcactnnsnnsnnsnnsnnsnnsgaactgtttaccccaatttccatcaactaccgcacagactacaag

SEQ ID NO: 220 - DNA, 97 bp, Artificial Sequence
  binding polypeptide
aaatagcggatgcgcgtttgttctgatcttccttatttatgtgatgatggtggtgatgcttgtcgtcgtcgtccttgtagtctgtgcggtagttgat

SEQ ID NO: 221 - DNA, 51 bp, Artificial Sequence
  binding polypeptide
ttttttttttttttttttttaaatagcggatgcgcgtttgttctgatcttc

SEQ ID NO: 222 - DNA, 21 bp, Artificial Sequence
  binding polypeptide
gcgcgtttgttctgatcttcc

SEQ ID NO: 223 - DNA, 73 bp, Artificial Sequence
  binding polypeptide
tgcctcctgtttcgccgtaagtgatgcggtaatagcgsnnsnnsnnsnnsnnsnnsnnccagctgatcagcag

SEQ ID NO: 224 - DNA, 69 bp, Artificial Sequence
  binding polypeptide
gatggtagctgtsnnsnnsnnsnnaggcacagtgaactcctggacagggctattgcctcctgtttcgcc

SEQ ID NO: 225 - DNA, 68 bp, Artificial Sequence
  binding polypeptide
gtgcggtaattaatggaaattggsnnsnnsnnsnnsnnsnnsnnsnnsnnsnnagtgacagcatacac

SEQ ID NO: 226 - DNA, 21 bp, Artificial Sequence
  binding polypeptide
cctcctgtttctccgtaagtg

SEQ ID NO: 227 - DNA, 21 bp, Artificial Sequence
  binding polypeptide
cacttacggagaaacaggagg

SEQ ID NO: 228 - DNA, 63 bp, Artificial Sequence
  binding polypeptide
acagctaccatcagcggccttaaacctggcgttgattataccatcactgtgtatgctgtcact

SEQ ID NO: 229 - DNA, 18 bp, Artificial Sequence
  binding polypeptide
agtgacagcatacacagt

SEQ ID NO: 230 - DNA, 75 bp, Artificial Sequence
  binding polypeptide
ttttttttttttttttttttaaatagcggatgccttgtcgtcgtcgtccttgtagtctgtgcggtaattaatgga

SEQ ID NO: 231 - DNA, 28 bp, Artificial Sequence
  binding polypeptide
tagagaattcatggagagcaaggtgctg

SEQ ID NO: 232 - DNA, 39 bp, Artificial Sequence
  binding polypeptide
agggagagcgtcaggatgagttccaagttcgtcttttcc

SEQ ID NO: 233 - DNA, 28 bp, Artificial Sequence
  binding polypeptide
tagagaattcatggagagcaaggcgctg

SEQ ID NO: 234 - DNA, 39 bp, Artificial Sequence
  binding polypeptide
agggagagcgtcaggatgagttccaagttggtcttttcc

SEQ ID NO: 235 - DNA, 32 bp, Artificial Sequence
  binding polypeptide
tagagaattcatgatgtcgtcctggataaggt

SEQ ID NO: 236 - DNA, 39 bp, Artificial Sequence
  binding polypeptide
agggagagcgtcaggatgagatgttcccgaccggtttta

SEQ ID NO: 237 - DNA, 39 bp, Artificial Sequence
  binding polypeptide
ggaaaagacgaacttggaactcatcctgacgctctccct

SEQ ID NO: 238 - DNA, 39 bp, Artificial Sequence
  binding polypeptide
ggaaaagaccaacttggaactcatcctgacgctctccct

SEQ ID NO: 239 - DNA, 31 bp, Artificial Sequence
  binding polypeptide
tagactcgagtcaagagcaagccacatagct

SEQ ID NO: 240 - DNA, 39 bp, Artificial Sequence
  binding polypeptide
taaaaccggtcgggaacatctcatcctgacgctctccct

SEQ ID NO: 241 - protein, 126 aa, Artificial Sequence
  binding polypeptide
EIVMTQSPGTLSLSPGERATLSCRASQSVSSNSVAWYRKKPGQAPRLLIYGASSRATGIPDRFSGSGSGTDFTLTISRLEPEDFAVYYCATIVAGDHYFDHWGQGTKVEIKADGSDYKDDDDKASA

SEQ ID NO: 242 - protein, 126 aa, Artificial Sequence
  binding polypeptide
EIVMTQSPGTLSLSPGERATLSCRASQSVSSNSVAWYRKKPGQAPRLLIYGASSRATGIPDRFSGSGSGTDFTLTISRLEPEDFAVYYCATIVAGDHYLDHWGQGTKVEIKADGSDYKDDDDKASA

SEQ ID NO: 243 - protein, 126 aa, Artificial Sequence; identical to SEQ ID NO: 242
  binding polypeptide

SEQ ID NO: 244 - protein, 126 aa, Artificial Sequence
  binding polypeptide
EIVMTQSPGTLSLSPGERATLSCRASQSVSSNSVAWYRKKPGQAPRLLIYGASSRATGIPDRFSGSGSGTDFTLTISRLEPEDFAAYYCATIVAGDHYFDHWGQGTKVEIIADGSDYKDDDDKASA

SEQ ID NO: 245 - protein, 126 aa, Artificial Sequence; identical to SEQ ID NO: 241
  binding polypeptide

SEQ ID NO: 246 - protein, 126 aa, Artificial Sequence
  binding polypeptide
EIVMTQSPGTLSLSPGERATLSCRASQSVSSNSVAWYRKKPGQAPRLLIYGASSRAAGIPDRFSGSGSGTDFTLTISRLEPEDFAVYYCATIVAGDHYFDHWGQGTKVEIKADGSDYKDDDDKASA

SEQ ID NO: 247 - protein, 126 aa, Artificial Sequence
  binding polypeptide
EIVLTQSPGTLSLSPGERATLSCRASQSVGSNSVAWYRKKPGQAPRLLIYGASSRATGIPDRFSGSGSGTDFTLTISRLEPEDFAVYYCATIVAGDHYFDHWGQGTKVEIKADGSDYKDDDDKASA

SEQ ID NO: 248 - protein, 126 aa, Artificial Sequence
  binding polypeptide
AIQMTQSPGTLSLSPGERATLSCRASQSVSSNSVAWYRKKPGQAPRLLIYGASSRATGIPDRFSGSGSGTDFTLTISRLEPEDFAVYYCATIVAGDHYFDHWGQGTKVEIKADGSDYKDDDDKASA

SEQ ID NO: 249 - protein, 126 aa, Artificial Sequence; identical to SEQ ID NO: 248
  binding polypeptide

SEQ ID NO: 250 - protein, 126 aa, Artificial Sequence; identical to SEQ ID NO: 241
  binding polypeptide

SEQ ID NO: 251 - protein, 126 aa, Artificial Sequence; identical to SEQ ID NO: 241
  binding polypeptide

SEQ ID NO: 252 - protein, 126 aa, Artificial Sequence; identical to SEQ ID NO: 241
  binding polypeptide

SEQ ID NO: 253 - protein, 126 aa, Artificial Sequence; identical to SEQ ID NO: 241
  binding polypeptide

SEQ ID NO: 254 - protein, 126 aa, Artificial Sequence
  binding polypeptide
EIVMTQSPGTLSLSPGERATLSCRTSQSVSSNSVAWYRKKPGQAPRLLIYGASSRATGIPDRFSGSGSGTDFTLTISRLEPEDFAVYYCATIVAGDHYFDHWGQGTKVEIKADGSDYKDDDDKASA

SEQ ID NO: 255 - protein, 126 aa, Artificial Sequence
  binding polypeptide
EIVMTQSPDTLSLSPGERATLSCRASQSVSSNSVAWYRKKPGQAPRLLIYGASSRATGIPDRFSGSGSGTDFTLTISRLEPEDFAVYYCATIVAGDHYFDHWGQGTKVEIKADGSDYKDDDDKASA

SEQ ID NO: 256 - protein, 126 aa, Artificial Sequence
  binding polypeptide
EIVMTQSPATLSVSPGGRATLSCRASQSVSSNSVAWYRKKPGQAPRLLIYGASSRATGIPDRFSGSGSGTDFTLTISRLEPEDFAVYYCATIVAGDHYFDHWGQGTKVEIKADGSDYKDDDDKASA

SEQ ID NO: 257 - protein, 125 aa, Artificial Sequence
  binding polypeptide
EIVMTQSPGTLSLSPGERATLSCRASQSVSSSSLAWYRQKPGQAPRLLIYGASNRATGIPDRFSGSGSGTDFTLTISSLQPEDFAVYYCAKLTYYGSGRNWGQGTKLEIKADGSDYKDDDDKASA

SEQ ID NO: 258 - protein, 125 aa, Artificial Sequence; identical to SEQ ID NO: 257
  binding polypeptide

SEQ ID NO: 259 - protein, 125 aa, Artificial Sequence; identical to SEQ ID NO: 257
  binding polypeptide

SEQ ID NO: 260 - protein, 125 aa, Artificial Sequence; identical to SEQ ID NO: 257
  binding polypeptide

SEQ ID NO: 261 - protein, 125 aa, Artificial Sequence
  binding polypeptide
EIVMTQSPGTLSLSPGERATLSCRASQSVSSSSLAWYRQKPGQAPRLLIYGASNRATGIPDRFSGSGSGTDFTLTISSLQPEDFAVYYCAKLTYYGSGRNWGQGTKLEIIADGSDYKDDDDKASA

SEQ ID NO: 262 - protein, 125 aa, Artificial Sequence
  binding polypeptide
EIVMTQSPGTLSLSPGDRATLSCRTSQSVSSSSLAWYQQKPGQAPRLLIYGASSRATGIPDRFSGSGPGTDFTLTVSRLEPEDFAVYYCAKLTYYGSGRNWGQGTKVEIKADGSDYKDDDDKASA

SEQ ID NO: 263 - protein, 121 aa, Artificial Sequence
  binding polypeptide
EIVMTQSPGTLSVSPGGRASLPCRASQTVTNNYLAWYQQKSGQAPRLLIFGASNRATGIPERFSGSGSGTDFTLIISRLEAEDFAVYYCVSLKGRDWGQGTKLEIKADGSDYKDDDDKASA

SEQ ID NO: 264 - protein, 126 aa, Artificial Sequence
  binding polypeptide
EIGMTQSPATLSVSPGKRATLSCRASQSVGIKLAGYQQKLGQAPRLLIYEASTRATGVPTRFNGSGSGTEFTLTINSLQSEDFAVYYCTGGTGNYYGWFDPWGQGTKLEIKAYGSDYEDDDDXASA

SEQ ID NO: 265 - protein, 123 aa, Artificial Sequence
  binding polypeptide
EIVLTQSPDFQSVTPKKKVTITCRASQSIVTSLHWYQQKPGQAPSLLIYGASNRATGIPARFSGCGSGTEFTLTISGLQPEDAAVYYCAGDFGGQLPYWGQGTKLEIKADGSDYKDDDDKASA

SEQ ID NO: 266 - protein, 128 aa, Artificial Sequence
  binding polypeptide
QPVLTQPASVSGSPGQSITISCTGTSSDVGGYNYVFWYQQHPGKAPKVMIYEDTERPSGVSNRFSGSKSGNTASLTISGLQAEDEAVYYCARRNYGSGSWIDYWGQGTKLTVLADGSDYKDDDDKASA

SEQ ID NO: 267 - protein, 126 aa, Artificial Sequence
  binding polypeptide
AIQMTQSPGTLSLSPGERATLSCRASQSVRTKLAWYQQKPGQSPRLLIYDASTRATGVPARFSGSGSGTEFTLTISSLQSEDFAVYYCTGGTGNYYGWFDPWGQGTKVRSKQMAATTRTTTTRHPL

SEQ ID NO: 268 - protein, 127 aa, Artificial Sequence
  binding polypeptide
QSVLTQPPSVSGAPGQRVTISCTGSSSNIGAGYDVHWYQQLPGTAPKLLIYGNSNRPSGVPDRFSGSKSGTSASLAITGLQAEDEAVYYCASAIPGTRILRDWGQGTKLTVLADGSDYRTTTTRHPL

SEQ ID NO: 269 - protein, 123 aa, Artificial Sequence
  binding polypeptide
EIVMTQSPATLSLSPGERATLSCRASQSVSSNLAWYQQRPGQAPRLLIYGASIRATGIPDRFSGSESGTDFTLTISRLEPEDFAVYYCATEFRGYAGYWGQGTKLEIKADGSDYKDDDDKASA

SEQ ID NO: 270 - protein, 125 aa, Artificial Sequence
  binding polypeptide
EIVMTQSPGTLSLSPGRRATLSCRASQSVSSSSLAWYRQKPGQAPRLLIYGASNRATGIPDRFSGSGSGTDFTLTISSLQPEDFAVYYCAKLTYYGSGRNWGQGTKLEIKADGSDYKDDDDKASA

SEQ ID NO: 271 - protein, 125 aa, Artificial Sequence
  binding polypeptide
GIVLTQSPGTLSLSPGERATLSCRASQSVSSSSLAWYRQKPGQAPRLLIYGASNRATGIPDRFSGSGSGTDFTLTISSLQPEDFAVYYCAKLTYYGSGRNWGQGTKLEIKADGSDYKDDDDKASA

SEQ ID NO: 272 - protein, 125 aa, Artificial Sequence
  binding polypeptide
EIVMTQSPGTLSLSPGERATLSCRASQSISSSSLAWYRQKPGQAPRLLIYGASTRATGIPDRFSGSGSGTDFSLTISSLQPEDAAVYYCAKLTYYGSGRNWGQGTKLEIKADGSDYKDDDDKASA

SEQ ID NO: 273 - protein, 126 aa, Artificial Sequence
  binding polypeptide
EIVMTQSPGALSLSPGEGATLSCRASQSVSSNSVAWYRKKPGQAPRLLIYGASSRATGIPDRFSGSGSGTDFTLTISRLEPEDFAVYYCATIVAGDHYFDHWGQGTKVEIKEDGSDYKDDDDKASA

SEQ ID NO: 274 - protein, 126 aa, Artificial Sequence
  binding polypeptide
EIVMTQSPGTLSLSPGEGATLSCRASQSVSSNSVAWYRKKPGQAPRLLIYGASSRATGIPDRFSGSGSGTDFTLTISRLEPEDFAVYYCATIVAGDHYFDHWGQGTKVEIKADGSDYKDDDDKASA

SEQ ID NO: 275 - protein, 126 aa, Artificial Sequence
  binding polypeptide
EIVMTQSPGTLSLSPGERATLSCRASQSVSSNSVAWYRKKPGLAPRLLIYGASSRATGIPDRFSGSGSGTDFTLTISRLEPEDFAVYYCATIVAGDHYFDHWGQGTKVEIKADGSDYKDDDDKASA

SEQ ID NO: 276 - protein, 126 aa, Artificial Sequence; identical to SEQ ID NO: 241
  binding polypeptide

SEQ ID NO: 277 - protein, 126 aa, Artificial Sequence
  binding polypeptide
EIVMTQSPGTLSLSPGERATLSCRASQSVSSNSVAWYRKKPGQAPRLLIYGASSRATSIPDRFSGSGSGTDFTLTISRLEPEDFAVYYCATIVAGDHYFDHWGQGTKVEIIADGSDYKDDDDKASA

SEQ ID NO: 278 - protein, 126 aa, Artificial Sequence
  binding polypeptide
AIQMTQSPGTLSLSPGGRATLSCRASQSVSSNSVAWYRKKPGQAPRLLIYGASSRATGIPDRFSGSGSGTDFTLTISRLEPEDFAVYYCATIVAGDHYFDHWGQGTKVEIKADGSDYKDDDDKASA

SEQ ID NO: 279 - protein, 126 aa, Artificial Sequence
  binding polypeptide
KIVMTQSPGTLSLSPGERATLSCRASQSVSSNSVAWYRKKPGQAPRLLIYGASSRATGIPDRFSGSGSGTDFTLTISRLEPEDFAVYYCATIVAGDHYFDHWGQGTKVEIKADGSDYKDDDDKASA

SEQ ID NO: 280 - protein, 126 aa, Artificial Sequence
  binding polypeptide
DIVMTQSPGTLSLSPGERATLSCRASQSVSSNSVAWYRKKPGQAPRLLIYGASSRATGIPDRFSGSGSGTDFTLTISRLEPEDFAVYYCATIVAGDHYFDHWGQGTKVEIKADGSDYKDDDDKASA

SEQ ID NO: 281 - protein, 126 aa, Artificial Sequence; identical to SEQ ID NO: 241
  binding polypeptide

SEQ ID NO: 282 - protein, 126 aa, Artificial Sequence; identical to SEQ ID NO: 241
  binding polypeptide

SEQ ID NO: 283 - protein, 126 aa, Artificial Sequence; identical to SEQ ID NO: 241
  binding polypeptide

SEQ ID NO: 284 - protein, 126 aa, Artificial Sequence
  binding polypeptide
EIVMTQSPATLSLSPGERATLSCRASQSVSSNSVAWYRKKPGQAPRLLIYGASSRATGIPDRFSGSGSGTDFTLTISRLEPEDFAVYYCATIVAGDHYFDHWGQGTKVEIKADGSDYKDDDDKASA

SEQ ID NO: 285 - protein, 126 aa, Artificial Sequence; identical to SEQ ID NO: 284
  binding polypeptide

SEQ ID NO: 286 - protein, 125 aa, Artificial Sequence; identical to SEQ ID NO: 257
  binding polypeptide

SEQ ID NO: 287 - protein, 125 aa, Artificial Sequence; identical to SEQ ID NO: 257
  binding polypeptide

SEQ ID NO: 288 - protein, 125 aa, Artificial Sequence; identical to SEQ ID NO: 257
  binding polypeptide

SEQ ID NO: 289 - protein, 125 aa, Artificial Sequence
  binding polypeptide
EIVMTQSPGTLSLSPGERATLSCRASQSVSSSSLAWYRQKPGQAPRLLIYGASNRATGIPDRFSGSGSGTDFTLTISSLQPEDFAVYYCAKLTYYGSGRNWGQGTKLEIKEDGSDYKDDDDKASA

SEQ ID NO: 290 - protein, 125 aa, Artificial Sequence; identical to SEQ ID NO: 257
  binding polypeptide

SEQ ID NO: 291 - protein, 125 aa, Artificial Sequence; identical to SEQ ID NO: 257
  binding polypeptide

SEQ ID NO: 292 - protein, 125 aa, Artificial Sequence
  binding polypeptide
EIVLTQSPGTLSLSPGERATLSCRASQSVSSSSLAWYRQKPGQAPRLLIYGASNRATGIPDRFSGSGSGTDFTLTISSLQPEDFAVYYCAKLTXYGSGRNWGQGTKLEIKADGSDYKDDDDKASA

SEQ ID NO: 293 - protein, 125 aa, Artificial Sequence
  binding polypeptide
EIVMTQSPGTLSLSPGERATLSCRASQSVSSSSLAWYQQKPGQAPRLLIYGASSRATGIPDRFSGSGSGTDFTLTISRLEPEDFAVYYCAKLTYYGSGRNWGQGTKLEIKADGSDYKDDDDKASA

SEQ ID NO: 294 - protein, 126 aa, Artificial Sequence
  binding polypeptide
EIVMTQSPATLSVSPGERATLSCRASQSVGIKLAWYQQKLGQAPRLLIYDASTRATGVPARFSGSGSGTEFTLTISSLQSEDFAVYYCTGGTGNYYGWFDPWGQGTKLEIKADGSDYKDDDDKASA

SEQ ID NO: 295 - protein, 126 aa, Artificial Sequence
  binding polypeptide
AIQMTQSPGTLSLSPGERATLSCRASQSVRTKLAWYQQKPGQSPRLLIYDASTRATGVPARFSGSGSGTEFTLTISSLQPEDFAVYYCTGGTGNYYGWFDPWGQGTKVEIKADGSDYKDDDDKASA

SEQ ID NO: 296 - protein, 125 aa, Artificial Sequence
  binding polypeptide
AIQMTQSPSSLSASVGDRVTITCRASQSISTYLNWYQLKPGEAPKRLIYAASNLESGVPSRFSGSGSGTEFTLTINSLQPEDFAVYYCAGSFIARGPLNYWGQGTKVEIKADGSDYKDDDDKASA

SEQ ID NO: 297 - protein, 125 aa, Artificial Sequence; identical to SEQ ID NO: 296
  binding polypeptide

SEQ ID NO: 298 - protein, 128 aa, Artificial Sequence
  binding polypeptide
SYELTQPASVSGSPGQSITISCTGTSSDVGGYDHVFWYQQHPGKAPKLMIYEDSKRPSGVSNRFSGSKSGNTASLTISGLQDEDEAVYYCAKRRYVNAGWFDYWGQGTKLTVLADGSDYKDDDDKASA

SEQ ID NO: 299 - protein, 128 aa, Artificial Sequence
  binding polypeptide
QSALTQPASVSGSPGQSITISCTGTSSDVGGYDHVFWYQQHPGKAPKLMIYEDSKRPSGVSNRFSGSKSGNTASLTISGLQAEDEAVYYCAKRRYVNAGWFDYWGQGTKLTVLADGSDYKDDDDKASA

SEQ ID NO: 300 - protein, 128 aa, Artificial Sequence
  binding polypeptide
QSVLTQPRSVSGSPGQSVAISCTGTSSDVGGYNYVFWYQRHPGKAPKLIIYEDNKRPSGVPDRFSGSKFGNTASLTISGLHTEDEAVYYCAKRRYVNAGWFDYWGQGTKVTVLADGSDYKDDDDKASA

SEQ ID NO: 301 - protein, 128 aa, Artificial Sequence; identical to SEQ ID NO: 300
  binding polypeptide

SEQ ID NO: 302 - protein, 128 aa, Artificial Sequence; identical to SEQ ID NO: 300
  binding polypeptide

SEQ ID NO: 303 - protein, 128 aa, Artificial Sequence; identical to SEQ ID NO: 300
  binding polypeptide

SEQ ID NO: 304 - protein, 128 aa, Artificial Sequence
  binding polypeptide
SYELTQPASVSGSPGQSVAISCTGTSSDVGGYNYVFWYQRHPGKAPKLIIYEDNKRPSGVPDRFSGSKFGNTASLTISGLHTEDEAVYYCAKRRYVNAGWFDYWGQGTKVTVLADGSDYKDDDDKASA

SEQ ID NO: 305 - protein, 127 aa, Artificial Sequence
  binding polypeptide
QSVLTQPPSVSGAPGQRVTISCTGSSSNIGAGYDVHWYQQLPGAAPKLLIYGNSNRPSGVPDRFSGSKSGTSASLAITGLQAEDEAVYYCASAIPGTRILRDWGQGTKLTVLADGSDYKDDDDKASA

SEQ ID NO: 306 - protein, 123 aa, Artificial Sequence
  binding polypeptide
EIVMTQSPATLSVSPGERAALSCRASQSVSDKLAWYQQKPGQAPRLLIYGASSRATGIPDRFSGSGSGTDFTLTISSLEPEDFAVYYCATEFRGYAGYWGQGTKLEIKADGSDYKDDDDKASA

SEQ ID NO: 307 - protein, 123 aa, Artificial Sequence
  binding polypeptide
EIVMTQSPATLSVSPGERATLSCRASQSVSSNLAWYQQKPGQAPRLLIYGASRRATGIPDRFSGSGSGTDFTLTISRLEPEDFAVYYCAGFDPYQPPRWGQGTKLEIKADGSDYKDDDDKASA

SEQ ID NO: 308 - protein, 123 aa, Artificial Sequence
  binding polypeptide
EIVLTQSPDFQSVTPKEKVTITCRASQSIVTSLHWYQQKPGQAPSLLIYGASNRATGIPARFSGSGSGTEFTLTISGLQPEDAAVYYCAGDFGGQLPYWGQGTKLEIKADGSDYKDDDDKASA

SEQ ID NO: 309 - protein, 129 aa, Artificial Sequence
  binding polypeptide
DIVMTQSPLSLPVTLGQPASISCRSSQSLLYSDGNTYLNWFQQRPGQSPQLLIYLGSNRASGVPDRFSGSGSGTDFTLKISRVEAEDVAVYYCARAGSSWKFDYWGQGTKVEIKADGSDYKDDDDKASA

SEQ ID NO: 310 - protein, 129 aa, Artificial Sequence
  binding polypeptide
DIVMTQSPLSLPVTLGQPASISCRSSQSLLLTSGDNYLNWYLQKPGQSPQLLIYLGSNRASGVPDRFSGSGSGTDFTLKISRVEAEDVGVYYCARMGAVAGYDYWGQGTKVEIKADGSDYKDDDDKASA

SEQ ID NO: 311 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVEQDGHVLYIPISINYRT

SEQ ID NO: 312 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMGKNGHELLTPISINYRT

SEQ ID NO: 313 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTPGPGDRELITPISINYRT

SEQ ID NO: 314 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTAGPGAHELLTPISINYRT

SEQ ID NO: 315 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMAQNNRELLTPISINYRT

SEQ ID NO: 316 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMAQYGRELLTPISINYRT

SEQ ID NO: 317 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAHNGNELLTPISINYRT

SEQ ID NO: 318 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAWNGHELMTPISINYRT

SEQ ID NO: 319 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGLRDRELFVPISINYRT

SEQ ID NO: 320 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTSGLNDRVLFIPISINYRT

SEQ ID NO: 321 - protein, 86 aa, Artificial Sequence; identical to SEQ ID NO: 87
  binding polypeptide

SEQ ID NO: 322 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGHNDRELLTPISINYRT

SEQ ID NO: 323 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGLNDRELMTPISINYRT

SEQ ID NO: 324 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMAQNGHKLMTPISINYRT

SEQ ID NO: 325 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVHWNGHELMTPISINYRT

SEQ ID NO: 326 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMGFMAHELMVPISINYRT

SEQ ID NO: 327 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTAGLNEHELLIPISINYRT

SEQ ID NO: 328 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLADNARELLTPISINYRT

SEQ ID NO: 329 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGKDVRELLTPISINYRT

SEQ ID NO: 330 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLSDSGHALFTPISINYRT

SEQ ID NO: 331 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGPYEHELLTPISINYRT

SEQ ID NO: 332 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTAGRHDHELIIPISINYRT

SEQ ID NO: 333 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTIGPNNHELLTPISINYRT

SEQ ID NO: 334 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVEQNGRELIIPISINYRT

SEQ ID NO: 335 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTAGLDEHELLIPISINYRT

SEQ ID NO: 336 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAPNGHELFTPISINYRT

SEQ ID NO: 337 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMAQNGHALFTPISINYRT

SEQ ID NO: 338 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGYNNRELLTPISINYRT

SEQ ID NO: 339 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQDGHFLYTPISINYRT

SEQ ID NO: 340 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTSGHNGHEVMTPISINYRT

SEQ ID NO: 341 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTFDQSDHELLTPISINYRT

SEQ ID NO: 342 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGPNERMLMTPISINYRT

SEQ ID NO: 343 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTGYYNDRELLTPISINYRT

SEQ ID NO: 344 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLTHNDHELLTPISINYRT

SEQ ID NO: 345 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGRNDRELLTPISINYRT

SEQ ID NO: 346 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTWAQNGRELLTPISINYRT

SEQ ID NO: 347 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGKNDHELLTPISINYRT

SEQ ID NO: 348 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGPNDHELMTPISINYRT

SEQ ID NO: 349 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTTGWNGNELFTPISINYRT

SEQ ID NO: 350 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAHNDHELLTPISINYRT

SEQ ID NO: 351 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLEQNDRVLLTPISINYRT

SEQ ID NO: 352 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTTGHHDHELIIPISINYRT

SEQ ID NO: 353 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAHENRELLTPISINYRT

SEQ ID NO: 354 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGLNDHELITPISINYRT

SEQ ID NO: 355 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTAGPNDHQLFTPISINYRT

SEQ ID NO: 356 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDAMYGRELMTPISINYRT

SEQ ID NO: 357 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAWDDHELLTPISINYRT

SEQ ID NO: 358 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMGQNDKELITPISINYRT

SEQ ID NO: 359 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNGHELYTPISINYRT

SEQ ID NO: 360 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTPGHNDHELMVPISINYRT

SEQ ID NO: 361 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEARNGRELLTPISINYRT

SEQ ID NO: 362 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAHNDRELLTPISINYRT

SEQ ID NO: 363 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMAHNDHELLTPISINYRT

SEQ ID NO: 364 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGQNDRQLLVPISINYRT

SEQ ID NO: 365 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTAGGNGHELLTPISINYRT

SEQ ID NO: 366 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTHGPYDQVLLTPISINYRT

SEQ ID NO: 367 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTIEQSGLQLMTPISINYRT

SEQ ID NO: 368 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNDRELLTPISINYRT

SEQ ID NO: 369 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAWDGRELFTPISINYRT

SEQ ID NO: 370 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAYNGREIITPISINYRT

SEQ ID NO: 371 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTWSQNNRELFTPISINYRT

SEQ ID NO: 372 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTETWNDHEIRTPISINYRT

SEQ ID NO: 373 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNGHQLFTPISINYRT

SEQ ID NO: 374 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTVTHNGHPLMTPISINYRT

SEQ ID NO: 375 - protein, 107 aa, Artificial Sequence
  binding polypeptide
EVVAATPTSLLISWRHPHFPTRYYRITYGETGGNSPVQEFTVPYYRITYGETGGNSPVQEFTVPLQPPTATISGLKPGVDYTITGYAVTFAQNDHQLFTPISINYRT

SEQ ID NO: 376 - protein, 107 aa, Artificial Sequence
  binding polypeptide
EVVAATPTSLLISWRHPHFPTRYYRITYGETGGNSPVQEFTVPYYRITYGETGGNSPVQEFTVPLQPPTATISGLKPGVDYTITGYAVTGGQMNRVLMTPISINYRT

SEQ ID NO: 377 - protein, 107 aa, Artificial Sequence
  binding polypeptide
EVVAATPTSLLISWRHPHFPTRYYRITYGETGGNSPVQEFTVPYYRITYGETGGNSPVQEFTVPLQPPTATISGLKPGVDYTITGYAVTLVHNDRELLTPISINYRT

SEQ ID NO: 378 - protein, 107 aa, Artificial Sequence
  binding polypeptide
EVVAATPTSLLISWRHPHFPTRYYRITYGETGGNSPVQEFTVPYYRITYGETGGNSPVQEFTVPLQPPTATISGLKPGVDYTITGYAVTVAQNGHELFTPISINYRT

SEQ ID NO: 379 - protein, 115 aa, Artificial Sequence
  binding polypeptide
VSDVPRDLEVVAATPTSLLISWRHPHFPTRYYRITYGETGGNSPVQEFTVPYYRITYGETGGNSPVQEFTVPLQPPTATISGLKPGVDYTITGYAVTVHWNGHELMTPISINYRT

SEQ ID NO: 380 - protein, 107 aa, Artificial Sequence
  binding polypeptide
EVVAATPTSLLISWRHPHFPTRYYRITYGETGGNSPVQEFTVPYYRITYGETGGNSPVQEFTVPLQPPTATISGLKPGVDYTITGYAVTLGWNDHELYIPISINYRT

SEQ ID NO: 381 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTAGHKDQELLTPISINYRT

SEQ ID NO: 382 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNNHELLTPISINYRT

SEQ ID NO: 383 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAWNDHEIYTPISINYRT

SEQ ID NO: 384 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQTGRELLTPISINYRT

SEQ ID NO: 385 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGWSGHELFVPISINYRT

SEQ ID NO: 386 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGHNDRELITPISINYRT

SEQ ID NO: 387 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTWNQNGQELFTPISINYRT

SEQ ID NO: 388 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTFGQNGHALLTPISINYRT

SEQ ID NO: 389 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTRGLNDGELLTPISINYRT

SEQ ID NO: 390 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTFGPSDHVLLTPISINYRT

SEQ ID NO: 391 - protein, 86 aa, Artificial Sequence; identical to SEQ ID NO: 382
  binding polypeptide

SEQ ID NO: 392 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNDHKLFIPISINYRT

SEQ ID NO: 393 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTRDQYEHELLTPISINYRT

SEQ ID NO: 394 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLALNGHELFTPISINYRT

SEQ ID NO: 395 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVESNGHALFVPISINYRT

SEQ ID NO: 396 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGQNNHELLTPISINYRT

SEQ ID NO: 397 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTWDQNGHVLLTPISINYRT

SEQ ID NO: 398 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEGLNDHELIIPISINYRT

SEQ ID NO: 399 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEGLNDHELMIPISINYRT

SEQ ID NO: 400 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEGQNDQLLFIPISINYRT

SEQ ID NO: 401 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNGHELLTPISINYRT

SEQ ID NO: 402 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNDRELLTPISINYRT

SEQ ID NO: 403 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNGHELFTPISINYRT

SEQ ID NO: 404 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNERELFTPISINYRT

SEQ ID NO: 405 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAWNDHMLMTPISINYRT

SEQ ID NO: 406 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGPNDRELMTPISINYRT

SEQ ID NO: 407 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTVGPNERMLMTPISINYRT

SEQ ID NO: 408 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAHNDHELLTPISINYRT

SEQ ID NO: 409 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAKNDHELLTPISINYRT

SEQ ID NO: 410 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTWAQNDHELLTPISINYRT

SEQ ID NO: 411 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTFAQNDHELLTPISINYRT

SEQ ID NO: 412 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMEQNGHELFTPISINYRT

SEQ ID NO: 413 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTDAPNGRELMVPISINYRT

SEQ ID NO: 414 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTGGRNGHTLLTPISINYRT

SEQ ID NO: 415 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLSQTDHELLTPISINYRT

SEQ ID NO: 416 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGQNEHELLTPISINYRT

SEQ ID NO: 417 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNGHELKTPISINYRT

SEQ ID NO: 418 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNDRELFTPISINYRT

SEQ ID NO: 419 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGQNHHELFTPISINYRT

SEQ ID NO: 420 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGPHDRELLTPISINYRT

SEQ ID NO: 421 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTMGFMAHELMVPISINYRT

SEQ ID NO: 422 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNDHELLTPISINYRT

SEQ ID NO: 423 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLVRNDHELLTPISINYRT

SEQ ID NO: 424 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQDDHELLTPISINYRT

SEQ ID NO: 425 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEDIRVLWLNTTISINYRT

SEQ ID NO: 426 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVTQNDHELLTPISINYRT

SEQ ID NO: 427 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGQNDHELLTPISINYRT

SEQ ID NO: 428 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMAQNDHKLFTPISINYRT

SEQ ID NO: 429 - protein, 86 aa, Artificial Sequence; identical to SEQ ID NO: 422
  binding polypeptide

SEQ ID NO: 430 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMAQNDHELLTPISINYRT

SEQ ID NO: 431 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNNHELLTPISINYRT

SEQ ID NO: 432 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNDRELITPISINYRT

SEQ ID NO: 433 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGQNNHELITPISINYRT

SEQ ID NO: 434 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNGHELITPISINYRT

SEQ ID NO: 435 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTTAHNGHELLTPISINYRT

SEQ ID NO: 436 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGYHDHALFTPISINYRT

SEQ ID NO: 437 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTWAWNDHELMTPISINYRT

SEQ ID NO: 438 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNDHELLTPISINYRT

SEQ ID NO: 439 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMAQNDHELMTPISINYRT

SEQ ID NO: 440 - protein, 86 aa, Artificial Sequence; identical to SEQ ID NO: 430
  binding polypeptide

SEQ ID NO: 441 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNGHELITPISINYRT

SEQ ID NO: 442 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTEGWIDHEIMIPISINYRT

SEQ ID NO: 443 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEGQNGSELIVPISINYRT

SEQ ID NO: 444 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMAQNDRELITPISINYRT

SEQ ID NO: 445 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGQNDHELFTPISINYRT

SEQ ID NO: 446 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQSDHELFTPISINYRT

SEQ ID NO: 447 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVDRNDHELFTPISINYRT

SEQ ID NO: 448 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNDHELMTPISINYRT

SEQ ID NO: 449 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNDHELFTPISINYRT

SEQ ID NO: 450 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGENDRKLITPISINYRT

SEQ ID NO: 451 - protein, 86 aa, Artificial Sequence; identical to SEQ ID NO: 438
  binding polypeptide

SEQ ID NO: 452 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEGPNGHELITPISINYRT

SEQ ID NO: 453 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMAQNVRELLTPISINYRT

SEQ ID NO: 454 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVTQNGHELITPISINYRT

SEQ ID NO: 455 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVTQNDHELFTPISINYRT

SEQ ID NO: 456 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNGHELLTPISINYRT

SEQ ID NO: 457 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNDRQLFTPISINYRT

SEQ ID NO: 458 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGPNDRELIVPISINYRT

SEQ ID NO: 459 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNEHELLTPISINYRT

SEQ ID NO: 460 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNNHELITPISINYRT

SEQ ID NO: 461 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTEAHHGHELMIPISINYRT

SEQ ID NO: 462 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTGDHNDRELMTPISINYRT

SEQ ID NO: 463 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTGGQMNRVLMTPISINYRT

SEQ ID NO: 464 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAHNDRELITPISINYRT

SEQ ID NO: 465 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVPQNGHELITMISINYRT

SEQ ID NO: 466 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNDHELFTPISINYRT

SEQ ID NO: 467 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVDQNDHELFTPISINYRT

SEQ ID NO: 468 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTVAWNDHMLMTPISINYRT

SEQ ID NO: 469 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTSGHNDHMLMIPISINYRT

SEQ ID NO: 470 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNGHVLITPISINYRT

SEQ ID NO: 471 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVTHNDHELLTPISINYRT

SEQ ID NO: 472 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGQNDHELMTPISINYRT

SEQ ID NO: 473 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNDHEIMTPISINYRT

SEQ ID NO: 474 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLAQNDHELITPISINYRT

SEQ ID NO: 475 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVSQQNHELLTPISINYRT

SEQ ID NO: 476 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNDHELMTPISINYRT

SEQ ID NO: 477 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAYNEHELYTPISINYRT

SEQ ID NO: 478 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQHDHELLTPISINYRT

SEQ ID NO: 479 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGQNDQELLTPISINYRT

SEQ ID NO: 480 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVARNDHELMTPISINYRT

SEQ ID NO: 481 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGPTDHELLTPISINYRT

SEQ ID NO: 482 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGLTDHVLLTPISINYRT

SEQ ID NO: 483 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQDDHELFTPISINYRT

SEQ ID NO: 484 - protein, 86 aa, Artificial Sequence; identical to SEQ ID NO: 466
  binding polypeptide

SEQ ID NO: 485 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGWNDHELITPISINYRT

SEQ ID NO: 486 - protein, 86 aa, Artificial Sequence; identical to SEQ ID NO: 449
  binding polypeptide

SEQ ID NO: 487 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGQENQELITPISINYRT

SEQ ID NO: 488 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTLAPSARELMTPISINYRT

SEQ ID NO: 489 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVVHNGHEILTPISINYRT

SEQ ID NO: 490 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMGYEDHELITPISINYRT

SEQ ID NO: 491 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTEGYQNHELSVPISINYRT

SEQ ID NO: 492 - protein, 86 aa, Artificial Sequence; identical to SEQ ID NO: 467
  binding polypeptide

SEQ ID NO: 493 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQSDHELMTPISINYRT

SEQ ID NO: 494 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGQNDHELITPISINYRT

SEQ ID NO: 495 - protein, 86 aa, Artificial Sequence; identical to SEQ ID NO: 476
  binding polypeptide

SEQ ID NO: 496 - protein, 86 aa, Artificial Sequence; identical to SEQ ID NO: 441
  binding polypeptide

SEQ ID NO: 497 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTRAQNDHELITPISINYRT

SEQ ID NO: 498 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQSNHELMTPISINYRT

SEQ ID NO: 499 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNDRELITPISINYRT

SEQ ID NO: 500 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLTHNEQYLFTPISINYRT

SEQ ID NO: 501 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTEIYNDHELMTPISINYRT

SEQ ID NO: 502 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTMAQNDHELITPISINYRT

SEQ ID NO: 503 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVSQYGHELITPISINYRT

SEQ ID NO: 504 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAKNDHELITPISINYRT

SEQ ID NO: 505 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNNHELITPISINYRT

SEQ ID NO: 506 - protein, 86 aa, Artificial Sequence; identical to SEQ ID NO: 478
  binding polypeptide

SEQ ID NO: 507 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTLSHYGKELRTPISINYRT

SEQ ID NO: 508 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNAHELMTPISINYRT

SEQ ID NO: 509 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLGQNDHELLTPISINYRT

SEQ ID NO: 510 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQNDHELKTPISINYRT

SEQ ID NO: 511 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTGEQNDYELLVPISINYRT

SEQ ID NO: 512 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTLTQHDHELLTPISINYRT

SEQ ID NO: 513 - protein, 86 aa, Artificial Sequence; identical to SEQ ID NO: 502
  binding polypeptide

SEQ ID NO: 514 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTEAPNGRELRTPISINYRT

SEQ ID NO: 515 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTVGPTDHELLTPISINYRT

SEQ ID NO: 516 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVGQYDHELITPISINYRT

SEQ ID NO: 517 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTVAQDEHELITPISINYRT

SEQ ID NO: 518 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTDAQNVQAPIAQISINYRT

SEQ ID NO: 519 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTSGQNDHALLIPISINYRT

SEQ ID NO: 520 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTEDIRVLWLNTTISINYRT

SEQ ID NO: 521 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTWDQNGHVLLTPISINYRT

SEQ ID NO: 522 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTLGLRDRELFVPISINYRT

SEQ ID NO: 523 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTVEPNGHKLAIPISINYRT

SEQ ID NO: 524 - protein, 86 aa, Artificial Sequence
  binding polypeptide
EVVAATPTSLLISWRHPHFPTRYYRITYGETGGNSPVQEFTVPLQPPTATISGLKPGVDYTITGYAVTFGQNGKEFRIPISINYRT

SEQ ID NO: 525 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTSGHNGHEVMTPISINYRT

SEQ ID NO: 526 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTLGWNDHELYIPISINYRT

SEQ ID NO: 527 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTLGKDVRELLTPISINYRT

SEQ ID NO: 528 - protein, 94 aa, Artificial Sequence
  binding polypeptide
VSDVPRDLEVVAATPTSLLISWRHPHFPTRYYRITYGETGGNSPVQEFTVPLQPPTATISGLKPGVDYTITGYAVTLALFDHELLTPISINYRT

(19) United States
(12) Patent Application Publication
(10) Pub. No.: US 2008/0220049 A1
(43) Pub. Date: Sep. 11, 2008

(54) Compositions and methods for intraocular delivery of fibronectin scaffold domain proteins

(75) Inventors: Yan Chen, Lexington, MA (US); Elena Getmanova, Lexington, MA (US); Martin C. Wright, Boston, MA (US); Alan S. Harris, Andover, MA (US); Ai Ching Lim, Newton, MA (US); Jochem Gokemeijer, Arlington, MA (US); Lin Sun, West Roxbury, MA (US); Michael Wittekind, Bainbridge Island, WA (US)

Correspondence Address: ROPES & GRAY LLP, PATENT DOCKETING 39/41, ONE INTERNATIONAL PLACE, BOSTON, MA, 02110-2624, US

(73) Assignee: Adnexus, A Bristol-Myers Squibb R&D Company, Waltham, MA (US)

(21) Appl. No.: 11/894,045

(22) Filed: Aug. 17, 2007

Related U.S. Application Data
(63) Continuation-in-part of application No. 11/448,171, filed on Jun. 5, 2006.
Application No. 11/448,171 is a continuation of application No. 11/101,954, filed on Apr. 7, 2005, now abandoned.
Application No. 11/101,954 is a continuation of application No. PCT/US04/40885, filed on Dec. 6, 2004.
(60) Provisional application No. 60/527,886, filed on Dec. 5, 2003.

(51) Int. Cl. A61F 9/00; A61K 39/395; A61K 38/16; A61P 27/02; A61P 9/10

(52) U.S. Cl. 424/428; 424/133.1; 424/130.1; 514/12

(57) ABSTRACT

The present disclosure relates to novel sustained-release intraocular drug delivery systems and improvements in the treatment of retinopathies. In particular, fibronectin scaffold domain proteins that selectively inhibit VEGFR-2 are contemplated.

### RELATED APPLICATIONS

This application is a continuation-in-part of U.S. patent application Ser. No. 11/448,171, entitled “Inhibitors of Type 2 Vascular Endothelial Growth Factor Receptors,” filed Jun. 5, 2006, which is a continuation of International Application PCT/US04/40885, entitled “Inhibitors of Type 2 Vascular Endothelial Growth Factor Receptors,” filed Dec. 6, 2004 and designating the U.S., which claims the benefit of U.S. Provisional Application No. 60/527,886, entitled “Inhibitors of Vascular Endothelial Growth Factor Receptors,” filed Dec. 5, 2003. All of the teachings of the above-referenced applications are incorporated herein by reference.

### BACKGROUND OF THE INVENTION

The present disclosure relates to novel sustained-release intraocular drug delivery systems and methods for using these systems to inhibit biological activities in the eye. In particular, the systems of the invention inhibit biological activities mediated by vascular endothelial growth factors (VEGFs).

Angiogenesis is the process by which new blood vessels are formed from pre-existing capillaries or post capillary venules; it is an important component of many physiological processes including ovulation, embryonic development, wound repair, and collateral vascular generation in the myocardium. Angiogenesis is also central to a number of pathological conditions such as tumor growth and metastasis, diabetic retinopathy, and macular degeneration. In many instances, the process begins with the activation of existing vascular endothelial cells in response to a variety of cytokines and growth factors. In cancer, tumor released cytokines or angiogenic factors stimulate vascular endothelial cells by interacting with specific cell surface receptors. The activated endothelial cells secrete enzymes that degrade the basement membrane of the vessels, allowing invasion of the endothelial cells into the tumor tissue. Once situated, the endothelial cells differentiate to form new vessel offshoots of pre-existing vessels. The new blood vessels provide nutrients to the tumor, facilitating further growth, and also provide a route for metastasis.

To date, numerous angiogenic factors have been identified, including the particularly potent factor VEGF. VEGF was initially purified from the conditioned media of folliculostellate cells and from a variety of cell lines. More recently a number of structural homologs and alternatively spliced forms of VEGF have been identified. The various forms of VEGF bind as high affinity ligands to a suite of VEGF receptors (VEGFRs). VEGFRs are tyrosine kinase receptors, many of which are important regulators of angiogenesis. The VEGFR family includes 3 major subtypes: VEGFR-1, VEGFR-2 (also known as Kinase Insert Domain Receptor, “KDR”, in humans), and VEGFR-3. Among VEGF forms, VEGF-A, VEGF-C and VEGF-D are known to bind and activate VEGFR-2.

VEGF, acting through its cognate receptors, can function as an endothelial specific mitogen during angiogenesis. In addition, there is substantial evidence that VEGF and VEGFRs are up-regulated in conditions characterized by inappropriate angiogenesis, such as cancer. As a result, a great deal of research has focused on the identification of therapeutics that target and inhibit VEGF or VEGFR.

Vascular diseases of the eye comprise a major cause of blindness and have only imperfect methods of treatment. These diseases include various retinopathies and macular degeneration. Retinopathy frequently results in blindness or severely limited vision due to unorganized growth and/or damage to retinal blood vessels. There are two major types of retinopathy: diabetic retinopathy and retinopathy of prematurity. Diabetic retinopathy affects nearly 80% of all diabetics who have had diabetes for more than 15 years. Retinopathy of prematurity is thought to result from oxygen toxicity, with about 15,000 premature infants a year being diagnosed with ROP in the United States alone. Macular degeneration results from the neovascular growth of the choroid vessel underneath the macula. There are two types of macular degeneration: dry and wet. While wet macular degeneration only comprises 15% of all macular degeneration, nearly all wet macular degeneration leads to blindness. In addition, wet macular degeneration nearly always results from dry macular degeneration. Once one eye is affected by wet macular degeneration, the condition almost always affects the other eye.

Current therapeutic approaches that target or inhibit VEGF or VEGFR include antibodies, peptides, and small molecule kinase inhibitors. Of these, antibodies are the most widely used for in vivo recognition and inhibition of ligands and cellular receptors. Highly specific antibodies have been used to block receptor-ligand interaction, thereby neutralizing the biological activity of the components, and also to specifically deliver toxic agents to cells expressing the cognate receptor on its surface. Although effective, antibodies are large, complex molecules that rely on expression in recombinant mammalian cells for production. Antibodies also cause a variety of side effects that are often undesirable, including activation of complement pathways and antibody-directed cellular cytotoxicity. As a result, there remains a need for effective therapeutics that can specifically inhibit VEGF/VEGFR pathways as a treatment for disorders characterized by inappropriate angiogenesis, in particular for the treatment of retinopathies. Additionally, long-lasting treatments are in need for intraocular treatments.

### SUMMARY OF THE INVENTION

The application provides sustained-release intraocular drug delivery systems comprising: a therapeutic component comprising an antiangiogenic polypeptide component; and a polymeric component associated with the therapeutic component to permit the therapeutic component to be released into the interior of an eye of an individual at a therapeutically effective dosage for a period of time after the drug delivery system is placed in the eye. The therapeutic and polymeric components may be combined in an implant device or as a plurality of particles.

In some embodiments, the antiangiogenic polypeptide component comprises an antibody, antibody fragment, or an artificial antibody, such as a scaffold region based upon a fibronectin, as well as the humanized versions thereof. An artificial antibody may comprise fibronectin based “addressable” therapeutic binding molecules (“FATBIM”), such as CT322, C7S100 and C7C100. In some embodiments, the therapeutic component is selected from the group consisting of C7S100 and C7C100; and a polymeric component associated with the therapeutic component to permit the therapeutic component to be released into the interior of an eye of an individual at a therapeutically effective dosage for a period of time after the drug delivery system is placed in the eye.

In some embodiments, the antiangiogenic polypeptide component comprises a sequence selected from SEQ ID NOs: 6-183, 186-197, 199 and 241-310. In exemplary embodiments the sequence is selected from SEQ ID NO: 194 or 195. In some embodiments the polypeptide component comprises PEG.

The application further provides novel methods of treatment. In one aspect, a method of treating a retinopathy is provided, the method comprising administering, to a patient in need thereof, a therapeutically effective amount of a polypeptide that binds to human VEGFR-2, the polypeptide comprising between about 80 and about 150 amino acids that have a structural organization comprising: i) at least five to seven beta strands or beta-like strands distributed among at least two beta sheets, and ii) at least one loop portion connecting two strands that are beta strands or beta-like strands, which loop portion participates in binding to VEGFR-2, wherein the polypeptide binds to an extracellular domain of the human VEGFR-2 protein with a dissociation constant (K_{D}) of less than 1×10⁻⁶ M and inhibits VEGFR-2 mediated angiogenesis. The methods of treatment also provide for administering to a patient in need thereof the sustained-release intraocular drug delivery systems of the invention.

The antiangiogenic polypeptide components may comprise single domain polypeptides. A single domain polypeptide described herein will generally be a polypeptide that binds to a target, such as VEGFR-2, and where target binding activity situated within a single structural domain, as differentiated from, for example, antibodies and single chain antibodies, where antigen binding activity is generally contributed by both a heavy chain variable domain and a light chain variable domain. The disclosure also provides larger proteins that may comprise single domain polypeptides that bind to target. For example, a plurality of single domain polypeptides may be connected to create a composite molecule with increased avidity. Likewise, a single domain polypeptide may be attached (e.g., as a fusion protein) to any number of other polypeptides. In certain aspects a single domain polypeptide may comprise at least five to seven beta or beta-like strands distributed among at least two beta sheets, as exemplified by immunoglobulin and immunoglobulin-like domains. A beta-like strand is a string of amino acids that participates in the stabilization of a single domain polypeptide but does not necessarily adopt a beta strand conformation. Whether a beta-like strand participates in the stabilization of the protein may be assessed by deleting the string or altering the sequence of the string and analyzing whether protein stability is diminished. Stability may be assessed by, for example, thermal denaturation and renaturation studies. Preferably, a single domain polypeptide will include no more than two beta-like strands. A beta-like strand will not usually adopt an alpha-helical conformation but may adopt a random coil structure. In the context of an immunoglobulin domain or an immunoglobulin-like domain, a beta-like strand will most often occur at the position in the structure that would otherwise be occupied by the most N-terminal beta strand or the most C-terminal beta strand. An amino acid string which, if situated in the interior of a protein sequence would normally form a beta strand, may, when situated at a position closer to an N- or C-terminus, adopt a conformation that is not clearly a beta strand and is referred to herein as a beta-like strand.

In certain embodiments, the disclosure provides single domain polypeptides that bind to VEGFR-2. Preferably the single domain polypeptides bind to human KDR, mouse Flk-1, or both. A single domain polypeptide may comprise between about 80 and about 150 amino acids that have a structural organization comprising: at least seven beta strands or beta-like strands distributed between at least two beta sheets, and at least one loop portion connecting two beta strands or beta-like strands, which loop portion participates in binding to VEGFR-2. In other words a loop portion may link two beta strands, two beta-like strands or one beta strand and one beta-like strand. Typically, one or more of the loop portions will participate in VEGFR-2 binding, although it is possible that one or more of the beta or beta-like strand portions will also participate in VEGFR-2 binding, particularly those beta or beta-like strand portions that are situated closest to the loop portions. A single domain polypeptide may comprise a structural unit that is an immunoglobulin domain or an immunoglobulin-like domain. A single domain polypeptide may bind to any part of VEGFR-2, although polypeptides that bind to an extracellular domain of a VEGFR-2 are preferred. Binding may be assessed in terms of equilibrium constants (e.g., dissociation, K_{D}) and in terms of kinetic constants (e.g., on rate constant, kₒₙ and off rate constant, k_{off}). A single domain polypeptide will typically be selected to bind to VEGFR-2 with a K_{D} of less than 10⁻⁶M, or less than 10⁻⁷M, 5×10⁻⁸M, 10⁻⁸M or less than 10⁻⁹M. VEGFR-2 binding polypeptides may compete for binding with one, two or more members of the VEGF family, particularly VEGF-A, VEGF-C and VEGF-D and may inhibit one or more VEGFR-2-mediated biological events, such as proliferation of endothelial cells, permeabilization of blood vessels and increased motility in endothelial cells. VEGFR-2 binding polypeptides may be used for therapeutic purposes as well as for any purpose involving the detection or binding of VEGFR-2. Polypeptides for therapeutic use will generally have a K_{D} of less than 5×10⁻⁸M, less than 10⁻⁸M or less than 10⁻⁹M, although higher K_{D} values may be tolerated where the k_{off} is sufficiently low or the kₒₙ is sufficiently high. In certain embodiments, a single domain polypeptide that binds to VEGFR-2 will comprise a consensus VEGFR-2 binding sequence selected from the group consisting of: SEQ ID NO:1, SEQ ID NO:2, SEQ ID NO:3 and SEQ ID NO:4. Preferably, such sequence will be situated in a loop, particularly the FG loop.

In certain embodiments, the single domain polypeptide comprises an immunoglobulin (Ig) variable domain. The Ig variable domain may, for example, be selected from the group consisting of: a human V_{L} domain, a human V_{H} domain and a camelid V_{HH} domain. One, two, three or more loops of the Ig variable domain may participate in binding to VEGFR-2, and typically any of the loops known as CDR1, CDR2 or CDR3 will participate in VEGFR-2 binding.

In certain embodiments, the single domain polypeptide comprises an immunoglobulin-like domain. One, two, three or more loops of the immunoglobulin-like domain may participate in binding to VEGFR-2. A preferred immunoglobulin-like domain is a fibronectin type III (Fn3) domain. Such domain may comprise, in order from N-terminus to C-terminus, a beta or beta-like strand, A; a loop, AB; a beta strand, B; a loop, BC; a beta strand C; a loop CD; a beta strand D; a loop DE; a beta strand F; a loop FG; and a beta or beta-like strand G. See FIG. 22 for an example of the structural organization. Optionally, any or all of loops AB, BC, CD, DE, EF and FG may participate in VEGFR-2 binding, although preferred loops are BC, DE and FG. A preferred Fn3 domain is an Fn3 domain derived from human fibronectin, particularly the 10^{th} Fn3 domain of fibronectin, referred to as ¹⁰Fn3. It should be noted that none of VEGFR-2 binding polypeptides disclosed herein have an amino acid sequence that is identical to native ¹⁰Fn3; the sequence has been modified to obtain VEGFR-2 binding proteins, but proteins having the basic structural features of ¹⁰Fn3, and particularly those retaining recognizable sequence homology to the native ¹⁰Fn3 are nonetheless referred to herein as “¹⁰Fn3 polypeptides”. This nomenclature is similar to that found in the antibody field where, for example, a recombinant antibody V_{L} domain generated against a particular target protein may not be identical to any naturally occurring V_{L} domain but nonetheless the protein is recognizably a V_{L} protein. A ¹⁰Fn3 polypeptide may be at least 60%, 65%, 70%, 75%, 80%, 85%, or 90% identical to the human ¹⁰Fn3 domain, shown in SEQ ID NO:5. Much of the variability will generally occur in one or more of the loops. Each of the beta or beta-like strands of a ¹⁰Fn3 polypeptide may consist essentially of an amino acid sequence that is at least 80%, 85%, 90%, 95% or 100% identical to the sequence of a corresponding beta or beta-like strand of SEQ ID NO: 5, provided that such variation does not disrupt the stability of the polypeptide in physiological conditions. A ¹⁰Fn3 polypeptide may have a sequence in each of the loops AB, CD, and EF that consists essentially of an amino acid sequence that is at least 80%, 85%, 90%, 95% or 100% identical to the sequence of a corresponding loop of SEQ ID NO:5. In many instances, any or all of loops BC, DE, and FG will be poorly conserved relative to SEQ ID NO:5. For example, all of loops BC, DE, and FG may be less than 20%, 10%, or 0% identical to their corresponding loops in SEQ ID NO:5.

In certain embodiments, the disclosure provides a non-antibody polypeptide comprising a domain having an immunoglobulin-like fold that binds to VEGFR-2. The non-antibody polypeptide may have a molecular weight of less than 20 kDa, or less than 15 kDa and will generally be derived (by, for example, alteration of the amino acid sequence) from a reference, or “scaffold”, protein, such as an Fn3 scaffold. The non-antibody polypeptide may bind VEGFR-2 with a K_{D} less than 10⁻⁶M, or less than 10⁻⁷M, less than 5×10⁻⁸M, less than 10⁻⁸M or less than 10⁻⁹M. The unaltered reference protein either will not meaningfully bind to VEGFR-2 or will bind with a K_{D} of greater than 10⁻⁶M. The non-antibody polypeptide may inhibit VEGF signaling, particularly where the non-antibody polypeptide has a K_{D} of less than 5×10⁻⁸M, less than 10⁻⁸M or less than 10⁻⁹M, although higher K_{D} values may be tolerated where the k_{off} is sufficiently low (e.g., less than 5×10⁻⁴ s⁻¹). The immunoglobulin-like fold may be a ¹⁰Fn3 polypeptide.

In certain embodiments, the disclosure provides a polypeptide comprising a single domain having an immunoglobulin fold that binds to VEGFR-2. The polypeptide may have a molecular weight of less than 20 kDa, or less than 15 kDa and will generally be derived (by, for example, alteration of the amino acid sequence) from a variable domain of an immunoglobulin. The polypeptide may bind VEGFR-2 with a K_{D} less than 10⁻⁶M, or less than 10⁻⁷M, less than 5×10⁻⁸M, less than 10⁻⁸M or less than 10⁻⁹M. The polypeptide may inhibit VEGF signaling, particularly where the polypeptide has a K_{D} of less than 5×10⁻⁸M, less than 10⁻⁸M or less than 10⁻⁹M, although higher K_{D} values may be tolerated where the k_{off} is sufficiently low or where the kₒₙ is sufficiently high. In certain preferred embodiments, a single domain polypeptide having an immunoglobulin fold derived from an immunoglobulin light chain variable domain and capable of binding to VEGFR-2 may comprise an amino acid sequence selected from the group consisting of: SEQ ID NOs:241-310.

In certain preferred embodiments, the disclosure provides VEGFR-2 binding polypeptides comprising the amino acid sequence of any of SEQ ID NOs:192-194. In the case of a polypeptide comprising the amino acid sequence of SEQ ID NO:194, a PEG moiety or other moiety of interest, may be covalently bound to the cysteine at position 93. The PEG moiety may also be covalently bonded to an amine moiety in the polypeptide. The amine moiety may be, for example, a primary amine found at the N-terminus of a polypeptide or an amine group present in an amino acid, such as lysine or arginine. In certain embodiments, the PEG moiety is attached at a position on the polypeptide selected from the group consisting of: a) the N-terminus; b) between the N-terminus and the most N-terminal beta strand or beta-like strand; c) a loop positioned on a face of the polypeptide opposite the target-binding site; d) between the C-terminus and the most C-terminal beta strand or beta-like strand; and e) at the C-terminus.

In certain aspects, the disclosure provides short peptide sequences that mediate VEGFR-2 binding. Such sequences may mediate VEGFR-2 binding in an isolated form or when inserted into a particular protein structure, such as an immunoglobulin or immunoglobulin-like domain. Examples of such sequences include those disclosed as SEQ ID NOs:1-4 and other sequences that are at least 85%, 90%, or 95% identical to SEQ ID NOs:1-4 and retain VEGFR-2 binding activity. Accordingly, the disclosure provides substantially pure polypeptides comprising an amino acid sequence that is at least 85% identical to the sequence of any of SEQ ID NOs:1-4, wherein said polypeptide binds to a VEGFR-2 and competes with a VEGF species for binding to VEGFR-2. Examples of such polypeptides include a polypeptide comprising an amino acid sequence that is at least 80%, 85%, 90%, 95% or 100% identical to an amino acid sequence at least 85% identical to the sequence of any of SEQ ID NOs:6-183, 186-197, 199 and 311-528. Preferably such polypeptide will inhibit a biological activity of VEGF and may bind to VEGFR-2 with a K_{D} less than 10⁻⁶M, or less than 10⁻⁷M, less than 5×10⁻⁸M, less than 10⁻⁸M or less than 10⁻⁹M.

In certain embodiments, any of the VEGFR-2 binding polypeptides described herein may be bound to one or more additional moieties, including, for example, a moiety that also binds to VEGFR-2 (e.g., a second identical or different VEGFR-2 binding polypeptide), a moiety that binds to a different target (e.g., to create a dual-specificity binding agent), a labeling moiety, a moiety that facilitates protein purification or a moiety that provides improved pharmacokinetics. Improved pharmacokinetics may be assessed according to the perceived therapeutic need. Often it is desirable to increase bioavailability and/or increase the time between doses, possibly by increasing the time that a protein remains available in the serum after dosing. In some instances, it is desirable to improve the continuity of the serum concentration of the protein over time (e.g., decrease the difference in serum concentration of the protein shortly after administration and shortly before the next administration). Moieties that tend to slow clearance of a protein from the blood include polyethylene glycol, sugars (e.g. sialic acid), and well-tolerated protein moieties (e.g., Fc fragment or serum albumin). The single domain polypeptide may be attached to a moiety that reduces the clearance rate of the polypeptide in a mammal (e.g., mouse, rat, or human) by greater than three-fold relative to the unmodified polypeptide. Other measures of improved pharmacokinetics may include serum half-life, which is often divided into an alpha phase and a beta phase. Either or both phases may be improved significantly by addition of an appropriate moiety. Where polyethylene glycol is employed, one or more PEG molecules may be attached at different positions in the protein, and such attachment may be achieved by reaction with amines, thiols or other suitable reactive groups. Pegylation may be achieved by site-directed pegylation, wherein a suitable reactive group is introduced into the protein to create a site where pegylation preferentially occurs. In a preferred embodiment, the protein is modified so as to have a cysteine residue at a desired position, permitting site directed pegylation on the cysteine. PEG may vary widely in molecular weight and may be branched or linear. Notably, the present disclosure establishes that pegylation is compatible with target binding activity of ¹⁰Fn3 polypeptides and, further, that pegylation does improve the pharmacokinetics of such polypeptides. Accordingly, in one embodiment, the disclosure provides pegylated forms of ¹⁰Fn3 polypeptides, regardless of the target that can be bound by such polypeptides.

In certain embodiments, the disclosure provides a formulation comprising any of the VEGFR-2 binding polypeptides disclosed herein. A formulation may be a therapeutic formulation comprising a VEGFR-2 binding polypeptide and a pharmaceutically acceptable carrier. A formulation may also be a combination formulation, comprising an additional active agent, such as an anti-cancer agent or an anti-angiogenic agent.

In certain aspects, the disclosure provides methods for using a VEGFR-2 binding protein to inhibit a VEGF biological activity in a cell or to inhibit a biological activity mediated by VEGFR-2. The cell may be situated in vivo or ex vivo, and may be, for example, a cell of a living organism, a cultured cell or a cell in a tissue sample. The method may comprise contacting said cell with any of the VEGFR-2-inhibiting polypeptides disclosed herein, in an amount and for a time sufficient to inhibit such biological activity.

In certain aspects, the disclosure provides methods for treating a subject having a condition which responds to the inhibition of VEGF or VEGFR-2. Such a method may comprise administering to said subject an effective amount of any of the VEGFR-2 inhibiting polypeptides described herein. A condition may be one that is characterized by inappropriate angiogenesis. A condition may be a hyperproliferative condition. Examples of conditions (or disorders) suitable for treatment include autoimmune disorders, inflammatory disorders, retinopathies (particularly proliferative retinopathies), and cancers. Any of the VEGFR-2 inhibiting polypeptides described herein may be used for the preparation of a medicament for the treatment of a disorder, particularly a disorder selected from the group consisting of: an autoimmune disorder, an inflammatory disorder, a retinopathy, and a cancer.

### BRIEF DESCRIPTION OF THE DRAWINGS

FIGS. 1A-1D are graphs and images depicting the characterization of KDR-binding single clones from Round 6 of KDR selection. FIG. 1A is a graph showing the specific binding of fibronectin-based binding proteins to 25 nM of KDR-Fc analyzed in radioactive equilibrium binding assay. FIG. 1B is a graph showing the inhibition of specific binding of KDR-Fc and selected fibronectin based binding proteins in the presence of 100-fold excess of VEGF₁₆₅. As shown in this figure, certain binding proteins bound KDR-Fc competitively with VEGF₁₆₅ while others, exemplified by clone 8, did not compete with VEGF₁₆₅. FIG. 1C is a graph showing the inhibition of KDR-Fc interaction with immobilized VEGF₁₆₅ in presence of selected fibronectin based binding proteins analyzed in BIAcore. FIG. 1D is an image showing binding of VR28 to KDR-expressing and control cells detected by immunofluorescence.

FIG. 2 is a graph showing the selection profile for the affinity maturation of VR28 KDR binder. Shown at left is binding of the VR28 clone to KDR-Fc and Flk1-Fc (very low, unlabeled bar). Shown at center is binding of a crude mutagenized pool and subsequent enrichment rounds to KDR-Fc. Shown at right is binding of further enrichment rounds to Flk-1-Fc. Binding was estimated in radioactive equilibrium binding assay as a percentage of input, using 1 nM KDR-Fc or Flk1-Fc.

FIGS. 3A and 3B are graphs depicting the characterization of KDR-binding single clones from Round 4 of anti-KDR affinity maturation of VR28 binder. FIG. 3A shows the saturation binding of VR28 (-▪-) and affinity matured K1 (-▴-), K6 (-▾-), K9 (-♦-), K10 (--), K12 [symbol] K13 (-Δ-), K14 (-∇-), K15 (-⋄-) to KDR-Fc in radioactive equilibrium binding assay FIG. 3B shows the binding of clones with and without N-terminal deletion to KDR-Fc. Deletion Δ1-8 in the N-terminus of fibronectin-based binding proteins improved binding to KDR-Fc. The data represents an average KDR-Fc binding of 23 independent clones with and without N-terminal deletion.

FIG. 4 is a graph showing the binding of the selected clones to KDR and Flk-1. Specific binding of VR28 and selected clones after four rounds of affinity maturation to human KDR (K clones) and seven rounds of affinity maturation to human (KDR) and mouse (flk-1) (E clones). VEGFR-2-Fc chimeras were compared in radioactive equilibrium binding assay. The data represents an average of 3 independent experiments. As shown here, maturation against both mouse and human VEGFR-2 proteins produces binders that bind to both proteins.

FIGS. 5A and 5B are graphs showing the characterization of VEGFR-2-binding single clones from Round 7 of affinity maturation of VR28 binder. Saturation binding of VR28 (-▪-) and specificity matured E3 (-▴-), E5 (-▾-), E6 (-♦-), E9 (--), E18 [symbol] E19 (-Δ-), E25 (-∇-), E26 (-⋄-), E28 (-◯-), E29 (-X-) clones to KDR (FIG. 5A) and Flk1 (FIG. 5B)-Fc chimeras was tested in radioactive equilibrium binding assay.

FIGS. 6A and 6B are graphs showing the characterization of VEGFR-2 binding by single clones from Round 7 of affinity maturation of the VR28 binder. FIG. 6A shows the importance of arginine at positions 79 and 82 in binders with dual specificity to human and mouse VEGFR-2 for binding to mouse VEGFR-2 (Flk1). When either of these positions was replaced by amino acid other than R (X79=E, Q, W, P; X82=L, K), binding to Flk1 but not to KDR significantly decreased. FIG. 6B shows the importance of all three variable loops (BC, DE and FG) of KDR fibronectin-based binding proteins for binding to the target in these proteins. Substitution of each loop at a time by NNS sequence affected binding to KDR and Flk1. The binding data is an average from E6 and E26 clones.

FIGS. 7A and 7B are graphs showing the binding of selected fibronectin-based binding proteins to CHO cells expressing human KDR receptor (FIG. 7A) and EpoR-Flk1 chimera (FIG. 7B). E18 (-▪-), E19 (-▴-), E26 (-▾-), E29 (-♦-) and WT [symbol] fibronectin-based scaffold proteins were tested. No binding to control CHO cells was observed (data not shown).

FIGS. 8A and 8B are graphs showing the inhibition of VEGF-induced proliferation of Ba/F3-KDR (FIG. 8A) and Ba/F3-Flk1 (FIG. 8B) cells, expressing KDR and Flk1 in the presence of different amounts of fibronectin-based binding proteins: E18 (-▪-), E19 (-▴-), E26 (-▴-), E29 (-♦-), M5 (--), WT [symbol] and anti-KDR or anti-flk-1 Ab (-Δ-). The data represents an average of 2 independent experiments.

FIG. 9 is a graph showing the results of a HUVEC proliferation assay in the presence of different amounts of fibronectin-based scaffold proteins: E18 (-▪-), E19 (-▴-), E26 (-▾-), E29 (-♦-), M5 (--), WT [symbol] The data represents an average of 2 independent experiments. As shown, the KDR binding proteins caused a decrease in proliferation by approximately 40%.

FIG. 10 is a set of graphs showing the reversible refolding of M5FL in optimized buffer.

FIG. 11 is an image showing SDS-PAGE analysis of pegylated forms of M5FL. M, molecular weight markers [Sea Blue Plus, Invitrogen]; -, M5FL alone; 20, M5FL with 20 kD PEG; 40, M5FL with 40 kD PEG.

FIG. 12 is a graph showing the inhibition of VEGF-induced proliferation of Ba/F3-KDR cells with differing amounts of M5FL (-♦-), M5FL PEG20 (-▪-) and M5FL PEG40(-▴-), respectively. Pegylation has little or no effect on M5FL activity in this assay.

FIG. 13 shows western analysis of VEGFR-2 signaling in endothelial cells. Phospho VEGFR-2—Visualization of phosphorylated VEGFR-2. VEGFR-2—Sample loading control. Phospho ERK1/2—Visualization of phosphorylated ERK1/2 (MAPK). ERK1-Sample loading control. The results demonstrated that 130 pM CT-01 blocks VEGFR-2 activation and signaling by VEGF-A.

FIG. 14 shows that various ¹⁰Fn3-derived molecules (e.g. M5FL, F10, CT-01) can block VEGF-A and VEGF-D signaling.

FIG. 15 shows a comparison of ¹²⁵I native, pegylated CT-01 administered i.v. & i.p. CT-01 is a 12 kDa protein. It is rapidly cleared from the blood. Addition of a 40 kDa PEG reduces its clearance rate and increases the AUC by 10 fold. Half life of 16 hr in rats is equivalent to 2× dosing per week in humans. Administration route: i.p. CT-01-PEG40 has an AUC that is only 50% of an i.v. administration.

FIG. 16 shows the tissue distribution of ¹²⁵I-CT01PEG40 in normal rats. Tissue distribution of ¹²⁵I-CT01PEG40 indicates secretion primarily via the liver and secondarily via the kidney. This is expected for the high molecular weight PEG form. No long term accumulation of CT-01PEG40 is detected.

FIG. 17 is a schematic view of the Miles Assay that measures vascular permeability.

FIG. 18 shows that CT-01 blocks VEGF in vivo using the Miles Assay. The results indicate that 5 mg/kg of CT01-PEG40 blocks VEGF challenge.

FIG. 19 shows that CT-01 inhibits tumor growth using the B16-F10 Murine Melanoma Tumor Assay.

FIG. 20 shows that CT-01 inhibits tumor growth using U87 Human Glioblastoma.

FIGS. 21A and 21B show the sequences of VEGFR binding polypeptides that are based on an antibody light chain framework/scaffold (SEQ ID NOs:241-310).

FIG. 22 shows the structural organization for a single domain polypeptide having an immunoglobulin fold (a V_{H} domain of an immunoglobulin, left side) and a single domain polypeptide having an immunoglobulin-like fold (a ¹⁰Fn3 domain, right side).

### DETAILED DESCRIPTION OF THE INVENTION

### Definitions

A “functional Fc region” possesses at least one “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature.

A “variant Fc region” comprises an amino acid sequence which differs from that of a native sequence Fc region by virtue of at least one amino acid modification. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The terms “Fc receptor” and “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2. The ALIGN-2 sequence comparison computer program was authored by Genentech, Inc. has been filed with user documentation in the U.S. Copyright Office, Washington D.C., 20559, where it is registered under U.S. Copyright Registration No. TXU510087, and is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

A “polypeptide chain” is a polypeptide wherein each of the domains thereof is joined to other domain(s) by peptide bond(s), as opposed to non-covalent interactions or disulfide bonds.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In preferred embodiments, the polypeptide will be purified (1) to greater than 95% by weight of polypeptide as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated polypeptide includes the polypeptide in situ within recombinant cells since at least one component of the polypeptide's natural environment will not be present. Ordinarily, however, isolated polypeptide will be prepared by at least one purification step.

Targets may also be fragments of said targets. Thus a target is also a fragment of said target, capable of eliciting an immune response. A target is also a fragment of said target, capable of binding to a single domain antibody raised against the full length target.

A fragment as used herein refers to less than 100% of the sequence (e.g., 99%, 90%, 80%, 70%, 60%, 50%, 40%, 30%, 20%, 10% etc.), but comprising 5, 6, 7, 8, 9, 10, 12, 13, 14, 15, 16, 17, 18, 19, 20, 21, 22, 23, 24, 25 or more amino acids. A fragment is of sufficient length such that the interaction of interest is maintained with affinity of 1×10⁻⁶M or better.

A fragment as used herein also refers to optional insertions, deletions and substitutions of one or more amino acids which do not substantially alter the ability of the target to bind to a single domain antibody raised against the wild-type target. The number of amino acid insertions deletions or substitutions is preferably up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

A protein of the invention that “induces cell death” is one which causes a viable cell to become nonviable. The cell is generally one which expresses the antigen to that the protein binds, especially where the cell overexpresses the antigen. Preferably, the cell is a cancer cell, e.g. a breast, ovarian, stomach, endometrial, salivary gland, lung, kidney, colon, thyroid, pancreatic or bladder cell. In vitro, the cell may be a SKBR3, BT474, Calu 3, MDA-MB453, MDA-MB-361 or SKOV3 cell. Cell death in vitro may be determined in the absence of complement and immune effector cells to distinguish cell death induced by antibody dependent cell-mediated cytotoxicity (ADCC) or complement dependent cytotoxicity (CDC). Thus, the assay for cell death may be performed using heat inactivated serum (i.e. in the absence of complement) and in the absence of immune effector cells. To determine whether the protein of the invention is able to induce cell death, loss of membrane integrity as evaluated by uptake of propidium iodide (PI), trypan blue (see Moore et al. Cytotechnology 17:1-11 (1995)) or 7AAD can be assessed relative to untreated cells.

A protein of the invention that “induces apoptosis” is one that induces programmed cell death as determined by binding of apoptosis related molecules or events, such as annexin V, fragmentation of DNA, cell shrinkage, dilation of endoplasmic reticulum, cell fragmentation, and/or formation of membrane vesicles (called apoptotic bodies). The cell is one which expresses the antigen to which the protein binds and may be one which overexpresses the antigen. The cell may be a tumor cell, e.g. a breast, ovarian, stomach, endometrial, salivary gland, lung, kidney, colon, thyroid, pancreatic or bladder cell. In vitro, the cell may be a SKBR3, BT474, Calu 3 cell, MDA-MB453, MDA-MB-361 or SKOV3 cell. Various methods are available for evaluating the cellular events associated with apoptosis. For example, phosphatidyl serine (PS) translocation can be measured by annexin binding; DNA fragmentation can be evaluated through DNA laddering as disclosed in the example herein; and nuclear/chromatin condensation along with DNA fragmentation can be evaluated by any increase in hypodiploid cells. Preferably, the protein that induces apoptosis is one which results in about 2 to 50 fold, preferably about 5 to 50 fold, and most preferably about 10 to 50 fold, induction of annexin binding relative to untreated cell in an annexin binding assay using cells expressing the antigen to which the protein of the invention binds.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the time to disease progression (TTP) and/or determining the response rates (RR).

The term “PK” is an acronym for “pharmokinetic” and encompasses properties of a compound including, by way of example, absorbtion, distribution, metabolism, and elimination by a subject. A “PK modulation protein” refers to any protein or peptide that affects the pharmokinetic properties of a biologically active molecule when fused to or administered together with the biologically active molecule. Examples of a PK modulation protein include PEG, as well as human serum albumin (HSA) binders as disclosed in US Pat. App. Nos. 20050287153 and 20070003549.

As used herein, “ocular” refers to the eye, surrounding tissues, and to bodily fluids in the region of the eye. Specifically, the term includes the cornea, the sclera, the uvea, the conjunctiva (e.g., bulbar conjunctiva, palpebral conjunctiva, and tarsal conjunctiva), anterior chamber, lacrimal sac, lacrimal canals, lacrimal ducts, medial canthus, nasolacrimal duct, and the eyelids (e.g., upper eyelid and lower eyelid). Additionally, the term includes the inner surface of the eye (conjunctiva overlying the sclera), and the inner surface of the eyelids (palpepral conjunctiva).

### Overview

The present application provides novel sustained-release intraocular drug delivery systems that are particularly useful in treating disorders of the eye. In exemplary embodiments, the drug delivery systems deliver VEGFR-2 specific inhibitors intraocularly.

In one aspect, sustained-release intraocular drug delivery system is provided comprising a therapeutic component and a polymeric component. The therapeutic component comprises an antiangiogenic polypeptide component such as an antibody, an antibody fragment, or an artificial antibody, as well as humanized versions.

### Polymeric Component

In some embodiments, the polymeric component of the sustained-release drug delivery system comprises monomers such as organic esters or ethers, which when degraded result in physiologically acceptable degradation products. Anhydrides, amides, orthoesters, or the like, by themselves or in combination with other monomers, may also be used. The polymers are generally condensation polymers. The polymers can be crosslinked or non-crosslinked. If crosslinked, they are usually not more than lightly crosslinked, and are less than 5% crosslinked, usually less than 1% crosslinked.

In addition to carbon and hydrogen, the polymers will include oxygen and nitrogen, particularly oxygen. The oxygen may be present as oxy, e.g., hydroxy or ether, carbonyl, e.g., non-oxo-carbonyl, such as carboxylic acid ester, and the like. The nitrogen can be present as amide, cyano, and amino. An exemplary list of biodegradable polymers that can be used are described in Heller, Biodegradable Polymers in Controlled Drug Delivery, In: “CRC Critical Reviews in Therapeutic Drug Carrier Systems,” Vol. 1. CRC Press, Boca Raton, Fla. (1987).

Of particular interest are polymers of hydroxyaliphatic carboxylic acids, either homo- or copolymers, and polysaccharides. Included among the polyesters of interest are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic)acid (PLGA) copolymer may be 0-100%, about 15-85%, about 25-75%, or about 35-65%. In certain variations, 25/75 PLGA and/or 50/50 PLGA copolymers are used. In other variations, PLGA copolymers are used in conjunction with polylactide polymers.

Biodegradable polymer matrices that include mixtures of hydrophilic and hydrophobic ended PLGA may also be employed, and are useful in modulating polymer matrix degradation rates. Hydrophobic ended (also referred to as capped or end-capped) PLGA has an ester linkage hydrophobic in nature at the polymer terminus. Typical hydrophobic end groups include, but are not limited to alkyl esters and aromatic esters. Hydrophilic ended (also referred to as uncapped) PLGA has an end group hydrophilic in nature at the polymer terminus. PLGA with a hydrophilic end groups at the polymer terminus degrades faster than hydrophobic ended PLGA because it takes up water and undergoes hydrolysis at a faster rate (Tracy et al., Biomaterials 20: 1057-1062 (1999)). Examples of suitable hydrophilic end groups that may be incorporated to enhance hydrolysis include, but are not limited to, carboxyl, hydroxyl, and polyethylene glycol. The specific end group will typically result from the initiator employed in the polymerization process. For example, if the initiator is water or carboxylic acid, the resulting end groups will be carboxyl and hydroxyl. Similarly, if the initiator is a monofunctional alcohol, the resulting end groups will be ester or hydroxyl.

Further polymers and polymer blends useful in the invention can be found in U.S. Patent Applications 20060210604, 20070088014, and 20070059336 hereby incorporated by reference.

### Antiangiogenic Polypeptides

In some embodiments, the antiangiogenic polypeptide component comprises one or more single domain polypeptides that may be derived from two related groups of protein structures: those proteins having an immunoglobulin fold, such as an antibody, and those proteins having an immunoglobulin-like fold, such as an artificial antibody. “Artificial antibody” is meant to include fibronectin based scaffold proteins such as the Adnectins™ or the “addressable” therapeutic binding molecules. By a “polypeptide” is meant any sequence of two or more amino acids, regardless of length, post-translation modification, or function. “Polypeptide,” “peptide,” and “protein” are used interchangeably herein. Polypeptides can include natural amino acids and non-natural amino acids such as those described in U.S. Pat. No. 6,559,126, incorporated herein by reference. Polypeptides can also be modified in any of a variety of standard chemical ways (e.g., an amino acid can be modified with a protecting group; the carboxy-terminal amino acid can be made into a terminal amide group; the amino-terminal residue can be modified with groups to, e.g., enhance lipophilicity; or the polypeptide can be chemically glycosylated or otherwise modified to increase stability or in vivo half-life). Polypeptide modifications can include the attachment of another structure such as a cyclic compound or other molecule to the polypeptide and can also include polypeptides that contain one or more amino acids in an altered configuration (i.e., R or S; or, L or D). The term “single domain polypeptide” is used to indicate that the target binding activity (e.g., VEGFR-2 binding activity) of the subject polypeptide is situated within a single structural domain, as differentiated from, for example, antibodies and single chain antibodies, where antigen binding activity is generally contributed by both a heavy chain variable domain and a light chain variable domain. It is contemplated that a plurality of single domain polypeptides of the sort disclosed herein could be connected to create a composite molecule with increased avidity. Likewise, a single domain polypeptide may be attached (e.g., as a fusion protein) to any number of other polypeptides, such as fluorescent polypeptides, targeting polypeptides and polypeptides having a distinct therapeutic effect.

Single domain polypeptides of either the immunoglobulin or immunoglobulin-like scaffold will tend to share certain structural features. For example, the polypeptide may comprise between about 80 and about 150 amino acids, which amino acids are structurally organized into a set of beta or beta-like strands, forming beta sheets, where the beta or beta-like strands are connected by intervening loop portions. The beta sheets form the stable core of the single domain polypeptides, while creating two “faces” composed of the loops that connect the beta or beta-like strands. As described herein, these loops can be varied to create customized ligand binding sites, and, with proper control, such variations can be generated without disrupting the overall stability of the protein. In antibodies, three of these loops are the well-known Complementarity Determining Regions (or “CDRs”).

Scaffolds for formation of a single domain polypeptides should be highly soluble and stable in physiological conditions. Examples of immunoglobulin scaffolds are the single domain V_{H} or V_{L} scaffold, as well as a single domain camelid V_{HH} domain (a form of variable heavy domain found in camelids) or other immunoglobulin variable domains found in nature or engineered in the laboratory. In the single domain format disclosed herein, an immunoglobulin polypeptide need not form a dimer with a second polypeptide in order to achieve binding activity. Accordingly, any such polypeptides that naturally contain a cysteine which mediates disulfide cross-linking to a second protein can be altered to eliminate the cysteine. Alternatively, the cysteine may be retained for use in conjugating additional moieties, such as PEG, to the single domain polypeptide.

Other scaffolds may be non-antibody scaffold proteins. By “non-antibody scaffold protein or domain” is meant a non-antibody polypeptide having an immunoglobulin-like fold. By “immunoglobulin-like fold” is meant a protein domain of between about 80-150 amino acid residues that includes two layers of antiparallel beta-sheets, and in which the flat, hydrophobic faces of the two beta-sheets are packed against each other. An example of such a scaffold is the “fibronectin-based scaffold protein”, by which is meant a polypeptide based on a fibronectin type III domain (Fn3). Fibronectin is a large protein which plays essential roles in the formation of extracellular matrix and cell-cell interactions; it consists of many repeats of three types (types I, II, and III) of small domains (Baron et al., 1991). Fn3 itself is the paradigm of a large subfamily which includes portions of cell adhesion molecules, cell surface hormone and cytokine receptors, chaperoning, and carbohydrate-binding domains for reviews, see Bork & Doolittle, Proc Natl Acad Sci USA. 1992 Oct. 1; 89(19):8990-4; Bork et al., J Mol Biol. 1994 Sep. 30; 242(4):309-20; Campbell & Spitzfaden, Structure. 1994 May 15; 2(5):333-7; Harpez & Chothia, J Mol Biol. 1994 May 13; 238(4):528-39).

Preferably, the fibronectin-based scaffold protein is a “¹⁰FN3” scaffold, by which is meant a polypeptide variant based on the tenth module of the human fibronectin type III protein in which one or more of the solvent accessible loops has been randomized or mutated, particularly one or more of the three loops identified as the BC loop (amino acids 23-30), DE loop (amino acids 52-56) and FG loop (amino acids 77-87) (the numbering scheme is based on the sequence on the tenth Type III domain of human fibronectin, with the amino acids Val-Ser-Asp-Val-Pro representing amino acids numbers 1-5). The amino acid sequence of the wild-type tenth module of the human fibronectin type III domain is:

VSDVPRDLEVVAATPTSLLISWDAPAVTVRYYRITYGETGGNSPVQEFTVPGSKS TATISGLKPGVDYTITGYAVTGRGDSPASSKPISINYRT (SEQ ID NO:5). Thus, the wild-type BC loop comprises the sequence of DAPAVTVR; the wild-type DE loop comprises the sequence of GSKST; the wild-type FG loop comprises the sequence of GRGDSPASSKP.

A variety of improved mutant ¹⁰Fn3 scaffolds have been identified. A modified Asp7, which is replaced by a non-negatively charged amino acid residue (e.g., Asn, Lys, etc.). Both of these mutations have the effect of promoting greater stability of the mutant ¹⁰Fn3 at neutral pH as compared to the wild-type form. A variety of additional alterations in the ¹⁰Fn3 scaffold that are either beneficial or neutral have been disclosed. See, for example, Batori et al. Protein Eng. 2002 December; 15(12):1015-20; Koide et al., Biochemistry 2001 Aug. 28; 40(34):10326-33.

Both the variant and wild-type ¹⁰Fn3 proteins are characterized by the same structure, namely seven beta-strand domain sequences (designated A through and six loop regions (AB loop, BC loop, CD loop, DE loop, EF loop, and FG loop) which connect the seven beta-strand domain sequences. The beta strands positioned closest to the N- and C-termini may adopt a beta-like conformation in solution. In SEQ ID NO:5, the AB loop corresponds to residues 15-16, the BC loop corresponds to residues 22-30, the CD loop corresponds to residues 39-45, the DE loop corresponds to residues 51-55, the EF loop corresponds to residues 60-66, and the FG loop corresponds to residues 76-87. The BC loop, DE loop, and FG loop are all located at the same end of the polypeptide. Similarly, immunoglobulin scaffolds tend to have at least seven beta or beta-like strands, and often nine beta or beta-like strands. Adnectins™ can include other Fn3 type fibronectin domains as long as they exhibit useful activities and properties of ¹⁰Fn3 type domains.

### VEGFR-2 Binding Proteins

In preferred embodiments of the invention, the antiangiogenic polypeptide component comprises a single domain polypeptide that is a VEGFR-2 specific binder. A single domain polypeptide disclosed herein may have at least five to seven beta or beta-like strands distributed between at least two beta sheets, and at least one loop portion connecting two beta or beta-like strands, which loop portion participates in binding to VEGFR-2, particularly KDR, with the binding characterized by a dissociation constant that is less than 1×10⁻⁶M, and preferably less than 1×10⁻⁸M. As described herein, polypeptides having a dissociation constant of less than 5×10⁻⁹M are particularly desirable for therapeutic use in vivo to inhibit VEGF signaling. Polypeptides having a dissociation constant of between 1×10⁻⁶ M and 5×10⁻⁹M may be desirable for use in detecting or labeling, ex vivo or in vivo, VEGFR-2 proteins.

Optionally, the VEGFR-2 binding protein will bind specifically to VEGFR-2 relative to other related proteins from the same species. By “specifically binds” is meant a polypeptide that recognizes and interacts with a target protein (e.g., VEGFR-2) but that does not substantially recognize and interact with other molecules in a sample, for example, a biological sample. In preferred embodiments a polypeptide of the invention will specifically bind a VEGFR with a K_{D} at least as tight as 500 nM. Preferably, the polypeptide will specifically bind a VEGFR with a K_{D} of 1 pM to 500 nM, more preferably 1 pM to 100 nM, more preferably 1 pM to 10 nM, and most preferably 1 pM to 1 nM or lower.

In general, a library of scaffold single domain polypeptides is screened to identify specific polypeptide variants that can bind to a chosen target. These libraries may be, for example, phage display libraries or PROfusion™ libraries.

In an exemplary embodiment, we have exploited a novel in vitro RNA-protein fusion display technology to isolate polypeptides that bind to both human (KDR) and mouse (Flk-1) VEGFR-2 and inhibit VEGF-dependent biological activities. These polypeptides were identified from libraries of fibronectin-based scaffold proteins (Koide et al, JMB 284:1141 (1998)) and libraries of V_{L} domains in which the diversity of CDR3 has been increased by swapping with CDR3 domains from a population of V_{H} molecules. ¹⁰Fn3 comprises approximately 94 amino acid residues, as shown in SEQ ID NO:5.

In addition, as described above, amino acid sequences at the N-terminus of ¹⁰Fn3 can also be mutated or deleted. For example, randomization of the BC, DE, and FG loops can occur in the context of a full-length ¹⁰Fn3 or in the context of a ¹⁰Fn3 having a deletion or mutation of 1-8 amino acids of the N-terminus. For example, the L at position 8 can be mutated to a Q. After randomization to create a diverse library, fibronectin-based scaffold proteins can be used in a screening assay to select for polypeptides with a high affinity for a protein, in this case the VEGFR. (For a detailed description of the RNA-protein fusion technology and fibronectin-based scaffold protein library screening methods see Szostak et al., U.S. Pat. Nos. 6,258,558; 6,261,804; 6,214,553; 6,281,344; 6,207,446; 6,518,018; PCT Publication Numbers WO 00/34784; WO 01/64942; WO 02/032925; and Roberts and Szostak, Proc Natl. Acad. Sci. 94:12297-12302, 1997, herein incorporated by reference.)

For the initial selection described herein, three regions of the ¹⁰Fn3 at positions 23-29, 52-55 and 77-86 were randomized and used for in vitro selection against the extracellular domain of human VEGFR-2 (amino acids 1-764 of KDR fused to human IgG1Fc). Using mRNA display (RNA-protein fusion) and in vitro selection, we sampled a ¹⁰Fn3-based library with approximately ten trillion variants. The initial selection identified polypeptides with moderate affinity (K_{D}=0-200 nM) that competed with VEGF for binding to KDR (human VEGFR-2). Subsequently, a single clone (K_{D}=11-13 nM) from the initial selection was subjected to mutagenesis and further selection. This affinity maturation process yielded new VEGFR binding polypeptides with dissociation constants between 60 pM to 2 nM. KDR binders are shown in Table 3. In addition, we also isolated polypeptides that could bind to Flk-1, the mouse KDR homolog, from mutagenized populations of KDR binders that initially had no detectable binding affinity to Flk-1, resulting in the isolation of polypeptides that exhibit dual specificities to both human and mouse VEGFR-2. These polypeptides are shown to bind cells that display KDR or Flk-1 extracellular domains. They also inhibited cell growth in a VEGF-dependent proliferation assay. Polypeptides that bind to KDR and Flk-1 are shown in Table 2, while a selection of preferred KDR binders and KDR/Flk-1 binders are shown in Table 1.

Using the VEGFR-2 binding polypeptides identified in these selections we determined FG loop amino acid consensus sequences required for the binding of the polypeptides to the VEGFR-2. The sequences are listed as SEQ ID NOs:1-4 below.

VEGFR-2 binding polypeptides, such as those of SEQ ID NOs:1-4, may be formulated alone (as isolated peptides), as part of a ¹⁰Fn3 single domain polypeptide, as part of a full-length fibronectin, (with a full-length amino terminus or a deleted amino terminus) or a fragment thereof, in the context of an immunoglobulin (particularly a single domain immunoglobulin), in the context of another protein having an immunoglobulin-like fold, or in the context of another, unrelated protein. The polypeptides can also be formulated as part of a fusion protein with a heterologous protein that does not itself bind to or contribute in binding to a VEGFR. In addition, the polypeptides of the invention can also be fused to nucleic acids. The polypeptides can also be engineered as monomers, dimers, or multimers.

Sequences of the Preferred Consensus VEGFR-2 Binding Peptides:

SEQ ID NO:1-

(L/M)GXN(G/D)(H/R)EL(L/M)TP

[X can be any amino acid; (/) represents alternative amino acid for the same position]

SEQ ID NO:2-

XERNGRXL(L/M/N)TP

[X can be any amino acid; (/) represents alternative amino acid for the same position]

SEQ ID NO:3-

(D/E)GXNXRXXIP

[X can be any amino acid; (/) represents alternative amino acid for the same position]

SEQ ID NO:4-

(D/E)G(R/P)N(G/E)R(S/L)(S/F)IP

[X can be any amino acid; (/) represents alternative amino acid for the same position]

Sequences of the Preferred VEGFR-2 Binding ¹⁰Fn3 Polypeptides:

SEQ ID NO:6

EVVAATPTSLLISWRHPHFPTRYYRITYGETGGNSPVQEFTVPLQPPTAT
ISGLKPGVDYTITGYAVTMGLYGHELLTPISTNYRT

SEQ ID NO:7

EVVAATPTSLLISWRHPHFPTRYYRITYGETGGNSPVQEFTVPLQPPTAT
ISGLKPGVDYTITGYAVTDGENGQFLLVPISINYRT

SEQ ID NO:8

EVVAATPTSLLISWRHPHFPTRYYRITYGETGGNSPVQEFTVPLQPPTAT
ISGLKPGVDYTITGYAVTMGPNDNELLTPISINYRT

SEQ ID NO:9

EVVAATPTSLLISWRHPHFPTRYYRITYGETGGNSPVQEFTVPLQPPTAT
ISGLKPGVDYTITGYAVTAGWDDHELFIPISINYRT

SEQ ID NO:10

EVVAATPTSLLISWRHPHFPTRYYRITYGETGGNSPVQEFTVPLQPPTAT
ISGLKPGVDYTITGYAVTSGHNDHMLMIPISINYRT

SEQ ID NO:11

EVVAATPTSLLISWRHPHFPTRYYRITYGETGGNSPVQEFTVPLQPPTAT
ISGLKPGVDYTITGYAVTAGYNDQILMTPISINYRT

SEQ ID NO:12

EVVAATPTSLLISWRHPHFPTRYYRITYGETGGNSPVQEFTVPLQPPTAT
ISGLKPGVDYTITGYAVTFGLYGKELLIPISINYRT

SEQ ID NO:13

EVVAATPTSLLISWRHPHFPTRYYRITYGETGGNSPVQEFTVPLQPPTAT
ISGLKPGVDYTITGYAVTTGPNDRLLFVPISINYRT

SEQ ID NO:14

EVVAATPTSLLISWRHPHFPTRYYRITYGETGGNSPVQEFTVPLQPPTAT
ISGLKPGVDYTITGYAVTDVYNDHEIKTPISINYRT

SEQ ID NO:15

EVVAATPTSLLISWRHPHFPTRYYRITYGETGGNSPVQEFTVPLQPPTAT
ISGLKPGVDYTITGYAVTDGKDGRVLLTPISINYRT

SEQ ID NO:16

EVVAATPTSLLISWRHPHFPTRYYRITYGETGGNSPVQEFTVPLQPPTAT
ISGLKPGVDYTITGYAVTEVHHDREIKTPISINYRT

SEQ ID NO:17

EVVAATPTSLLISWRHPHFPTRYYRITYGETGGNSPVQEFTVPLQPPTAT
ISGLKPGVDYTITGYAVTQAPNDRVLYTPISINYRT

SEQ ID NO:18

EVVAATPTSLLISWRHPHFPTRYYRITYGETGGNSPVQEFTVPLQPPTAT
ISGLKPGVDYTITGYAVTREENDHELLIPISINYRT

SEQ ID NO:19

EVVAATPTSLLISWRHPHFPTRYYRITYGETGGNSPVQEFTVPLQPPTAT
ISGLKPGVDYTITGYAVTVTHNGHPLMTPISINYRT

SEQ ID NO:20

EVVAATPTSLLISWRHPHFPTRYYRITYGETGGNSPVQEFTVPLQPPTAT
ISGLKPGVDYTITGYAVTLALKGHELLTPISINYRT

SEQ ID NO:21

VSDVPRDLEVVAATPTSLLISWRHPHFPTRYYRITYGETGGNSPVQEFTV
PLQPPTATISGLKPGVDYTITGYAVTVAQNDHELITPISINYRT

SEQ ID NO:22

VSDVPRDL/QEVVAATPTSLLISWRHPHFPTRYYRITYGETGGNSPVQEF
TVPLQPPAATISGLKPGVDYTITGYAVTMAQSGHELFTPISINYRT

SEQ ID NO:24

EVVAATPTSLLISWRHPHFPTRYYRITYGETGGNSPVQEFTVPLQPPTAT
ISGLKPGVDYTITGYAVTVERNGRVLMTPISINYRT

SEQ ID NO:25

EVVAATPTSLLISWRHPHFPTRYYRITYGETGGNSPVQEFTVPLQPPTAT
ISGLKPGVDYTITGYAVTVERNGRHLMTPISINYRT

SEQ ID NO:33

EVVAATPTSLLISWRHPHFPTRYYRITYGETGGNSPVQEFTVPLQPPTAT
ISGLKPGVDYTITGYAVTLERNGRELMTPISINYRT

SEQ ID NO:45

EVVAATPTSLLISWRHPHFPTRYYRITYGETGGNSPVQEFTVPLQPPTAT
ISGLKPGVDYTITGYAVTEERNGRTLRTPISINYRT

SEQ ID NO:53

EVVAATPTSLLISWRHPHFPTRYYRITYGETGGNSPVQEFTVPLQPPTAT
ISGLKPGVDYTITGYAVTVERNDRVLFTPISINYRT

SEQ ID NO:57

EVVAATPTSLLISWRHPHFPTRYYRITYGETGGNSPVQEFTVPLQPPTAT
ISGLKPGVDYTITGYAVTVERNGRELMTPISINYRT

SEQ ID NO:62

EVVAATPTSLLISWRHPHFPTRYYRITYGETGGNSPVQEFTVPLQPPTAT
ISGLKPGVDYTITGYAVTLERNGRELMVPISINYRT

SEQ ID NO:63

EVVAATPTSLLISWRHPHFPTRYYRITYGETGGNSPVQEFTVPLQPPTAT
ISGLKPGVDYTITGYAVTDGRNDRKLMVPISINYRT

SEQ ID NO:68

EVVAATPTSLLISWRHPHFPTRYYRITYGETGGNSPVQEFTVPLQPPTAT
ISGLKPGVDYTITGYAVTDGQNGRLLNVPISINYRT

SEQ ID NO:91

EVVAATPTSLLISWRHHPHFPTRYYRITYGETGGNSPVQEFTVPLQPPTA
TISGLKPGVDYTITGYAVTVHWNGRELMTPISINYRT

SEQ ID NO:92

EVVAATPTSLLISWRHPHFPTRYYRITYGETGGNSPVQEFTVPLQPPTAT
ISGLKPGVDYTITGYAVTEEWNGRVLMTPISINYRT

SEQ ID NO:93

EVVAATPTSLLISWRHPHFPTRYYRITYGETGGNSPVQEFTVPLQPPTAT
ISGLKPGVDYTITGYAVTVERNGHTLMTPISINYRT

SEQ ID NO:94

EVVAATPTSLLISWRHPHFPTRYYRITYGETGGNSPVQEFTVPLQPPTAT
ISGLKPGVDYTITGYAVTVEENGRQLMTPISINYRT

SEQ ID NO:95

EVVAATPTSLLISWRHPHFPTRYYRITYGETGGNSPVQEFTVPLQPPTAT
ISGLKPGVDYTITGYAVTLERNGQVLFTPISINYRT

SEQ ID NO:96

EVVAATPTSLLISWRHPHFPTRYYRITYGETGGNSPVQEFTVPLQPPTAT
ISGLKPGVDYTITGYAVTVERNGQVLYTPISINYRT

SEQ ID NO:97

EVVAATPTSLLISWRHPHFPTRYYRITYGETGGNSPVQEFTVPLQPPTAT
ISGLKPGVDYTITGYAVTWGYKDHELLIPISINYRT

SEQ ID NO:98

EVVAATPTSLLISWRHPHFPTRYYRITYGETGGNSPVQEFTVPLQPPTAT
ISGLKPGVDYTITGYAVTLGRNDRELLTPISINYRT

SEQ ID NO:99

EVVAATPTSLLISWRHPHFPTRYYRITYGETGGNSPVQEFTVPLQPPTAT
ISGLKPGVDYTITGYAVTDGPNDRLLNIPISINYRT

SEQ ID NO:100

EVVAATPTSLLISWRHPHFPTRYYRITYGETGGNSPVQEFTVPLQPPTAT
ISGLKPGVDYTITGYAVTFARDGHEILTPISINYRT

SEQ ID NO:101

EVVAATPTSLLISWRHPHFPTRYYRITYGETGGNSPVQEFTVPLQPPTAT
ISGLKPGVDYTITGYAVTLEQNGRELMTPISINYRT

SEQ ID NO:102

EVVAATPTSLLISWRHPHFPTRYYRITYGETGGNSPVQEFTVPLQPPTAT
ISGLKPGVDYTITGYAVTVEENGRVLNTPISINYRT

SEQ ID NO:103

EVVAATPTSLLISWRHPHFPTRYYRITYGETGGNSPVQEFTVPLQPPTAT
ISGLKPGVDYTITGYAVTLEPNGRYLMVPISINYRT

SEQ ID NO:104

EVVAATPTSLLISWRHPHFPTRYYRITYGETGGNSPVQEFTVPLQPPTAT
ISGLKPGVDYTITGYAVTEGRNGRELFIPISINYRT

SEQ ID NO:154

VSDVPRDLEVVAATPTSLLISWRHPHFPTRYYRITYGETGGNSPVQEFTV
PLQPPAATISGLKPGVDYTITGYAVTWERNGRELFTPISINYRT

SEQ ID NO:156

VSDVPRDLEVVAATPTSLLISWRHPHFPTRYYRITYGETGGNSPVQEFTV
PLQPPAATISGLKPGVDYTITGYAVTKERNGRELFTPISINYRT

SEQ ID NO:172

VSDVPRDLEVVAATPTSLLISWRHPHFPTHYYRITYGETGGNSPVQEFTV
PLQPPAATISGLKPGVDYTITGYAVTTERTGRELFTPISINYRT

SEQ ID NO:173

VSDVPRDLEVVAATPTSLLISWRHPHFPTHYYRITYGETGGNSPVQEFTV
PLQPPAATISGLKPGVDYTITGYAVTKERSGRELFTPISINYRT

SEQ ID NO:175

VSDVPRDLEVVAATPTSLLISWRHPHFPTHYYRITYGETGGNSPVQEFTV
PLQPPAATISGLKPGVDYTITGYAVTLERDGRELFTPISINYRT

SEQ ID NO:177

VSDVPRDLEVVAATPTSLLISWRHPHFPTRYYRITYGETGGNSPVQEFTV
PLQPPLATISGLKPGVDYTITG/VYAVTKERNGRELFTPISINYRT

SEQ ID NO:180

VSDVPRDLEVVAATPTSLLISWRHPHFPTRYYRITYGETGGNSPVQEFTV
PLQPTTATISGLKPGVDYTITGYAVTWERNGRELFTPISINYRT

SEQ ID NO:181

VSDVPRDLEVVAATPTSLLISWRHPHFPTRYYRITYGETGGNSPVQEFTV
PLQPTVATISGLKPGVDYTITGYAVTLERNDRELFTPISINYRT

SEQ ID NO:186

MGEVVAATPTSLLISWRHPHFPTRYYRITYGETGGNSPVQEFTVPLQPPT
ATISGLKPGVDYTITVYAVTDGRNGRLLSIPISINYRTEIDKPSQ

SEQ ID NO:187

MGEVVAATPTSLLISWRHPHFPTRYYRITYGETGGNSPVQEFTVPLQPPT
ATISGLKPGVDYTITVYAVTDGRNGRLLSIPISINYRTEIDKPCQ

SEQ ID NO:188

MVSDVPRDLEVVAATPTSLLISWRHPHFPTRYYRITYGETGGNSPVQEFT
VPLQPPTATISGLKPGVDYTITVYAVTDGRNGRLLSIPISINYRTEIDKPSQ

SEQ ID NO:189

MGEVVAATPTSLLISWRHPHFPTRYYRITYGETGGNSPVQEFTVPLQPPT
ATISGLKPGVDYTITVYAVTDGWNGRLLSIPISINYRT

SEQ ID NO:190

MGEVVAATPTSLLISWRHPHFPTRYYRITYGETGGNSPVQEFTVPLQPPT
ATISGLKPGVDYTITVYAVTEGPNERSLFIPISINYRT

SEQ ID NO:191

MVSDVPRDLEVVAATPTSLLISWRHPHFPTRYYRITYGETGGNSPVQEFT
VPLQPPTATISGLKPGVDYTITVYAVTEGPNERSLFIPISINYRT

SEQ ID NO:192

(A core form of the polypeptide referred to herein
as CT-01):
EVVAATPTSLLISWRHPHFPTRYYRITYGETGGNSPVQEFTVPLQPPTAT
ISGLKPGVDYTITVYAVTDGRNGRLLSIPISINYRT

The CT-01 molecule above has a deletion of the first 8 amino acids and may include additional amino acids at the N- or C-termini. For example, an additional MG sequence may be placed at the N-terminus. The M will usually be cleaved off, leaving a GEV . . . sequence at the N-terminus. The re-addition of the normal 8 amino acids at the N-terminus also produces a KDR binding protein with desirable properties. The N-terminal methionine is generally cleaved off to yield a sequence:

(SEQ ID NO:193)

VSDVPRDLEVVAATPTSLLISWRHPHFPTRYYRITYGETGGNSPVQEFTV
PLQPPTATISGLKPGVDYTITVYAVTDGRNGRLLSIPISINYRT.

A polypeptide disclosed herein may be modified by one or more conservative substitutions, particularly in portions of the protein that are not expected to interact with a target protein. It is expected that as many as 5%, 10%, 20% or even 30% or more of the amino acids in an immunoglobulin or immunoglobulin-like domain may be altered by a conservative substitution without substantially altering the affinity of the protein for target. It may be that such changes will alter the immunogenicity of the polypeptide in vivo, and where the immunogenicity is decreased, such changes will be desirable. As used herein, “conservative substitutions” are residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., Atlas of Protein Sequence and Structure 5:345-352 (1978 & Supp.). Examples of conservative substitutions are substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine.

Polypeptides disclosed herein may also be modified in order to improve potency, bioavailability, chemical stability, and/or efficacy. For example, within one embodiment of the invention D-amino acid peptides, or retroenantio peptide sequences may be generated in order to improve the bioactivity and chemical stability of a polypeptide structure (see, e.g., Juvvadi et al., J. Am. Chem. Soc. 118: 8989-8997, 1996; Freidinger et al., Science, 210: 656-658, 1980). Lactam constraints (see Freidinger, supra), and/or azabicycloalkane amino acids as dipeptide surrogates can also be utilized to improve the biological and pharmacological properties of the native peptides (see, e.g., Hanessian et al., Tetrahedron 53:12789-12854, 1997).

Amide bond surrogates, such as thioamides, secondary and tertiary amines, heterocycles among others (see review in Spatola, A. F. in “Chemistry and Biochemistry of Amino Acids, Peptides and Proteins” Wenstein, B. Ed. Marcel Dekker, New York, 1983 Vol. 7, pp 267-357) can also be utilized to prevent enzymatic degradation of the polypeptide backbone thereby resulting in improved activity. Conversion of linear polypeptides to cyclic polypeptide analogs can also be utilized to improve metabolic stability, since cyclic polypeptides are much less sensitive to enzymatic degradation (see generally, Veber, et al. Nature 292:55-58, 1981).

Polypeptides can also be modified utilizing end group capping as esters and amides in order to slow or prevent metabolism and enhance lipophilicity. Dimers of the peptide attached by various linkers may also enhance activity and specificity (see for example: Y. Shimohigashi et al, in Peptide Chemistry 1988, Proceedings of the 26th Symposium on Peptide Chemistry, Tokyo, October 24-26, pgs. 47-50, 1989). For additional examples of polypeptide modifications, such as non-natural amino acids, see U.S. Pat. No. 6,559,126.

For use in vivo, a form suitable for pegylation may be generated. For example, a C-terminal tail comprising a cysteine was added and expressed, as shown below for a CT-01 form lacking the eight N-terminal amino acids (EIDKPCQ is added at the C-terminus).

GEVVAATPTSLLISWRHPHFPTRYYRITYGETGGNSPVQEFTVPLQPPTATISGLK PGVDYTITVYAVTDGRNGRLLSIPISINYRTEIDKPCQ (SEQ ID NO:194). The pegylated form of this molecule is used in the in vivo experiments described below. A control form with a serine instead of a cysteine was also used:

(SEQ ID NO:195)

GEVVAATPTSLLISWRHPHFPTRYYRITYGETGGNSPVQEFTVPLQPPTA
TISGLKPGVDYTITVYAVTDGRNGRLLSIPISINYRTEIDKPSQ.

The same C-terminal tails may also be added to CT-01 forms having the N-terminal eight amino acids, such as is shown in SEQ ID NO:193.

Additional variants with desirable KDR binding properties were isolated. The following core sequence has a somewhat different FG loop, and may be expressed with, for example, an N-terminal MG sequence, an N-terminal sequence that restores the 8 deleted amino acids, and/or a C-terminal tail to provide a cysteine for pegylation.

EVVAATPTSLLISWRHPHFPTRYYRITYGETGGNSPVQEFTVPLQPPTATISGLKP GVDYTITVYAVTEGPNERSLFIPISINYRT (SEQ ID NO:196). Another such variant has the core sequence:

(SEQ ID NO:197)

VSDVPRDLEVVAATPTSLLISWRHPHFPTRYYRITYGETGGNSPVQEFTV
PLQPPTATISGLKPGVDYTITVYAVTEGPNERSLFIPISTNYRT.

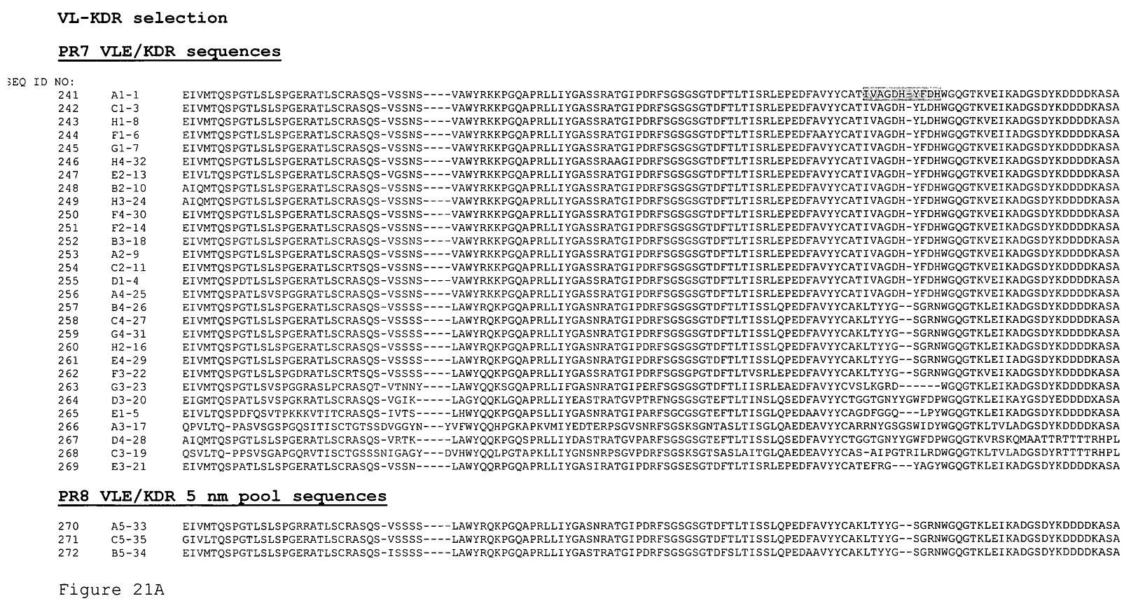

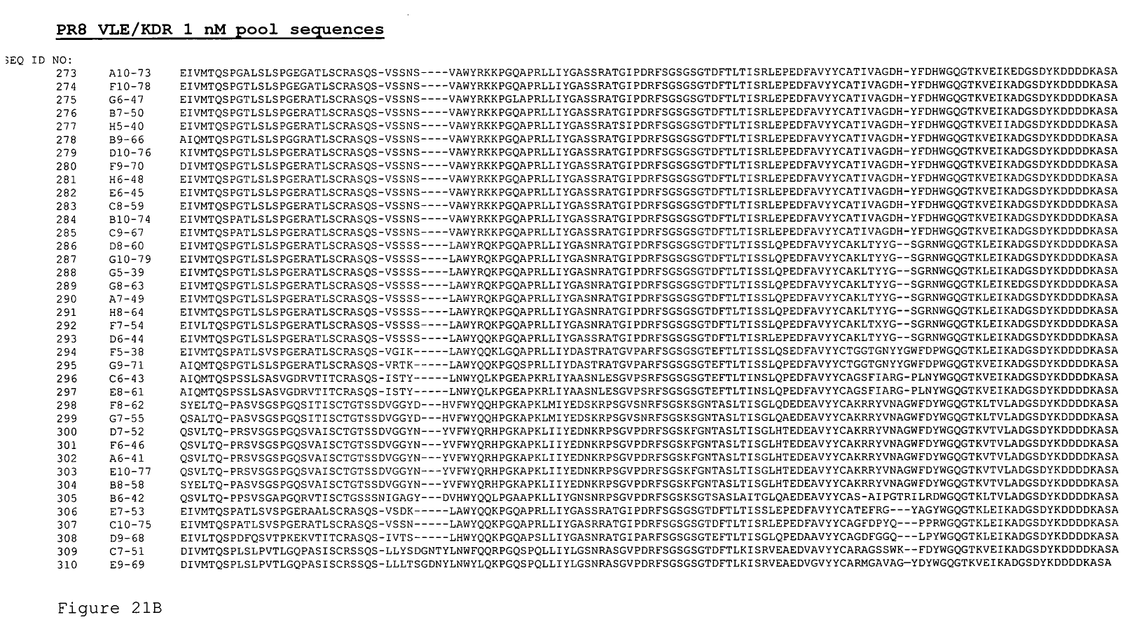

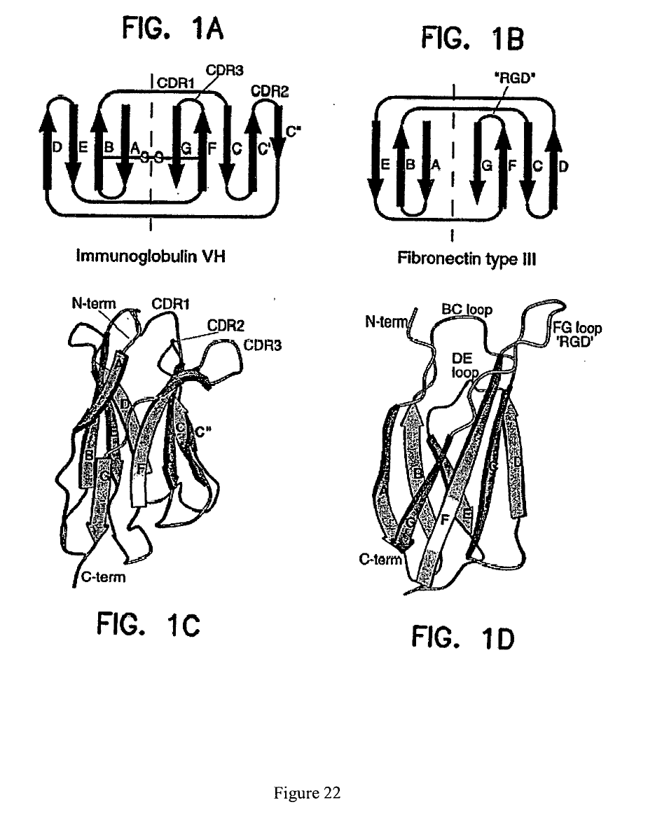

Additionally, preferred single domain immunoglobulin polypeptides in a V_{L} framework were isolated by similar methodology and are disclosed in FIG. 21.

### ADDITIONAL PROTEIN EMBODIMENTS

Proteins of the invention include a single domain polypeptide as described herein, generally a polypeptide that binds to a target, such as VEGFR-2, and where target binding activity situated within a single structural domain, as differentiated from, for example, antibodies and single chain antibodies, where antigen binding activity is generally contributed by both a heavy chain variable domain and a light chain variable domain. The disclosure also provides larger proteins that may comprise single domain polypeptides that bind to target. For example, a plurality of single domain polypeptides may be connected to create a composite molecule with increased avidity or multivalency. Likewise, a single domain polypeptide may be attached (e.g., as a fusion protein) to any number of other polypeptides. In certain aspects a single domain polypeptide may comprise at least five to seven beta or beta-like strands distributed among at least two beta sheets, as exemplified by immunoglobulin and immunoglobulin-like domains. A beta-like strand is a string of amino acids that participates in the stabilization of a single domain polypeptide but does not necessarily adopt a beta strand conformation. Whether a beta-like strand participates in the stabilization of the protein may be assessed by deleting the string or altering the sequence of the string and analyzing whether protein stability is diminished. Stability may be assessed by, for example, thermal denaturation and renaturation studies. Preferably, a single domain polypeptide will include no more than two beta-like strands. A beta-like strand will not usually adopt an alpha-helical conformation but may adopt a random coil structure. In the context of an immunoglobulin domain or an immunoglobulin-like domain, a beta-like strand will most often occur at the position in the structure that would otherwise be occupied by the most N-terminal beta strand or the most C-terminal beta strand. An amino acid string which, if situated in the interior of a protein sequence would normally form a beta strand, may, when situated at a position closer to an N- or C-terminus, adopt a conformation that is not clearly a beta strand and is referred to herein as a beta-like strand.

In certain embodiments, the disclosure provides single domain polypeptides that bind to VEGFR-2. Preferably the single domain polypeptides bind to human VEGFR-2 or a model species VEGFR-2. A single domain polypeptide may comprise between about 80 and about 150 amino acids that have a structural organization comprising: at least seven beta strands or beta-like strands distributed between at least two beta sheets, and at least one loop portion connecting two beta strands or beta-like strands, which loop portion participates in binding to VEGFR-2. In other words a loop portion may link two beta strands, two beta-like strands or one beta strand and one beta-like strand. Typically, one or more of the loop portions will participate in VEGFR-2 binding, although it is possible that one or more of the beta or beta-like strand portions will also participate in VEGFR-2 binding, particularly those beta or beta-like strand portions that are situated closest to the loop portions. A single domain polypeptide may comprise a structural unit that is an immunoglobulin domain or an immunoglobulin-like domain. A single domain polypeptide may bind to any part of VEGFR-2, although polypeptides that bind to an extracellular domain of a VEGFR-2 are preferred. Binding may be assessed in terms of equilibrium constants (e.g., dissociation, K_{D}) and in terms of kinetic constants (e.g., on rate constant, kₒₙ and off rate constant, k_{off}). A single domain polypeptide will typically be selected to bind to VEGFR-2 with a K_{D} of less than about 10⁻⁶M, or less than about 10⁻⁷M, about 5×10⁻⁸M, about 10⁻⁸M or less than about 10⁻⁹M. VEGFR-2 binding polypeptides may compete for binding with one, or two or more members of the VEGF family, particularly VEGF-A, VEGF-C, and/or VEGF-D, and may inhibit one or more VEGFR-2-mediated biological events, such as proliferation of cancer cells and cancer metastasis. VEGFR-2 binding polypeptides may be used for therapeutic purposes as well as for any purpose involving the detection or binding of VEGFR-2. Polypeptides for therapeutic use will generally have a K_{D} of less than 5×10⁻⁸M, less than 10⁻⁸M or less than 10⁻⁹M, although higher K_{D} values may be tolerated where the k_{off} is sufficiently low or the kₒₙ is sufficiently high.

In certain embodiments, the single domain polypeptide comprises an immunoglobulin (Ig) variable domain. The Ig variable domain may, for example, be selected from the group consisting of: a human V_{L} domain, a human V_{H} domain and a camelid V_{HH} domain. One, two, three or more loops of the Ig variable domain may participate in binding to VEGFR-2, and typically any of the loops known as CDR1, CDR2 or CDR3 will participate in VEGFR-2 binding.

In certain embodiments, the single domain polypeptide comprises an immunoglobulin-like domain. One, two, three or more loops of the immunoglobulin-like domain may participate in binding to VEGFR-2. A preferred immunoglobulin-like domain is a fibronectin type III (Fn3) domain. Such domain may comprise, in order from N-terminus to C-terminus, a beta or beta-like strand, A; a loop, AB; a beta strand, B; a loop, BC; a beta strand C; a loop CD; a beta strand D; a loop DE; a beta strand F; a loop FG; and a beta or beta-like strand G.

Optionally, any or all of loops AB, BC, CD, DE, EF and FG may participate in VEGFR-2 binding, although preferred loops are BC, DE and FG. A preferred Fn3 domain is an Fn3 domain derived from human fibronectin, particularly the 10^{th} Fn3 domain of fibronectin, referred to as ¹⁰Fn3. It should be noted that none of VEGFR-2 binding polypeptides disclosed herein have an amino acid sequence that is identical to native ¹⁰Fn3; the sequence has been modified to obtain VEGFR-2 binding proteins, but proteins having the basic structural features of ¹⁰Fn3, and particularly those retaining recognizable sequence homology to the native ¹⁰Fn3 are nonetheless referred to herein as “¹⁰Fn3 polypeptides”. This nomenclature is similar to that found in the antibody field where, for example, a recombinant antibody V_{L} domain generated against a particular target protein may not be identical to any naturally occurring V_{L} domain but nonetheless the protein is recognizably a V_{L} protein. A ¹⁰Fn3 polypeptide may be at least 60%, 65%, 70%, 75%, 80%, 85%, or 90% identical to the human ¹⁰Fn3 domain, shown in SEQ ID NO:5. Much of the variability will generally occur in one or more of the loops. Each of the beta or beta-like strands of a ¹⁰Fn3 polypeptide may consist essentially of an amino acid sequence that is at least 80%, 85%, 90%, 95% or 100% identical to the sequence of a corresponding beta or beta-like strand of SEQ ID NO: 5, provided that such variation does not disrupt the stability of the polypeptide in physiological conditions. A ¹⁰Fn3 polypeptide may have a sequence in each of the loops AB, CD, and EF that consists essentially of an amino acid sequence that is at least 80%, 85%, 90%, 95% or 100% identical to the sequence of a corresponding loop of SEQ ID NO:5. In many instances, any or all of loops BC, DE, and FG will be poorly conserved relative to SEQ ID NO:5. For example, all of loops BC, DE, and FG may be less than 20%, 10%, or 0% identical to their corresponding loops in SEQ ID NO:5.

In certain embodiments, the disclosure provides a non-antibody polypeptide comprising a domain having an immunoglobulin-like fold that binds to VEGFR-2. The non-antibody polypeptide may have a molecular weight of less than 20 kDa, or less than 15 kDa and will generally be derived (by, for example, alteration of the amino acid sequence) from a reference, or “scaffold”, protein, such as an Fn3 scaffold. The non-antibody polypeptide may bind VEGFR-2 with a K_{D} less than 10⁻⁶M, or less than 10⁻⁷M, less than 5×10⁻⁸M, less than 10⁻⁸M or less than 10⁻⁹M. The unaltered reference protein either will not meaningfully bind to VEGFR-2 or will bind with a K_{D} of greater than 10⁻⁶M. The non-antibody polypeptide may inhibit VEGFR-2 signaling, particularly where the non-antibody polypeptide has a K_{D} of less than 5×10⁻⁸M, less than 10⁻⁸M or less than 10⁻⁹M, although higher K_{D} values may be tolerated where the k_{off} is sufficiently low (e.g., less than 5×10⁻⁴s⁻¹). The immunoglobulin-like fold may be a ¹⁰Fn3 polypeptide.

In certain embodiments, the disclosure provides a polypeptide comprising a single domain having an immunoglobulin fold that binds to VEGFR-2. The polypeptide may have a molecular weight of less than 20 kDa, or less than 15 kDa and will generally be derived (by, for example, alteration of the amino acid sequence) from a variable domain of an immunoglobulin. The polypeptide may bind VEGFR-2 with a K_{D} less than 10⁻⁶ M, or less than 10⁻⁷M, less than 5×10⁻⁸M, less than 10⁻⁸M or less than 10⁻⁹M. The polypeptide may inhibit VEGFR-2 signaling, particularly where the polypeptide has a K_{D} of less than 5×10⁻⁸M, less than 10⁻⁸M or less than 10⁻⁹M, although higher K_{D} values may be tolerated where the k_{off} is sufficiently low or where the kₒₙ is sufficiently high. In some embodiments, the polypeptide comprises an amino acid sequence that is at least 80% identical to SEQ NO: 5. In some embodiments, the polypeptide comprises an amino acid sequence selected from the group consisting of any of SEQ ID NOs: 6-183, 186-197, 199 and 241-310. In some embodiments, the polypeptide further comprises PEG.

In certain aspects, the disclosure provides sustained-release delivery systems that deliver short peptide sequences that mediate VEGFR-2 binding. Such sequences may mediate VEGFR-2 binding in an isolated form or when inserted into a particular protein structure, such as an immunoglobulin or immunoglobulin-like domain. Examples of such sequences include those disclosed (such as SEQ ID NOs: 6-183, 186-197, 199 and 241-310) and other sequences that are at least 85%, 90%, or 95% identical to SEQ ID NO:5 to such sequences and retain VEGFR-2 binding activity. Accordingly, the disclosure provides substantially pure polypeptides comprising an amino acid sequence that is at least 85% identical to the sequence of any of such sequences, wherein said polypeptide binds to a VEGFR-2 and competes with an VEGF species for binding to VEGFR-2. Examples of such polypeptides include a polypeptide comprising an amino acid sequence that is at least 80%, 85%, 90%, 95% or 100% identical to an amino acid sequence of SEQ ID: 6-183, 186-197, 199 and 241-310. Preferably such polypeptides will inhibit a biological activity of a VEGF and may bind to VEGFR-2 with a K_{D} less than 10⁻⁶ M, or less than 10⁻⁷M, less than 5×10⁻⁸M, less than 10⁻⁸M or less than 10⁻⁹M.

In certain embodiments, any of the VEGFR-2 binding polypeptides described herein may be bound to one or more additional moieties, including, for example, a moiety that also binds to VEGFR-2 (e.g., a second identical or different VEGFR-2 binding polypeptide), a moiety that binds to a different target (e.g., to create a dual-specificity binding agent), a labeling moiety, a moiety that facilitates protein purification or a moiety that provides improved pharmacokinetics. Improved pharmacokinetics may be assessed according to the perceived therapeutic need. Often it is desirable to increase bioavailability and/or increase the time between doses, possibly by increasing the time that a protein remains available in the serum after dosing. In some instances, it is desirable to improve the continuity of the serum concentration of the protein over time (e.g., decrease the difference in serum concentration of the protein shortly after administration and shortly before the next administration). Moieties that tend to slow clearance of a protein from the blood include polyethylene glycol, sugars (e.g. sialic acid), and well-tolerated protein moieties (e.g., Fc fragment or serum albumin). The single domain polypeptide may be attached to a moiety that reduces the clearance rate of the polypeptide in a mammal (e.g., mouse, rat, or human) by greater than three-fold relative to the unmodified polypeptide. Other measures of improved pharmacokinetics may include serum half-life, which is often divided into an alpha phase and a beta phase. Either or both phases may be improved significantly by addition of an appropriate moiety. Where polyethylene glycol is employed, one or more PEG molecules may be attached at different positions in the protein, and such attachment may be achieved by reaction with amines, thiols or other suitable reactive groups. Pegylation may be achieved by site-directed pegylation, wherein a suitable reactive group is introduced into the protein to create a site where pegylation preferentially occurs. In a preferred embodiment, the protein is modified so as to have a cysteine residue at a desired position, permitting site directed pegylation on the cysteine. PEG may vary widely in molecular weight and may be branched or linear. Notably, the present disclosure establishes that pegylation is compatible with target binding activity of ¹⁰Fn3 polypeptides and, further, that pegylation does improve the pharmacokinetics of such polypeptides. Accordingly, in one embodiment, the disclosure provides pegylated forms of ¹⁰Fn3 polypeptides, regardless of the target that can be bound by such polypeptides.

### Nucleic Acids and Production of Polypeptides

Polypeptides of the present invention can be produced using any standard methods known in the art. In one example, the polypeptides are produced by recombinant DNA methods by inserting a nucleic acid sequence (e.g., a cDNA) encoding the polypeptide into a recombinant expression vector and expressing the DNA sequence under conditions promoting expression.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc Natl Acad Sci USA. 2003 Jan. 21; 100(2):438-42; Sinclair et al. Protein Expr Purif. 2002 October; 26(1):96-105; Connell N D. Curr Opin Biotechnol. 2001 October; 12(5):446-9; Makrides et al. Microbiol. Rev. 1996 September; 60(3):512-38; and Sharp et al. Yeast. 1991 October; 7(7):657-78.

Examples of nucleic acid sequences encoding a CT-01 polypeptide disclosed herein are:

SEQ ID NO:184

atgggcgaagttgttgctgcgacccccaccagcctactgatcagctggcg
ccacccgcacttcccgactagatattacaggatcacttacggagaaacag
gaggaaatagccctgtccaggagttcactgtgcctctgcagccccccaca
gctaccatcagcggccttaaacctggagttgattataccatcactgtgta
tgctgtcactgacggccggaacgggcgcctcctgagcatcccaatttcca
ttaattaccgcacagaaattgacaaaccatgccag

SEQ ID NO:185

atgggcgaagttgttgctgcgacccccaccagcctactgatcagctggcg
ccacccgcacttcccgactagatattacaggatcacttacggagaaacag
gaggaaatagccctgtccaggagttcactgtgcctctgcagccccccaca
gctaccatcagcggccttaaacctggagttgattataccatcactgtgta
tgctgtcactgacggccggaacgggcgcctcctgagcatcccaatttcca
ttaattaccgcaca

General techniques for nucleic acid manipulation are described for example in Sambrook et al., Molecular Cloning: A Laboratory Manual, Vols. 1-3, Cold Spring Harbor Laboratory Press, 2 ed., 1989, or F. Ausubel et al., Current Protocols in Molecular Biology (Green Publishing and Wiley-Interscience: New York, 1987) and periodic updates, herein incorporated by reference. The DNA encoding the polypeptide is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The ability to replicate in a host, usually conferred by an origin of replication, and a selection gene to facilitate recognition of transformants are additionally incorporated.

The recombinant DNA can also include any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, New York, 1985), the relevant disclosure of which is hereby incorporated by reference.

The expression construct is introduced into the host cell using a method appropriate to the host cell, as will be apparent to one of skill in the art. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells include prokaryotes, yeast, mammalian cells, or bacterial cells. Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides disclosed herein would make expression in E. coli as the preferred method for expression. The protein is then purified from culture media or cell extracts.

Proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

VEGFR-binding polypeptides can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

The polypeptide of the present invention can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified polypeptide is preferably at least 85% pure, more preferably at least 95% pure, and most preferably at least 98% pure. Regardless of the exact numerical value of the purity, the polypeptide is sufficiently pure for use as a pharmaceutical product. The polypeptide is in particular free of endotoxins

### Post-Translational Modifications of Polypeptides

In certain embodiments, the binding polypeptides of the invention may further comprise post-translational modifications. Exemplary post-translational protein modification include phosphorylation, acetylation, methylation, ADP-ribosylation, ubiquitination, glycosylation, carbonylation, sumoylation, biotinylation or addition of a polypeptide side chain or of a hydrophobic group. As a result, the modified soluble polypeptides may contain non-amino acid elements, such as lipids, poly- or mono-saccharide, and phosphates. A preferred form of glycosylation is sialylation, which conjugates one or more sialic acid moieties to the polypeptide. Sialic acid moieties improve solubility and serum half-life while also reducing the possible immunogeneticity of the protein. See, e.g., Raju et al. Biochemistry. 2001 Jul. 31; 40(30):8868-76. Effects of such non-amino acid elements on the functionality of a polypeptide may be tested for its antagonizing role in VEGFR-2 or VEGF function, e.g., its inhibitory effect on angiogenesis or on tumor growth.

In one specific embodiment of the present invention, modified forms of the subject soluble polypeptides comprise linking the subject soluble polypeptides to nonproteinaceous polymers. In one specific embodiment, the polymer is polyethylene glycol (“PEG”), polypropylene glycol, or polyoxyalkylenes, in the manner as set forth in U.S. Pat. No. 4,640,835; 4,496,689; 4,301,144; 4,670,417; 4,791,192 or 4,179,337. Examples of the modified polypeptide of the invention include PEGylated CT-322.

PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula:

X—O(CH₂CH₂O)ₙ₋₁CH₂CH₂OH (1),

where n is 20 to 2300 and X is H or a terminal modification, e.g., a C₁₋₄ alkyl. In one embodiment, the PEG of the invention terminates on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-A 0 473 084 and U.S. Pat. No. 5,932,462. One form of PEGs includes two PEG side-chains (PEG2) linked via the primary amino groups of a lysine (Monfardini, C., et al., Bioconjugate Chem. 6 (1995) 62-69).

In a preferred embodiment, the pegylated ¹⁰Fn3 polypeptide is produced by site-directed pegylation, particularly by conjugation of PEG to a cysteine moiety at the N- or C-terminus. Accordingly, the present disclosure provides a target-binding ¹⁰Fn3 polypeptide with improved pharmacokinetic properties, the polypeptide comprising: a ¹⁰Fn3 domain having from about 80 to about 150 amino acids, wherein at least one of the loops of said ¹⁰Fn3 domain participate in target binding; and a covalently bound PEG moiety, wherein said ¹⁰Fn3 polypeptide binds to the target with a K_{D} of less than 100 nM and has a clearance rate of less than 30 mL/hr/kg in a mammal. The PEG moiety may be attached to the ¹⁰Fn3 polypeptide by site directed pegylation, such as by attachment to a Cys residue, where the Cys residue may be positioned at the N-terminus of the ¹⁰Fn3 polypeptide or between the N-terminus and the most N-terminal beta or beta-like strand or at the C-terminus of the ¹⁰Fn3 polypeptide or between the C-terminus and the most C-terminal beta or beta-like strand. A Cys residue may be situated at other positions as well, particularly any of the loops that do not participate in target binding. A PEG moiety may also be attached by other chemistry, including by conjugation to amines.

PEG conjugation to peptides or proteins generally involves the activation of PEG and coupling of the activated PEG-intermediates directly to target proteins/peptides or to a linker, which is subsequently activated and coupled to target proteins/peptides (see Abuchowski, A. et al, J. Biol. Chem., 252, 3571 (1977) and J. Biol. Chem., 252, 3582 (1977), Zalipsky, et al., and Harris et. al., in: Poly(ethylene glycol) Chemistry: Biotechnical and Biomedical Applications; (J. M. Harris ed.) Plenum Press: New York, 1992; Chap. 21 and 22). It is noted that a binding polypeptide containing a PEG molecule is also known as a conjugated protein, whereas the protein lacking an attached PEG molecule can be referred to as unconjugated.

A variety of molecular mass forms of PEG can be selected, e.g., from about 1,000 Daltons (Da) to 100,000 Da (n is 20 to 2300), for conjugating to VEGFR-2 binding polypeptides. The number of repeating units “n” in the PEG is approximated for the molecular mass described in Daltons. It is preferred that the combined molecular mass of PEG on an activated linker is suitable for pharmaceutical use. Thus, in one embodiment, the molecular mass of the PEG molecules does not exceed 100,000 Da. For example, if three PEG molecules are attached to a linker, where each PEG molecule has the same molecular mass of 12,000 Da (each n is about 270), then the total molecular mass of PEG on the linker is about 36,000 Da (total n is about 820). The molecular masses of the PEG attached to the linker can also be different, e.g., of three molecules on a linker two PEG molecules can be 5,000 Da each (each n is about 110) and one PEG molecule can be 12,000 Da (n is about 270).

In a specific embodiment of the invention, a VEGFR-2 binding polypeptide is covalently linked to one poly(ethylene glycol) group of the formula: —CO—(CH₂)ₓ—(OCH₂CH₂)ₘ—OR, with the —CO (i.e. carbonyl) of the poly(ethylene glycol) group forming an amide bond with one of the amino groups of the binding polypeptide; R being lower alkyl; x being 2 or 3; m being from about 450 to about 950; and n and m being chosen so that the molecular weight of the conjugate minus the binding polypeptide is from about 10 to 40 kDa. In one embodiment, an binding polypeptide's ε-amino group of a lysine is the available (free) amino group.

The above conjugates may be more specifically presented by formula (II): P—NHCO—(CH₂)ₓ—(OCH₂CH₂)ₘ—OR (II), wherein P is the group of a binding polypeptide as described herein, (i.e. without the amino group or amino groups which form an amide linkage with the carbonyl shown in formula (II); and wherein R is lower alkyl; x is 2 or 3; m is from about 450 to about 950 and is chosen so that the molecular weight of the conjugate minus the binding polypeptide is from about 10 to about 40 kDa. As used herein, the given ranges of “m” have an orientational meaning. The ranges of “m” are determined in any case, and exactly, by the molecular weight of the PEG group.

One skilled in the art can select a suitable molecular mass for PEG, e.g., based on how the pegylated binding polypeptide will be used therapeutically, the desired dosage, circulation time, resistance to proteolysis, immunogenicity, and other considerations. For a discussion of PEG and its use to enhance the properties of proteins, see N. V. Katre, Advanced Drug Delivery Reviews 10: 91-114 (1993).

In one embodiment of the invention, PEG molecules may be activated to react with amino groups on a binding polypeptide, such as with lysines (Bencham C. O. et al., Anal. Biochem., 131, 25 (1983); Veronese, F. M. et al., Appl. Biochem., 11, 141 (1985).; Zalipsky, S. et al., Polymeric Drugs and Drug Delivery Systems, adrs 9-110 ACS Symposium Series 469 (1999); Zalipsky, S. et al., Europ. Polym. J., 19, 1177-1183 (1983); Delgado, C. et al., Biotechnology and Applied Biochemistry, 12, 119-128 (1990)).

In one specific embodiment, carbonate esters of PEG are used to form the PEG-binding polypeptide conjugates. N,N′-disuccinimidylcarbonate (DSC) may be used in the reaction with PEG to form active mixed PEG-succinimidyl carbonate that may be subsequently reacted with a nucleophilic group of a linker or an amino group of a binding polypeptide (see U.S. Pat. No. 5,281,698 and U.S. Pat. No. 5,932,462). In a similar type of reaction, 1,1′-(dibenzotriazolyl)carbonate and di-(2-pyridyl)carbonate may be reacted with PEG to form PEG-benzotriazolyl and PEG-pyridyl mixed carbonate (U.S. Pat. No. 5,382,657), respectively.

Pegylation of a ¹⁰Fn3 polypeptide can be performed according to the methods of the state of the art, for example by reaction of the binding polypeptide with electrophilically active PEGs (supplier: Shearwater Corp., USA, www.shearwatercorp.com). Preferred PEG reagents of the present invention are, e.g., N-hydroxysuccinimidyl propionates (PEG-SPA), butanoates (PEG-SBA), PEG-succinimidyl propionate or branched N-hydroxysuccinimides such as mPEG2—NHS (Monfardini, C., et al., Bioconjugate Chem. 6 (1995) 62-69). Such methods may used to pegylated at an E-amino group of a binding polypeptide lysine or the N-terminal amino group of the binding polypeptide.

In another embodiment, PEG molecules may be coupled to sulfhydryl groups on a binding polypeptide (Sartore, L., et al., Appl. Biochem. Biotechnol., 27, 45 (1991); Morpurgo et al., Biocon. Chem., 7, 363-368 (1996); Goodson et al., Bio/Technology (1990) 8, 343; U.S. Pat. No. 5,766,897). U.S. Pat. Nos. 6,610,281 and 5,766,897 describes exemplary reactive PEG species that may be coupled to sulfhydryl groups.

In some embodiments where PEG molecules are conjugated to cysteine residues on a binding polypeptide, the cysteine residues are native to the binding polypeptide, whereas in other embodiments, one or more cysteine residues are engineered into the binding polypeptide. Mutations may be introduced into an binding polypeptide coding sequence to generate cysteine residues. This might be achieved, for example, by mutating one or more amino acid residues to cysteine. Preferred amino acids for mutating to a cysteine residue include serine, threonine, alanine and other hydrophilic residues. Preferably, the residue to be mutated to cysteine is a surface-exposed residue. Algorithms are well-known in the art for predicting surface accessibility of residues based on primary sequence or a protein. Alternatively, surface residues may be predicted by comparing the amino acid sequences of binding polypeptides, given that the crystal structure of the framework based on which binding polypeptides are designed and evolved has been solved (see Himanen et al., Nature. (2001) 20-27; 414(6866):933-8) and thus the surface-exposed residues identified. In one embodiment, cysteine residues are introduced into binding polypeptides at or near the N- and/or C-terminus, or within loop regions.

In some embodiments, the pegylated binding polypeptide comprises a PEG molecule covalently attached to the alpha amino group of the N-terminal amino acid. Site specific N-terminal reductive amination is described in Pepinsky et al., (2001) JPET, 297, 1059, and U.S. Pat. No. 5,824,784. The use of a PEG-aldehyde for the reductive amination of a protein utilizing other available nucleophilic amino groups is described in U.S. Pat. No. 4,002,531, in Wieder et al., (1979) J. Biol. Chem. 254, 12579, and in Chamow et al., (1994) Bioconjugate Chem. 5, 133.

In another embodiment, pegylated binding polypeptide comprises one or more PEG molecules covalently attached to a linker, which in turn is attached to the alpha amino group of the amino acid residue at the N-terminus of the binding polypeptide. Such an approach is disclosed in U.S. Patent Publication No. 2002/0044921 and in WO94/01451.

In one embodiment, a binding polypeptide is pegylated at the C-terminus. In a specific embodiment, a protein is pegylated at the C-terminus by the introduction of C-terminal azido-methionine and the subsequent conjugation of a methyl-PEG-triarylphosphine compound via the Staudinger reaction. This C-terminal conjugation method is described in Cazalis et al., C-Terminal Site-Specific PEGylation of a Truncated Thrombomodulin Mutant with Retention of Full Bioactivity, Bioconjug Chem. 2004; 15(5): 1005-1009.

Monopegylation of a binding polypeptide can also be produced according to the general methods described in WO 94/01451. WO 94/01451 describes a method for preparing a recombinant polypeptide with a modified terminal amino acid alpha-carbon reactive group. The steps of the method involve forming the recombinant polypeptide and protecting it with one or more biologically added protecting groups at the N-terminal alpha-amine and C-terminal alpha-carboxyl. The polypeptide can then be reacted with chemical protecting agents to selectively protect reactive side chain groups and thereby prevent side chain groups from being modified. The polypeptide is then cleaved with a cleavage reagent specific for the biological protecting group to form an unprotected terminal amino acid alpha-carbon reactive group. The unprotected terminal amino acid alpha-carbon reactive group is modified with a chemical modifying agent. The side chain protected terminally modified single copy polypeptide is then deprotected at the side chain groups to form a terminally modified recombinant single copy polypeptide. The number and sequence of steps in the method can be varied to achieve selective modification at the N- and/or C-terminal amino acid of the polypeptide.

The ratio of a binding polypeptide to activated PEG in the conjugation reaction can be from about 1:0.5 to 1:50, between from about 1:1 to 1:30, or from about 1:5 to 1:15. Various aqueous buffers can be used in the present method to catalyze the covalent addition of PEG to the binding polypeptide. In one embodiment, the pH of a buffer used is from about 7.0 to 9.0. In another embodiment, the pH is in a slightly basic range, e.g., from about 7.5 to 8.5. Buffers having a pKa close to neutral pH range may be used, e.g., phosphate buffer.

Conventional separation and purification techniques known in the art can be used to purify PEGylated binding polypeptide, such as size exclusion (e.g. gel filtration) and ion exchange chromatography. Products may also be separated using SDS-PAGE. Products that may be separated include mono-, di-, tri- poly- and un-pegylated binding polypeptide, as well as free PEG. The percentage of mono-PEG conjugates can be controlled by pooling broader fractions around the elution peak to increase the percentage of mono-PEG in the composition. About ninety percent mono-PEG conjugates represents a good balance of yield and activity. Compositions in which, for example, at least ninety-two percent or at least ninety-six percent of the conjugates are mono-PEG species may be desired. In an embodiment of this invention the percentage of mono-PEG conjugates is from ninety percent to ninety-six percent.

In one embodiment, PEGylated binding polypeptide of the invention contain one, two or more PEG moieties. In one embodiment, the PEG moiety(ies) are bound to an amino acid residue which is on the surface of the protein and/or away from the surface that contacts the target ligand. In one embodiment, the combined or total molecular mass of PEG in PEG-binding polypeptide is from about 3,000 Da to 60,000 Da, optionally from about 10,000 Da to 36,000 Da. In a one embodiment, the PEG in pegylated binding polypeptide is a substantially linear, straight-chain PEG.

In one embodiment of the invention, the PEG in pegylated binding polypeptide is not hydrolyzed from the pegylated amino acid residue using a hydroxylamine assay, e.g., 450 mM hydroxylamine (pH 6.5) over 8 to 16 hours at room temperature, and is thus stable. In one embodiment, greater than 80% of the composition is stable mono-PEG-binding polypeptide, more preferably at least 90%, and most preferably at least 95%.

In another embodiment, the pegylated binding polypeptides of the invention will preferably retain at least 25%, 50%, 60%, 70% least 80%, 85%, 90%, 95% or 100% of the biological activity associated with the unmodified protein. In one embodiment, biological activity refers to its ability to bind to VEGFR-2, as assessed by K_{D}, kₒₙ, or k_{off}. In one specific embodiment, the pegylated binding polypeptide protein shows an increase in binding to VEGFR relative to unpegylated binding polypeptide.

The serum clearance rate of PEG-modified polypeptide may be decreased by about 10%, 20%, 30%, 40%, 50%, 60%, 70%, 80%, or even 90%, relative to the clearance rate of the unmodified binding polypeptide. The PEG-modified polypeptide may have a half-life (t_{1/2}) which is enhanced relative to the half-life of the unmodified protein. The half-life of PEG-binding polypeptide may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the unmodified binding polypeptide. In some embodiments, the protein half-life is determined in vitro, such as in a buffered saline solution or in serum. In other embodiments, the protein half-life is an in vivo half life, such as the half-life of the protein in the serum or other bodily fluid of an animal.

### Therapeutic Formulations and Modes of Administration

The present invention provides sustained-release intraocular drug delivery systems that are useful, in particular, for inhibiting VEGF biological activity. Techniques and dosages for administration vary depending on the type of specific polypeptide and the specific condition being treated but can be readily determined by the skilled artisan. In general, regulatory agencies require that a protein reagent to be used as a therapeutic be formulated so as to have acceptably low levels of pyrogens. Accordingly, therapeutic formulations will generally be distinguished from other formulations in that they are substantially pyrogen free, or at least contain no more than acceptable levels of pyrogen as determined by the appropriate regulatory agency (e.g., FDA). A pyrogen may be an endotoxin or exotoxin. In some embodiments, the drug delivery system is substantially endotoxin free.

Therapeutic compositions of the present invention may be administered with a pharmaceutically acceptable diluent, carrier, or excipient, in unit dosage form. Methods well known in the art for making formulations are found, for example, in “Remington: The Science and Practice of Pharmacy” (20th ed., ed. A. R. Gennaro A R., 2000, Lippincott Williams & Wilkins, Philadelphia, Pa.). Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. The concentration of the compound in the formulation varies depending upon a number of factors, including the dosage of the drug to be administered, and the route of administration.

The antiangiogenic polypeptide may be optionally administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like. In one example, the polypeptide is formulated in the presence of sodium acetate to increase thermal stability.

The antiangiogenic polypeptides may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

A therapeutically effective dose refers to a dose that produces the therapeutic effects for which it is administered. The exact dose will depend on the disorder to be treated, and may be ascertained by one skilled in the art using known techniques. In addition, as is known in the art, adjustments for age as well as the body weight, general health, sex, diet, time of administration, drug interaction, and the severity of the disease may be necessary, and will be ascertainable with routine experimentation by those skilled in the art.

In some embodiments, the sustained-release drug delivery system is a liquid or a gel composition, suitable for injection into the ocular region of a patient. In some embodiments, the sustained release drug delivery system is a biodegradable implant. The drug system is injected intraocularly, such as an intravitreal, subconjunctival injection, or subtenon injection; and the resulting implant releases drug over a predetermined interval of time. Typically, the implant biodegrades at the same rate that the drug is released; therefore, the injection site essentially resolves in time for the next injection.

Sustained-release drug delivery systems also include semipermeable matrices of solid hydrophobic polymers containing the proteins of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated proteins of the invention may remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

In some embodiments, the sustained release drug delivery system utilizes the Atrigel™ system comprising lactide/glycolide copolymers as described in U.S. Patent Application 20060210604. In some embodiments, the sustained release drug delivery system utilizes a biodegradable PLGA intravitreal implant as described in U.S. Patent Application 20050244469.

Other drug delivery systems have been previously described and may be used to deliver the antiangiogenic polypeptide component. The following is a list of suitable implants that may be used in the drug delivery system of the invention. U.S. Pat. No. 5,501,856 discloses controlled release pharmaceutical preparations for intraocular implants to be applied to the interior of the eye after a surgical operation for disorders in retina/vitreous body or for glaucoma. U.S. Pat. No. 5,869,079 discloses combinations of hydrophilic and hydrophobic entities in a biodegradable sustained release implant, and describes a polylactic acid polyglycolic acid (PLGA) copolymer implant comprising dexamethasone. As shown by in vitro testing of the drug release kinetics, the 100-120 .mu.g 50/50 PLGA/dexamethasone implant disclosed did not show appreciable drug release until the beginning of the fourth week, unless a release enhancer, such as HPMC was added to the formulation. U.S. Pat. No. 5,824,072 discloses implants for introduction into a suprachoroidal space or an avascular region of the eye, and describes a methylcellulose (i.e. non-biodegradable) implant comprising dexamethasone. WO 9513765 discloses implants comprising active agents for introduction into a suprachoroidal or an avascular region of an eye for therapeutic purposes. U.S. Pat. Nos. 4,997,652 and 5,164,188 disclose biodegradable ocular implants comprising microencapsulated drugs, and describes implanting microcapsules comprising hydrocortisone succinate into the posterior segment of the eye. U.S. Pat. No. 5,164,188 discloses encapsulated agents for introduction into the suprachoroid of the eye, and describes placing microcapsules and plaques comprising hydrocortisone into the pars plana. U.S. Pat. Nos. 5,443,505 and 5,766,242 disclose implants comprising active agents for introduction into a suprachoroidal space or an avascular region of the eye, and describes placing microcapsules and plaques comprising hydrocortisone into the pars plana. Zhou et al. disclose a multiple-drug implant comprising 5-fluorouridine, triamcinolone, and human recombinant tissue plasminogen activator for intraocular management of proliferative vitreoretinopathy (PVR). Zhou, T, et al. (1998). Development of a multiple-drug delivery implant for intraocular management of proliferative vitreoretinopathy, Journal of Controlled Release 55: 281-295. U.S. Pat. No. 6,369,116 discusses an implant with a release modifier inserted within a scleral flap. EP 0 654256 discusses use of a scleral plug after surgery on a vitreous body, for plugging an incision. U.S. Pat. No. 4,863,457 discusses the use of a bioerodible implant to prevent failure of glaucoma filtration surgery by positioning the implant either in the subconjunctival region between the conjunctival membrane overlying it and the sclera beneath it or within the sclera itself within a partial thickness sclera flap. EP 488 401 discusses intraocular implants, made of certain polylactic acids, to be applied to the interior of the eye after a surgical operation for disorders of the retina/vitreous body or for glaucoma. EP 430539 discusses use of a bioerodible implant which is inserted in the suprachoroid.

The amount of drug delivery system administered will typically depend upon the desired properties of the biodegradable implant For example, the amount of drug delivery system can influence the length of time in which the antiangiogenic polypeptide component is released from the biodegradable implant Additionally, the amount of drug delivery system administered will typically depend upon the specific intended use (e.g., nature and stage/progression of the disease or disorder).

Specifically, the drug delivery system can be formulated to provide an implant that releases therapeutically effective amounts of an antiangiogenic polypeptide for at least one week, two weeks, one month, two months, three months, four months, five months, six months, nine months, twelve months or more. Specifically, the drug delivery system can be formulated for administration less than about once per day. More specifically, the drug delivery system can be formulated for administration less than about once per week, less than about once per month, more than about once per year, about once per week to about once per year, or about once per month to about once per year.

In some embodiments, less than 5 ml, 4 ml, 3 ml, 2 ml, 1 ml, 0.1 ml, 0.01 ml, or 0.001 ml is administered. Specifically, the drug delivery system administered can range from about 0.01 mL to about 10.0 mL, about 0.05 mL to about 1.5 mL, about 0.1 mL to about 1.0 mL, or about 0.2 mL to about 0.8 mL.

The antiangiogenic polypeptide component can be present in any effective, suitable and appropriate amount. For example, polypeptide component can be present up to about 70 wt. % of the drug delivery system, up to about 60 wt. % of the drug delivery system, up to about 40 wt. % of the drug delivery system, up to about 20 wt. % of the drug delivery system, 10 wt. % of the drug delivery system, up to about 5 wt. % of the drug delivery system, up to about 1 wt. % of the drug delivery system, or up to about 0.1 wt. % of the drug delivery system.

The drug delivery system will effectively deliver the antiangiogenic polypeptide component to mammalian tissue at a suitable, effective, safe, and appropriate dosage. For example, the drug delivery system can effectively deliver the antiangiogenic polypeptide component to mammalian tissue at a dosage of more than about 0.001 picogram/kilogram/day, more than about 0.01 picogram/kilogram/day, more than about 0.1 picogram/kilogram/day, or more than about 1 picogram/kilogram/day. Alternatively, the drug delivery system can effectively deliver the antiangiogenic polypeptide component to mammalian tissue at a dosage of up to about 100 milligram/kilogram/day, up to about 50 milligram/kilogram/day, up to about 10 milligram/kilogram/day, or up to about 1 milligram/kilogram/day.

More specifically, the drug delivery system can effectively deliver the antiangiogenic polypeptide component to mammalian tissue at a dosage of about 0.001 picogram/kilogram/day to about 100 milligram/kilogram/day; about 0.01 picogram/kilogram/day to about 50 milligram/kilogram/day; about 0.1 picogram/kilogram/day to about 10 milligram/kilogram/day; or about 1 picogram/kilogram/day to about 1 milligram/kilogram/day.

The sustained-release intraocular drug delivery system can further comprise analgesics, anesthetics, anti-infective agents, or anti-steroidal agents. Suitable analgesics include, e.g., acetaminophen, phenylpropanolamine HCl, chlorpheniramine maleate, hydrocodone bitartrate, acetaminophen elixir, diphenhydramine HCl, pseudoephedrine HCl, dextromethorphan HBr, guaifenesin, doxylamine succinate, pamabron, clonidine hydrochloride, tramadol hydrochloride, carbamazepine, sodium hyaluronate, lidocaine, hylan, Arnica Montana, radix (mountain arnica), Calendula officinalis (marigold), Hamamelis (witch hazel), Millefolium (milfoil), Belladonna (deadly nightshade), Aconitum napellus (monkshood), Chamomilla (chamomile), Symphytum officinale (comfrey), Bellis perennis (daisy), Echinacea angustifolia (narrow-leafed cone flower), Hypericum perforatum (St. John's wort), Hepar sulphuris calcareum (calcium sulfide), buprenorphine hydrochloride, nalbuphine hydrochloride, pentazocine hydrochloride, acetylsalicylic acid, salicylic acid, naloxone hydrochloride, oral transmucosal fentanyl citrate, morphine sulfate, propoxyphene napsylate, propoxyphene hydrochloride, meperidine hydrochloride, hydromorphone hydrochloride, fentanyl transdermal system, levorphanol tartrate, promethazine HCl, oxymorphone hydrochloride, levomethadyl acetate hydrochloride, oxycodone HCl, oxycodone, codeine phosphate, isometheptene mucate, dichloralphenazone, butalbital, naproxen sodium, diclofenac sodium, misoprostol, diclofenac potassium, celecoxib, sulindac, oxaprozin, salsalate, diflunisal, naproxen, piroxicam, indomethacin, indomethacin sodium trihydrate, etodolac, meloxicam, ibuprofen, fenoprofen calcium, ketoprofen, mefenamic acid, nabumetone, tolmetin sodium, ketorolac tromethamine, choline magnesium trisalicylate, and rofecoxib.

Suitable anesthetics include: propofol, halothane, desflurane, midazolam HCl, epinephrine, levobupivacaine, etidocaine hydrochloride, ropivacaine HCl, chloroprocaine HCl, bupivacaine HCl, and lidocaine HCl.

Suitable anti-infective agents include, e.g., trimethoprim, sulfamethoxazole, clarithromycin, ganciclovir sodium, ganciclovir, daunorubicin citrate liposome, fluconazole, doxorubicin HCl liposome, foscamet sodium, interferon alfa-2b, atovaquone, rifabutun, trimetrexate glucoronate, itraconazole, ciclofovir, azithromycin, delavirdine mesylate, efavirenz, nevirapine, lamivudine/zidovudine, zalcitabine, didanosine, stavudine, abacavir sulfate, amprenavir, indinavir sulfate, saquinavir, saquinavir mesylate, ritonavir, nelfinavir, chloroquine hydrochloride, metronidazole, metronidazole hydrochloride, iodoquinol, albendazole, praziquantel, thiabendazole, ivermectin, mebendazole sulfate, tobramycin sulfate, tobramycin, azetreonam, cefotetan disodium, cefotetan, loracarbef, cefoxitin, meropenem, imipenemand cilastatin, cefazolin, cefaclor, ceftibuten, ceftizoxime, cefoperazone, cefuroxumeaxetil, cefprozil, ceftazidime, cefotaxime sodium, cefadroxil monohydrate, cephalexin, cephalexin hydrochloride, cefuroxime, cefazolin, cefamandole nafate, cefapime hydrochloride, cefdinir, ceftriaxone sodium, cefixme, cefpodoxime proxetil, dirithromycin, erythromycin, erythromycin ethylsuccinate, erythromycin stearate, erythromycin, sulfisoxazole acetyl, troleandomycin, azithromycin, clindamycin, clindamycin hydrochloride, colistimethate sodium, quinupristin/dalfopristin, vancomycin hydrochloride, amoxicillin, amoxicillin/calvulanate/potassium, penicillin G benzathine, penicillin G procaine, penicillin G potassium, carbenicillin indanyl sodium, piperacillin sodium, ticarcillin disodium, clavulanate potassium, ampicillin sodium/sulbactam sodium, tazobactam sodium, tetracycline HCl, demeclocycline hydrochloride, doxycycline hyclate, minocycline HCl, doxycycline monohydrate, oxytetracycline HCl, hydrocortisone acetate, doxycycline calcium, amphotericin B lipid, flucytosine, griseofulvin, terbinafine hydrochloride, ketoconazole, chloroquine hydrochloride, chloroquine phosphate, pyrimethamine, mefloquine hydrochloride, atovaquone and proguanil hydrochloride, hydroxychloroquine sulfate, ethambutol hydrochloride, aminosalicylic acid, rifapentine, rifampin, isoniazid, pyrazinamide, ethionamide, interferon alfa-n3, famciclovir, rimantadine hydrochloride, foscamet sodium, interferon alfacon-1, ribavirin, zanamivir, amantadine hydrochloride, palivizumab, oseltamivir phosphate, valacyclovir hydrochloride, nelfinavir mesylate, stavudine, acyclovir, acyclovir sodium, rifabutin, trimetrexate glucuronate, linezolid, moxifloxacin, moxifloxacin hydrochloride, ciprofloxacin, ciprofloxacin hydrochloride, ofloxacin, levofloxacin, lomefloxacin hydrochloride, nalidixic acid, norfloxacin, enoxacin, gatifloxacin, trovafloxacin mesylate, alatrofloxacin, sparfloxacin, aztreonam, nitrofurantoin monohydrate/macrocrystals, cefepime hydrochloride, fosfomycin tromethamine, neomycin sulfate-polymyxin B sulfate, imipenem, cilastatin, methenamine, methenamine mandelate, phenyl salicylate, atropine sulfate, hyoscyamine sulfate, benzoic acid, oxytetracycline hydrochloride, sulfamethizole, phenazopyridine hydrochloride, and sodium acid phosphate, monohydrate.

The steroidal anti-inflammatory agents that may be used in the ocular implants include, but are not limited to, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and any of their derivatives.

### Exemplary Uses

The small size and stable structure of the disclosed polypeptides can be particularly valuable with respect to manufacturing of the drug, rapid clearance from the body for certain applications where rapid clearance is desired or formulation into novel delivery systems that are suitable or improved using a molecule with such characteristics.

On the basis of their efficacy as inhibitors of VEGF biological activity, the polypeptides of the invention are effective against a number of conditions associated with inappropriate angiogenesis, including but not limited to autoimmune disorders (e.g., rheumatoid arthritis, inflammatory bowel disease or psoriasis); cardiac disorders (e.g., atherosclerosis or blood vessel restenosis); retinopathies (e.g., proliferative retinopathies generally, diabetic retinopathy, age-related macular degeneration or neovascular glaucoma), renal disease (e.g., diabetic nephropathy, malignant nephrosclerosis, thrombotic microangiopathy syndromes; transplant rejection; inflammatory renal disease; glomerulonephritis; mesangioproliferative glomerulonephritis; haemolytic-uraemic syndrome; and hypertensive nephrosclerosis); hemangioblastoma; hemangiomas; thyroid hyperplasias; tissue transplantations; chronic inflammation; Meigs's syndrome; pericardial effusion; pleural effusion; autoimmune diseases; diabetes; endometriosis; chronic asthma; undesirable fibrosis (particularly hepatic fibrosis) and cancer, as well as complications arising from cancer, such as pleural effusion and ascites. Preferably, the VEGFR-binding polypeptides of the invention can be used for the treatment of prevention of hyperproliferative diseases or cancer and the metastatic spread of cancers. Non-limiting examples of cancers include bladder, blood, bone, brain, breast, cartilage, colon kidney, liver, lung, lymph node, nervous tissue, ovary, pancreatic, prostate, skeletal muscle, skin, spinal cord, spleen, stomach, testes, thymus, thyroid, trachea, urogenital tract, ureter, urethra, uterus, or vaginal cancer. Additional treatable conditions can be found in U.S. Pat. No. 6,524,583, herein incorporated by reference. Other references describing uses for VEGFR-2 binding polypeptides include: McLeod D S et al., Invest Opthalmol V is Sci. 2002 February; 43(2):474-82; Watanabe et al. Exp Dermatol. 2004 Nov.; 13(11):671-81; Yoshiji H et al., Gut. 2003 September; 52(9):1347-54; Verheul et al., Oncologist. 2000; 5 Suppl 1:45-50; Boldicke et al., Stem Cells. 2001; 19(1):24-36.

As described herein, angiogenesis-associated diseases include, but are not limited to, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; inflammatory disorders such as immune and non-immune inflammation; chronic articular rheumatism and psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation and wound healing; telangiectasia psoriasis scleroderma, pyogenic granuloma, cororany collaterals, ischemic limb angiogenesis, corneal diseases, rubeosis, arthritis, diabetic neovascularization, fractures, vasculogenesis, hematopoiesis.

In particular, the sustained-release intraocular drug delivery system is useful for the treatment of retinopathies, such as retinal vein occlusion, diabetic macular edema, diabetic retinopathy, retinopathy of prematurity, macular degeneration, age-related macular degeneration, corneal graft rejection, neovascular glaucoma, retrolental fibroplasia, and rubeosis.

In one embodiment, the drug delivery system administers a therapeutic component to ameliorate inflammation, and thus to control, reduce or prevent an inflammatory response or ameliorate the effects of an inflammatory response. In one embodiment, the therapeutic component is used to enhance reabsorption of inflammatory exudates. Decreasing the level of exudates in the eye reduces the inflammatory process and the ensuing hyperpermeable state that occurs with allergies, infection, responses to ocular photodynamic therapy (PDT) and laser treatments, after ocular surgery or trauma, etc.

In one embodiment, the therapeutic component is administered to ameliorate the scarring and adhesions that are a part of the inflammatory process. Adhesions are bands of scar tissue that bind two internal body surfaces. They are an inflammatory response to tissue damage, and occur as a normal part of any healing process. As one example, adhesions frequently occur during the post-surgical healing process during which tissues have experienced mechanical trauma. However, adverse effects can occur when internal surfaces bind, and adhesions may persist even after the original trauma has healed. Surgery to repair adhesions itself results in recurrent or additional adhesions. The presence of adhesions may also complicate surgical procedures, for example, ocular conjunctival adhesions may complicate subsequent glaucoma surgery.

Adhesions can occur following any type of trauma or surgery, including but not limited to ocular surgery. Examples of ocular surgery that may result in adhesions include glaucoma filtration operations (i.e., iridencleisis and trephination, pressure control valves), extraocular muscle surgery, diathermy or scleral buckling surgery for retinal detachment, and vitreous surgery. Examples of ocular trauma include penetrating ocular injuries, intraocular foreign body, procedures such as PDT, scatter laser threshold coagulation, refractive surgery, and blunt trauma.

In one embodiment, the therapeutic component ameliorates disorders with both a vascular proliferative component and a scarring component. As one example, the invention may be used in patients with the ocular disease pterygia. In these patients, fibrovascular proliferation results in scarring of the conjunctiva. An elevated, superficial, external ocular mass, termed a pterygium, forms and extends onto the corneal surface. Patients may experience symptoms of inflammation (e.g., redness, swelling, itching, irritation) and blurred vision. The mass itself may become inflamed, resulting in redness and ocular irritation. Left untreated, pterygia can distort the corneal topography, obscure the optical center of the cornea, and result in altered vision.

The process whereby scar tissue forms (scarring) can occur without new blood vessels being formed (neovascularization). However, the neovascularization process always results in scarring because of the cell proliferation that occurs with the formation of new vessels also results in the proliferation of fibroblasts, glial cells, etc. that result in scar tissue formation. The inventive method may be used to ameliorate the scarring process.

In one embodiment, the therapeutic component is administered to ameliorate inflammation of uveal tissues (uveitis, an inflammation of tissues in the middle layer of the eye, mainly the iris (iritis) and the ciliary body). Ocular inflammation may be associated with underlying systemic disease or autoimmunity, or may occur as a direct result of ocular trauma or infectious agents (bacterial, viral, fungal, etc.). Inflammatory reactions in adjacent tissues, e.g., keratitis, can induce a secondary uveitis. There are both acute and chronic forms of uveitis. The chronic form is frequently associated with many systemic disorders and most likely occurs due to immunopathological mechanisms.

Uveitis presents with ocular pain, photophobia and hyperlacrimation, with decreased visual acuity ranging from mild blur to significant vision loss. Hallmark signs of anterior uveitis are cells and flare in the anterior chamber. If the anterior chamber reaction is significant, small gray to brown endothelial deposits known as keratic precipitates may arise, leading to endothelial cell dysfunction and corneal edema. There may be adhesions to the lens capsule (posterior synechia) or the peripheral cornea (anterior synechia). Granulomatous nodules may appear on the surface of the iris stroma. Intraocular pressure is initially reduced due to secretory hypotony of the ciliary body but, as the reaction persists, inflammatory by-products may accumulate in the trabeculum. If this debris builds significantly, and if the ciliary body resumes its normal secretory output, the pressure may rise sharply, resulting in a secondary uveitic glaucoma.

A VEGFR-2 binding polypeptide can be administered alone or in combination with one or more additional therapies such as chemotherapy radiotherapy, immunotherapy, surgical intervention, or any combination of these. Long-term therapy is equally possible as is adjuvant therapy in the context of other treatment strategies, as described above.

In certain embodiments of such methods, one or more polypeptide therapeutic agents can be administered, together (simultaneously) or at different times (sequentially). In addition, polypeptide therapeutic agents can be administered with another type of compounds for treating cancer or for inhibiting angiogenesis.

In certain embodiments, the subject therapeutic agents of the invention can be used alone. Alternatively, the subject agents may be used in combination with conventional anti-cancer therapeutic approaches directed to treatment or prevention of proliferative disorders (e.g., tumor). For example, such methods can be used in prophylactic cancer prevention, prevention of cancer recurrence and metastases after surgery, and as an adjuvant of other conventional cancer therapy. The present invention recognizes that the effectiveness of conventional cancer therapies (e.g., chemotherapy, radiation therapy, phototherapy, immunotherapy, and surgery) can be enhanced through the use of a subject polypeptide therapeutic agent.

A wide array of conventional compounds have been shown to have anti-neoplastic activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When the drug delivery system of the present invention is administered in combination with a conventional anti-neoplastic agent, either concomitantly or sequentially, such drug delivery system may be found to enhance the therapeutic effect of the anti-neoplastic agent or overcome cellular resistance to such anti-neoplastic agent. This allows decrease of dosage of an anti-neoplastic agent, thereby reducing the undesirable side effects, or restores the effectiveness of an anti-neoplastic agent in resistant cells.

The therapeutic agents that can be combined with the sustained-release drug delivery system of the invention include diverse agents used in oncology practice (Reference: Cancer, Principles & Practice of Oncology, DeVita, V. T., Hellman, S., Rosenberg, S. A., 6th edition, Lippincott-Raven, Philadelphia, 2001), such as, merely to illustrate: abarelix, altretamine, aminoglutethimide, amsacrine, anastrozole, antide, asparaginase, AZD2171 (Recentin™), Bacillus Calmette-Guerin/BCG (TheraCys™, TICE™), bevacizumab (see U.S. Pat. No. 6,054,297; Avastin™), bicalutamide, bleomycin, bortezomib (Velcade™), buserelin, busulfan, campothecin, capecitabine, carboplatin, carmustine, cetuximab (Erbitux™), chlorambucil, cisplatin, cladribine, clodronate, colchicine, cyclophosphamide, cyproterone, cytarabine, dacarbazine, dactinomycin, dasatinib ((see U.S. Pat. No. 6,596,746 Sprycel™), daunorubicin, dienestrol, diethylstilbestrol, dexamethasone, docetaxel (Taxotere™), doxorubicin, Abx-EGF, epothilones, epirubicin, erlonitib (Tarceva™), estradiol, estramustine, etoposide, exemestane, 5-fluorouracil, filgrastim, fludarabine, fludrocortisone, fluorouracil, fluoxymesterone, flutamide, fulvestrant, gefitinib (Iressa™), gemcitabine (see U.S. Pat. No. 4,808,614; Gemzar™), genistein, goserelin, hydroxyurea, idarubicin, ifosfamide, imatinib mesylate (see U.S. Pat. No. 5,521,184; Gleevac™), interferon, irinotecan, ibritumomab (Zevalin™), ironotecan, ixabepilone (BMS-247550), lapatinib (see U.S. Pat. No. 6,391,874; Tykreb™), letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, motesanib diphosphate (AMG 706) nilutamide, nocodazole, octreotide, oxaliplatin, paclitaxel (Taxol™), pamidronate, pentostatin, plicamycin, porfimer, procarbazine, raltitrexed, rapamycin, rituximab (Rituxan™), sorafenib (Nexavar™/Bayer BAY43-9006), streptozocin, suramin, sunitinib malate (see U.S. Pat. No. 6,573,293; Sutent™), tamoxifen, temsirolimus (see U.S. Pat. No. 5,362,718; CCl-779), temozolomide (see U.S. Pat. No. 5,260,291; Temodar™), teniposide, testosterone, thioguanine, thiotepa, titanocene dichloride, topotecan, toremifene, tositumomab (Bexxar™), trastuzumab (U.S. Pat. No. 5,821,337; Herceptin™), tretinoin, VEGF Trap (aflibercept; preparation described in U.S. Pat. No. 5,844,099), vinblastine, vincristine, vindesine, and vinorelbine, zoledronate.

Certain chemotherapeutic anti-tumor compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes—dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (e.g., VEGF inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In certain embodiments, pharmaceutical compounds that may be used for combinatory anti-angiogenesis therapy include: (1) inhibitors of release of “angiogenic molecules,” such as bFGF (basic fibroblast growth factor); (2) neutralizers of angiogenic molecules, such as an anti-βbFGF antibodies; and (3) inhibitors of endothelial cell response to angiogenic stimuli, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D₃ analogs, alpha-interferon, and the like. For additional proposed inhibitors of angiogenesis, see Blood et al., Bioch. Biophys. Acta., 1032:89-118 (1990), Moses et al., Science, 248:1408-1410 (1990), Ingber et al., Lab. Invest., 59:44-51 (1988), and U.S. Pat. Nos. 5,092,885, 5,112,946, 5,192,744, 5,202,352, and 6573256. In addition, there are a wide variety of compounds that can be used to inhibit angiogenesis, for example, endostatin protein or derivatives, lysine binding fragments of angiostatin, melanin or melanin-promoting compounds, plasminogen fragments (e.g., Kringles 1-3 of plasminogen), tropoin subunits, antagonists of vitronectin αᵥβ₃, peptides derived from Saposin B, antibiotics or analogs (e.g., tetracycline, or neomycin), dienogest-containing compositions, compounds comprising a MetAP-2 inhibitory core coupled to a peptide, the compound EM-138, chalcone and its analogs, and naaladase inhibitors. See, for example, U.S. Pat. Nos. 6,395,718, 6,462,075, 6,465,431, 6,475,784, 6,482,802, 6,482,810, 6,500,431, 6,500,924, 6,518,298, 6,521,439, 6,525,019, 6,538,103, 6,544,758, 6,544,947, 6,548,477, 6,559,126, and 6,569,845.

Depending on the nature of the combinatory therapy, administration of the drug delivery system of the invention may be continued while the other therapy is being administered and/or thereafter. Administration of the drug delivery system may be made in a single dose, or in multiple doses. In some instances, administration of the drug delivery system is commenced at least several days prior to the conventional therapy, while in other instances, administration is begun either immediately before or at the time of the administration of the conventional therapy.

### EXAMPLES

The following examples are for the purposes of illustrating the invention, and should not be construed as limiting.

### Example 1

### Initial Identification of KDR Binding Molecules

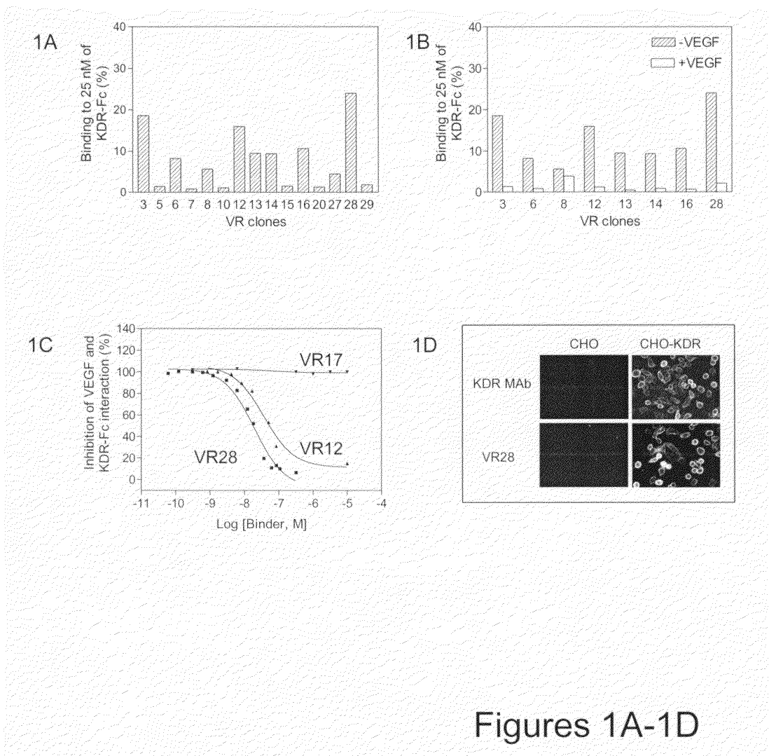

A library of approximately 1013 RNA-protein fusion variants was constructed based on the scaffold of the tenth type 3 domain of human fibronectin with three randomized regions at positions 23-29, 52-55 and 77-86 (amino acid nos. are referenced to SEQ ID NO:5) (three loop library; Xu et al, Chemistry & Biology 9:933-942, 2002). Similar libraries were constructed containing randomized regions only at positions 23-29 and 77-86 (two loop library) or only at positions 77-86 (one loop library). A mixture of these three libraries was used for in vitro selection against the extracellular domain of human VEGFR-2 (KDR, extracellular domain, residues 1-764 fused to human IgG1 Fc). For the purposes of this application, the amino acid positions of the loops will be defined as residues 23-30 (BC Loop), 52-56 (DE Loop) and 77-87 (FG Loop). The target binding population was analyzed by DNA sequencing after six rounds of selection and was found to be diverse, with some replicates present. Proteins encoded by fifteen independent clones were screened for binding to KDR, (FIG. 1A) and the best binders were subsequently analyzed for inhibition of target binding in the presence of VEGF (FIG. 1B). Multiple clones were identified that inhibited KDR-VEGF binding, suggesting that these clones bound KDR at or near the natural ligand (VEGF) binding site. The ability of two of the binding molecules (VR28 and VR12) to directly inhibit VEGF-KDR interaction was evaluated in a BIAcore assay using immobilized VEGF and a mobile phase containing KDR-Fc with or without a selected binding protein. VR28 and, to a lesser extent, VR12, but not a non-competing clone (VR17), inhibited KDR binding to VEGF in a dose dependent manner (FIG. 1C). Finally, in addition to binding to purified recombinant KDR, VR28 also appeared to bind to KDR-expressing recombinant CHO cells, but not to control CHO cells (FIG. 1D).

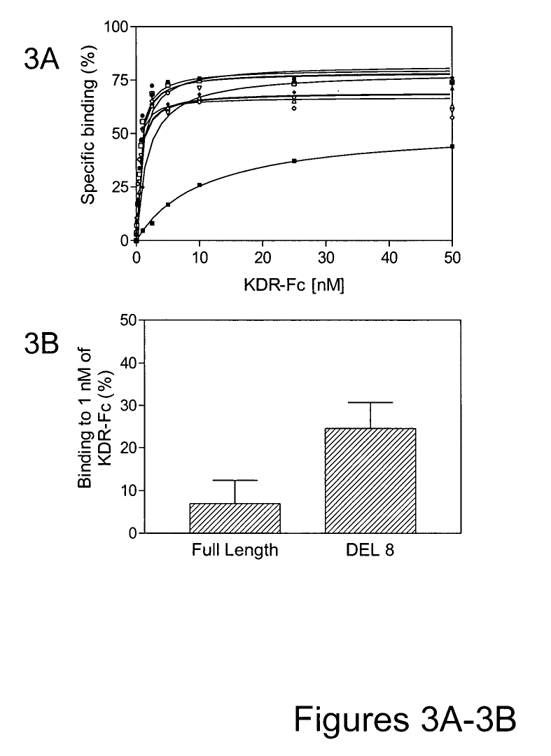

The sequence of the binding loops of the VR28 clone is shown in the first row of Table 4. While VR28 was not the most abundant clone in the sequenced binding population (one copy out of 28 sequenced clone), its binding affinity to KDR was the best among the tested clones from this binding population, with a dissociation constant of 11-13 nM determined in a radioactive equilibrium binding assay (FIG. 3 and Table 5) and BIAcore assays (Table 7). There were no changes from wild type ¹⁰Fn3 in the remaining scaffold portion of the molecule (following correction of an incidental scaffold change at position 69 that had no effect on binding). However, VR28 showed little inhibition of VEGF-KDR signaling in a VEGF-dependent cell proliferation assay. Thus, while the selection from the naïve library yielded antibody mimics that interfered with the interaction between VEGF and KDR in biochemical binding studies, affinity improvements were useful for neutralizing function in a biological signal transduction assay.

### Example 2

### Affinity Maturation of Clone VR28

A mutagenesis strategy focusing on altering sequences only in the binding loops was employed. To initially test which loops were more likely to result in improvement, loop-directed hypermutagenic PCR was carried out to introduce up to 30% mutations independently into each loop of VR28. After three rounds of selection against KDR, multiple clones with improved binding to KDR-Fc were observed. Sequence analysis of the selection pools revealed that the majority of mutations were accumulated in the FG loop while the BC and DE loops remained almost intact. This result indicated that the FG loop was the most suitable target for further modification.

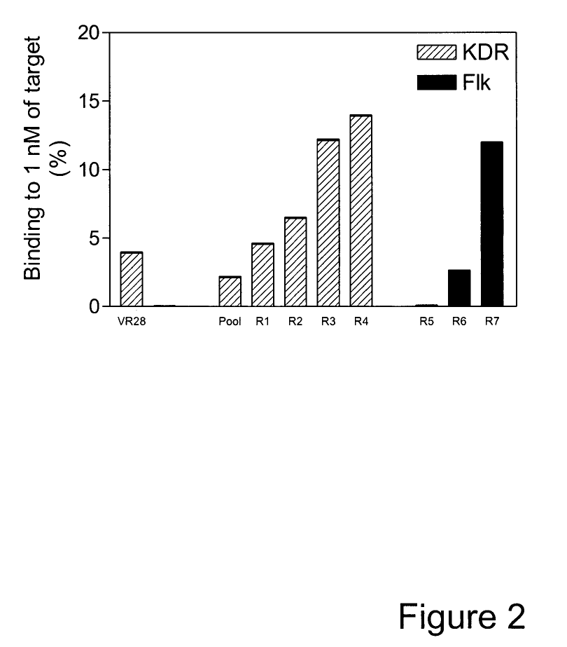

Consequently, a new library of approximately 1012 variants was constructed by altering the sequence of VR28 in the FG loop using oligonucleotide mutagenesis. For each of the FG loop positions (residues 77-86 [VAQNDHELIT (SEQ ID NO:198)] as well as the following Proline [residue 87]), a 50:50 mixture of the VR28-encoding DNA and NNS was introduced at each position. DNA sequence analysis of a random sample of approximately 80 clones revealed an average of six amino acid changes per clone as expected. Lower KDR-Fc concentrations were utilized during selection to favor clones with better affinities to the target. The profile of target binding during the four rounds of selection is shown in FIG. 2. After four rounds of selection the binding population was subcloned and analyzed. Table 5 and FIG. 3A summarize affinity measurements of individual binding clones. The measured binding constants to KDR-Fc ranged from <0.4 to <1.8 nM, a 10-30 improvement over VR28 (11 nM).

Sequence analysis, some of which is shown in Table 4 (K clones), revealed that while the binding population was diverse, several consensus motifs could be identified among the clones. Most noticeably, Pro87 and Leu84 were found in nearly all clones (as in VR28), suggesting that these residues may be essential for the structure of the binding site. A positively charged amino acid at position 82 appears to be required since only H82K or H82R changes were seen in the sequenced clones and an aliphatic amino acid was predominant at position 78. D81 was often mutated to a G, resulting in the loss of negative charge at this position and a gain in flexibility. In addition, the overall mutation rate in the selected population was comparable to the pool prior to selection, which suggested that the FG loop is very open to changes.

Several residues in the N-terminus of the ¹⁰Fn3 domain of human fibronectin are located in close proximity to the FG loop, as suggested by structural determinations (Main et al, Cell 71:671-678, 1992). The close proximity of the two regions could potentially have a negative impact on target binding. Two incidental mutations in the N-terminal region, L8P and L8Q, resulted in better binding to KDR in a number of selected clones, presumably due to a change of the location of the N-terminus relative to the FG loop. To further test the impact of the N-terminus, we created binding molecules for 23 different KDR binders in which the N-terminal first eight residues before the β-sheet were deleted. We then compared target binding to the non-deleted counterparts. On average, binding to KDR-Fc was about 3-fold better with the deletion, as shown in FIG. 3B.

### Example 3

### Selection of Binders with Dual Specificities to Human (KDR) and Mouse (Flk-1) VEGFR-2

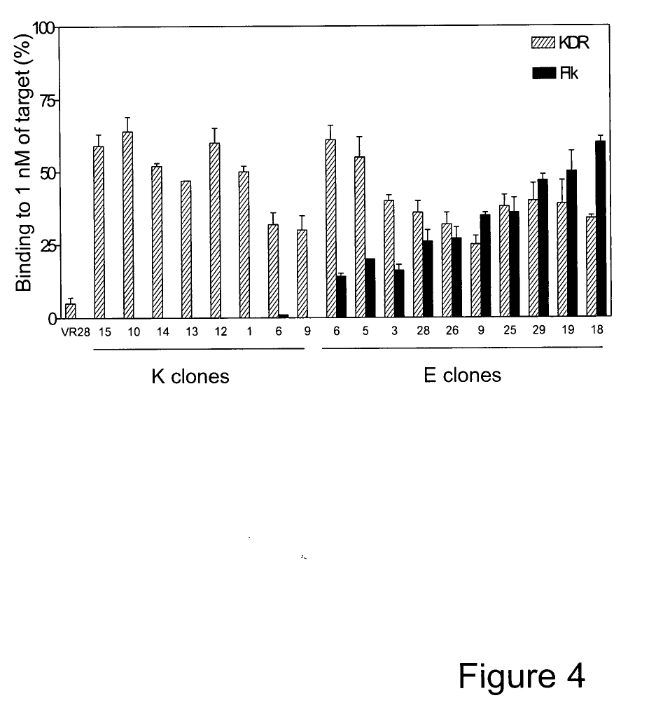

VR28 and most of the affinity matured variants (K clones) failed to bind the mouse homolog of KDR, Flk1, as shown in FIG. 4. However, since KDR and Flk1 share a high level of sequence identity (85%, Claffey et al., J. Biol. Chem. 267:16317-16322 (1992), Shima et al., J. Biol. Chem. 271:3877-3883 (1996)), it is conceivable to isolate antibody mimics that can bind both KDR and Flk1. Such dual binders were desirable because they would allow the same molecule to be tested in functional studies in animal models and subsequently in humans.

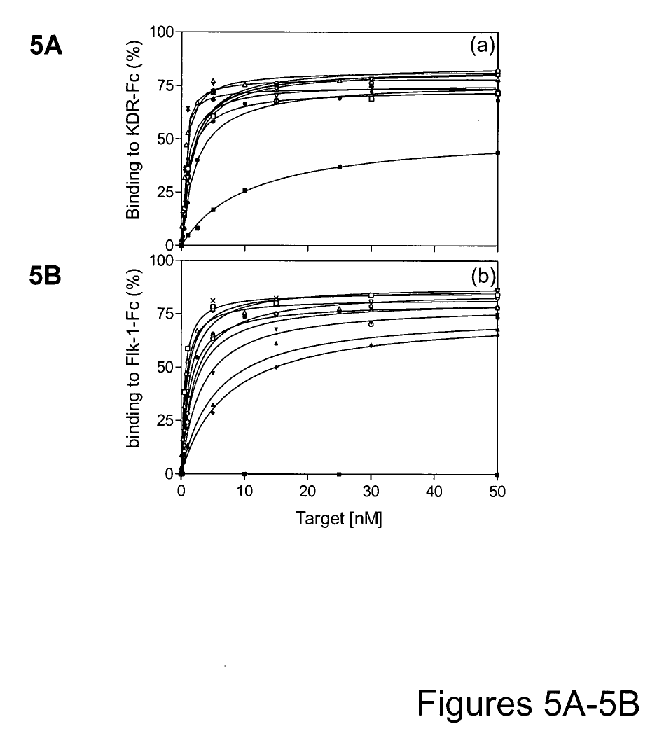

The population of clones following FG loop mutagenesis and selection against KDR for four rounds was further selected against Flk1 for an additional three rounds. As shown in FIG. 2 an increase in binding to Flk1 was observed from Round 5 to Round 7, indicating enrichment of Flk1 binders. Analysis of binding for multiple individual clones revealed that in contrast to the clones selected against KDR only (K clones), most clones derived from additional selection against Flk1 (E clones) are able to interact with both KDR and Flk1. The binding constants to both targets, as determined using a radioactive equilibrium binding assay (Table 6 and FIG. 5) and BIAcore (Table 7), indicate that individual clones were able to bind both targets with high affinities.

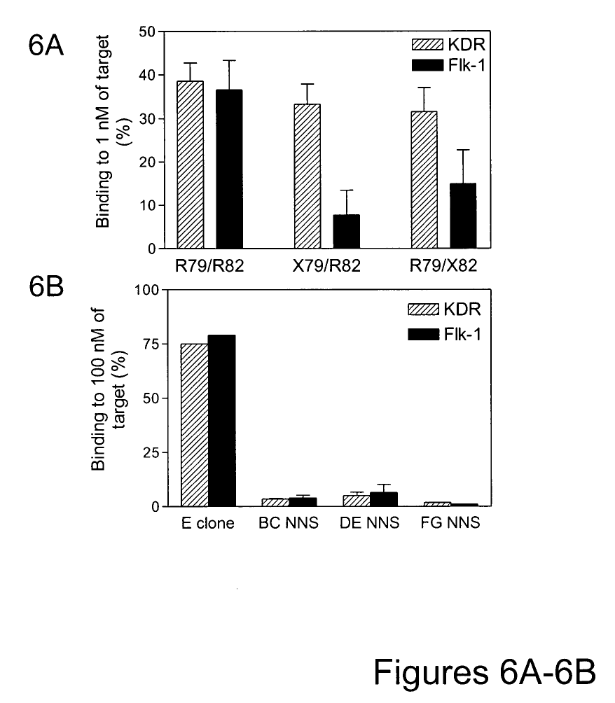

For example, E19 has a Kd of 60 pM to KDR, and 340 pM to Flk-1. These results demonstrate that a simple target switch strategy in the selection process, presumably through selection pressures exerted by both targets, has allowed the isolation of molecules with dual binding specificities to both KDR and Flk-1 from a mutagenized population of VR28, a moderate KDR binder that was not able to bind Flk-1. The selected fibronectin-based binding proteins are highly specific to VEGFR-2 (KDR) as no substantial binding to VEGFR1 was observed at high target concentration. Sequence analysis revealed some motifs similar to those observed in the KDR binder pool (Leu and Pro at residues 84 and 87 respectively; positively charged amino acid at residue 82, predominantly Arg) and some that were not maintained (aliphatic at position 78). In addition, the motif ERNGR (residues 78-82) was present in almost all clones binding to Flk-1 (Table 4); this motif was barely discernable in the KDR binding pool. R79 and R82 appear to be particularly important for high affinity binding to Flk-1, since binding to Flk-1, but not KDR, is greatly reduced when a different residue is present at this position (FIG. 6A). To determine the importance of each loop in binding to KDR and Flk-1, the loops of clones E6 and E26 shown in Table 4, were substituted one loop at a time by NNS randomized sequence. As shown in FIG. 6B, after the substitution, the proteins are no longer able to bind either KDR or Flk-1. These results indicate that each loop is required for binding to the targets, suggesting a cooperative participation of all three loops in interacting with the targets.

An alternative mutagenesis strategy was independently employed to produce clones capable of binding to both targets. The clone 159Q(8)L (Table 4), the product of hypermutagenic PCR affinity maturation of VR28 that binds KDR with high affinity (Kd=2 nM; Table 7) and Flk-1 with poor affinity (Kd>3000 nM), was chosen as a starting point. The first six amino acids of the FG loop were fully randomized (NNS), leaving the following five residues (ELFTP) intact. After six rounds of selection against Flk-1, the binding pool was re-randomized at the DE loop (positions 52-56) and the selection was performed for three additional rounds against Flk-1 and one round against KDR. A number of high affinity binding molecules to both KDR and Flk-1 were thus obtained (Tables 4 and FIG. 4). For example, clone M5FL, while retaining high binding affinity to KDR (Kd=890 pM), can bind Flk-1 at a Kd of 2.1 nM, a 1000-fold improvement over the original clone. Interestingly, the ERNGR motif, found in Flk-1 binding molecules selected from a mutagenized population of VR28, was also present in multiple clones derived from clone 159Q(8)L mutagenesis and selection, despite a full randomization of this region of the FG loop. The isolation of similar binding molecules from two independent libraries suggests that the affinity maturation process is robust for isolating optimal Flk-1 binding motifs located in the FG loop.

### Example 4

### Cell Surface Binding and Neutralization of VEGF Activity In Vitro

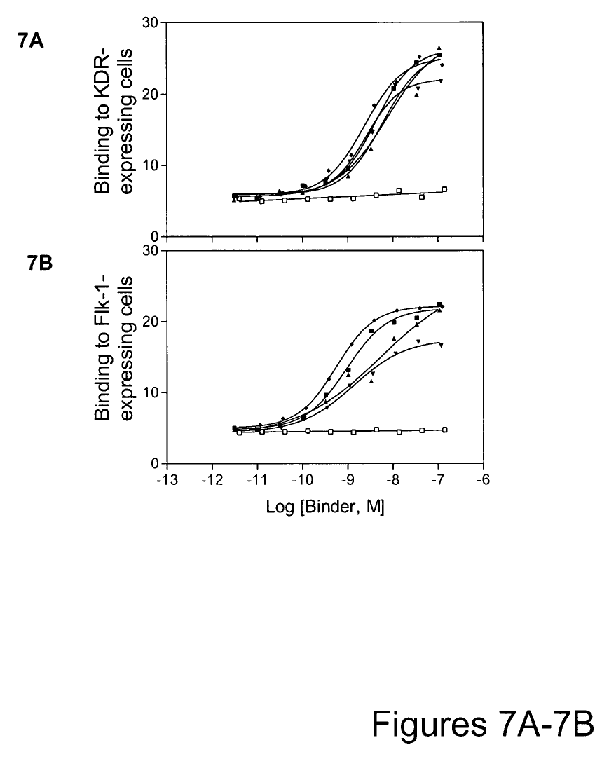

The functionality of KDR and Flk-1 binding molecules in a cell culture model system was evaluated with E. coli produced binding molecules. Using a detection system consisting of anti-His6 tag murine antibody (the E. coli expressed proteins were expressed with a His tag) and an anti-murine fluorescently labeled antibody the binding molecules were shown to bind specifically to mammalian cells expressing KDR or Flk-1 with low nanomolar EC50s, (FIG. 7 and Table 8).

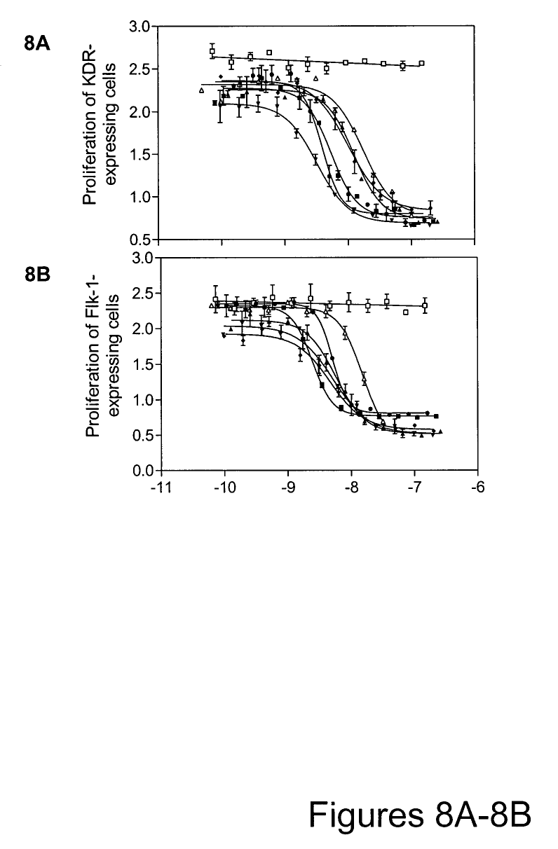

More importantly, using recombinant BA/F3 cells (DSMZ-Deutsche Sammlung von Mikroorganismen und Zellkulturen GmbH) expressing the extracellular KDR or Flk-1 domain linked to the erythropoietin receptor signaling domain, these molecules inhibited VEGF-stimulated cell proliferation in a dose dependent fashion, with IC50 3-12 nM for KDR expressing cells, and 2-5 nM for Flk-1 expressing cells. The potency of inhibition appears to be similar to control anti-KDR and anti-Flk-1 monoclonal antibodies, as shown in FIG. 8 and Table 9.

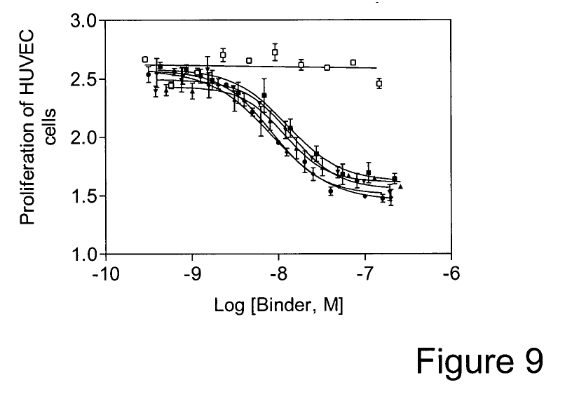

A number of clones were further tested for VEGF-inhibition of the growth of HUVEC cells (Human Umbilical Vein Endothelial Cells). HUVEC cells are natural human cells that are closely related to cells in the body that respond to VEGF. As shown in FIG. 9 and Table 10, the fibronectin-based binding proteins were also active in inhibiting VEGF activity in this human-derived cell system while the wild type fibronectin-based scaffold protein was inactive.

### Example 5

### Thermal Stability and Reversible Refolding of M5FL Protein

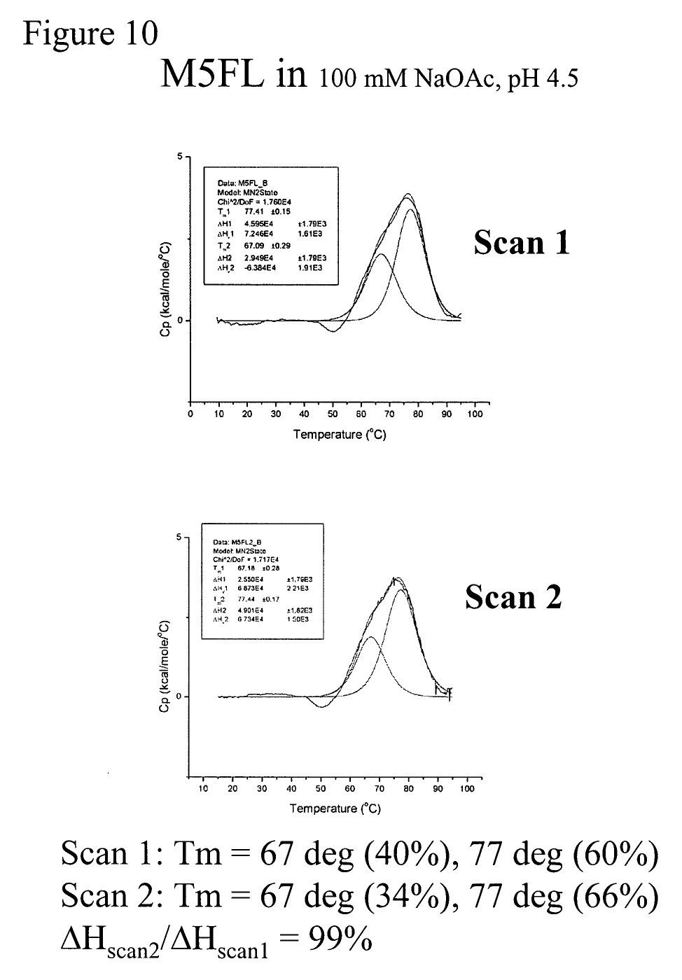

The thermal stability of KDR-binder M5FL was established using differential scanning calorimetry (DSC). Under standard PBS buffer conditions (sodium phosphate pH 7.4, 150 mM NaCl), M5FL was found to have a single non-reversible thermal melting transition at 56° C. Subsequently, sodium acetate pH 4.5 was identified as a favorable buffer for M5FL protein solubility. DSC experiments in this buffer (100 mM) demonstrated that M5FL is more stable under these conditions (Tm=67-77° C.) and that the melting transition is reversible (FIG. 10). Reversible thermal transitions have been used to identify favorable conditions that support long-term storage of protein therapeutics (Remmele et al, Biochemistry 38:5241 (1999), so Na-acetate pH 4.5 has been identified as an optimized buffer for storing the M5FL protein.

### Example 6

### In Vitro Binding and Cell-Based Activity of PEGylated M5FL Protein

The M5FL protein was produced in an E. coli expression system with a C-terminal extension to yield the following protein sequence (C-terminal extension underlined with Cys100 shaded; a significant percentage of protein is produced with the initial methionine removed):

(SEQ ID NO:199)

MGVSDVPRDLEVVAATPTSLLISWRHPHFPTRYYRITYGETGGNSPVQEF
TVPLQPPLATISGLKPGVDYTITVYAVTKERNGRELFTPISINYRTEIDK
PCQHHHHHH

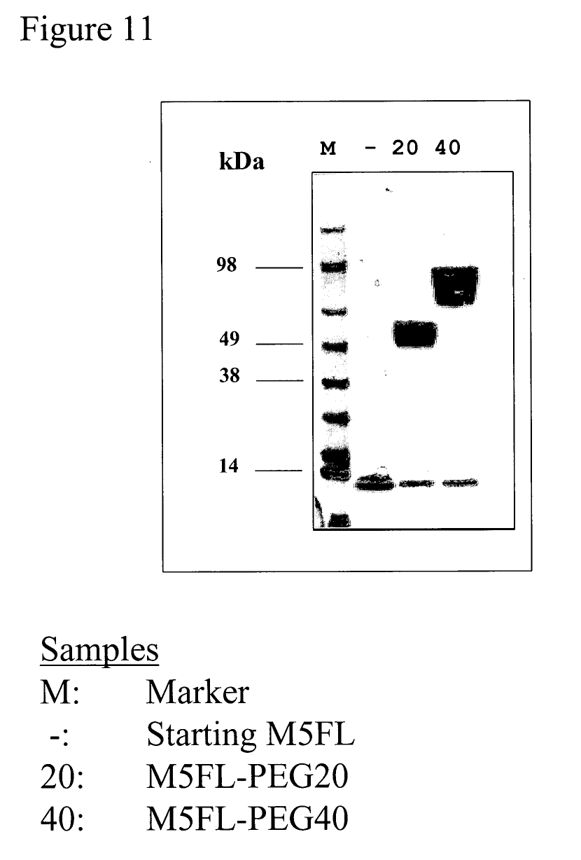

The single sulfhydryl of the cysteine residue at position 100 was used to couple to PEG variants using standard maleimide chemistry to yield two different PEGylated forms of M5FL. Both a linear 20 kD PEG and a branched 40 kD PEG (Shearwater Corporation) were conjugated to M5FL to produce M5FL-PEG20 and M5FL-PEG40, respectively. The PEGylated protein forms were purified from unreacted protein and PEG by cation exchange chromatography. Covalent linkage of the two PEG forms of M5FL was verified by SDS-PAGE (FIG. 11) and mass spectroscopy.

In vitro affinity measurements were made using surface plasmon resonance (SPR) (BIAcore) with both the human and mouse VEGF-receptor target proteins immobilized via amide chemistry on the BIAcore chip. For both target proteins, both the 20 and 40 kD PEGylated M5FL forms were found to have slower on-rates (ka) relative to unmodified M5FL with little effect on off-rates (kd; Table 11).

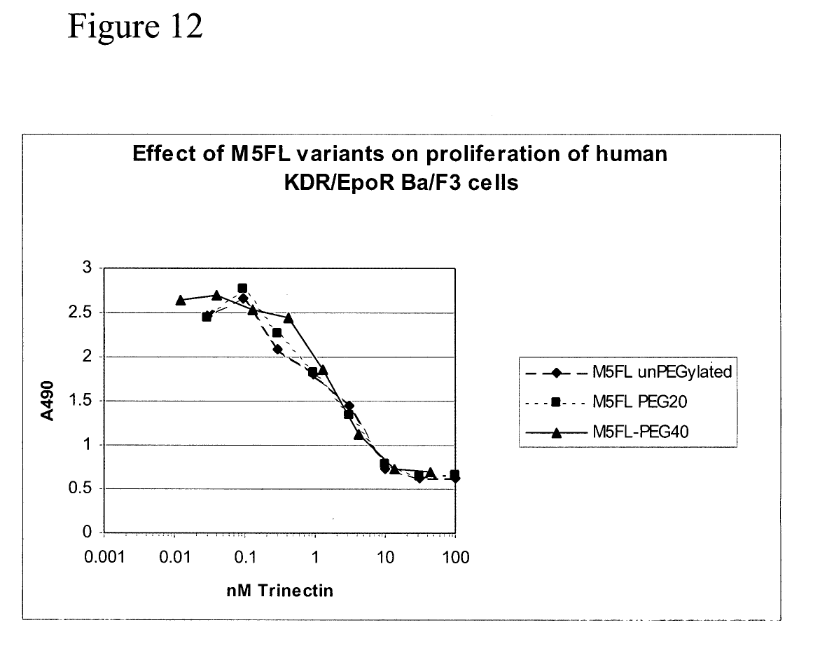

The functionality of the PEGylated M5FL preparations was tested using the Ba/F3 system described in Example 4. FIG. 12 shows a plot of A490 (representing the extent of cell proliferation) as a function of concentration of each of the binders. The curves were nearly identical, indicating there was little effect of PEGylation on the biological activity of either of the PEGylated forms.

The kₒₙ, k_{off} and K_{D} were analyzed for a subset of KDR-binding polypeptides and compared to the EC50 for the BaF3 cell-based VEGF inhibition assay. Scatter plots showed that the kon was well-correlated with the EC50, while k_{off} was poorly correlated. Greater than 90% of KDR-binding proteins with a kon of 105s⁻¹ or greater had an EC50 of 10 nM or less. K_{D} is a ratio of kon and koff, and, as expected, exhibits an intermediate degree of correlation with EC50.

Many of the KDR-binding proteins, including CT-01, were assessed for binding to VEGFR-1, VEGFR-2 and VEGFR-3. The proteins showed a high degree of selectivity for VEGFR-2.

### Example 6

### Preparation of KDR Binding Protein CT-01 Blocks VEGFR-2 Signaling in Human Endothelial Cells

Following the methodologies described in the preceding Examples, additional ¹⁰Fn3-based KDR binding proteins were generated. As described for the development of the M5FL protein in Example 5, above, proteins were tested for K_{D} against human KDR and mouse Flk-1 using the BIAcore binding assay and for IC50 in a Ba/F3 assay. A protein termed CT-01 exhibited desirable properties in each of these assays and was used in further analysis.

The initial clone from which CT-01 was derived had a sequence:

GEVVAATPTSLLISWRHPHFPTRYYRITYGETGGNSPVQEFTVPLQPPTATISGLK PGVDYTITVYAVTDGWNGRLLSIPISINYRT (SEQ ID NO:200). The FG loop sequence is underlined.

Affinity maturation as described above produced a core form of CT-01:

(SEQ ID NO:192)

EVVAATPTSLLISWRHPHFPTRYYRITYGETGGNSPVQEFTVPLQPPTAT
ISGLKPGVDYTITVYAVTDGRNGRLLSIPISINYRT.

The CT-01 molecule above has a deletion of the first 8 amino acids and may include additional amino acids at the N- or C-termini. For example, an additional MG sequence may be placed at the N-terminus. The M will usually be cleaved off, leaving a GEV . . . sequence at the N-terminus. The re-addition of the normal 8 amino acids at the N-terminus also produces a KDR binding protein with desirable properties. The N-terminal methionine is generally cleaved off to yield a sequence:

(SEQ ID NO:193)

VSDVPRDLEVVAATPTSLLISWRHPHFPTRYYRITYGETGGNSPVQEFTV
PLQPPTATISGLKPGVDYTITVYAVTDGRNGRLLSIPISTNYRT.

For use in vivo, a form suitable for PEGylation may be generated. For example, a C-terminal tail comprising a cysteine was added and expressed, as shown below for a form lacking the eight N-terminal amino acids.

GEVVAATPTSLLISWRHPHFPTRYYRITYGETGGNSPVQEFTVPLQPPTATISGLK PGVDYTITVYAVTDGRNGRLLSIPISINYRTEIDKPCQ (SEQ ID NO:194). The PEGylated form of this molecule is used in the in vivo experiments described below. A control form with a serine instead of a cysteine was also used:

(SEQ ID NO:195)

GEVVAATPTSLLISWRHPHFPTRYYRITYGETGGNSPVQEFTVPLQPPTA
TISGLKPGVDYTITVYAVTDGRNGRLLSIPISTNYRTEIDKPSQ.

The same C-terminal tails may also be added to CT-01 forms having the N-terminal eight amino acids, such as is shown in SEQ ID NO:193.

Additional variants with desirable KDR binding properties were isolated. The following core sequence has a somewhat different FG loop, and may be expressed with, for example, an N-terminal MG sequence, an N-terminal sequence that restores the 8 deleted amino acids, and/or a C-terminal tail to provide a cysteine for PEGylation.

EVVAATPTSLLISWRHPHFPTRYYRITYGETGGNSPVQEFTVPLQPPTATISGLKP GVDYTITVYAVTEGPNERSLFIPISINYRT (SEQ ID NO:196). Another such variant has the core sequence:

(SEQ ID NO:197)

VSDVPRDLEVVAATPTSLLISWRHPHFPTRYYRITYGETGGNSPVQEFTV
PLQPPTATISGLKPGVDYTITVYAVTEGPNERSLFIPISINYRT.

A comparison of these variants shows a consensus sequence for the FG loop of: D/E)GXNXRXXIP (SEQ ID NO:3). With greater particularity, the consensus sequence may be expressed as (D/E)G(R/P)N(G/E)R(S/L)(S/F)IP (SEQ ID NO:4).

### Example 7

### CT-01 Blocks VEGFR-2 Signaling in Human Endothelial Cells

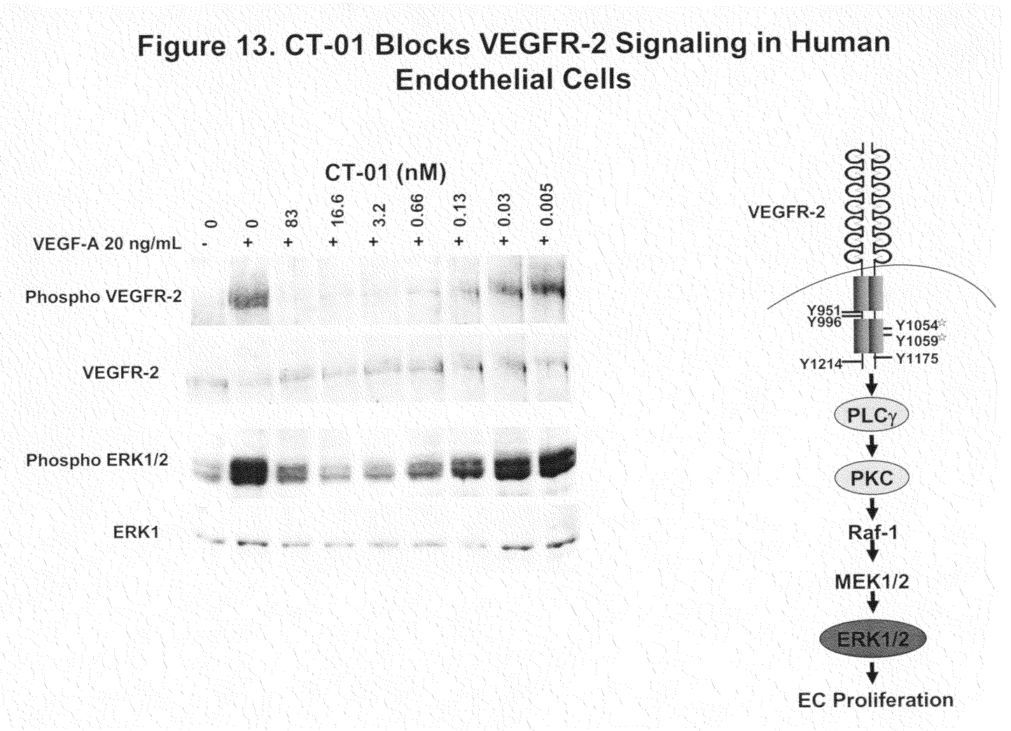

As shown in FIG. 13, VEGF-A signaling through VEGFR-2 is mediated by phosphorylation of the intracellular domain of VEGFR-2, followed by activation of pathway involving phospholipase C gamma (PLCγ), Protein Kinase C(PKC), Raf-1, MEK1/2, ERK1/2, leading to endothelial cell proliferation.

To assess whether KDR binders disclosed herein inhibited activation of this signaling pathway, Human Microvascular Endothelial Cells were treated with a VEGFR binding polypeptide (e.g., CT-01) for 30 min and stimulated with VEGF-A for 5 min. Total cell lysates were analyzed by SDS-PAGE and western analysis, using antibodies specific to phospho-VEGFR-2, non-phospho-VEGFR-2, phosphor-ERK1/2 and non-phospho-ERK1/2.

As shown in FIG. 13, 130 pM CT-01 inhibits formation of phosphor-VEGFR-2 and also decreases the formation of the downstream phosphorylated ERK1/2. Phosphorylated ERK1/2 is not entirely eliminated, probably due to the fact that ERK1/2 receives signals from a number of additional signaling pathways.

### Example 8

### Fibronectin-based KDR Binding Proteins Disrupt Signaling by VEGF-A and VEGF-D

VEGFR-2 is a receptor for three VEGF species, VEGF-A, VEGF-C and VEGF-D.

Experiments were conducted to evaluate the effects of fibronectin-based KDR binding proteins on VEGF-A and VEGF-D mediated signaling through KDR.

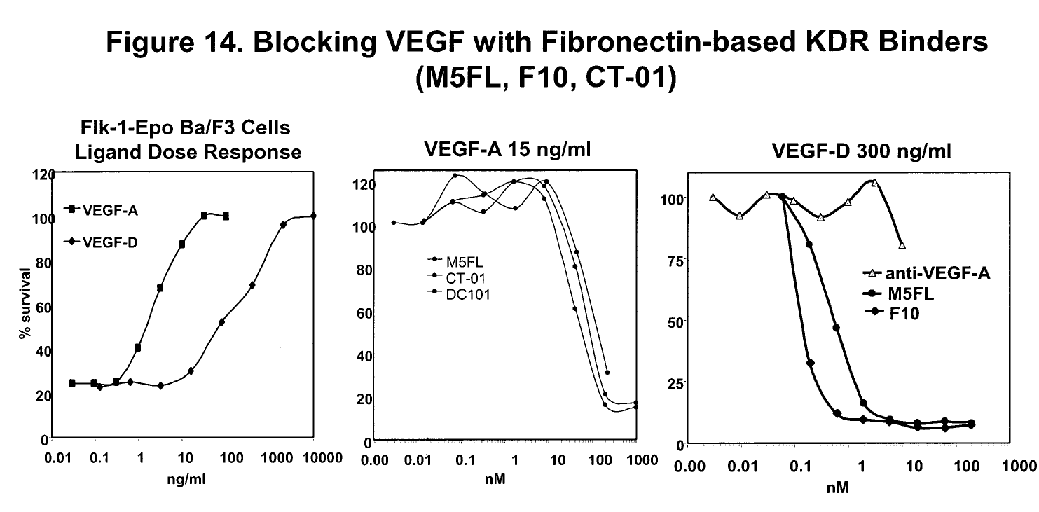

A Ba/F3 cell line dependent on Flk-1 mediated signaling was generated. As shown in the left panel of FIG. 14, cell viability could be maintained by treating the cells with VEGF-A or VEGF-D, although significantly higher levels of VEGF-D were required.

As shown in the middle panel of FIG. 14, cells were maintained in the presence of 15 ng/ml of VEGF-A and contacted with the M5FL or CT-01 proteins disclosed herein, or with the DC-101 anti-Flk-1 antibody. Each reagent reversed the VEGF-A-mediated cell viability, indicating that VEGF-A signaling through Flk-1 was blocked.

As shown in the right panel of FIG. 14, cells were maintained in the presence of 300 ng/ml of VEGF-D and contacted with the M5FL or F10 proteins disclosed herein, or with an anti-VEGF-A antibody. M5FL and F10 reversed the VEGF-D-mediated cell viability, indicating that VEGF-D signaling through Flk-1 was blocked. The anti-VEGF-A antibody had no effect, demonstrating the specificity of the assay.

### Example 9

### Pharmacokinetics

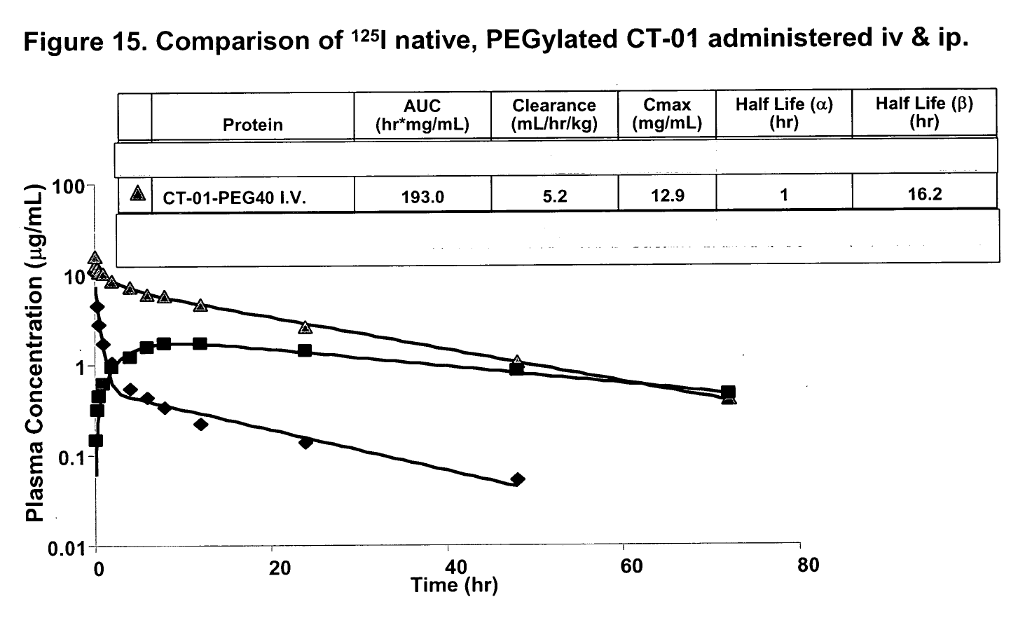

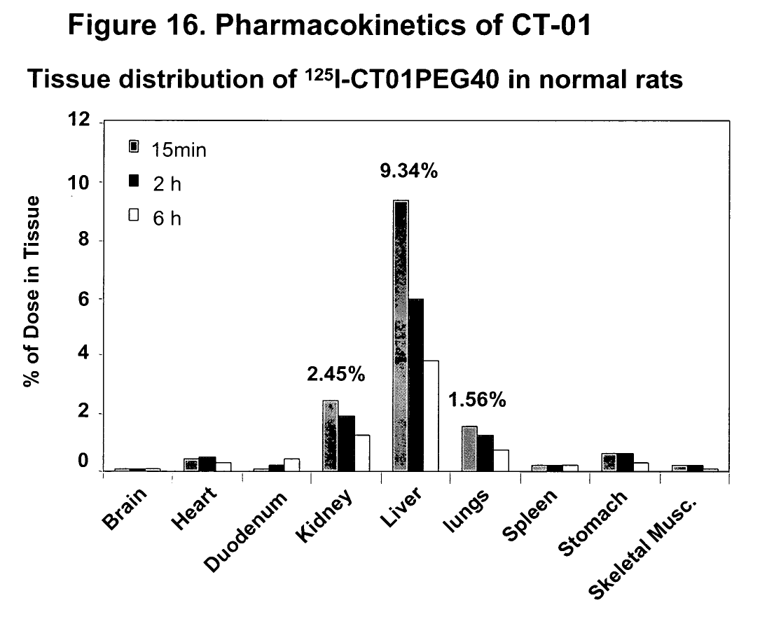

Pharmacokinetic Studies: Native CT-01 or a pegylated form (40 kDa PEG, CT-01PEG40) were iodinated with ¹²⁵I. 10-20 mCi of iodinated proteins were injected into adult male rats either i.v. or i.p. and iodinated proteins levels were determined at the indicated times. For tissue distribution studies, rats were sacrificed at 15 min, 2 hr and 6 hr and radioactivity levels determined. See FIGS. 15 and 16. Unmodified CT-01 is a 12 kDa protein that is rapidly cleared from the blood. The area-under-curve value (AUC) value is 14.6 hr*mg/mL with a clearance of 69.9 mL/hr/kg, a maximum serum concentration of 9.1 mg/ml. The initial half-life (a) is 0.3 hours and the second phase half-life (β) is 13.5 hours. By comparison, i.v. PEGylated CT-01 has greatly increased presence in the blood, mostly because of a dramatic decrease in the initial phase of clearance. The AUC is increased greater than 10 fold to 193, the clearance rate is decreased by greater than 10 fold to 5.2, the Cmax is 12.9 mg/mL. The α half-life is increased to 1 hour, and the β is increased to 16.2 hours. These pharmacokinetics in rats are equivalent to a twice-weekly dosing regimen in humans, a rate of dosing that is well within acceptable ranges.

Intraperitoneal (i.p.) administration of PEGylated CT-01 had reservoir-like pharmacokinetics. There was no initial spike in the blood concentration of CT-01. Instead, the amount of CT-01 built up more slowly and decreased slowly. Such pharmacokinetics may be desirable where there is concern about side effects from the initial spike in CT-01 concentration upon intravenous administration. It is likely that other ¹⁰FN3-based agents would exhibit similar behavior in i.p. administration.

Accordingly, this may be a generalizable mode for achieving a time-delayed dosing effect with ¹⁰FN3-based agents.

As shown in FIG. 16, the liver is the primary route for secretion of the PEGylated form of CT-01. No long term accumulation of CT-01 was detected.

Similar results were obtained using a CT-01 conjugated to a 20 kDa PEG moiety.

### Example 10

### In Vivo Efficacy of CT-01

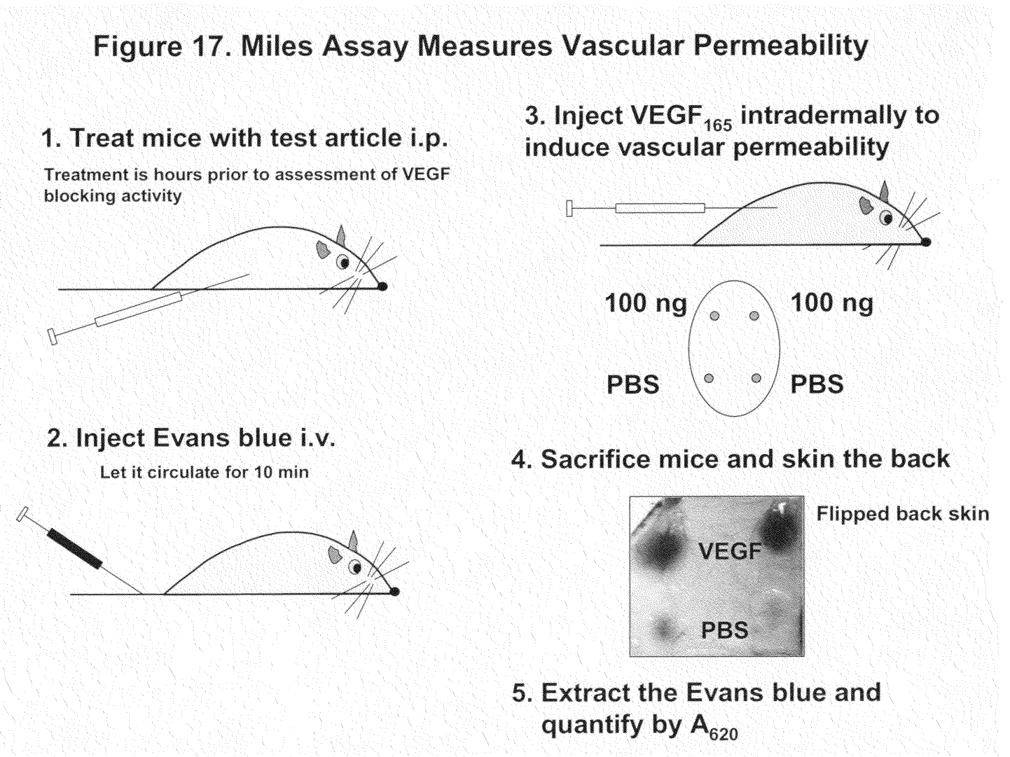

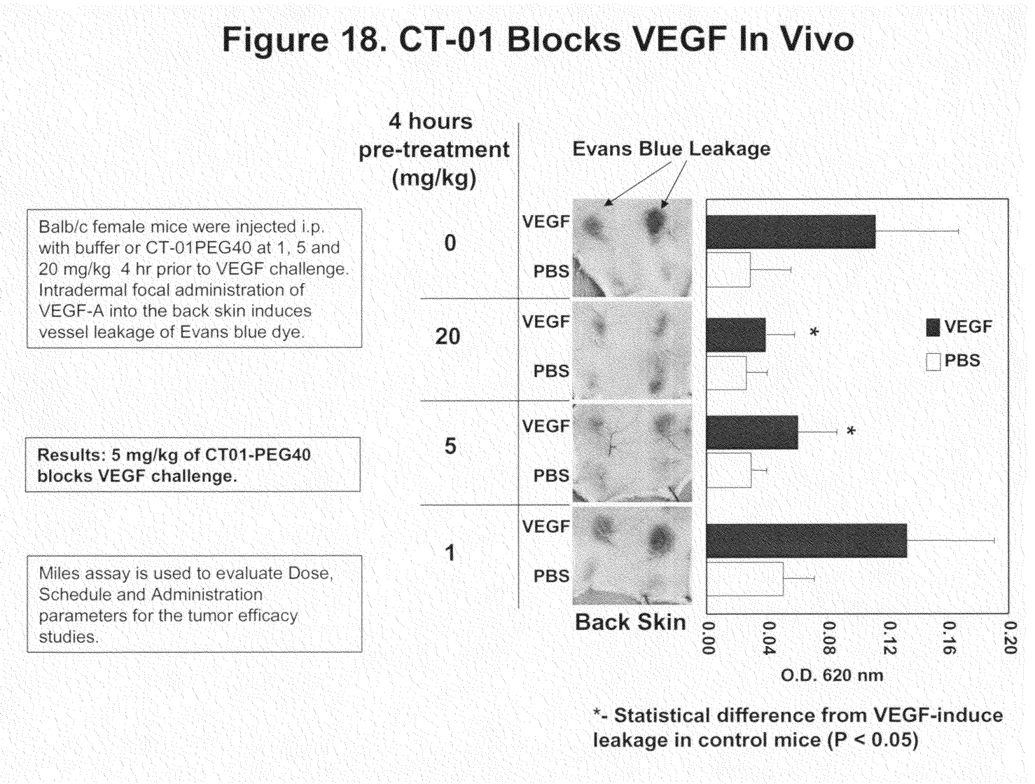

The Miles assay, as outlined in FIG. 17, is used to evaluate Dose, Schedule and Administration parameters for the tumor efficacy studies. Balb/c female mice were injected i.p. with buffer or CT-01PEG40 at 1, 5 and 20 mg/kg 4 hr prior to VEGF challenge. Intradermal focal administration of VEGF-A into the back skin induces vessel leakage of Evans blue dye (FIGS. 17 and 18).

Mice treated with a KDR binding agent showed a statistically significant decrease in the level of VEGF-mediated vessel leakage. Both 5 mg/kg and 20 mg/kg dosages with CT-01 showed significant results. Therefore, a 5 mg/kg dosage was selected for mouse tumor model studies.

### Example 11

### CT-01 Inhibits Tumor Growth

### B 16-F10 Murine Melanoma Tumor Assay:

2×10⁶ B16-F10 murine melanoma tumor cells were implanted subcutaneously into C57/BL male mice at Day 1. At day 6 a palpable mass was detected. On day 8 when tumors were of measurable size, daily i.p. injections of either Vehicle control, 5, 15, or 40 mg/kg CT-01PEG40 were started. The lowest dose 5 mg/kg decreased tumor growth.

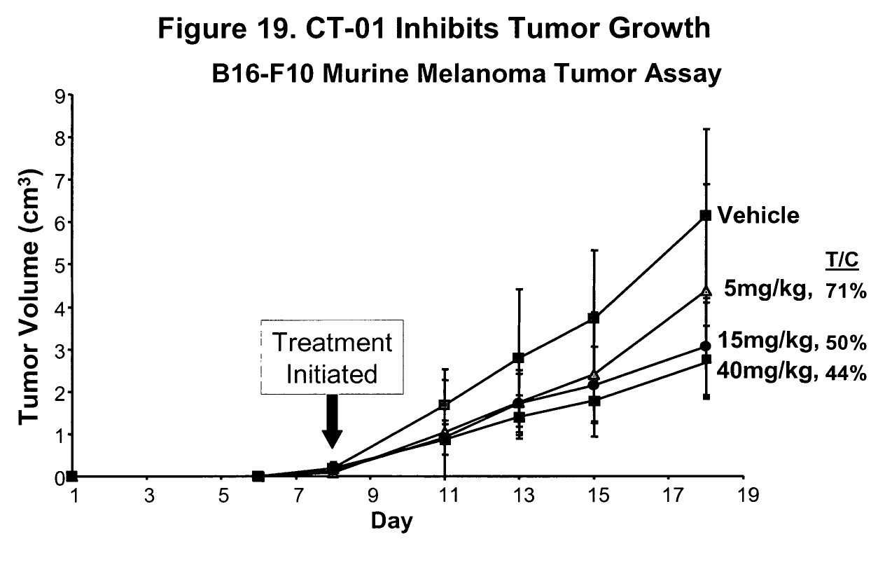

At day 18, mice treated with 15 and 40 mg/kg showed 50% and 66% reduction in tumor growth. See FIG. 19.

### U87 Human Glioblastoma Assay:

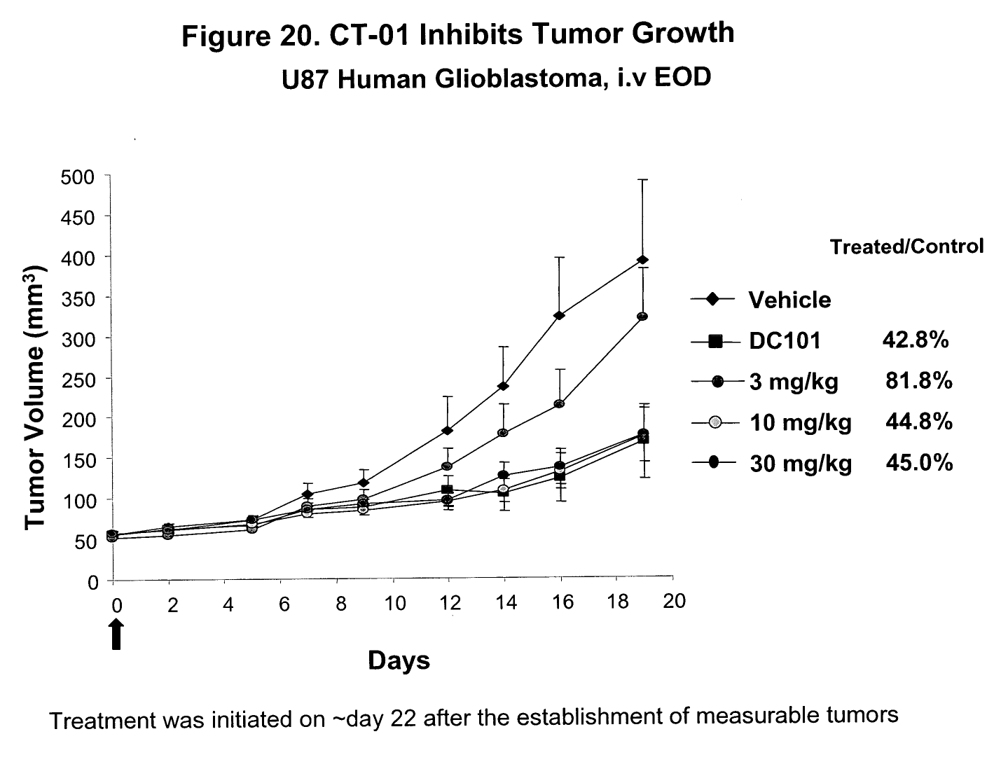

5×10⁶ U87 human glioblastoma tumor cells were implanted subcutaneously into nude male mice. When tumor volume reached approximately 50 mm3 treatment started (day 0). Vehicle control, 3, 10, or 30 mg/kg CT-01PEG40 were injected i.v. every other day (EOD). The anti-Flk-1 antibody DC101 was injected at 40 mg/kg twice a week as published for its optimal dose schedule. The lowest dose 3 mg/kg decreased tumor growth. At day 12, mice treated with 10 and 30 mg/kg showed 50% reduction in tumor growth. See FIG. 20. Effectiveness is comparable to that of the anti-Flk-1 antibody.

The following materials and methods were used for the experiments described in Examples 1-11.

### Recombinant Proteins:

Recombinant human VEGF₁₆₅, murine VEGF₁₆₄, human neurotrophin-4 (NT4), human and mouse vascular endothelial growth factor receptor-2 Fc chimeras (KDR-Fc and Flk-1-Fc) were purchased from R&D systems (Minneapolis, Minn.). Biotinylation of the target proteins was carried out in 1×PBS at 4° C. for 2 hours in the presence of EZ-Lik™ Sulfo-NHS-LC-LC-Biotin (Pierce, Ill.). Excess of EZ-Link™ Sulfo-NHS-LC-LC-Biotin was removed by dialysis against 1×PBS. The level of biotinylation was determined by mass spectroscopy and target protein concentrations were determined using Coomassie Protein Plus Assay (Pierce, Ill.).

### Primers:

The following oligonucleotides were prepared by chemical synthesis for eventual use in library construction and mutagenesis of selected clones.

T7 TMV Fn:
5′ GCG TAA TAC GAC TCA CTA TAG GGA CAA TTA CTA TTT
ACA ATT ACA ATG GTT TCT GAT GTT CCG AGG 3′
(SEQ ID NO:201)
T7 TMV N-terminus deletion:
5′ GCG TAA TAC GAC TCA CTA TAG GGA CAA TTA CTA TTT
ACA ATT ACA ATG GAA GTT GTT GCT GCG ACC CCC ACC
AGC CTA 3′ (SEQ ID NO:202)
MK165-4 A20:
5′ TTT TTT TTT TTT TTT TTT TTA AAT AGC GGA TGC CTT
GTC GTC GTC GTC CTT GTA GTC 3′ (SEQ ID NO:203)
N-terminus forward:
5′ ATG GTT TCT GAT GTT CCG AGG GAC CTG GAA GTT GTT
GCT GCG ACC CCC ACC AGC CTA CTG ATC AGC TGG 3′
(SEQ ID NO:204)
BCDE reverse:
5′ AGG CAC AGT GAA CTC CTG GAC AGG GCT ATT TCC TCC
TGT TTC TCC GTA AGT GAT CCT GTA ATA TCT 3′
(SEQ ID NO:205)
BCDE forward:
5′ AGA TAT TAC AGG ATC ACT TAC GGA GAA ACA GGA GGA
AAT AGC CCT GTC CAG GAG TTC ACT GTG CCT 3′
(SEQ ID NO:206)
DEFG reverse:
5′ AGT GAC AGC ATA CAC AGT GAT GGT ATA ATC AAC TCC
AGG TTT AAG GCC GCT GAT GGT AGC TGT 3′
(SEQ ID NO:207)
DEFG forward:
5′ ACA GCT ACC ATC AGC GGC CTT AAA CCT GGA GTT GAT
TAT ACC ATC ACT GTG TAT GCT GTC ACT 3′
(SEQ ID NO:208)
C-terminus polyA:
5′ TTT TTT TTT TTT TTT TTT TAA ATA GCG GAT GCC TTG
TCG TCG TCG TCC TTG TAG TCT GTT CGG TAA TTA ATG
GAA AT 3′ (SEQ ID NO:209)
Hu3′FLAGSTOP:
5′ TTT TAA ATA GCG GAT GCC TTG TCG TCG TCG TCC TTG
TAG TCT GTT CGG TAA TTA ATG G 3′ (SEQ ID NO:210)
R28FG-50:
5′ GTG TAT GCT GTC ACT 123 145 463 665 165 465 163
425 625 645 447 ATT TCC ATT AAT TAC 3′ ,
(SEQ ID NO:211), where 1 = 62.5%G + 12.5%A + 12.5%
T + 12.5%C; 2 = 2.5%G + 12.5%A + 62.5%T + 12.5%C;
3 = 75%G + 25%C; 4 = 12.5%G + 12.5%A + 12.5%T +
62.5%C; 5 = 25%G + 75%C; 6 = 12.5%G + 62.5%A +
12.5%T + 12.5%C; 7: 25%G + 50%A + 25%C
F1U2:
5′ TAA TAC GAC TCA CTA TAG GGA CAA TTA CTA TTT ACA
ATT CTA TCA ATA CAA TGG TGT CTG ATG TG CCG 3′
(SEQ ID NO:212)
F2:
5′ CCA GGA GAT CAG CAG GGA GGT CGG GGT GGC AGC CAC
CAC TTC CAG GTC GCG CGG CAC ATC AGA CAC CAT TGT 3′
(SEQ ID NO:213)
F3159:
5′ ACC TCC CTG CTG ATC TCC TGG CGC CAT CCG CAT TTT
CCG ACC CGC TAT TAC CGC ATC ACT TAC G 3′
(SEQ ID NO:214)
F4:
5′ CAC AGT GAA CTC CTG GAC CGG GCT ATT GCC TCC TGT
TTC GCC GTA AGT GAT GCG GTA ATA GCG 3′
(SEQ ID NO:215)
F5159:
5′ CGG TCC AGG AGT TCA CTC TGC CGC TGC AGC CGC CGG
CGG CTA CCA TCA GCG GCC TTA AAC C 3′
(SEQ ID NO:216)
F5-X5:
5′ CG GTC CAG GAG TTC ACT GTG CCG NNS NNS NNS NNS
NNS GCT ACC ATC AGC GGC CTT AAA CC 3′
(SEQ ID NO:217)
F6:
5′ AGT GAC AGC ATA CAC AGT GAT GGT ATA ATC AAC GCC
AGG TTT AAG GCC GCT GAT GGT AG 3′ (SEQ ID NO:218)
F7X6159:
5′ ACC ATC ACT GTG TAT GCT GTC ACT NNS NNS NNS NNS
NNS NNS GAA CTG TTT ACC CCA ATT TCC ATC AAC TAC
CGC ACA GAC TAC AAG 3′ (SEQ ID NO:219)
F8:
5′ AAA TAG CGG ATG CGC GTT TGT TCT GAT CTT CCT TAT
TTA TGT GAT GAT GGT GGT GAT GCT TGT CGT CGT CGT
CCT TGT AGT CTG TGC GGT AGT TGA T 3′
(SEQ ID NO:220)
G2asaiA20:
5′ TTT TTT TTT TTT TTT TTT TTA AAT AGC GGA TGC GCG
TTT GTT CTG ATC TTC 3′ (SEQ ID NO:221)
C2RT:
5′ GCG CGT TTG TTC TGA TCT TCC 3′ (SEQ ID NO:222)
hf01 BC reverse:
5′ TGCC TCC TGT TTC GCC GTA AGT GAT GCG GTA ATA
GCG SNN SNN SNN SNN SNN SNN SNN CCA GCT GAT CAG
CAG 3′ (SEQ ID NO:223)
hf01 DE reverse:
5′ GAT GGT AGC TGT SNN SNN SNN SNN AGG CAC AGT GAA
CTC CTG GAC AGG GCT ATT GCC TCC TGT TTC GCC 3′
(SEQ ID NO:224)
hf01 FG reverse:
5′ GT GCG GTA ATT AAT GGA AAT TGG SNN SNN SNN SNN
SNN SNN SNN SNN SNN SNN AGT GAC AGC ATA CAC 3′
(SEQ ID NO:225)
BCDE rev:
5′ CCT CCT GTT TCT CCG TAA GTG 3′ (SEQ ID NO:226)
BCDEfor:
5′ CAC TTA CGG AGA AAC AGG AGG 3′ (SEQ ID NO:227)
hf01 DE-FG forward:
5′ ACA GCT ACC ATC AGC GGC CTT AAA CCT GGC GTT GAT
TAT ACC ATC ACT GTG TAT GCT GTC ACT 3′
(SEQ ID NO:228)
Front FG reverse:
5′ AGT GAC AGC ATA CAC AGT 3′ (SEQ ID NO:229)
hf01 RT Flag PolyA reverse:
5′ TTT TTT TTT TTT TTT TTT TTA AAT AGC GGA TGC CTT
GTC GTC GTC GTC CTT GTA GTC TGT GCG GTA ATT AAT
GGA 3′ (SEQ ID NO:230)
5-RI-hKDR-1B:
5′ TAG AGA ATT CAT GGA GAG CAA GGT GCTG 3′
(SEQ ID NO:231)
3-EPO/hKDR-2312B:
5′ AGG GAG AGC GTC AGG ATG AGT TCC AAG TTC GTC TTT
TCC 3′ (SEQ ID NO:232)
5-RI-mKDR-1:
5′ TAG AGA ATT CAT GGA GAG CAA GGC GCT G 3′
(SEQ ID NO:233)
3-EPO/mKDR-2312:
5′ AGG GAG AGC GTC AGG ATG AGT TCC AAG TTG GTC TTT
TCC 3′ (SEQ ID NO:234)
5-RI-hTrkB-1:
5′ TAG AGA ATT CAT GAT GTC GTC CTG GAT AAG GT 3′
(SEQ ID NO:235)
3-EpoR/hTrkB-1310:
5′ AGG GAG AGC GTC AGG ATG AGA TGT TCC CGA CCG GTT
TTA 3′ (SEQ ID NO:236)
5-hKDR/EPO-2274B:
5′ GGA AAA GAC GAA CTT GGA ACT CAT CCT GAC GCT CTC
CCT 3′ (SEQ ID NO:237)
5-mKDR/EPO-2274:
5′ GGA AAA GAC CAA CTT GGA ACT CAT CCT GAC GCT CTC
CCT 3′ (SEQ ID NO:238)
3-XHO-EpoR-3066:
5′ TAG ACT CGA GTC AAG AGC AAG CCA CAT AGCT 3′
(SEQ ID NO:239)
5′hTrkB/EpoR-1274:
5′ TAA AAC CGG TCG GGA ACA TCT CAT CCT GAC GCT CTC
CCT 3′ (SEQ ID NO:240)

### Buffers

The following buffers were utilized in the experiments described herein. Buffer A (100 mM Tris HCl, 1M NaCl, 0.05% Tween-20, pH 8.0); Buffer B (1×PBS, 0.02% Triton X100); Buffer C (100 mM Tris HCl, 60 mM EDTA, 1M NaCl, 0.05% Triton X100, pH 8.0); Buffer Ca (100 mM Tris HCl, 1M NaCl, 0.05% Triton X100, pH 8.0); Buffer D (2M NaCl, 0.05% Triton); Buffer E (1×PBS, 0.05% Triton X100, pH 7.4); Buffer F (1×PBS, 0.05% Triton X100, 100 mM imidazole, pH 7.4); Buffer G (50 mM HEPES, 150 mM NaCl, 0.02% TritonX-100, 1 mg/ml bovine serum albumin, 0.1 mg/ml salmon sperm DNA, pH 7.4); Buffer H (50 mM HEPES, 150 mM NaCl, 0.02% TritonX-100, pH 7.4); Buffer I (1×PBS, 0.02% TritonX-100, 1 mg/ml bovine serum albumin, 0.1 mg/ml salmon sperm DNA, pH 7.4); Buffer J (1×PBS, 0.02% TritonX-100, pH 7.4); Buffer K (50 mM NaH₂PO₄, 0.5 M NaCl, 5% glycerol, 5 mM CHAPS, 25 mM imidazole, 1× Complete™ Protease Inhibitor Cocktail (Roche), pH 8.0); Buffer L (50 mM NaH₂PO₄, 0.5 M NaCl, 5% glycerol, 25 mM imidazole, pH 8.0); Buffer M (1×PBS, pH 7.4, 25 mM imidazole, pH 7.4); Buffer N (1×PBS, 250 mM imidazole, pH 7.4); Buffer 0 (10 mM HEPES, 150 mM NaCl, 0.005% Tween 20, pH 7.4).

### Primary Library Construction:

The construction of the library using the tenth domain of human fibronectin as a scaffold was previously described (Xu et al, 2002, supra). Three loop regions, corresponding to positions 23-29, 52-55, and 77-86, respectively, were randomized using NNS (standard nucleotide mixtures, where N=equimolar mixture of A, G, T, C; S=equimolar mixture of G and C) as the coding scheme. Similar libraries were constructed containing randomized regions only at positions 23-29 and 77-86 (two loop library) or only at positions 77-86 (one loop library). These libraries were mixed in approximately equimolar amounts. This mixed library contained ˜1×10¹³ clones and was used in the KDR selection that identified VR28.

### Mutagenic Library Construction:

Hypermutagenic PCR. Scaffold mutation T(69)I in VR28 clone was corrected back to wild type sequence by PCR (see below) and no change in binding characteristics of VR28 binder to KDR was observed. Mutations were introduced into the loop regions of VR28 using conditions described previously (Vartanian et al, Nuc. Acid Res. 24:2627-2631, 1996). Three rounds of hypermutagenic PCR were conducted on a VR28 template using primer pairs flanking each loop (N-terminus forward/BCDE reverse, BCDE forward/DEFG reverse, DEFG forward/C-terminus polyA). The resulting fragments were assembled using overlap extension and PCR with flanking primers T7TMV Fn and MK165-4 A20. DNA sequencing of the clones from the final PCR reaction confirmed correct assembly of the library. Up to 30% mutagenesis rate was observed in the loop regions, as compared to 1.5% in the scaffold regions.

Oligo mutagenesis. Oligo mutagenesis of the FG loop of VR28 by PCR utilized the VR28FG-50 primer, DEFG reverse primer and flanking primers. At each nucleotide position encoding the FG loop, primer VR28FG-50 contained 50% of the VR28 nucleotide and 50% of an equimolar mixture of all four nucleotides (N) or of G or C(S). This scheme was designed to result in approximately 67% of the amino acids of the VR28 FG loop being randomly replaced by another amino acid which was confirmed by DNA sequencing.

159 (Q8L) randomized sub-libraries. Oligo mutagenesis of the FG loop of Clone 159 (Q8L) clone, a three-step extension and amplification was performed. For the first extension, pairs of primers (a: F1U2/F2, b: F3159/F4, c: F5159/F6, d: F7X6159/F8) were mixed in equal concentrations (100 pmol each) and amplified for 10 cycles. For the second extension, 1/20 of the first reactions were combined (a/b and c/d) and amplification was continued for another 10 cycles. To bias the amplification in favor of extension rather than re-annealing of fully complementary fragments, a linear amplification of the half-construct products (0.5 pmol each) was performed for an additional 20 cycles using 50 pmol of either F1U2 forward primer for fragment ab, or the C2asaiA20 reverse primer for fragment cd. Finally, the extended half-construct fragments ab and cd were combined and amplified for 20 cycles without any additional components. Primer F7X6159 contained NNS at each of the first 6 coding positions of Clone 159 (Q8L) and was otherwise identical to Clone 159 (Q8L). Correct assembly of the library 159 (Q8L)-FGX6 was confirmed by DNA sequencing of clones from the final PCR reaction. The sub-library contained ˜1×10⁹ clones.

For randomization of the DE loop of post round 6 (PR6) selection pool of the 159 (Q8L)-FGX6 library, two half-construct fragments were prepared by PCR using primers F1U2/F4 and F5X5/C2asaiA20. The F5X5 primer contained NNS at the four positions of the DE loop as well as at position 56. Then, the extended fragments ab and cd were combined and amplified for 20 cycles without any additional components.

Introduction of point mutations, deletion and random (NNS) loop sequences into fibronectin-based scaffold proteins:

Scaffold mutation T(69)I of VR28 binder was corrected back to wild type sequence in two-step PCR using VR28 clone as a template. Half-construct fragments, obtained with primers N-terminus forward/DEFG reverse and DEFG forward/C-terminus polyA, were combined and the whole VR28 (169T) clone (designated as VR28 in the text) was constructed using primers T7TMV Fn and MK165-4 A20. Correction of N-terminus mutations in clone 159 (Q8 to L) was performed by PCR with primers N-terminus forward/C-terminus polyA followed by extension with primers T7TMV Fn and MK165-4 A20.

Introduction of deletion Δ1-8 into the N-terminus of fibronectin-based scaffold proteins was performed by amplification using primers T7 TMV N-terminus deletion and MK165-4 A20.

Construction of the chimeras of E clones containing NNS loop sequences was performed by two-step PCR. Loop regions were amplified using primers T7 TMV N-terminus deletion/BCDE rev (a: BC loop of E clones); N-terminus forward/hf01 BC reverse (b: BC NNS); BCDE for/Front FG reverse (c: DE loop of E clones); BCDE for/hf01 DE reverse (d: DE NNS); hf01 DE-FG forward/hf01 RT-Flag PolyA reverse (e: FG of E clones); hf01 DE-FG forward/hf01 FG reverse (f: FG NNS). Fragments b/c/e, a/d/e, a/c/f were combined and the whole pools were constructed by extension and amplification using primers T7Tmv N-terminus deletion and hf01 RTFlag PolyA reverse.

All constructs were verified and/or analyzed by DNA sequencing. All constructs and mutagenic libraries contained T7 TMV promoter at the 5′ flanking region and Flag tag or His₆ tag sequences at 3′ flanking region for RNA-protein fusion production and purification in vitro.

### RNA-Protein Fusion Production

For each round of selection PCR DNA was transcribed using MegaScript transcription kit (Ambion) at 37° C. for 4 hours. Template DNA was removed by DNase I (Ambion) digestion at 37° C. for 20 minutes. RNA was purified by phenol/chloroform extraction followed by gel filtration on a NAP-25 column (Amersham). The puromycin linker PEG 6/10 (5′ Pso u agc gga ugc XXX XXX CC Pu 3′, where Pso=C6-Psoralen, u,a,g,c=2′OMe-RNA, C=standard amidities, X: Spacer Phosphoramidite 9 (9-O-Dimethoxytrityl-triethylene glycol, 1-[(2-cyanoethyl)-(N,N-diisopropyl)]-phosphoramidite); Pu=Puromycin-CPG) was synthesized as described previously (Kurz et al, Nuc. Acid Res. 28:83, 2000). The linker was annealed to the library RNA in 0.1 M NaCl, 25 mM Tris HCl, pH 7.0, by gradient temperature decrease from 85° C. to 4° C. The linker and RNA were then cross linked by exposing to UV light (365 nm) for 15 minutes. The cross-linked mixture (600 pmol RNA) was included in an in vitro translation reaction using rabbit reticulocyte lysate translation kit (Ambion) in the presence of ³⁵S-labeled methionine at 30° C. for 60 minutes. To enhance fusion formation, 0.5 M KCl and 0.05 M MgCl₂ were added to the reaction and incubated for 30 minutes at 4° C. Fusion molecules were purified using oligo-dT cellulose (Sigma) chromatography as follows. The translation and fusion mix was diluted into buffer A (100 mM Tris HCl, 1M NaCl, 0.05% Tween-20, pH 8.0) and added to oligo dT cellulose. The slurry was rotated at 4° C. for 1 hour and transferred to a spin column. Oligo dT cellulose beads were washed on the column with 10 column volumes of buffer A and eluted with 3 column volumes of H₂O. Reverse transcription reaction was conducted with SuperScript II Reverse Transcription kit (Invitrogen) for 1 hour at 42° C. using primer Hu3′FLAGSTOP. To decrease potential non-specific binding through reactive cysteines the thiol groups were reacted with 1 mM of 2-nitro-5-thiocyanatobenzoic acid (NTCB) or N-ethylmaleimide (NEM) alternatively over the course of the selection. The reaction was carried out for 1 hour at room temperature. Fusion molecules were further purified by anti-FLAG affinity chromatography using M2 agarose (Sigma). The M2 beads were added to the reaction and rotated in buffer B (1×PBS, 0.02% Triton X100) for 1 hour at 4° C. Then the beads were applied to a spin column, washed with 5 column volumes of buffer B and fusion molecules were eluted with 3 column volumes of 100 μM Flag peptide DYKDDDDK (Sigma) in buffer G. Fusion yield was calculated based on specific activity measured by scintillation counting of ³⁵S-methionine in the samples.

For the 159 (Q8L) randomized library, RNA-protein fusion was prepared using a streamlined, semi-automated procedure in a Kingfisher™ (ThermoLabSystems). The steps were similar to the procedure described above except for several steps described below. Purification of the RNA-protein fusion molecules was performed in buffer C (100 mM Tris HCl, 60 mM EDTA, 1M NaCl, 0.05% Triton X100, pH 8.0) on magnetic oligo dT beads (GenoVision). The beads were washed with 10 reaction volumes of buffer Ca (100 mM Tris HCl, 1M NaCl, 0.05% Triton X100, pH 8.0) and fusion proteins were eluted with one volume of H₂O. Reverse transcription (RT) was conducted using primer C2RT. Fusion proteins were further purified by His-tag affinity chromatography using Ni-NTA magnetic beads (Qiagen). The RT reaction was incubated with Ni-NTA beads in buffer D (2M NaCl, 0.05% Triton) for 20 minutes at room temperature, the beads were then washed with 10 reaction volumes of buffer E (1×PBS, 0.05% Triton X100, pH 7.4) and fusion molecules were eluted with one volume of buffer F (1×PBS, 0.05% Triton X100, 100 mM imidazole, pH 7.4).

### Selection:

Primary selection against KDR. Fusion library (˜10¹³ clones in 1 ml) was incubated with 150 μl of Protein A beads (Dynal) which was pre-immobilized with 200 nM of human IgG1 for 1 hour at 30° C. prior to selection to reduce non-specific binding to both Protein A baeds and Fc protein (preclear). The supernatant was then incubated in buffer G (50 mM HEPES, 150 mM NaCl, 0.02% TritonX-100, 1 mg/ml bovine serum albumin, 0.1 mg/ml salmon sperm DNA, pH 7.4) with KDR-Fc chimera for 1 hour at 30° C. with end-over-end rotation. Final concentrations of KDR-Fc were 250 nM for Round 1, 100 nM for rounds 2-4 and 10 nM for rounds 5 and 6. The target was captured on 300 μl of Protein A beads (Round 1) or 100 μl of Protein A beads (Rounds 2-6) for 30 minutes at 30° C. with end-over-end rotation and beads were washed 5 times with 1 ml of buffer G (50 mM HEPES, 150 mM NaCl, 0.02% TritonX-100, pH 7.4). Bound fusion molecules were eluted with 100 μl of 0.1 M KOH into 50 μl of 1 M Tris HCl, pH 8.0. DNA was amplified from elution by PCR using flanking primers T7TMV Fn and MK165-4 A20.

Affinity and specificity maturation of KDR binder VR28. Clone VR28 was mutagenized by hypermutagenic PCR or oligo-directed mutagenesis as described above and fusion sub-libraries were constructed. Following pre-clear with Protein A beads selection was performed in buffer I (1×PBS, 0.02% TritonX-100, 1 mg/ml bovine serum albumin, 0.1 mg/ml salmon sperm DNA, pH 7.4) for four rounds according to procedure described above. DNA was amplified from elution by PCR using primers T7TMV Fn and MK165-4 A20. Lower target concentrations (0.1 nM KDR for first four rounds of selection) were used for libraries derived from oligo mutagenesis and then 1 nM mouse VEGF-R2 (Flk-1) was introduced for three additional rounds of selection. Primers T7 TMV N-terminus deletion and MK165-4 A20 were used for PCR in the last 3 rounds. For specificity maturation of KDR binder 159 first 6 positions of the FG loop of clone 159 Q(8)L were randomized by PCR as described above. Binding of the fusion sub-library to biotinylated mouse VEGF-R2 (70 nM) was performed in buffer I at room temperature for 30 minutes. The rest of the selection procedure was continued in Kingfisher™ (ThermoLabSystems). The biotinylated target was captured on 50 μl of streptavidin-coated magnetic beads (Dynal) and the beads were washed with 10 volumes of buffer I and one volume of buffer J (1×PBS, 0.02% TritonX-100, pH 7.4). Bound fusion molecules were eluted with 100 μl of 0.1 M KOH into 5.01 of 1 M Tris HCl, pH 8.0. DNA was amplified from elution by PCR using primers F1U2 and C2asaiA20. After four rounds of selection an off-rate/rebinding selection against 7 nM Flk-1 was applied for another two rounds as follows. After the binding reaction with biotinylated mouse Flk-1 had progressed for 30 minutes, a 100-fold excess of non-biotinylated Flk-1 was added and the reaction continued for another 6 hours to allow time for the weak binders to dissociate. The biotinylated target was captured on 50 μl of streptavidin beads (Dynal) and beads were washed 5 times with 1 ml of buffer J. Bound fusion molecules were eluted by incubation at 75° C. for 5 minutes. Supernatant was subjected to re-binding to 7 nM Flk-1 and standard selection procedure was continued. DNA from the final elution pool was subjected to DE loop randomization (see above) and fusion sub-library was selected against 7 nM mouse VEGF-R2 for three rounds. At the fourth round an off-rate selection was applied with re-binding to 1 nM human VEGF-R2. Final DNA was amplified from elution by PCR using primers F1U2 and C2asaiA20.

### Radioactive Equilibrium Binding Assay

To prepare ³⁵S-labeled binding proteins for analysis, mRNA was prepared as described above for RNA-protein fusion production but the linker ligation step was omitted. The mRNA was expressed using rabbit reticulocyte lysate translation kit (Ambion) in the presence of ³⁵S-labeled Met at 30° C. for 1 hour. Expressed protein was purified on M2-agarose Flag beads (Sigma) as described above. This procedure produced the encoded protein without the nucleic acid tail. In a direct binding assay, VEGF-R2-Fc fusions in concentrations ranging from 0 to 200 nM were added to a constant concentration of the purified protein (0.2 or 0.5 nM) and incubated at 30° C. for 1 hour in buffer B. The receptor-binder complexes were captured using Protein A magnetic beads for another 10 minutes at room temperature using a Kingfisher™. The beads were washed with six reaction volumes of buffer B. The protein was eluted from the beads with 100 μL of 0.1 M KOH. 50 μL of the reaction mixture and elution were dried onto a LumaPlate-96 (Packard) and the amount of ³⁵S on the plate was measured using a TopCount NXT instrument (Packard). The amount of fibronectin-based scaffold protein bound to the target was estimated as a percent of radioactivity eluted from Protein A magnetic beads compare to radioactivity in the reaction mixture. Nonspecific binding of fibronectin-based scaffold proteins to the beads was determined by measuring binding in the absence of KDR-Fc and represented less than 1-2% of the input. Specific binding was obtained through subtraction of nonspecific binding from total binding. Data was analyzed using the GraphPad Prizm software (GraphPad Software, Inc, San Diego, Calif.), fitted using a one site, non-linear binding equation.

### Expression and Purification of Soluble Fibronectin-Based Scaffold Protein Binders:

For expression in E. coli residues 1-101 of each clone followed by the His₆ tag were cloned into a pET9d-derived vector and expressed in E. coli BL21 (DE3) pLysS cells (Invitrogen). 20 ml of overnight culture was used to inoculate 1 liter of LB medium containing 50 μg/mL kanamycin and 34 μg/mL chloromphenicol. The culture was grown at 37° C. until A₆₀₀ 0.4-0.6. After induction with 1 mM isopropyl-β-thiogalactoside (IPTG, Invitrogen) the culture was grown for another 3 hours at 37° C. and harvested by centrifugation for 30 minutes at 3,000 g at 4° C. The cell pellet was resuspended in 50 mL of lysis buffer K (50 mM NaH₂PO₄, 0.5 M NaCl, 5% glycerol, 5 mM CHAPS, 25 mM imidazole, 1× Complete™ Protease Inhibitor Cocktail (Roche), pH 8.0) Buffer L and sonicated on ice at 80 W for four 15 second pulses separated by ten-second pauses. The soluble fraction was separated by centrifugation for 30 minutes at 30,000 g at 4° C. The supernatant was rotated for 1 hour at 4° C. with 10 mL of TALON™ Superflow™ Metal Affinity Resin (Clontech) pre-equilibrated with wash buffer L (50 mM NaH₂PO₄, 0.5 M NaCl, 5% glycerol, 25 mM imidazole, pH 8.0). The resin was then washed with 10 column volumes of buffer L and 30 column volumes of buffer M (1×PBS, pH 7.4, 25 mM imidazole, pH 7.4). Protein was eluted with 5 column volumes of buffer N (1×PBS, 250 mM imidazole, pH 7.4) and dialyzed against 1×PBS at 4° C. Any precipitate was removed by filtering at 0.22 μm (Millipore).

### BIAcore Analysis of the Soluble Fibronectin-Based Scaffold Proteins:

The binding kinetics of fibronectin-based scaffold proteins binding proteins to the target was measured using BIAcore 2000 biosensor (Pharmacia Biosensor). Human and mouse VEGF-R2-Fc fusions were immobilized onto a CM5 sensor chip and soluble binding proteins were injected at concentrations ranging from 0 to 100 nM in buffer 0 (10 mM HEPES, 150 mM NaCl, 0.005% Tween 20, pH 7.4). Sensorgrams were obtained at each concentration and were evaluated using a program, BIA Evaluation 2.0 (BIAcore), to determine the rate constants kₐ (kₒₙ) and k_{d} (k_{off}) The affinity constant, K_{D} was calculated from the ratio of rate constants k_{off}/kₒₙ.

For inhibition experiments, human VEGF₁₆₅ was immobilized on a surface of CM-5 chip and KDR-Fc was injected at a concentration of 20 nM in the presence of different concentrations of soluble binding proteins ranging from 0 to 100 nM. IC₅₀ was determined at a concentration when only 50% of KDR-Fc binding to the chip was observed.

### Reversible Refolding of a VEGFR Binding Polypeptide:

Differential scanning calorimetry (DSC) analysis was performed on M5FL protein in 100 mM sodium acetate buffer (pH 4.5). An initial DSC run (Scan 1) was performed in a N-DSC II calorimeter (Calorimetry Sciences Corp) by ramping the temperature from 5-95° C. at a rate of 1 degree per minute, followed by a reverse scan (not shown) back to 10 degrees, followed by a second run (Scan 2). Under these conditions, data were best fit using a two transition model (Tm=77° C. and 67° C. using Orgin software (OrginLab Corp)). See FIG. 10.

### PEGylation of the M5FL Protein:

The C100-form of the M5FL protein, which has the complete sequence of M5FL with the Ser at position 100 mutated to a Cysteine including the additional C-terminal His-tag used to purify the protein. The purified M5FL-C100 protein was modified at the single cysteine residue by conjugating various maleimide-derivatized PEG forms (Shearwater). The resulting reacted proteins were run on a 4-12% polyacrylamide gel (FIG. 11).

### Construction of Cell Lines:

Plasmid construction. Plasmids, encoding chimeric receptors composed of the transmembrane and cytoplasmic domains of the human erythropoietin receptor (EpoR) fused to the extracellular domains of KDR, Flk-1, or human TrkB were constructed by a two-step PCR procedure. PCR products encoding the extracellular domains were amplified from plasmids encoding the entire receptor gene: KDR (amino acids 1 to 764) was derived from clone PR1371_H11 (OriGene Technologies, Rockville, Md.) with primers 5-RI-hKDR-1B/3-EPO/hKDR-2312B, flk-1 (amino acids 1 to 762) was derived from clone #4238984 (IMAGE) with primers 5-RI-mKDR-1/3-EPO/mKDR-2312, and human TrkB (from amino acids 1 to 430) from clone #X75958 (Invitrogen Genestorm) with primers 5-RI-hTrkB-1/3-EpoR/hTrkB-1310. PCR products encoding the EpoR transmembrane and cytoplasmic domains (amino acids 251 to 508) were amplified from clone #M60459 (Invitrogen Genestorm) with the common primer 3-XHO-EpoR-3066 and one of three gene-specific primers 5-hKDR/EPO-2274B (KDR), 5-mKDR/EPO-2274 (flk-1), and 5′hTrkB/EpoR-1274 (human TrkB), which added a short sequence complementary to the end of the receptor fragment PCR product. Second, PCR products encoding the two halves of the chimeric genes were mixed and amplified with 3-XHO-EpoR-3066 and the 5′ primers (5-RI-hKDR-1B, 5-RI-mKDR-1, and 5-RI-hTrkB-1) specific for each gene used in the previous cycle of amplification. The resulting PCR products were digested with EcoRI and XhoI and cloned into pcDNA3.1(+) (Invitrogen) to generate the plasmids phKE8 (human KDR/EpoR fusion), pmKE2 (flk-1/EpoR fusion), and phTE (TrkB/EpoR fusion).

Construction of cell lines for flow cytometry. CHO-K1 cells (American Type Culture Collection, Manassas, Va.) were stably transfected using Lipofectamine 2000 (Invitrogen) with either pcDNA 3.1 (Invitrogen) alone, pmKE2 alone, or a mixture of pcDNA 3.1 and a plasmid encoding full-length human KDR (Origene Inc., clone PR1371-H11). Stable transfectants were selected and maintained in the presence of 0.5 mg/ml of Geneticin (Invitrogen). The human KDR-expressing clone designated CHO-KDR and the murine VEGFR-2/EpoR-chimera-expressing population designated CHO-Flk were obtained by fluorescence activated cell sorting of the transfected population following staining with an anti-KDR polyclonal antiserum (R&D Systems). CHO-KDR and CHO-Flk cell lines were grown routinely in Dulbecco's modified Eagle's medium (DMEM; Gibco) supplemented with 10% (v/v) fetal bovine serum (FBS), 0.5 mg/ml Geneticin, 100 U/ml penicillin, 0.25 μg/ml amphotericin B, 100 μg/ml streptomycin and 2 mM L-glutamine.

Construction of Ba/F3 cell lines. Cell lines that would proliferate in response to VEGF binding by VEGFR-2 were constructed by transfection of the murine pre-B cell line Ba/F3 (DSMZ, Braunschweig, Germany) with phKE8 or pmKE2, receptor chimeras consisting of the extracellular domains of human or murine VEGFR-2 fused to the transmembrane and cytoplasmic domains of the human erythropoietin receptor (see above). Ba/F3 cells were maintained in minimal Ba/F3 medium (RPMI-1640 (Gibco) containing 10% FBS, 100 U/ml penicillin, 0.25 μg/ml amphotericin B, 100 μg/ml streptomycin and 2 mM L-glutamine) supplemented with 10% conditioned medium from WEHI-3B cells (DSMZ; grown in Iscove's modified Dulbecco's medium (Gibco)/10% FBS/25 μM β-mercaptoethanol) as a source of essential growth factors. Following electroporation with the plasmids pmKE2 or phKE8, stable transfectants were selected in 0.75 mg/ml Geneticin. Geneticin-resistant populations were transferred to minimal Ba/F3 medium containing 100 ng/ml of human VEGF₁₆₅ (R&D Systems), and the resulting VEGF-dependent populations were designated Ba/F3-Flk and Ba/F3-KDR. Control cell line expressing a chimeric TrkB receptor (Ba/F3-TrkB) that would be responsive to stimulation by NT-4, the natural ligand for TrkB was similarly constructed using the plasmid phTE and human NT-4 (R&D Systems).

### Analysis of Cell Surface Binding of Fibronectin-Based Scaffold Proteins:

Binding of fibronectin-based scaffold protein to cell-surface KDR and Flk-1 was analyzed simultaneously on VEGF-R2-expressing and control cells by flow cytometry. CHO-pcDNA3 cells (control) were released from their dishes with trypsin-EDTA, washed in Dulbecco's PBS without calcium and magnesium (D-PBS⁻; Invitrogen), and stained for 30 minutes at 37° C. with 1.5 μM CMTMR (5-(and-6)-(((4-chloromethyl)benzoyl)amino)-tetramethylrhodamine) (Molecular Probes). The cells were washed in D-PBS⁻ and incubated for a further 30 minutes at 37° C., and then resuspended in blocking buffer (D-PBS⁻/10% fetal bovine serum) on ice. CHO-KDR or CHO-Flk cells were treated identically except that CMTMR was omitted. 75,000 of CMTMR-stained CHO-pcDNA3 cells were mixed with an equal number of unstained CHO-KDR or CHO-Flk cells in each well of a V-bottom 96-well plate. All antibodies and fibronectin-based scaffold proteins were diluted in 25 μl/well of blocking buffer, and each treatment was conducted for 1 hour at 4° C. Cell mixtures were stained with His₆-tagged fibronectin-based scaffold proteins, washed twice with cold D-PBS⁻, and then treated with 2.5 μg/ml anti-His₆ MAb (R&D Systems), washed, and stained with 4 μg/ml Alexa Fluor 488-conjugated anti-mouse antibody (Molecular Probes). For cells treated with an anti-KDR mouse monoclonal antibody (Accurate Chemical, Westbury, N.Y.) or an anti-flk-1 goat polyclonal antibody (R&D Systems), the anti-His₆ step was omitted, and antibody binding was detected with the species-appropriate Alexa Fluor 488 conjugated secondary antibody (Molecular Probes). Following staining, cells were resuspended in 200 μl/well D-PBS⁻/1% FBS/1 μg/ml 7-aminoactinomycin D (7-AAD; Molecular Probes) and analyzed by flow cytometry on a FACSCalibur (Becton Dickinson, San Jose, Calif.) equipped with a 488 nM laser. Following gating to exclude dead cells (7-AAD positive), VEGFR-2-expressing cells and CHO-pcDNA3 cells were measured independently for Alexa Fluor 488 fluorescence by gating on the CMTMR-negative or -positive populations, respectively. Control experiments showed that staining with CMTMR did not interfere with the detection of Alexa Fluor 488-conjugated antibodies on the surface of the stained cells.

Cell-surface binding was also assessed by fluorescence microscopy using the secondary antibodies described above. For these studies, antibodies were diluted in D-PBS containing calcium and magnesium (D-PBS⁺)/10% FBS. Cells were grown on 24- or 96-well plates, and following staining were kept in D-PBS⁺ for observation on an inverted fluorescence microscope.

### Ba/F3 Cell Proliferation Assay:

Ba/F3 cells were washed three times in minimal Ba/F3 medium and resuspended in the same medium containing 15.8 ng/ml of proliferation factor (human VEGF₁₆₅, murine VEGF₁₆₄, or hNT-4 for Ba/F3-KDR, Ba/F3-Flk, or Ba/F3-TrkB cells, respectively), and 95 μl containing 5×10⁴ Ba/F3-KDR cells or 2×10⁴ Ba/F3-Flk or Ba/F3-TrkB cells were added per well to a 96-well tissue culture plate. 5 μl of serial dilutions of test protein in PBS was added to each well for a final volume of 100 μl Ba/F3 medium/5% PBS/15 ng/ml growth factor. After incubation for 72 hours at 37° C., proliferation was measured by addition of 20 μl of CellTiter 96® Aqueous One Solution Reagent (Promega) to each well, incubation for 4 hours at 37° C., and measurement of the absorbance at 490 nm using a microtiter plate reader (Molecular Dynamics).

### HUVEC Cell Proliferation Assay:

HUVEC cells (Clonetics, Walkersville, Md.) from passage 2-6 were grown in EGM-2 medium (Clonetics). 5000 cells/well were resuspended in 200 μl starvation medium (equal volumes of DMEM (Gibco) and F-12K medium (ATCC), supplemented with 0.2% fetal bovine serum and 1× penicillin/streptomycin/fungizone solution (Gibco)), plated in 96-well tissue culture plates and incubated for 48 hours. Fibronectin-based binding proteins were added to the wells and incubated for 1 hour at 37°, and then human VEGF₁₆₅ was added to a final concentration of 16 ng/ml. After 48 hours incubation, cell viability was measured by addition of 30 μl/well of a mixture of 1.9 mg/ml CellTiter96® AQueous MTS reagent (Promega) with 44 μg/ml phenazine methosulfate (Sigma) and measurement of absorbance at 490 nm as described above for Ba/F3 cells.

### Example 12

### Antibody Light Chain-Based VEGFR Binding Polypeptides

FIGS. 21A and 21B show amino acid sequences of VEGFR binding polypeptides (SEQ ID NOs:241-310) based on an antibody light chain variable region (VL) framework/scaffold.

Light chain variable domain proteins were generated using the PROfusion™ system, as described above for use with ¹⁰Fn3-derived proteins.

All references cited herein are hereby incorporated by reference in their entirety.

TABLE 1
Preferred Specific Peptide Sequences

| SEQ | | | | | | | | | | | | | | | | Binding | Kd | Binding | Kd |
|---|---|---|---|---|---|---|---|---|---|---|---|---|---|---|---|---|---|---|---|
| ID | Clone | N- | | DE | | | | | | | | | | | | to 1 nM | KDR, | to 1 nM | FLK, |
| NO | Name | terminus | BC Loop | Loop | FG Loop | | | | | | | | | | | KDR, % | nM | FLK, % | nM |
| | | KDR Binders | | | | | | | | | | | | | | | | | |
| 6 | K1 | Del 1-8 | RHPHFPTR | LQPPT | M | G | L | Y | G | H | E | L | L | T | P | 48 | 0.55 | | |
| 7 | K2 | Del 1-8 | RHPHFPTR | LQPPT | D | G | E | N | G | Q | F | L | L | V | P | 48 | 1.19 | | |
| 8 | K5 | Del 1-8 | RHPHFPTR | LQPPT | M | G | P | N | D | N | E | L | L | T | P | 47 | 1.54 | | |
| 9 | K3 | Del 1-8 | RHPHFPTR | LQPPT | A | G | W | D | D | H | E | L | F | I | P | 45 | 1.15 | | |
| 10 | K7 | Del 1-8 | RHPHFPTR | LQPPT | S | G | H | N | D | H | M | L | M | I | P | 40 | 2.2 | | |
| 11 | K4 | Del 1-8 | RHPHFPTR | LQPPT | A | G | Y | N | D | Q | I | L | M | T | P | 38 | 1.95 | | |
| 12 | K9 | Del 1-8 | RHPHFPTR | LQPPT | F | G | L | Y | G | K | E | L | L | I | P | 35 | 1.8 | | |
| 13 | K10 | Del 1-8 | RHPHFPTR | LQPPT | T | G | P | N | D | R | L | L | F | V | P | 33 | 0.57 | | |
| 14 | K12 | Del 1-8 | RHPHFPTR | LQPPT | D | V | Y | N | D | H | E | I | K | T | P | 29 | 0.62 | | |
| 15 | K6 | Del 1-8 | RHPHFPTR | LQPPT | D | G | K | D | G | R | V | L | L | T | P | 27 | 0.93 | | |
| 16 | K15 | Del 1-8 | RHPHFPTR | LQPPT | E | V | H | H | D | R | E | I | K | T | P | 25 | 0.35 | | |
| 17 | K11 | Del 1-8 | RHPHFPTR | LQPPT | Q | A | P | N | D | R | V | L | Y | T | P | 24 | 1.16 | | |
| 18 | K14 | Del 1-8 | RHPHFPTR | LQPPT | R | E | E | N | D | H | E | L | L | I | P | 20 | 0.57 | | |
| 19 | K8 | Del 1-8 | RHPHFPTR | LQPPT | V | T | H | N | G | H | P | L | M | T | P | 18 | 3.3 | | |
| 20 | K13 | Del 1-8 | RHPHFPTR | LQPPT | L | A | L | K | G | H | E | L | L | T | P | 17 | 0.58 | | |
| 21 | VR28 | WT | RHPHFPTR | LQPPT | V | A | Q | N | D | H | E | L | I | T | P | 3 | 11 | | |
| 22 | 159 | WT | RHPHFPTR | LQPPA | M | A | Q | S | G | H | E | L | F | T | P | | | | |
| | | KDR and FLK Binders | | | | | | | | | | | | | | | | | |
| 24 | E29 | Del 1-8 | RHPHFPTR | LQPPT | V | E | R | N | G | R | V | L | M | T | P | 41 | 44 | 1.51 | 0.91 |
| 25 | E19 | Del 1-8 | RHPHFPTR | LQPPT | V | E | R | N | G | R | H | L | M | T | P | 38 | 40 | 1.3 | 0.66 |
| 33 | E25 | Del 1-8 | RHPHFPTR | LQPPT | L | E | R | N | G | R | E | L | M | T | P | 41 | 28 | 1.58 | 1.3 |
| 45 | E9 | Del 1-8 | RHPHFPTR | LQPPT | E | E | R | N | G | R | T | L | R | T | P | 24 | 34 | 2.37 | 1.4 |
| 50 | E24 | Del 1-8 | RHPHFPTR | LQPPT | V | E | R | N | D | R | V | L | F | T | P | 24 | 29 | | |
| 54 | E26 | Del 1-8 | RHPHFPTR | LQPPT | V | E | R | N | G | R | E | L | M | T | P | 27 | 20 | 1.66 | 2.05 |
| 59 | E28 | Del 1-8 | RHPHFPTR | LQPPT | L | E | R | N | G | R | E | L | M | V | P | 19 | 21 | 1.63 | 2.1 |
| 60 | E3 | Del 1-8 | RHPHFPTR | LQPPT | D | G | R | N | D | R | K | L | M | V | P | 37 | 14 | 0.96 | 5.4 |
| 65 | E5 | Del 1-8 | RHPHFPTR | LQPPT | D | G | Q | N | G | R | L | L | N | V | P | 26 | 10 | 0.4 | 3.2 |
| 91 | E23 | Del 1-8 | RHHPHFPTR | LQPPT | V | H | W | N | G | R | E | L | M | T | P | 36 | 7 | | |
| 92 | E8 | Del 1-8 | RHPHFPTR | LQPPT | E | E | W | N | G | R | V | L | M | T | P | 51 | 10 | | |
| 93 | E27 | Del 1-8 | RHPHFPTR | LQPPT | V | E | R | N | G | H | T | L | M | T | P | 37 | 9 | | |
| 94 | E16 | Del 1-8 | RHPHFPTR | LQPPT | V | E | E | N | G | R | Q | L | M | T | P | 35 | 0 | | |
| 95 | E14 | Del 1-8 | RHPHFPTR | LQPPT | L | E | R | N | G | Q | V | L | F | T | P | 33 | 11 | | |
| 96 | E20 | Del 1-8 | RHPHFPTR | LQPPT | V | E | R | N | G | Q | V | L | Y | T | P | 43 | 11 | | |
| 97 | E21 | Del 1-8 | RHPHFPTR | LQPPT | W | G | Y | K | D | H | E | L | L | I | P | 47 | 1 | | |
| 98 | E22 | Del 1-8 | RHPHFPTR | LQPPT | L | G | R | N | D | R | E | L | L | T | P | 45 | 3 | | |
| 99 | E2 | Del 1-8 | RHPHFPTR | LQPPT | D | G | P | N | D | R | L | L | N | I | P | 53 | 10 | | |
| 100 | E12 | Del 1-8 | RHPHFPTR | LQPPT | F | A | R | D | G | H | E | I | L | T | P | 36 | 1 | | |
| 101 | E13 | Del 1-8 | RHPHFPTR | LQPPT | L | E | Q | N | G | R | E | L | M | T | P | 38 | 1 | | |
| 102 | E17 | Del 1-8 | RHPHFPTR | LQPPT | V | E | E | N | G | R | V | L | N | T | P | 32 | 10 | | |
| 103 | E15 | Del 1-8 | RHPHFPTR | LQPPT | L | E | P | N | G | R | Y | L | M | V | P | 52 | 2 | | |
| 104 | E10 | Del 1-8 | RHPHFPTR | LQPPT | E | G | R | N | G | R | E | L | F | I | P | 53 | 3 | | |
| 154 | M2 | WT | RHPHFPTR | LQPPA | W | E | R | N | G | R | E | L | F | T | P | | | | |
| 156 | M3 | WT | RHPHFPTR | LQPPA | K | E | R | N | G | R | E | L | F | T | P | | | | |
| 172 | M4 | WT | RHPHFPTH | LQPPA | T | E | R | T | G | R | E | L | F | T | P | | | | |
| 173 | M8 | WT | RHPHFPTH | LQPPA | K | E | R | S | G | R | E | L | F | T | P | | | | |
| 175 | M6 | WT | RHPHFPTH | LQPPA | L | E | R | D | G | R | E | L | F | T | P | | | | |
| 180 | M7 | WT | RHPHFPTR | LQPTT | W | E | R | N | G | R | E | L | F | T | P | | | | |
| 181 | M1 | WT | RHPHFPTR | LQPTV | L | E | R | N | D | R | E | L | F | T | P | | | | |
| 177 | M5FL | WT | RHPHFPTR | LQPPL | K | E | R | N | G | R | E | L | F | T | P | | | | |

TABLE 2
KDR & FLK binders

| | | | | | | |
|---|---|---|---|---|---|---|
| SEQ | | | | | | |
| ID | | | | | | DE |
| NO | Clone Name | N-terminus | N-Terminus Framework 1 | BC Loop | Framework 2 | Loop |

| | | | | | | |
|---|---|---|---|---|---|---|
| 23 | D12 | Del 1-8 | EVVAATPTSLLISW | RHPHFPTR | YYRITYGETGGNSPVQEFTVP | LQPPT |
| 24 | E29 | Del 1-8 | EVVAATPTSLLISW | RHPHFPTR | YYRITYGETGGNSPVQEFTVP | LQPPT |
| 25 | E19 | Del 1-8 | EVVAATPTSLLISW | RHPHFPTR | YYRITYGETGGNSPVQEFTVP | LQPPT |
| 26 | D1 | Del 1-8 | EVVAATPTSLLISW | RHPHFPTR | YYRITYGETGGNSPVQEFTVP | LQPPT |
| 27 | C6 | Del 1-8 | EVVAATPTSLLISW | RHPHFPTR | YYRITYGETGGNSPVQEFTVP | LQPPT |
| 28 | EGE5 | Del 1-8 | EVVAATPTSLLISW | RHPHFPTR | YYRITYGETGGNSPVQEFTVP | LQPPT |
| 29 | EGE2 | Del 1-8 | EVVAATPTSLLISW | RHPHFPTR | YYRITYGETGGNSPVQEFTVP | LQPPT |
| 30 | D4 | Del 1-8 | EVVAATPTSLLISW | RHPHFPTR | YYRITYGETGGNSPVQEFTVP | LQPPT |
| 31 | E25 | Del 1-8 | EVVAATPTSLLISW | RHPHFPTR | YYRITYGETGGNSPVQEFTVP | LQPPT |
| 32 | EGE6 | Del 1-8 | EVVAATPTSLLISW | RHPHFPTR | YYRITYGETGGNSPVQEFTVP | LQPPT |
| 33 | C7 | Del 1-8 | EVVAATPTSLLISW | RHPHFPTR | YYRITYGETGGNSPVQEFTVP | LQPPT |
| 34 | D9 | Del 1-8 | EVVAATPTSLLISW | RHPHFPTR | YYRITYGETGGNSPVQEFTVP | LQPPT |
| 35 | EGE3 | Del 1-8 | EVVAATPTSLLISW | RHPHFPTR | YYRITYGETGGNSPVQEFTVP | LQPPT |
| 36 | D3 | Del 1-8 | EVVAATPTSLLISW | RHPHFPTR | YYRITYGETGGNSPVQEFTVP | LQPPT |
| 37 | D2 | Del 1-8 | EVVAATPTSLLISW | RHPHFPTR | YYRITYGETGGNSPVQEFTVP | LQPPT |
| 38 | C8 | Del 1-8 | EVVAATPTSLLISW | RHPHFPTR | YYRITYGETGGNSPVQEFTVP | LQPPT |
| 39 | EGE4 | Del 1-8 | EVVAATPTSLLISW | RHPHFPTR | YYRITYGETGGNSPVQEFTVP | LQPPT |
| 40 | D7 | Del 1-8 | EVVAATPTSLLISW | RHPHFPTR | YYRITYGETGGNSPVQEFTVP | LQPPT |
| 41 | D5 | Del 1-8 | EVVAATPTSLLISW | RHPHFPTR | YYRITYGETGGNSPVQEFTVP | LQPPT |
| 42 | B3 | Del 1-8 | EVVAATPTSLLISW | RHPHFPTR | YYRITYGETGGNSPVQEFTVP | LQPPT |
| 43 | E9 | Del 1-8 | EVVAATPTSLLISW | RHPHFPTR | YYRITYGETGGNSPVQEFTVP | LQPPT |
| 44 | D6 | Del 1-8 | EVVAATPTSLLISW | RHPHFPTR | YYRITYGETGGNSPVQEFTVP | LQPPT |
| 45 | EGE7 | Del 1-8 | EVVAATPTSLLISW | RHPHFPTR | YYRITYGETGGNSPVQEFTVP | LQPPT |
| 46 | EGE1 | Del 1-8 | EVVAATPTSLLISW | RHPHFPTR | YYRITYGETGGNSPVQEFTVP | LQPPT |
| 47 | F9 | Del 1-8 | EVVAATPTSLLISW | RHPHFPTR | YYRITYGETGGNSPVQEFTVP | LQPPT |
| 48 | E24 | Del 1-8 | EVVAATPTSLLISW | RHPHFPTR | YYRITYGETGGNSPVQEFTVP | LQPPT |
| 49 | B11 | Del 1-8 | EVVAATPTSLLISW | RHPHFPTR | YYRITYGETGGNSPVQEFTVP | LQPPT |
| 50 | B12 | Del 1-8 | EVVAATPTSLLISW | RHPHFPTR | YYRITYGETGGNSPVQEFTVP | LQPPT |
| 51 | B5 | Del 1-8 | EVVAATPTSLLISW | RHPHFPTR | YYRITYGETGGNSPVQEFTVP | LQPPT |
| 52 | E26 | Del 1-8 | EVVAATPTSLLISW | RHPHFPTR | YYRITYGETGGNSPVQEFTVP | LQPPT |
| 53 | C12 | Del 1-8 | EVVAATPTSLLISW | RHPHFPTR | YYRITYGETGGNSPVQEFTVP | LQPPT |
| 54 | F4 | Del 1-8 | EVVAATPTSLLISW | RHPHFPTR | YYRITYGETGGNSPVQEFTVP | LQPPT |
| 55 | E18 | Del 1-8 | EVVAATPTSLLISW | RHPHFPTR | YYRITYGETGGNSPVQEFTVP | LQPPT |
| 56 | C11 | Del 1-8 | EVVAATPTSLLISW | RHPHFPTR | YYRITYGETGGNSPVQEFTVP | LQPPT |
| 57 | E28 | Del 1-8 | EVVAATPTSLLISW | RHPHFPTR | YYRITYGETGGNSPVQEFTVP | LQPPT |
| 58 | E3 | Del 1-8 | EVVAATPTSLLISW | RHPHFPTR | YYRITYGETGGNSPVQEFTVP | LQPPT |
| 59 | F8 | Del 1-8 | EVVAATPTSLLISW | RHPHFPTR | YYRITYGETGGNSPVQEFTVP | LQPPT |
| 60 | F3 | Del 1-8 | EVVAATPTSLLISW | RHPHFPTR | YYRITYGETGGNSPVQEFTVP | LQPPT |
| 61 | B10 | Del 1-8 | EVVAATPTSLLISW | RHPHFPTR | YYRITYGETGGNSPVQEFTVP | LQPPT |
| 62 | E6 | Del 1-8 | EVVAATPTSLLISW | RHPHFPTR | YYRITYGETGGNSPVQEFTVP | LQPPT |
| 63 | E5 | Del 1-8 | EVVAATPTSLLISW | RHPHFPTR | YYRITYGETGGNSPVQEFTVP | LQPPT |
| 64 | G4 | Del 1-8 | EVVAATPTSLLISW | RHPHFPTR | YYRITYGETGGNSPVQEFTVP | LQPPT |
| 65 | A3 | Del 1-8 | EVVAATPTSLLISW | RHPHFPTR | YYRITYGETGGNSPVQEFTVP | LQPPT |
| 66 | A4 | Del 1-8 | EVVAATPTSLLISW | RHPHFPTR | YYRITYGETGGNSPVQEFTVP | LQPPT |
| 67 | A6 | Del 1-8 | EVVAATPTSLLISW | RHPHFPTR | YYRITYGETGGNSPVQEFTVP | LQPPT |
| 68 | A7 | Del 1-8 | EVVAATPTSLLISW | RHPHFPTR | YYRITYGETGGNSPVQEFTVP | LQPPT |
| 69 | A8 | Del 1-8 | EVVAATPTSLLISW | RHPHFPTR | YYRITYGETGGNSPVQEFTVP | LQPPT |
| 70 | A9 | Del 1-8 | EVVAATPTSLLISW | RHPHFPTR | YYRITYGETGGNSPVQEFTVP | LQPPT |
| 71 | A10 | Del 1-8 | EVVAATPTSLLISW | RHPHFPTR | YYRITYGETGGNSPVQEFTVP | LQPPT |
| 72 | EGE11 | Del 1-8 | EVVAATPTSLLISW | RHPHFPTR | YYRITYGETGGNSPVQEFTVP | LQPPT |
| 73 | A11 | Del 1-8 | EVVAATPTSLLISW | RHPHFPTR | YYRITYGETGGNSPVQEFTVP | LQPPT |
| 74 | A12 | Del 1-8 | EVVAATPTSLLISW | RHPHFPTR | YYRITYGETGGNSPVQEFTVP | LQPPT |
| 75 | B4 | Del 1-8 | EVVAATPTSLLISW | RHPHFPTR | YYRITYGETGGNSPVQEFTVP | LQPPT |
| 76 | B6 | Del 1-8 | EVVAATPTSLLISW | RHPHFPTR | YYRITYGETGGNSPVQEFTVP | LQPPT |
| 77 | B7, B8 | Del 1-8 | EVVAATPTSLLISW | RHPHFPTR | YYRITYGETGGNSPVQEFTVP | LQPPT |
| 78 | B11 | Del 1-8 | EVVAATPTSLLISW | RHPHFPTR | YYRITYGETGGNSPVQEFTVP | LQPPT |
| 79 | C1 | Del 1-8 | EVVAATPTSLLISW | RHPHFPTR | YYRITYGETGGNSPVQEFTVP | LQPPT |
| 80 | C2 | Del 1-8 | EVVAATPTSLLISW | RHPHFPTR | YYRITYGETGGNSPVQEFTVP | LQPPT |
| 81 | C3 | Del 1-8 | EVVAATPTSLLISW | RHPHFPTR | YYRITYGETGGNSPVQEFTVP | LQPPT |
| 82 | C9 | Del 1-8 | EVVAATPTSLLISW | RHPHFPTR | YYRITYGETGGNSPVQEFTVP | LQPPT |
| 83 | C10 | Del 1-8 | EVVAATPTSLLISW | RHPHFPTR | YYRITYGETGGNSPVQEFTVP | LQPPT |
| 84 | D11 | Del 1-8 | EVVAATPTSLLISW | RHPHFPTR | YYRITYGETGGNSPVQEFTVP | LQPPT |
| 85 | EGE8 | Del 1-8 | EVVAATPTSLLISW | RHPHFPTR | YYRITYGETGGNSPVQEFTVP | LQPPT |
| 86 | EGE9 | Del 1-8 | EVVAATPTSLLISW | RHPHFPTR | YYRITYGETGGNSPVQEFTVP | LQPPT |
| 87 | EGE10 | Del 1-8 | EVVAATPTSLLISW | RHPHFPTR | YYRITYGETGGNSPVQEFTVP | LQPPT |
| 88 | EGE11 | Del 1-8 | EVVAATPTSLLISW | RHPHFPTR | YYRITYGETGGNSPVQEFTVP | LQPPT |
| 89 | E23 | Del 1-8 | EVVAATPTSLLISW | RHPHFPTR | YYRITYGETGGNSPVQEFTVP | LQPPT |
| 90 | E8 | Del 1-8 | EVVAATPTSLLISW | RHPHFPTR | YYRITYGETGGNSPVQEFTVP | LQPPT |
| 91 | E27 | Del 1-8 | EVVAATPTSLLISW | RHPHFPTR | YYRITYGETGGNSPVQEFTVP | LQPPT |
| 92 | E16 | Del 1-8 | EVVAATPTSLLISW | RHPHFPTR | YYRITYGETGGNSPVQEFTVP | LQPPT |
| 93 | E14 | Del 1-8 | EVVAATPTSLLISW | RHPHFPTR | YYRITYGETGGNSPVQEFTVP | LQPPT |
| 94 | E20 | Del 1-8 | EVVAATPTSLLISW | RHPHFPTR | YYRITYGETGGNSPVQEFTVP | LQPPT |
| 95 | E21 | Del 1-8 | EVVAATPTSLLISW | RHPHFPTR | YYRITYGETGGNSPVQEFTVP | LQPPT |
| 96 | E22 | Del 1-8 | EVVAATPTSLLISW | RHPHFPTR | YYRITYGETGGNSPVQEFTVP | LQPPT |
| 97 | E2 | Del 1-8 | EVVAATPTSLLISW | RHPHFPTR | YYRITYGETGGNSPVQEFTVP | LQPPT |
| 98 | E12 | Del 1-8 | EVVAATPTSLLISW | RHPHFPTR | YYRITYGETGGNSPVQEFTVP | LQPPT |
| 99 | E13 | Del 1-8 | EVVAATPTSLLISW | RHPHFPTR | YYRITYGETGGNSPVQEFTVP | LQPPT |
| 100 | E17 | Del 1-8 | EVVAATPTSLLISW | RHPHFPTR | YYRITYGETGGNSPVQEFTVP | LQPPT |
| 101 | E15 | Del 1-8 | EVVAATPTSLLISW | RHPHFPTR | YYRITYGETGGNSPVQEFTVP | LQPPT |
| 102 | E10 | Del 1-8 | EVVAATPTSLLISW | RHPHFPTR | YYRITYGETGGNSPVQEFTVP | LQPPT |
| 103 | F1 | Del 1-8 | EVVAATPTSLLISW | RHPHFPTR | YYRITYGETGGNSPVQEFTVP | LQPPT |
| 104 | F5 | Del 1-8 | EVVAATPTSLLISW | RHPHFPTR | YYRITYGETGGNSPVQEFTVP | LQPPT |
| 105 | F6 | Del 1-8 | EVVAATPTSLLISW | RHPHFPTR | YYRITYGETGGNSPVQEFTVP | LQPPT |
| 106 | F7 | Del 1-8 | EVVAATPTSLLISW | RHPHFPTR | YYRITYGETGGNSPVQEFTVP | LQPPT |
| 107 | F10 | Del 1-8 | EVVAATPTSLLISW | RHPHFPTR | YYRITYGETGGNSPVQEFTVP | LQPPT |
| 108 | F11 | Del 1-8 | EVVAATPTSLLISW | RHPHFPTR | YYRITYGETGGNSPVQEFTVP | LQPPT |
| 109 | F12 | Del 1-8 | EVVAATPTSLLISW | RHPHFPTR | YYRITYGETGGNSPVQEFTVP | LQPPT |
| 110 | G1 | Del 1-8 | EVVAATPTSLLISW | RHPHFPTR | YYRITYGETGGNSPVQEFTVP | LQPPT |
| 111 | G2 | Del 1-8 | EVVAATPTSLLISW | RHPHFPTR | YYRITYGETGGNSPVQEFTVP | LQPPT |
| 112 | G3 | Del 1-8 | EVVAATPTSLLISW | RHPHFPTR | YYRITYGETGGNSPVQEFTVP | LQPPT |
| 113 | MWF10 | WT | VSDVPRDLEVVAATPTSLLISW | RHPHFPTR | YYRITYGETGGNSPVQEFTVP | LQPPA |
| 114 | MWA10 | WT | VSDVPRDLEVVAATPTSLLISW | RHPHFPTR | YYRITYGETGGNSPVQEFTVP | LQPPA |
| 115 | MWA2 | WT | VSDVPRDLEVVAATPTSLLISW | RHPHFPTR | YYRITYGETGGNSPVQEFTVP | LQPPA |
| 116 | MWC10 | WT | VSDVPRDLEVVAATPTSLLISW | RHPHFPTR | YYRITYGETGGNSPVQEFTVP | LQPPA |
| 117 | MWB7 | WT | VSDVPRDLEVVAATPTSLLISW | RHPHFPTR | YYRITYGETGGNSPVQEFTVP | LQPPA |
| 118 | MWH8 | WT | VSDVPRDLEVVAATPTSLLISW | RHPHFPTR | YYRITYGETGGNSPVQEFTVP | LQPPA |
| 119 | MWA10 | WT | VSDVPRDLEVVAATPTSLLISW | RHPHFPTR | YYRITYGETGGNSPVQEFTVP | LQPPA |
| 120 | MWB2 | WT | VSDVPRDLEVVAATPTSLLISW | RHPHFPTR | YYRITYGETGGNSPVQEFTVP | LQPPA |
| 121 | MWC3-f1 | WT | VSDVPRDLEVVAATPTSLLISW | RHPHFPTR | YYRITYGETGGNSPVQEFTVP | LQPPA |
| 122 | MWG11 | WT | VSDVPRDLEVVAATPTSLLISW | RHPHFPTR | YYRITYGETGGNSPVQEFTVP | LQPPA |
| 123 | MWG11 | WT | VSDVPRDLEVVAATPTSLLISW | RHPHFPTR | YYRITYGETGGNSPVQEFTVP | LQPPA |
| 124 | MWD3-f1 | WT | VSDVPRDLEVVAATPTSLLISW | RHPHFPTR | YYRITYGETGGNSPVQEFTVP | LQPPA |
| 125 | MWE11 | WT | VSDVPRDLEVVAATPTSLLISW | RHPHFPTR | YYRITYGETGGNSPVQEFTVP | LQPPA |
| 126 | MWD10 | WT | VSDVPRDLEVVAATPTSLLISW | RHPHFPTR | YYRITYGETGGNSPVQEFTVP | LQPPA |
| 127 | MWC1 | WT | VSDVPRDLEVVAATPTSLLISW | RHPHFPTR | YYRITYGETGGNSPVQEFTVP | LQPPA |
| 128 | MWA12 | WT | VSDVPRDLEVVAATPTSLLISW | RHPHFPTR | YYRITYGETGGNSPVQEFTVP | LQPPA |
| 129 | MWB3-f1 | WT | VSDVPRDLEVVAATPTSLLISW | RHPHFPTR | YYRITYGETGGNSPVQEFTVP | LQPPA |
| 130 | MWA11 | WT | VSDVPRDLEVVAATPTSLLISW | RHPHFPTR | YYRITYGETGGNSPVQEFTVP | LQPPA |
| 131 | MWG12 | WT | VSDVPRDLEVVAATPTSLLISW | RHPHFPTR | YYRITYGETGGNSPVQEFTVP | LQPPA |
| 132 | MWH11 | WT | VSDVPRDLEVVAATPTSLLISW | RHPHFPTR | YYRITYGETGGNSPVQEFTVP | LQPPA |
| 133 | MWD12 | WT | VSDVPRDLEVVAATPTSLLISW | RHPHFPTR | YYRITYGETGGNSPVQEFTVP | LQPPA |
| 134 | MWH5 | WT | VSDVPRDLEVVAATPTSLLISW | RHPHFPTR | YYRITYGETGGNSPVQEFTVP | LQPPA |
| 135 | MWA1 | WT | VSDVPRDLEVVAATPTSLLISW | RHPHFPTR | YYRITYGETGGNSPVQEFTVP | LQPPA |
| 136 | MWG4-f1 | WT | VSDVPRDLEVVAATPTSLLISW | RHPHFPTR | YYRITYGETGGNSPVQEFTVP | LQPPA |
| 137 | MWA12 | WT | VSDVPRDLEVVAATPTSLLISW | RHPHFPTR | YYRITYGETGGNSPVQEFTVP | LQPPA |
| 138 | MWG11 | WT | VSDVPRDLEVVAATPTSLLISW | RHPHFPTR | YYRITYGETGGNSPVQEFTVP | LQPPA |
| 139 | MWC12 | WT | VSDVPRDLEVVAATPTSLLISW | RHPHFPTR | YYRITYGETGGNSPVQEFTVP | LQPPA |
| 140 | MWF11 | WT | VSDVPRDLEVVAATPTSLLISW | RHPHFPTR | YYRITYGETGGNSPVQEFTVP | LQPPA |
| 141 | MWE11 | WT | VSDVPRDLEVVAATPTSLLISW | RHPHFPTR | YYRITYGETGGNSPVQEFTVP | LQPPA |
| 142 | MWD10 | WT | VSDVPRDLEVVAATPTSLLISW | RHPHFPTR | YYRITYGETGGNSPVQEFTVP | LQPPA |
| 143 | MWC4-f1 | WT | VSDVPRDLEVVAATPTSLLISW | RHPHFPTR | YYRITYGETGGNSPVQEFTVP | LQPPA |
| 144 | MWF3 | WT | VSDVPRDLEVVAATPTSLLISW | RHPHFPTR | YYRITYGETGGNSPVQEFTVP | LQPPA |
| 145 | MWB2 | WT | VSDVPRDLEVVAATPTSLLISW | RHPHFPTR | YYRITYGETGGNSPVQEFTVP | LQPPA |
| 146 | MWE10 | WT | VSDVPRDLEVVAATPTSLLISW | RHPHFPTR | YYRITYGETGGNSPVQEFTVP | LQPPA |
| 147 | MWD9 | WT | VSDVPRDLEVVAATPTSLLISW | RHPHFPTR | YYRITYGETGGNSPVQEFTVP | LQPPA |
| 148 | MWH3-f1 | WT | VSDVPRDLEVVAATPTSLLISW | RHPHFPTR | YYRITYGETGGNSPVQEFTVP | LQPPA |
| 149 | MWG10 | WT | VSDVPRDLEVVAATPTSLLISW | RHPHFPTR | YYRITYGETGGNSPVQEFTVP | LQPPA |
| 150 | MWH11 | WT | VSDVPRDLEVVAATPTSLLISW | RHPHFPTR | YYRITYGETGGNSPVQEFTVP | LQPPA |
| 151 | MWF11 | WT | VSDVPRDLEVVAATPTSLLISW | RHPHFPTR | YYRITYGETGGNSPVQEFTVP | LQPPA |
| 152 | M2 | WT | VSDVPRDLEVVAATPTSLLISW | RHPHFPTR | YYRITYGETGGNSPVQEFTVP | LQPPA |
| 153 | MWB09-f1 | WT | VSDVPRDLEVVAATPTSLLISW | RHPHFPTR | YYRITYGETGGNSPVQEFTVP | LQPPA |
| 154 | M3 | WT | VSDVPRDLEVVAATPTSLLISW | RHPHFPTR | YYRITYGETGGNSPVQEFTVP | LQPPA |
| 155 | MWA3 | WT | VSDVPRDLEVVAATPTSLLISW | LHPHFPTH | YYRITYGETGGNSPVQEFTVP | LQPPA |
| 156 | MWE10 | WT | VSDVPRDLEVVAATPTSLLISW | RHPHFPTH | YYRITYGETGGNSPVQEFTVP | LQPPA |
| 157 | MWG3 | WT | VSDVPRDLEVVAATPTSLLISW | LHPHFPTH | YYRITYGETGGNSPVQEFTVP | LQPPA |
| 158 | MWD5 | WT | VSDVPRDLEVVAATPTSLLISW | RHPHFPTH | YYRITYGETGGNSPVQEFTVP | LQPPA |
| 159 | MWC3 | WT | VSDVPRDLEVVAATPTSLLISW | LHPHFPTH | YYRITYGETGGNSPVQEFTVP | LQPPA |
| 160 | MWH3 | WT | VSDVPRDLEVVAATPTSLLISW | LHPHFPTH | YYRITYGETGGNSPVQEFTVP | LQPPA |
| 161 | MWC2 | WT | VSDVPRDLEVVAATPTSLLISW | LHPHFPTH | YYRITYGETGGNSPVQEFTVP | LQPPA |
| 162 | MWE2 | WT | VSDVPRDLEVVAATPTSLLISW | LHPHFPTH | YYRITYGETGGNSPVQEFTVP | LQPPA |
| 163 | MWA2 | WT | VSDVPRDLEVVAATPTSLLISW | FHPHFPTH | YYRITYGETGGNSPVQEFTVP | LQPPA |
| 164 | MWD3 | WT | VSDVPRDLEVVAATPTSLLISW | LHPHFPTH | YYRITYGETGGNSPVQEFTVP | LQPPA |
| 165 | MWE3 | WT | VSDVPRDLEVVAATPTSLLISW | LHPHFPTH | YYRITYGETGGNSPVQEFTVP | LQPPA |
| 166 | MWB3 | WT | VSDVPRDLEVVAATPTSLLISW | FHPHFPTH | YYRITYGETGGNSPVQEFTVP | LQPPA |
| 167 | MWD2 | WT | VSDVPRDLEVVAATPTSLLISW | LHPHFPTH | YYRITYGETGGNSPVQEFTVP | LQPPA |
| 168 | MWC11 | WT | VSDVPRDLEVVAATPTSLLISW | RHPHFPTH | YYRITYGETGGNSPVQEFTVP | LQPPA |
| 169 | MWH12 | WT | VSDVPRDLEVVAATPTSLLISW | RHPHFPTH | YYRITYGETGGNSPVQEFTVP | LQPPA |
| 170 | M4 | WT | VSDVPRDLEVVAATPTSLLISW | RHPHFPTH | YYRITYGETGGNSPVQEFTVP | LQPPA |
| 171 | M8 | WT | VSDVPRDLEVVAATPTSLLISW | RHPHFPTH | YYRITYGETGGNSPVQEFTVP | LQPPA |
| 172 | MWF10-f1 | WT | VSDVPRDLEVVAATPTSLLISW | RHPHFPTH | YYRITYGETGGNSPVQEFTVP | LQPPA |
| 173 | M6 | WT | VSDVPRDLEVVAATPTSLLISW | RHPHFPTH | YYRITYGETGGNSPVQEFTVP | LQPPA |
| 174 | MWB6 | WT | VSDVPRDLEVVAATPTSLLISW | RHPHFPTR | YYRITYGETGGNSPVQEFTVP | LQPPT |
| 175 | M5FL | WT | VSDVPRDLEVVAATPTSLLISW | RHPHFPTR | YYRITYGETGGNSPVQEFTVP | LQPPL |
| 176 | MWG10-f1 | WT | VSDVPRDLEVVAATPTSLLISW | RHPHFPTR | YYRITYGETGGNSPVQEFTVP | LQPPI |
| 177 | MWD08-f1; | WT | VSDVPRDLEVVAATPTSLLISW | RHPHFPTR | YYRITYGETGGNSPVQEFTVP | LQPPI |
| | N42G | | | | | |
| 178 | M7 | WT | VSDVPRDLEVVAATPTSLLISW | RHPHFPTR | YYRITYGETGGNSPVQEFTVP | LQPTT |
| 179 | M1 | WT | VSDVPRDLEVVAATPTSLLISW | RHPHFPTR | YYRITYGETGGNSPVQEFTVP | LQPTV |
| 180 | MWA07-f1 | WT | VSDVPRDLEVVAATPTSLLISW | RPPHFPTR | YYRITYGETGGNSPVQEFTVP | LQPTV |
| 181 | MWH11-f1 | WT | VSDVPRDLEVVAATPTSLLISW | RHPHFPTR | YYRITYGETGGNSPVQEFTVP | PQPPA |
| 182 | MWF09- | WT | VSDVPRDLEVVAATPTSLLISW | RHPHFPTR | YYRITYGETGGNSPVQEFTVP | PQPPA |
| | f1;F48S | | | | | |
| 183 | MWG12-f1 | WT | VSDVPRDLEVVAATPTSLLISW | CHPHFPTR | YYRITYGETGGNSPVQEFTVP | LQPPI |

| | | | | | | | |
|---|---|---|---|---|---|---|---|
| SEQ | | | | Binding to | Binding | Kd | Kd |
| ID | | | | 1 nM | to 1 nM | KDR, | Flk, |
| NO | Framework 3 | FG Loop | Framework 4 | KDR, % | Flk-1, % | nM | nM |

| | | | | | | | |
|---|---|---|---|---|---|---|---|
| 23 | ATISGLKPGVDYTITGYAVT | V E R N G R K L M T P | ISINYRT | 46 | 47 | | |
| 24 | ATISGLKPGVDYTITGYAVT | V E R N G R V L M T P | ISINYRT | 41 | 44 | 1.51 | 0.91 |
| 25 | ATISGLKPGVDYTITGYAVT | V E R N G R H L M T P | ISINYRT | 38 | 40 | 1.3 | 0.66 |
| 26 | ATISGLKPGVDYTITGYAVT | V E R N G R M L M T P | ISINYRT | 38 | 38 | | |
| 27 | ATISGLKPGVDYTITGYAVT | L E R N G R V L M T P | ISINYRT | 36 | 49 | | |
| 28 | ATISGLKPGVDYTITGYAVT | L E R N G R V L N T P | ISINYRT | 32 | 47 | | |
| 29 | ATISGLKPGVDYTITGYAVT | V E R N G R Q L M T P | ISINYRT | 42 | 33 | | |
| 30 | ATISGLKPGVDYTITGYAVT | V E R N G R T L F T P | ISINYRT | 27 | 44 | | |
| 31 | ATISGLKPGVDYTITGYAVT | L E R N G R E L M T P | ISINYRT | 41 | 28 | 1.58 | 1.3 |
| 32 | ATISGLKPGVDYTITGYAVT | L E R N G R L L N T P | ISINYRT | 33 | 40 | | |
| 33 | ATISGLKPGVDYTITGYAVT | H E R N G R V L M T P | ISINYRT | 32 | 40 | | |
| 34 | ATISGLRPGVDYTITGYAVT | E E R N G R V L F T P | ISINYRT | 31 | 40 | | |
| 35 | ATISGLKPGVDYTITGYAVT | V E R N G R Q L Y T P | ISINYRT | 34 | 38 | | |
| 36 | ATISGLKPGVDYTITGYAVT | V E R N G R A L M T P | ISINYRT | 36 | 30 | | |
| 37 | ATISGLKPGVDYTITGYAVT | V E R N G R N L M T P | ISINYRT | 35 | 30 | | |
| 38 | ATISGLKPGVDYTITGYAVT | L E R N G R V L I T P | ISINYRT | 30 | 34 | | |
| 39 | ATISGLKPGVDYTITGYAVT | V E R N G R V L N T P | ISINYRT | 26 | 41 | | |
| 40 | ATISGLKPGVDYTITGYAVT | V E R N G K V L M T P | ISINYRT | 39 | 27 | | |
| 41 | ATISGLKPGVDYTITGYAVT | V E R N G R T L M M P | ISINYRT | 38 | 23 | | |
| 42 | ATISGLKPGVDYTITGYAVT | M E R N G R E L M T P | ISINYRT | 33 | 27 | | |
| 43 | ATISGLKPGVDYTITGYAVT | E E R N G R T L R T P | ISINYRT | 24 | 34 | 2.37 | 1.4 |
| 44 | ATISGLKPGVDYTITGYAVT | V E R N G K T L M T P | ISINYRT | 32 | 30 | | |
| 45 | ATISGLKPGVDYTITGYAVT | L E R N D R V L L T P | ISINYRT | 31 | 30 | | |
| 46 | ATISGLKPGVDYTITGYAVT | L E R N G R K L M T P | ISINYRT | 30 | 29 | | |
| 47 | ATISGLKPGVDYTITGYAVT | V E P N G R V L N T P | ISINYRT | 32 | 23 | | |
| 48 | ATISGLKPGVDYTITGYAVT | V E R N D R V L F T P | ISINYRT | 24 | 29 | | |
| 49 | ATISGLKPGVDYTITGYAVT | V E R N G R E L K T P | ISINYRT | 29 | 21 | | |
| 50 | ATISGLKPGVDYTITGYAVT | V E R N G R E L R T P | ISINYRT | 29 | 21 | | |
| 51 | ATISGLKPGVDYTITGYAVT | Q E R N G R E L M T P | ISINYRT | 27 | 24 | | |
| 52 | ATISGLKPGVDYTITGYAVT | V E R N G R E L M T P | ISINYRT | 27 | 20 | 1.66 | 2.05 |
| 53 | ATISGLKPGVDYTITGYAVT | V E R N G R V L S V P | ISINYRT | 24 | 20 | | |
| 54 | ATISGLKPGVDYTITGYAVT | V E R D G R T L R T P | ISINYRT | 31 | 18 | | |
| 55 | ATISGLKPGVDYTITGYAVT | V E R N G R E L N T P | ISINYRT | 17 | 29 | 1.2 | 0.53 |
| 56 | ATISGLKPGVDYTITGYAVT | V E R N G R V L I V P | ISINYRT | 19 | 21 | | |
| 57 | ATISGLKPGVDYTITGYAVT | L E R N G R E L M V P | ISINYRT | 19 | 21 | 1.63 | 2.1 |
| 58 | ATISGLKPGVDYTITGYAVT | D G R N D R K L M V P | ISINYRT | 37 | 14 | 0.96 | 5.4 |
| 59 | ATISGLKPGVDYTITGYAVT | V E H N G R T S F T P | ISINYRT | 33 | 13 | | |
| 60 | ATISGLKPGVDYTITGYAVT | V E R D G R K L Y T P | ISINYRT | 27 | 15 | | |
| 61 | ATISGLKPGVDYTITGYAVT | L E R N G R E L N T P | ISINYRT | 15 | 23 | | |
| 62 | ATISGLKPGVDYTITGYAVT | D G W N G R L L S I P | ISINYRT | 36 | 7 | 0.35 | 7.1 |
| 63 | ATISGLKPGVDYTITGYAVT | D G Q N G R L L N V P | ISINYRT | 26 | 10 | 0.4 | 3.2 |
| 64 | ATISGLKPGVDYTITGYAVT | I E K N G R H L N I P | ISINYRT | 21 | 12 | | |
| 65 | ATISGLKPGVDYTITGYAVT | D G W N G K M L S V P | ISINYRT | 33 | 7 | | |
| 66 | ATISGLKPGVDYTITGYAVT | D G Y N D R L L F I P | ISINYRT | 46 | 2 | | |
| 67 | ATISGLKPGVDYTITGYAVT | D G P N D R L L N I P | ISINYRT | 18 | 2 | | |
| 68 | ATISGLKPGVDYTITGYAVT | D G P N N R E L I V P | ISINYRT | 18 | 2 | | |
| 69 | ATISGLKPGVDYTITGYAVT | D G L N G K Y L F V P | ISINYRT | 38 | 4 | | |
| 70 | ATISGLKPGVDYTITGYAVT | E G W N D R E L F V P | ISINYRT | 31 | 4 | | |
| 71 | ATISGLKPGVDYTITGYAVT | F G W N G R E L L T P | ISINYRT | 34 | 4 | | |
| 72 | ATISGLKPGVDYTITGYAVT | F G W N D R E L L I P | ISINYRT | 50 | 0 | | |
| 73 | ATISGLKPGVDYTITGYAVT | L E W N N R V L M T P | ISINYRT | 26 | 6 | | |
| 74 | ATISGLKPGVDYTITGYAVT | V E W N G R V L M T P | ISINYRT | 40 | 10 | | |
| 75 | ATISGLKPGVDYTITGYAVT | N E R N G R E L M T P | ISINYRT | 19 | 12 | | |
| 76 | ATISGLKPGVDYTITGYAVT | L E R N G K E L M T P | ISINYRT | 23 | 11 | | |
| 77 | ATISGLKPGVDYTITGYAVT | V E R N G R E L L T P | ISINYRT | 18 | 10 | | |
| 78 | ATISGLKPGVDYTITGYAVT | V E R N G R E L K T P | ISINYRT | 29 | 21 | | |
| 79 | ATISGLKPGVDYTITGYAVT | Q E R N G R E L R T P | ISINYRT | 28 | 13 | | |
| 80 | ATISGLKPGVDYTITGYAVT | V E R N G R E L L W P | ISINYRT | 40 | 16 | | |
| 81 | ATISGLKPGVDYTITGYAVT | L E R N G R E L M I P | ISINYRT | 31 | 17 | | |
| 82 | ATISGLKPGVDYTITGYAVT | V E R N G L V L M T P | ISINYRT | 33 | 7 | | |
| 83 | ATISGLKPGVDYTITGYAVT | V E R N G R V L I I P | ISINYRT | 24 | 17 | | |
| 84 | ATISGLKPGVDYTITGYAVT | V E R N G H K L F T P | ISINYRT | 24 | 3 | | |
| 85 | ATISGLKPGVDYTITGYAVT | V E R N E R V L M T P | ISINYRT | 26 | 20 | | |
| 86 | ATISGLKPGVDYTITGYAVT | F G P N D R E L L T P | ISINYRT | 32 | 1 | | |
| 87 | ATISGLKPGVDYTITGYAVT | M G P N D R E L L T P | ISINYRT | 37 | 1 | | |
| 88 | ATISGLKPGVDYTITGYAVT | M G K N D R E L L T P | ISINYRT | 32 | 1 | | |
| 89 | ATISGLKPGVDYTITGYAVT | V H W N G R E L M T P | ISINYRT | 36 | 7 | | |
| 90 | ATISGLKPGVDYTITGYAVT | E E W N G R V L M T P | ISINYRT | 51 | 10 | | |
| 91 | ATISGLKPGVDYTITGYAVT | V E R N G H T L M T P | ISINYRT | 37 | 9 | | |
| 92 | ATISGLKPGVDYTITGYAVT | V E E N G R Q L M T P | ISINYRT | 35 | 0 | | |
| 93 | ATISGLKPGVDYTITGYAVT | L E R N G Q V L F T P | ISINYRT | 33 | 11 | | |
| 94 | ATISGLKPGVDYTITGYAVT | V E R N G Q V L Y T P | ISINYRT | 43 | 11 | | |
| 95 | ATISGLKPGVDYTITGYAVT | W G Y K D H E L L I P | ISINYRT | 47 | 1 | | |
| 96 | ATISGLKPGVDYTITGYAVT | L G R N D R E L L T P | ISINYRT | 45 | 3 | | |
| 97 | ATISGLKPGVDYTITGYAVT | D G P N D R L L N I P | ISINYRT | 53 | 10 | | |
| 98 | ATISGLKPGVDYTITGYAVT | F A R D G H E I L T P | ISINYRT | 36 | 1 | | |
| 99 | ATISGLKPGVDYTITGYAVT | L E Q N G R E L M T P | ISINYRT | 38 | 1 | | |
| 100 | ATISGLKPGVDYTITGYAVT | V E E N G R V L N T P | ISINYRT | 32 | 10 | | |
| 101 | ATISGLKPGVDYTITGYAVT | L E P N G R Y L M V P | ISINYRT | 52 | 2 | | |
| 102 | ATISGLKPGVDYTITGYAVT | E G R N G R E L F I P | ISINYRT | 53 | 3 | | |
| 103 | ATISGLKPGVDYTITGYAVT | S G R N D R E L L V P | ISINYRT | 18 | 2 | | |
| 104 | ATISGLRPGVDYTITGYAVT | V E R D G R E L N I P | ISINYRT | 12 | 8 | | |
| 105 | ATISGLKPGVDYTITGYAVT | V E Q N G R V L M T P | ISTNYRT | 37 | 2 | | |
| 106 | ATISGLKPGVDYTITGYAVT | V E H N G R V L N I P | ISINYRT | 30 | 7 | | |
| 107 | ATISGLKPGVDYTITGYAVT | M A P N G R E L L T P | ISINYRT | 29 | 1 | | |
| 108 | ATISGLKPGVDYTITGYAVT | V E Q N G R V L N T P | ISINYRT | 20 | 8 | | |
| 109 | ATISGLKPGVDYTITGYAVT | D G R N G H E L M T P | ISINYRT | 17 | 1 | | |
| 110 | ATISGLKPGVDYTITGYAVT | E G R N G R E L M V P | ISINYRT | 22 | 2 | | |
| 111 | ATISGLKPGVDYTITGYAVT | L E R N N R E L L T P | ISiNYRT | 25 | 9 | | |
| 112 | ATISGLKPGVDYTITGYAVT | M E R S G R E L M T P | ISINYRT | 28 | 10 | | |
| 113 | ATISGLKPGVDYTITGYAVT | R A L L S I E L F T P | ISINYRT | | | | |
| 114 | ATISGLKPGVDYTITGYAVT | F A R K G T E L F T P | ISINYRT | | | | |
| 115 | ATISGLKPGVDYTITGYAVT | L E R C G R E L F T P | ISINYRT | | | | |
| 116 | ATISGLKPGVDYTITGYAVT | R E R N G R E L F T P | ISINYRT | | | | |
| 117 | ATISGLKPGVDYTITGYAVT | K E R N G R E L F T P | ISINYRT | | | | |
| 118 | ATISGLKPGVDYTITGYAVT | C E R N G R E L F T P | ISINYRT | | | | |
| 119 | ATISGLKPGVDYTITGYAVT | L E R T G R E L F T P | ISTNYRT | | | | |
| 120 | ATISGLKPGVDYTITGYAVT | W E R T G K E L F T P | ISINYRT | | | | |
| 121 | ATISGLKPGVDYTITGYAVT | I E R T C R E L F T P | ISINYRT | | | | |
| 122 | ATISGLKPGVDYTITGYAVT | G G M I V R E L F T P | ISINYRT | | | | |
| 123 | ATISGLKPGVDYTITGYAVT | F G R S S R E L F T P | ISINYRT | | | | |
| 124 | ATISGLKPGVDYTITGYAVT | R H K S R G E L F T P | ISINYRT | | | | |
| 125 | ATISGLKPGVDYTITGYAVT | R H R D K R E L F T P | ISINYRT | | | | |
| 126 | ATISGLKPGVDYTITGYAVT | Y H R G R G E L F T P | ISINYRT | | | | |
| 127 | ATISGLKPGVDYTITGYAVT | R H R G C R E L F T P | ISINYRT | | | | |
| 128 | ATISGLKPGVDYTITGYAVT | S H R L R K E L F T P | ISINYRT | | | | |
| 129 | ATISGLKPGVDYTITGYAVT | M H R Q R G E L F T P | ISINYRT | | | | |
| 130 | ATISGLKPGVDYTITGYAVT | F H R R R G E L F T P | ISINYRT | | | | |
| 131 | ATISGLKPGVDYTITGYAVT | F H R R R G E L F T P | ISINYRT | | | | |
| 132 | ATISGLKPGVDYTITGYAVT | S H R R R N E L F T P | ISTNYRT | | | | |
| 133 | ATISGLKPGVDYTITGYAVT | L H R R V R E L F T P | ISTNYRT | | | | |
| 134 | ATISGLKPGVDYTITGYAVT | R H R R R G E L F T P | ISINYRT | | | | |
| 135 | ATISGLKPGVDYTITGYAVT | W H R S R K E L F T P | ISINYRT | | | | |
| 136 | ATISGLKPGVDYTITGYAVT | R H R S R G E L F T P | ISINYRT | | | | |
| 137 | ATISGLKPGVDYTITGYAVT | V H R T G R E L F T P | ISINYRT | | | | |
| 138 | ATISGLKPGVDYTITGYAVT | W H R V R G E L F T P | ISINYRT | | | | |
| 139 | ATISGLKPGVDYTITGYAVT | W H R V R G E L F T P | ISINYRT | | | | |
| 140 | ATISGLKPGVDYTITGYAVT | W H R W R G E L F T P | ISTNYRT | | | | |
| 141 | ATISGLKPGVDYTITGYAVT | W K R S G G E L F T P | ISINYRT | | | | |
| 142 | ATISGLKPGVDYTITGYAVT | R L X N X V E L F T P | ISINYRT | | | | |
| 143 | ATISGLKPGVDYTITGYAVT | W R T P H A E L F T P | ISINYRT | | | | |
| 144 | ATISGLKPGVDYTITGYAVT | L S P H S V E L F T P | ISINYRT | | | | |
| 145 | ATISGLKPGVDYTITGYAVT | V S R Q K A E L F T P | ISINYRT | | | | |
| 146 | ATISGLKPGVDYTITGYAVT | S S Y S K L E L F T P | ISINYRT | | | | |
| 147 | ATISGLKPGVDYTITGYAVT | L T D R G S E L F T P | ISINYRT | | | | |
| 148 | ATISGLKPGVDYTITGYAVT | G T R T R S E L F T P | ISINYRT | | | | |
| 149 | ATISGLKPGVDYTITGYAVT | P V A G C S E L F T P | ISINYRT | | | | |
| 150 | ATISGLKPGVDYTITGYAVT | W W Q T P R E L F T P | ISINYRT | | | | |
| 151 | ATISGLKPGVDYTITGYAVT | W W Q T P R E L F T P | ISINYRT | | | | |
| 152 | ATISGLKPGVDYTITGYAVT | W E R N G R E L F T P | ISINYRT | | | | |
| 153 | ATISGLKPGVDYTITGYAVT | W E W N G R E L F T P | ISINYRT | | | | |
| 154 | ATISGLKPGVDYTITGYAVT | K E R N G R E L F T P | ISINYRT | | | | |
| 155 | ATISGLKPGVDYTITGYAVT | G A L N T S E L F T P | ISINYRT | | | | |
| 156 | ATISGLKPGVDYTITGYAVT | F G R E R R E L F T P | ISINYRT | | | | |
| 157 | ATISGLKPGVDYTITGYAVT | S G R V S F E L F T P | ISTNYRT | | | | |
| 158 | ATISGLKPGVDYTITGYAVT | F H R R R G E L F T P | ISINYRT | | | | |
| 159 | ATISGLKPGVDYTITGYAVT | L I R M N T E L F T P | ISINYRT | | | | |
| 160 | ATISGLKPGVDYTITGYAVT | C L H L I T E L F T P | ISINYRT | | | | |
| 161 | ATISGLKPGVDYTITGYAVT | V L K L T L E L F T P | ISINYRT | | | | |
| 162 | ATISGLKPGVDYTITGYAVT | V L K L T L E L F T P | ISINYRT | | | | |
| 163 | ATISGLKPGVDYTITGYAVT | V L K L T L E L F T P | ISINYRT | | | | |
| 164 | ATISGLKPGVDYTITGYAVT | A L M A S G E L F T P | ISINYRT | | | | |
| 165 | ATISGLKPGVDYTITGYAVT | S M K N R L E L F T P | ISINYRT | | | | |
| 166 | ATISGLKPGVDYTITGYAVT | L R C L I P E L F T P | ISINYRT | | | | |
| 167 | ATISGLKPGVDYTITGYAVT | V S R Q K A E L F T P | ISINYRT | | | | |
| 168 | ATISGLKPGVDYTITGYAVT | W S R T G R E L F T P | ISINYRT | | | | |
| 169 | ATISGLKPGVDYTITGYAVT | V W R T G R E L F T P | ISTNYRT | | | | |
| 170 | ATISGLKPGVDYTITGYAVT | T E R T G R E L F T P | ISINYRT | | | | |
| 171 | ATISGLKPGVDYTITGYAVT | K E R S G R E L F T P | ISINYRT | | | | |
| 172 | ATISGLKPGVDYTITGYAVT | L E R N D R E L F T P | ISINYRT | | | | |
| 173 | ATISGLKPGVDYTITGYAVT | L E R D G R E L F T P | ISINYRT | | | | |
| 174 | ATISGLKPGVDYTITGYAVT | Q G R H K R E L F T P | ISINYRT | | | | |
| 175 | ATISGLKPGVDYTITGYAVT | K E R N G R E L F T P | ISINYRT | | | | |
| 176 | ATISGLKPGVDYTITGYAVT | M A Q N D H E L I T P | ISINYRT | | | | |
| 177 | ATISGLKPGVDYTITGYAVT | M A Q N D H E L I T P | ISINYRT | | | | |
| 178 | ATISGLKPGVDYTITGYAVT | W E R N G R E L F T P | ISINYRT | | | | |
| 179 | ATISGLKPGVDYTITGYAVT | L E R N D R E L F T P | ISINYRT | | | | |
| 180 | ATISGLKPGVDYTITGYAVT | L E R N D R E L F T P | ISINYRT | | | | |
| 181 | ATISGLKPGVDYTITGYAVT | K E R S G R E L F T P | ISINYRT | | | | |
| 182 | ATISGLKPGVDYTITGYAVT | L E R N D R E L F T P | ISINYRT | | | | |
| 183 | ATISGLKPGVDYTITGYAVT | M A Q N D H E L I T P | ISINYRT | | | | |

TABLE 3
KDR binders

| | | | | | | | | | | | | | | | | Binding to | Kd |
|---|---|---|---|---|---|---|---|---|---|---|---|---|---|---|---|---|---|
| SEQ ID | | | | | | | | | | | | | | | | 1 nM | KDR, |
| NO | Clone Name | N-terminus | BC Loop | DE Loop | FG Loop | | | | | | | | | | | KDR, % | nM |
| 6 | K1 | Del 1-8 | RHPHFPTR | LQPPT | M | G | L | Y | G | H | E | L | L | T | P | 48 | 0.55 |
| 7 | K2 | Del 1-8 | RHPHFPTR | LQPPT | D | G | E | N | G | Q | F | L | L | V | P | 48 | 1.19 |
| 8 | K5 | Del 1-8 | RHPHFPTR | LQPPT | M | G | P | N | D | N | E | L | L | T | P | 47 | 1.54 |
| 9 | K3 | Del 1-8 | RHPHFPTR | LQPPT | A | G | W | D | D | H | E | L | F | I | P | 45 | 1.15 |
| 311 | 3′E9 PR4 | Del 1-8 | RHPHFPTR | LQPPT | V | E | Q | D | G | H | V | L | Y | I | P | 44 | |
| 312 | 2′Del E6 PR4 | Del 1-8 | RHPHFPTR | LQPPT | M | G | K | N | G | H | E | L | L | T | P | 43 | |
| 313 | 3′D3 PR4 | Del 1-8 | RHPHFPTR | LQPPT | P | G | P | G | D | R | E | L | I | T | P | 42 | |
| 314 | 2′Del F8 PR4 | Del 1-8 | RHPHFPTR | LQPPT | A | G | P | G | A | H | E | L | L | T | P | 42 | |
| 315 | 4′B3 PR4 | Del 1-8 | RHPHFPTR | LQPPT | M | A | Q | N | N | R | E | L | L | T | P | 42 | |
| 316 | 3′E3 PR4 | Del 1-8 | RHPHFPTR | LQPPT | M | A | Q | Y | G | R | E | L | L | T | P | 41 | |
| 10 | K7 | Del 1-8 | RHPHFPTR | LQPPT | S | G | H | N | D | H | M | L | M | I | P | 40 | 2.2 |
| 317 | 3′H11 PR4 | Del 1-8 | RHPHFPTR | LQPPT | L | A | H | N | G | N | E | L | L | T | P | 39 | |
| 318 | 3′B4 PR4 | Del 1-8 | RHPHFPTR | LQPPT | V | A | W | N | G | H | E | L | M | T | P | 38 | |
| 11 | K4 | Del 1-8 | RHPHFPTR | LQPPT | A | G | Y | N | D | Q | I | L | M | T | P | 38 | 1.95 |
| 319 | 2′Del F7 PR4 | Del 1-8 | RHPHFPTR | LQPPT | L | G | L | R | D | R | E | L | F | V | P | 38 | |
| 320 | 2′Del D3 PR4 | Del 1-8 | RHPHFPTR | LQPPT | S | G | L | N | D | R | V | L | F | I | P | 38 | |
| 321 | 3′C6 PR4 | Del 1-8 | RHPHFPTR | LQPPT | M | G | P | N | D | R | E | L | L | T | P | 37 | |
| 322 | 3′F3 PR4 | Del 1-8 | RHPHFPTR | LQPPT | L | G | H | N | D | R | E | L | L | T | P | 37 | |
| 323 | 3′H3 PR4 | Del 1-8 | RHPHFPTR | LQPPT | L | G | L | N | D | R | E | L | M | T | P | 36 | |
| 324 | 1′Del G10 PR4 | Del 1-8 | RHPHFPTR | LQPPT | M | A | Q | N | G | H | K | L | M | T | P | 36 | |
| 12 | K9 | Del 1-8 | RHPHFPTR | LQPPT | F | G | L | Y | G | K | E | L | L | I | P | 35 | 1.8 |
| 325 | 2′DelE4 PR4 | Del 1-8 | RHPHFPTR | LQPPT | V | H | W | N | G | H | E | L | M | T | P | 34 | |
| 326 | 2′Del C6 PR4 | Del 1-8 | RHPHFPTR | LQPPT | M | G | F | M | A | H | E | L | M | V | P | 34 | |
| 327 | 2′Del C11 PR4 | Del 1-8 | RHPHFPTR | LQPPT | A | G | L | N | E | H | E | L | L | I | P | 34 | |
| 328 | 2′Del D10 PR4 | Del 1-8 | RHPHFPTR | LQPPT | L | A | D | N | A | R | E | L | L | T | P | 34 | |
| 329 | 2′Del H5 PR4 | Del 1-8 | RHPHFPTR | LQPPT | L | G | K | D | V | R | E | L | L | T | P | 34 | |
| 330 | 3′A7 PR4 | Del 1-8 | RHPHFPTR | LQPPT | L | S | D | S | G | H | A | L | F | T | P | 34 | |
| 331 | 2′Del E3 PR4 | Del 1-8 | RHPHFPTR | LQPPT | L | G | P | Y | E | H | E | L | L | T | P | 33 | |
| 13 | K10 | Del 1-8 | RHPHFPTR | LQPPT | T | G | P | N | D | R | L | L | F | V | P | 33 | 0.57 |
| 332 | 2′Del B5 PR4 | Del 1-8 | RHPHFPTR | LQPPT | A | G | R | H | D | H | E | L | I | I | P | 33 | |
| 333 | 3′C12 PR4 | Del 1-8 | RHPHFPTR | LQPPT | I | G | P | N | N | H | E | L | L | T | P | 33 | |
| 334 | 2′Del G9 PR4 | Del 1-8 | RHPHFPTR | LQPPT | V | E | Q | N | G | R | E | L | I | I | P | 33 | |
| 335 | 2′Del C1 PR4 | Del 1-8 | RHPHFPTR | LQPPT | A | G | L | D | E | H | E | L | L | I | P | 32 | |
| 336 | 3′E1 PR4 | Del 1-8 | RHPHFPTR | LQPPT | V | A | P | N | G | H | E | L | F | T | P | 32 | |
| 337 | 3′C3 PR4 | Del 1-8 | RHPHFPTR | LQPPT | M | A | Q | N | G | H | A | L | F | T | P | 32 | |
| 338 | 2′DelB7 PR4 | Del 1-8 | RHPHFPTR | LQPPT | V | G | Y | N | N | R | E | L | L | T | P | 32 | |
| 339 | 3′F1 PR4 | Del 1-8 | RHPHFPTR | LQPPT | V | A | Q | D | G | H | F | L | Y | T | P | 31 | |
| 340 | 2′Del B4 PR4 | Del 1-8 | RHPHFPTR | LQPPT | S | G | H | N | G | H | E | V | M | T | P | 31 | |
| 341 | 3′G3 PR4 | Del 1-8 | RHPHFPTR | LQPPT | F | D | Q | S | D | H | E | L | L | T | P | 31 | |
| 342 | 2′DelH4 PR4 | Del 1-8 | RHPHFPTR | LQPPT | V | G | P | N | E | R | M | L | M | T | P | 30 | |
| 343 | 3′D9 PR4 | Del 1-8 | RHPHFPTR | LQPPT | G | Y | Y | N | D | R | E | L | L | T | P | 30 | |
| 344 | 3′G10 PR4 | Del 1-8 | RHPHFPTR | LQPPT | L | T | H | N | D | H | E | L | L | T | P | 30 | |
| 345 | 3′B2 PR4 | Del 1-8 | RHPHFPTR | LQPPT | V | G | R | N | D | R | E | L | L | T | P | 29 | |
| 346 | 2′DelC3 PR4 | Del 1-8 | RHPHFPTR | LQPPT | W | A | Q | N | G | R | E | L | L | T | P | 29 | |
| 347 | 3′F2 PR4 | Del 1-8 | RHPHFPTR | LQPPT | L | G | K | N | D | H | E | L | L | T | P | 29 | |
| 348 | 4′C9 PR4 | Del 1-8 | RHPHFPTR | LQPPT | L | G | P | N | D | H | E | L | M | T | P | 29 | |
| 349 | 2′Del B2 PR4 | Del 1-8 | RHPHFPTR | LQPPT | T | G | W | N | G | N | E | L | F | T | P | 29 | |
| 14 | K12 | Del 1-8 | RHPHFPTR | LQPPT | D | V | Y | N | D | H | E | I | K | T | P | 29 | 0.62 |
| 350 | 4′H7 PR4 | Del 1-8 | RHPHFPTR | LQPPT | L | A | H | N | D | H | E | L | L | T | P | 29 | |
| 351 | 2′Del D1 PR4 | Del 1-8 | RHPHFPTR | LQPPT | L | E | Q | N | D | R | V | L | L | T | P | 28 | |
| 352 | 2′Del H6 PR4 | Del 1-8 | RHPHFPTR | LQPPT | T | G | H | H | D | H | E | L | I | I | P | 28 | |
| 353 | 3′B12 PR4 | Del 1-8 | RHPHFPTR | LQPPT | V | A | H | E | N | R | E | L | L | T | P | 28 | |
| 354 | 4′C5 PR4 | Del 1-8 | RHPHFPTR | LQPPT | L | G | L | N | D | H | E | L | I | T | P | 27 | |
| 15 | K6 | Del 1-8 | RHPHFPTR | LQPPT | D | G | K | D | G | R | V | L | L | T | P | 27 | 0.93 |
| 355 | 3′D8 PR4 | Del 1-8 | RHPHFPTR | LQPPT | A | G | P | N | D | H | Q | L | F | T | P | 27 | |
| 356 | 3′C5 PR4 | Del 1-8 | RHPHFPTR | LQPPT | D | A | M | Y | G | R | E | L | M | T | P | 27 | |
| 357 | 3′A8 PR4 | Del 1-8 | RHPHFPTR | LQPPT | V | A | W | D | D | H | E | L | L | T | P | 27 | |
| 358 | 2′Del F11 PR4 | Del 1-8 | RHPHFPTR | LQPPT | M | G | Q | N | D | K | E | L | I | T | P | 27 | |
| 359 | 4′D8 PR4 | Del 1-8 | RHPHFPTR | LQPPT | L | A | Q | N | G | H | E | L | Y | T | P | 26 | |
| 360 | 2′Del C5 PR4 | Del 1-8 | RHPHFPTR | LQPPT | P | G | H | N | D | H | E | L | M | V | P | 26 | |
| 16 | K15 | Del 1-8 | RHPHFPTR | LQPPT | E | V | H | H | D | R | E | I | K | T | P | 25 | 0.35 |
| 361 | 3′B1 PR4 | Del 1-8 | RHPHFPTR | LQPPT | E | A | R | N | G | R | E | L | L | T | P | 25 | |
| 362 | 3′A9 PR4 | Del 1-8 | RHPHFPTR | LQPPT | L | A | H | N | D | R | E | L | L | T | P | 25 | |
| 363 | 4′B11 PR4 | Del 1-8 | RHPHFPTR | LQPPT | M | A | H | N | D | H | E | L | L | T | P | 25 | |
| 17 | K11 | Del 1-8 | RHPHFPTR | LQPPT | Q | A | P | N | D | R | V | L | Y | T | P | 24 | 1.16 |
| 364 | 3D12 PR3 | Del 1-8 | RHPHFPTR | LQPPT | L | G | Q | N | D | R | Q | L | L | V | P | 24 | |
| 365 | 2′Del H12 PR4 | Del 1-8 | RHPHFPTR | LQPPT | A | G | G | N | G | H | E | L | L | T | P | 24 | |
| 366 | 3′H9 PR4 | Del 1-8 | RHPHFPTR | LQPPT | H | G | P | Y | D | Q | V | L | L | T | P | 24 | |
| 367 | 3′F6 PR4 | Del 1-8 | RHPHFPTR | LQPPT | I | E | Q | S | G | L | Q | L | M | T | P | 24 | |
| 368 | 1′DelE6 PR4 | Del 1-8 | RHPHFPTR | LQPPT | L | A | Q | N | D | R | E | L | L | T | P | 24 | |
| 369 | 3′E5 PR4 | Del 1-8 | RHPHFPTR | LQPPT | V | A | W | D | G | R | E | L | F | T | P | 23 | |
| 370 | 3A3 PR3 | Del 1-8 | RHPHFPTR | LQPPT | L | A | Y | N | G | R | E | I | I | T | P | 23 | |
| 371 | 3A2 PR3 | Del 1-8 | RHPHFPTR | LQPPT | W | S | Q | N | N | R | E | L | F | T | P | 23 | |
| 372 | 3′B11 PR4 | Del 1-8 | RHPHFPTR | LQPPT | E | T | W | N | D | H | E | I | R | T | P | 23 | |
| 373 | 1′DelA2 PR4 | Del 1-8 | RHPHFPTR | LQPPT | V | A | Q | N | G | H | Q | L | F | T | P | 23 | |
| 374 | 2′D6-PR4 | WT | RHPHFPTR | LQPPT | V | T | H | N | G | H | P | L | M | T | P | 22 | |
| 375 | 3′H1 PR4 | Del 1-8 | RHPHFPTR | LQPPT | F | A | Q | N | D | H | Q | L | F | T | P | 22 | |
| 376 | 2′Del G11 PR4 | Del 1-8 | RHPHFPTR | LQPPT | G | G | Q | M | N | R | V | L | M | T | P | 22 | |
| 377 | 2′Del F5 PR4 | Del 1-8 | RHPHFPTR | LQPPT | L | V | H | N | D | R | E | L | L | T | P | 22 | |
| 378 | 1′DelE7 PR4 | Del 1-8 | RHPHFPTR | LQPPT | V | A | Q | N | G | H | E | L | F | T | P | 22 | |
| 379 | 2′E4-PR4 | WT | RHPHFPTR | LQPPT | V | H | W | N | G | H | E | L | M | T | P | 22 | |
| 380 | 2′Del F6 PR4 | Del 1-8 | RHPHFPTR | LQPPT | L | G | W | N | D | H | E | L | Y | I | P | 22 | |
| 381 | 3′E10 PR4 | Del 1-8 | RHPHFPTR | LQPPT | A | G | H | K | D | Q | E | L | L | T | P | 21 | |
| 382 | 4′A9 PR4 | Del 1-8 | RHPHFPTR | LQPPT | L | A | Q | N | N | H | E | L | L | T | P | 21 | |
| 383 | 4′G12 PR4 | Del 1-8 | RHPHFPTR | LQPPT | V | A | W | N | D | H | E | I | Y | T | P | 21 | |
| 384 | 3′B10 PR4 | Del 1-8 | RHPHFPTR | LQPPT | L | A | Q | T | G | R | E | L | L | T | P | 21 | |
| 385 | 2′DelH9 PR4 | Del 1-8 | RHPHFPTR | LQPPT | V | G | W | S | G | H | E | L | F | V | P | 20 | |
| 386 | 3′H8 PR4 | Del 1-8 | RHPHFPTR | LQPPT | V | G | H | N | D | R | E | L | I | T | P | 20 | |
| 387 | 2′DelA5 PR4 | Del 1-8 | RHPHFPTR | LQPPT | W | N | Q | N | G | Q | E | L | F | T | P | 20 | |
| 388 | 3B5 PR3 | Del 1-8 | RHPHFPTR | LQPPT | F | G | Q | N | G | H | A | L | L | T | P | 20 | |
| 389 | 3C7 PR3 | Del 1-8 | RHPHFPTR | LQPPT | R | G | L | N | D | G | E | L | L | T | P | 20 | |
| 390 | 3G2 PR3 | Del 1-8 | RHPHFPTR | LQPPT | F | G | P | S | D | H | V | L | L | T | P | 20 | |
| 18 | K14 | Del 1-8 | RHPHFPTR | LQPPT | R | E | E | N | D | H | E | L | L | I | P | 20 | 0.57 |
| 391 | 4′B12 PR4 | Del 1-8 | RHPHFPTR | LQPPT | L | A | Q | N | N | H | E | L | L | T | P | 20 | |
| 392 | 4′B8 PR4 | Del 1-8 | RHPHFPTR | LQPPT | V | A | Q | N | D | H | K | L | F | I | P | 20 | |
| 393 | 2′Del F1 PR4 | Del 1-8 | RHPHFPTR | LQPPT | R | D | Q | Y | E | H | E | L | L | T | P | 20 | |
| 394 | 3′G1 PR4 | Del 1-8 | RHPHFPTR | LQPPT | L | A | L | N | G | H | E | L | F | T | P | 19 | |
| 395 | 3′D2 PR4 | Del 1-8 | RHPHFPTR | LQPPT | V | E | S | N | G | H | A | L | F | V | P | 19 | |
| 396 | 2′DelG5 PR4 | Del 1-8 | RHPHFPTR | LQPPT | V | G | Q | N | N | H | E | L | L | T | P | 19 | |
| 397 | 2′DelC7 PR4 | Del 1-8 | RHPHFPTR | LQPPT | W | D | Q | N | G | H | V | L | L | T | P | 19 | |
| 398 | 2′Del E5 PR4 | Del 1-8 | RHPHFPTR | LQPPT | E | G | L | N | D | H | E | L | I | I | P | 19 | |
| 399 | 3′C8 PR4 | Del 1-8 | RHPHFPTR | LQPPT | E | G | L | N | D | H | E | L | M | I | P | 19 | |
| 400 | 3′G7 PR4 | Del 1-8 | RHPHFPTR | LQPPT | E | G | Q | N | D | Q | L | L | F | I | P | 19 | |
| 401 | 3′A6 PR4 | Del 1-8 | RHPHFPTR | LQPPT | L | A | Q | N | G | H | E | L | L | T | P | 19 | |
| 402 | 4′G4 PR4 | Del 1-8 | RHPHFPTR | LQPPT | V | A | Q | N | D | R | E | L | L | T | P | 19 | |
| 403 | 4′H5 PR4 | Del 1-8 | RHPHFPTR | LQPPT | L | A | Q | N | G | H | E | L | F | T | P | 18 | |
| 404 | 1′DelH12 PR4 | Del 1-8 | RHPHFPTR | LQPPT | V | A | Q | N | E | R | E | L | F | T | P | 18 | |
| 19 | K8 | Del 1-8 | RHPHFPTR | LQPPT | V | T | H | N | G | H | P | L | M | T | P | 18 | 3.3 |
| 405 | 2′Del D5 PR4 | Del 1-8 | RHPHFPTR | LQPPT | V | A | W | N | D | H | M | L | M | T | P | 18 | |
| 406 | 3′F9 PR4 | Del 1-8 | RHPHFPTR | LQPPT | L | G | P | N | D | R | E | L | M | T | P | 18 | |
| 407 | 2′H4-PR4 | WT | RHPHFPTR | LQPPT | V | G | P | N | E | R | M | L | M | T | P | 17 | |
| 408 | 4′H12 PR4 | Del 1-8 | RHPHFPTR | LQPPT | V | A | H | N | D | H | E | L | L | T | P | 17 | |
| 409 | 1′DelD2 PR4 | Del 1-8 | RHPHFPTR | LQPPT | V | A | K | N | D | H | E | L | L | T | P | 17 | |
| 410 | 4′E7 PR4 | Del 1-8 | RHPHFPTR | LQPPT | W | A | Q | N | D | H | E | L | L | T | P | 17 | |
| 411 | 1′DelH10 PR4 | Del 1-8 | RHPHFPTR | LQPPT | F | A | Q | N | D | H | E | L | L | T | P | 17 | |
| 20 | K13 | Del 1-8 | RHPHFPTR | LQPPT | L | A | L | K | G | H | E | L | L | T | P | 17 | 0.58 |
| 412 | 3C3 PR3 | Del 1-8 | RHPHFPTR | LQPPT | M | E | Q | N | G | H | E | L | F | T | P | 17 | |
| 413 | 2′Del B3 PR4 | Del 1-8 | RHPHFPTR | LQPPT | D | A | P | N | G | R | E | L | M | V | P | 17 | |
| 414 | 2′Del A2 PR4 | Del 1-8 | RHPHFPTR | LQPPT | G | G | R | N | G | H | T | L | L | T | P | 17 | |
| 415 | 3′F12 PR4 | Del 1-8 | RHPHFPTR | LQPPT | L | S | Q | T | D | H | E | L | L | T | P | 17 | |
| 416 | 3B4 PR3 | Del 1-8 | RHPHFPTR | LQPPT | V | G | Q | N | E | H | E | L | L | T | P | 17 | |
| 417 | 3F8 PR3 | Del 1-8 | RHPHFPTR | LQPPT | V | A | Q | N | G | H | E | L | K | T | P | 17 | |
| 418 | 1′DelH5 PR4 | Del 1-8 | RHPHFPTR | LQPPT | V | A | Q | N | D | R | E | L | F | T | P | 17 | |
| 419 | 1′DelD5 PR4 | Del 1-8 | RHPHFPTR | LQPPT | V | G | Q | N | H | H | E | L | F | T | P | 17 | |
| 420 | 3′E11 PR4 | Del 1-8 | RHPHFPTR | LQPPT | V | G | P | H | D | R | E | L | L | T | P | 17 | |
| 421 | 2′C6-PR4 | WT | RHPHFPTR | LQPPT | M | G | F | M | A | H | E | L | M | V | P | 16 | |
| 422 | 4C9 PR3 | Del 1-8 | RHPHFPTR | LQPPT | L | A | Q | N | D | H | E | L | L | T | P | 16 | |
| 423 | 3C9 PR3 | Del 1-8 | RHPHFPTR | LQPPT | L | V | R | N | D | H | E | L | L | T | P | 16 | |
| 424 | 3F10 PR3 | Del 1-8 | RHPHFPTR | LQPPT | L | A | Q | D | D | H | E | L | L | T | P | 16 | |
| 425 | 2′Del A11 PR4 | Del 1-8 | RHPHFPTR | LQPPT | E | D | I | R | V | L | W | L | N | T | T | 16 | |
| 426 | 1′DelD1 PR4 | Del 1-8 | RHPHFPTR | LQPPT | V | T | Q | N | D | H | E | L | L | T | P | 16 | |
| 427 | 1′DelE2 PR4 | Del 1-8 | RHPHFPTR | LQPPT | V | G | Q | N | D | H | E | L | L | T | P | 16 | |
| 428 | 1′DelF3 PR4 | Del 1-8 | RHPHFPTR | LQPPT | M | A | Q | N | D | H | K | L | F | T | P | 16 | |
| 429 | 4′A5 PR4 | Del 1-8 | RHPHFPTR | LQPPT | L | A | Q | N | D | H | E | L | L | T | P | 16 | |
| 430 | 1′DelB8 PR4 | Del 1-8 | RHPHFPTR | LQPPT | M | A | Q | N | D | H | E | L | L | T | P | 16 | |
| 431 | 4′B7 PR4 | Del 1-8 | RHPHFPTR | LQPPT | V | A | Q | N | N | H | E | L | L | T | P | 16 | |
| 432 | 4F4 PR3 | Del 1-8 | RHPHFPTR | LQPPT | L | A | Q | N | D | R | E | L | I | T | P | 15 | |
| 433 | 4B11 PR3 | Del 1-8 | RHPHFPTR | LQPPT | V | G | Q | N | N | H | E | L | I | T | P | 15 | |
| 434 | 3′G2 PR4 | Del 1-8 | RHPHFPTR | LQPPT | L | A | Q | N | G | H | E | L | I | T | P | 15 | |
| 435 | 2′Del C8 PR4 | Del 1-8 | RHPHFPTR | LQPPT | T | A | H | N | G | H | E | L | L | T | P | 15 | |
| 436 | 3′B8 PR4 | Del 1-8 | RHPHFPTR | LQPPT | L | G | Y | H | D | H | A | L | F | T | P | 14 | |
| 437 | 3′H10 PR4 | Del 1-8 | RHPHFPTR | LQPPT | W | A | W | N | D | H | E | L | M | T | P | 14 | |
| 438 | 1′DelA1 PR4 | Del 1-8 | RHPHFPTR | LQPPT | V | A | Q | N | D | H | E | L | L | T | P | 14 | |
| 439 | 4′D6 PR4 | Del 1-8 | RHPHFPTR | LQPPT | M | A | Q | N | D | H | E | L | M | T | P | 14 | |
| 440 | 4F9 PR3 | Del 1-8 | RHPHFPTR | LQPPT | M | A | Q | N | D | H | E | L | L | T | P | 14 | |
| 441 | 4H5 PR3 | Del 1-8 | RHPHFPTR | LQPPT | V | A | Q | N | G | H | E | L | I | T | P | 14 | |
| 442 | 2D12 PR3 | WT | RHPHFPTR | LQPPT | E | G | W | I | D | H | E | I | M | I | P | 14 | |
| 443 | 3′F7 PR4 | Del 1-8 | RHPHFPTR | LQPPT | E | G | Q | N | G | S | E | L | I | V | P | 14 | |
| 444 | 4C11 PR3 | Del 1-8 | RHPHFPTR | LQPPT | M | A | Q | N | D | R | E | L | I | T | P | 14 | |
| 445 | 4B6 PR3 | Del 1-8 | RHPHFPTR | LQPPT | V | G | Q | N | D | H | E | L | F | T | P | 14 | |
| 446 | 1′DelE12 PR4 | Del 1-8 | RHPHFPTR | LQPPT | V | A | Q | S | D | H | E | L | F | T | P | 13 | |
| 447 | 1′DelC2 PR4 | Del 1-8 | RHPHFPTR | LQPPT | V | D | R | N | D | H | E | L | F | T | P | 13 | |
| 448 | 1′DelA9 PR4 | Del 1-8 | RHPHFPTR | LQPPT | L | A | Q | N | D | H | E | L | M | T | P | 13 | |
| 449 | 1′DelA4 PR4 | Del 1-8 | RHPHFPTR | LQPPT | V | A | Q | N | D | H | E | L | F | T | P | 13 | |
| 450 | 3G5 PR3 | Del 1-8 | RHPHFPTR | LQPPT | L | G | E | N | D | R | K | L | I | T | P | 13 | |
| 451 | 4A12 PR3 | Del 1-8 | RHPHFPTR | LQPPT | V | A | Q | N | D | H | E | L | L | T | P | 13 | |
| 452 | 2′Del E12 PR4 | Del 1-8 | RHPHFPTR | LQPPT | E | G | P | N | G | H | E | L | I | T | P | 13 | |
| 453 | 3G1 PR3 | Del 1-8 | RHPHFPTR | LQPPT | M | A | Q | N | V | R | E | L | L | T | P | 13 | |
| 454 | 4F12 PR3 | Del 1-8 | RHPHFPTR | LQPPT | V | T | Q | N | G | H | E | L | I | T | P | 13 | |
| 455 | 4B7 PR3 | Del 1-8 | RHPHFPTR | LQPPT | V | T | Q | N | D | H | E | L | F | T | P | 13 | |
| 456 | 4′G8 PR4 | Del 1-8 | RHPHFPTR | LQPPT | V | A | Q | N | G | H | E | L | L | T | P | 13 | |
| 457 | 3′E8 PR4 | Del 1-8 | RHPHFPTR | LQPPT | V | A | Q | N | D | R | Q | L | F | T | P | 12 | |
| 458 | 3′E4 PR4 | Del 1-8 | RHPHFPTR | LQPPT | V | G | P | N | D | R | E | L | I | V | P | 12 | |
| 459 | 1′DelC6 PR4 | Del 1-8 | RHPHFPTR | LQPPT | V | A | Q | N | E | H | E | L | L | T | P | 12 | |
| 460 | 1′DelD3 PR4 | Del 1-8 | RHPHFPTR | LQPPT | L | A | Q | N | N | H | E | L | I | T | P | 12 | |
| 461 | 3A8 PR3 | Del 1-8 | RHPHFPTR | LQPPT | E | A | H | H | G | H | E | L | M | I | P | 12 | |
| 462 | 3C5 PR3 | Del 1-8 | RHPHFPTR | LQPPT | G | D | H | N | D | R | E | L | M | T | P | 12 | |
| 463 | 2′G11-PR4 | WT | RHPHFPTR | LQPPT | G | G | Q | M | N | R | V | L | M | T | P | 12 | |
| 464 | 3′D4 PR4 | Del 1-8 | RHPHFPTR | LQPPT | L | A | H | N | D | R | E | L | I | T | P | 12 | |
| 465 | 3E6 PR3 | Del 1-8 | RHPHFPTR | LQPPT | V | P | Q | N | G | H | E | L | I | T | M | 12 | |
| 466 | 1′DelA11 PR4 | Del 1-8 | RHPHFPTR | LQPPT | L | A | Q | N | D | H | E | L | F | T | P | 12 | |
| 467 | 4′D12 PR4 | Del 1-8 | RHPHFPTR | LQPPT | V | D | Q | N | D | H | E | L | F | T | P | 12 | |
| 468 | 2′D5-PR4 | WT | RHPHFPTR | LQPPT | V | A | W | N | D | H | M | L | M | T | P | 11 | |
| 469 | 2′A1-PR4 | WT | RHPHFPTR | LQPPT | S | G | H | N | D | H | M | L | M | I | P | 11 | |
| 470 | 1′DelG11 PR4 | Del 1-8 | RHPHFPTR | LQPPT | L | A | Q | N | G | H | V | L | I | T | P | 11 | |
| 471 | 2′DelB10 PR4 | Del 1-8 | RHPHFPTR | LQPPT | V | T | H | N | D | H | E | L | L | T | P | 11 | |
| 472 | 2′DelB11 PR4 | Del 1-8 | RHPHFPTR | LQPPT | V | G | Q | N | D | H | E | L | M | T | P | 11 | |
| 473 | 1′DelC5 PR4 | Del 1-8 | RHPHFPTR | LQPPT | L | A | Q | N | D | H | E | I | M | T | P | 11 | |
| 474 | 4′B6 PR4 | Del 1-8 | RHPHFPTR | LQPPT | L | A | Q | N | D | H | E | L | I | T | P | 11 | |
| 475 | 3H9 PR3 | Del 1-8 | RHPHFPTR | LQPPT | V | S | Q | Q | N | H | E | L | L | T | P | 11 | |
| 476 | 4E10 PR3 | Del 1-8 | RHPHFPTR | LQPPT | V | A | Q | N | D | H | E | L | M | T | P | 11 | |
| 477 | 3F5 PR3 | Del 1-8 | RHPHFPTR | LQPPT | V | A | Y | N | E | H | E | L | Y | T | P | 11 | |
| 478 | 4A9 PR3 | Del 1-8 | RHPHFPTR | LQPPT | V | A | Q | H | D | H | E | L | L | T | P | 11 | |
| 479 | 1′DelH7 PR4 | Del 1-8 | RHPHFPTR | LQPPT | V | G | Q | N | D | Q | E | L | L | T | P | 11 | |
| 480 | 1′DelB10 PR4 | Del 1-8 | RHPHFPTR | LQPPT | V | A | R | N | D | H | E | L | M | T | P | 11 | |
| 481 | 2′DelB9 PR4 | Del 1-8 | RHPHFPTR | LQPPT | V | G | P | T | D | H | E | L | L | T | P | 11 | |
| 482 | 3F11 PR3 | Del 1-8 | RHPHFPTR | LQPPT | V | G | L | T | D | H | V | L | L | T | P | 10 | |
| 483 | 4C4 PR3 | Del 1-8 | RHPHFPTR | LQPPT | V | A | Q | D | D | H | E | L | F | T | P | 10 | |
| 484 | 4B5 PR3 | Del 1-8 | RHPHFPTR | LQPPT | L | A | Q | N | D | H | E | L | F | T | P | 10 | |
| 485 | 3D4 PR3 | Del 1-8 | RHPHFPTR | LQPPT | V | G | W | N | D | H | E | L | I | T | P | 10 | |
| 486 | 4A4 PR3 | Del 1-8 | RHPHFPTR | LQPPT | V | A | Q | N | D | H | E | L | F | T | P | 10 | |
| 487 | 3D11 PR3 | Del 1-8 | RHPHFPTR | LQPPT | L | G | Q | E | N | Q | E | L | I | T | P | 10 | |
| 488 | 2H10 PR3 | WT | RHPHFPTR | LQPPT | L | A | P | S | A | R | E | L | M | T | P | 10 | |
| 489 | 3G10 PR3 | Del 1-8 | RHPHFPTR | LQPPT | V | V | H | N | G | H | E | I | L | T | P | 10 | |
| 490 | 3F4 PR3 | Del 1-8 | RHPHFPTR | LQPPT | M | G | Y | E | D | H | E | L | I | T | P | 10 | |
| 491 | 2H12 PR3 | WT | RHPHFPTR | LQPPT | E | G | Y | Q | N | H | E | L | S | V | P | 10 | |
| 492 | 4C2 PR3 | Del 1-8 | RHPHFPTR | LQPPT | V | D | Q | N | D | H | E | L | F | T | P | 10 | |
| 493 | 1′DelG9 PR4 | Del 1-8 | RHPHFPTR | LQPPT | V | A | Q | S | D | H | E | L | M | T | P | 10 | |
| 494 | 1′DelH9 PR4 | Del 1-8 | RHPHFPTR | LQPPT | V | G | Q | N | D | H | E | L | I | T | P | 10 | |
| 495 | 1′DelB3 PR4 | Del 1-8 | RHPHFPTR | LQPPT | V | A | Q | N | D | H | E | L | M | T | P | 10 | |
| 496 | 1′DelH1 PR4 | Del 1-8 | RHPHFPTR | LQPPT | V | A | Q | N | G | H | E | L | I | T | P | 9 | |
| 497 | 3′A3 PR4 | Del 1-8 | RHPHFPTR | LQPPT | R | A | Q | N | D | H | E | L | I | T | P | 9 | |
| 498 | 1′DelC4 PR4 | Del 1-8 | RHPHFPTR | LQPPT | V | A | Q | S | N | H | E | L | M | T | P | 9 | |
| 499 | 1′DelE11 PR4 | Del 1-8 | RHPHFPTR | LQPPT | V | A | Q | N | D | R | E | L | I | T | P | 9 | |
| 500 | 3F1 PR3 | Del 1-8 | RHPHFPTR | LQPPT | L | T | H | N | E | Q | Y | L | F | T | P | 9 | |
| 501 | 2G9 PR3 | WT | RHPHFPTR | LQPPT | E | I | Y | N | D | H | E | L | M | T | P | 9 | |
| 502 | 3′D11 PR4 | Del 1-8 | RHPHFPTR | LQPPT | M | A | Q | N | D | H | E | L | I | T | P | 9 | |
| 503 | 2′DelH2 PR4 | Del 1-8 | RHPHFPTR | LQPPT | V | S | Q | Y | G | H | E | L | I | T | P | 8 | |
| 504 | 1′DelC10 PR4 | Del 1-8 | RHPHFPTR | LQPPT | V | A | K | N | D | H | E | L | I | T | P | 8 | |
| 505 | 4D2 PR3 | Del 1-8 | RHPHFPTR | LQPPT | V | A | Q | N | N | H | E | L | I | T | P | 8 | |
| 506 | 4A9 PR3 | Del 1-8 | RHPHFPTR | LQPPT | V | A | Q | H | D | H | E | L | L | T | P | 8 | |
| 507 | 2F3 PR3 | WT | RHPHFPTR | LQPPT | L | S | H | Y | G | K | E | L | R | T | P | 8 | |
| 508 | 4A2 PR3 | Del 1-8 | RHPHFPTR | LQPPT | V | A | Q | N | A | H | E | L | M | T | P | 8 | |
| 509 | 4G4 PR3 | Del 1-8 | RHPHFPTR | LQPPT | L | G | Q | N | D | H | E | L | L | T | P | 8 | |
| 510 | 1′DelB7 PR4 | Del 1-8 | RHPHFPTR | LQPPT | V | A | Q | N | D | H | E | L | K | T | P | 8 | |
| 511 | 3′D12 PR4 | Del 1-8 | RHPHFPTR | LQPPT | G | E | Q | N | D | Y | E | L | L | V | P | 7 | |
| 512 | 2′Del F12 PR4 | Del 1-8 | RHPHFPTR | LQPPT | L | T | Q | H | D | H | E | L | L | T | P | 7 | |
| 513 | 4E2 PR3 | Del 1-8 | RHPHFPTR | LQPPT | M | A | Q | N | D | H | E | L | I | T | P | 7 | |
| 514 | 2C9 PR3 | WT | RHPHFPTR | LQPPT | E | A | P | N | G | R | E | L | R | T | P | 7 | |
| 515 | 2′B9-PR4 | WT | RHPHFPTR | LQPPT | V | G | P | T | D | H | E | L | L | T | P | 7 | |
| 516 | 1′DelH6 PR4 | Del 1-8 | RHPHFPTR | LQPPT | V | G | Q | Y | D | H | E | L | I | T | P | 6 | |
| 517 | 4A3 PR3 | Del 1-8 | RHPHFPTR | LQPPT | V | A | Q | D | E | H | E | L | I | T | P | 6 | |
| 518 | 2C12 PR3 | WT | RHPHFPTR | LQPPT | D | A | Q | N | V | Q | A | P | I | A | Q | 6 | |
| 519 | 2G12 PR3 | WT | RHPHFPTR | LQPPT | S | G | Q | N | D | H | A | L | L | I | P | 6 | |
| 520 | 2′A11-PR4 | WT | RHPHFPTR | LQPPT | E | D | I | R | V | L | W | L | N | T | T | 6 | |
| 521 | 2′C7-PR4 | WT | RHPHFPTR | LQPPT | W | D | Q | N | G | H | V | L | L | T | P | 5 | |
| 522 | 2′F7-PR4 | WT | RHPHFPTR | LQPPT | L | G | L | R | D | R | E | L | F | V | P | 5 | |
| 523 | 2C6 PR3 | WT | RHPHFPTR | LQPPT | V | E | P | N | G | H | K | L | A | I | P | 5 | |
| 524 | 3′E6 PR4 | Del 1-8 | RHPHFPTR | LQPPT | F | G | Q | N | G | K | E | F | R | I | P | 5 | |
| 525 | 2′B4-PR4 | WT | RHPHFPTR | LQPPT | S | G | H | N | G | H | E | V | M | T | P | 4 | |
| 526 | 2′F6-PR4 | WT | RHPHFPTR | LQPPT | L | G | W | N | D | H | E | L | Y | I | P | 4 | |
| 527 | 2′H5-PR4 | WT | RHPHFPTR | LQPPT | L | G | K | D | V | R | E | L | L | T | P | 3 | |
| 528 | 2F10 PR3 | WT | RHPHFPTR | LQPPT | L | A | L | F | D | H | E | L | L | T | P | 3 | |
| 21 | VR28 | WT | RHPHFPTR | LQPPT | V | A | Q | N | D | H | E | L | I | T | P | 3 | 11 |
| 22 | 159 | WT | RHPHFPTR | LQPPA | M | A | Q | S | G | H | E | L | F | T | P | | |

TABLE 4
Sequences of characterized VEGF-R2 binding clones

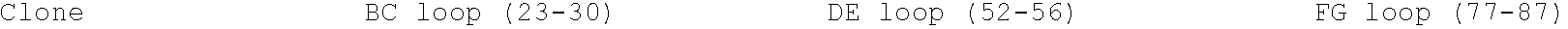

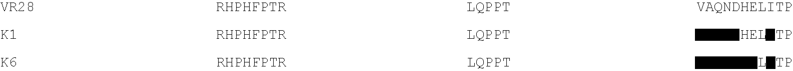

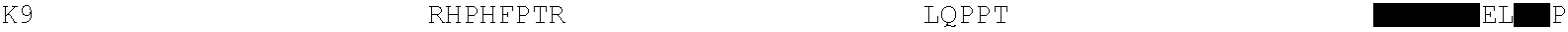

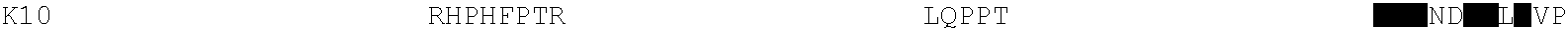

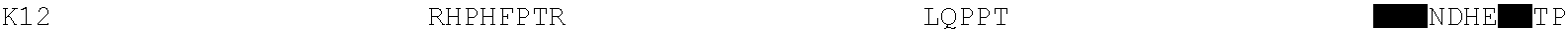

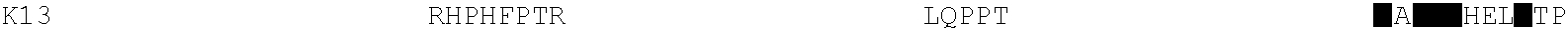

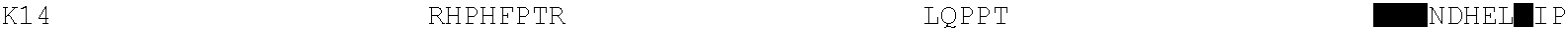

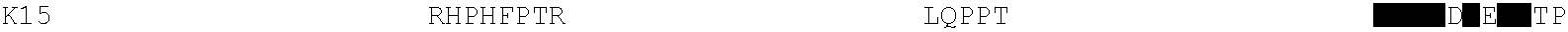

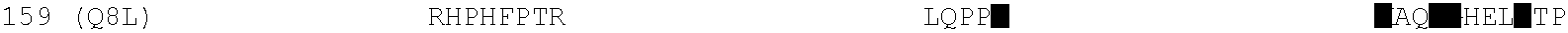

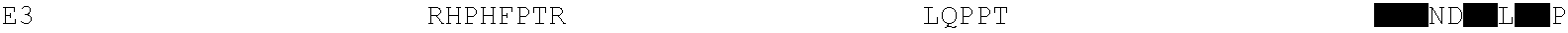

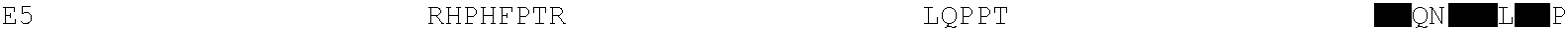

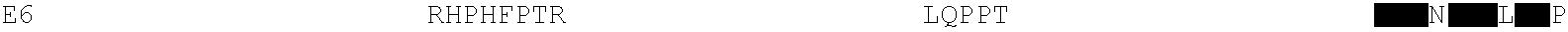

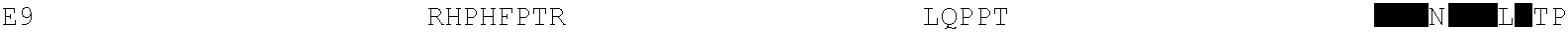

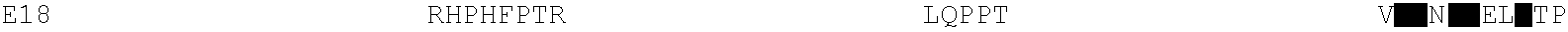

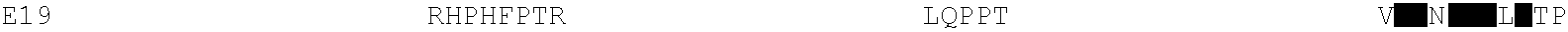

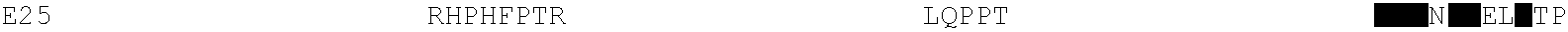

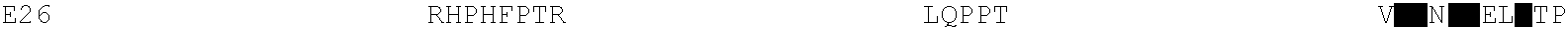

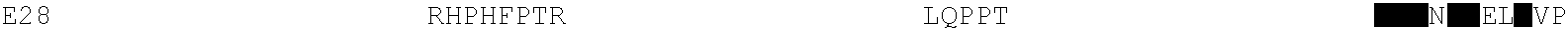

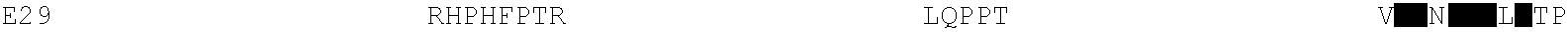

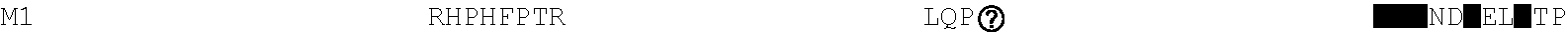

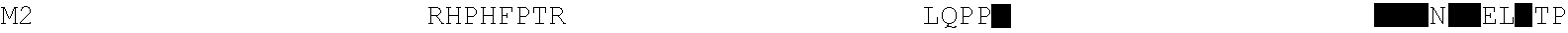

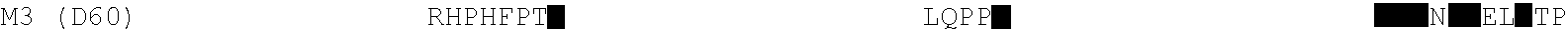

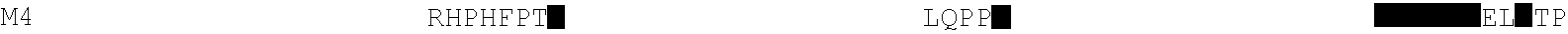

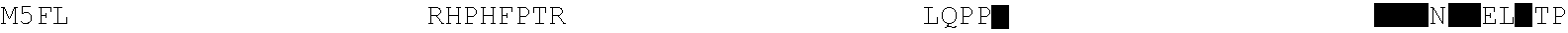

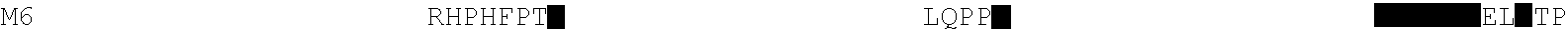

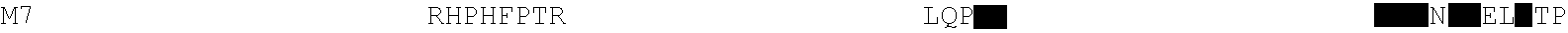

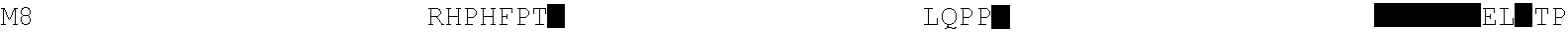

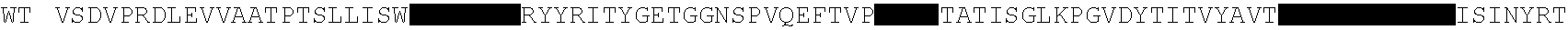

TABLE 5
Affinities of the trinectin binders to KDR-Fc
determined in radioactive equilibrium binding assay

| Clone | KDR (Kd, nM) |
|---|---|
| VR28 | 11.0 ± 0.5 |
| K1 | <0.6 ± 0.1 |
| K6 | <0.9 ± 0.1 |
| K9 | <1.8 ± 0.4 |
| K10 | <0.6 ± 0.1 |
| K12 | <0.6 ± 0.1 |
| K13 | <0.6 ± 0.1 |
| K14 | <0.6 ± 0.1 |
| K15 | <0.4 ± 0.1 |

TABLE 6
Affinities of the trinectin binders to KDR and Flk-1
determined in radioactive equilibrium binding assay

| Clone | KDR (Kd, nM) | Flk-1 (Kd, nM) |
|---|---|---|
| VR28 | 11.0 ± 0.5 | nd* |
| E3 | <1.0 ± 0.2 | 5.4 ± 1.5 |
| E5 | <0.4 ± 0.1 | 3.2 ± 0.3 |
| E6 | <0.4 ± 0.1 | 7.1 ± 1.1 |
| E9 | 2.4 ± 0.3 | <1.4 ± 0.1 |
| E18 | <1.2 ± 0.2 | <0.5 ± 0.1 |
| E19 | <1.3 ± 0.2 | <0.7 ± 0.1 |
| E25 | <1.6 ± 0.4 | <1.3 ± 0.2 |
| E26 | <1.7 ± 0.4 | 2.0 ± 0.3 |
| E28 | <1.6 ± 0.4 | 2.1 ± 0.6 |
| E29 | <1.5 ± 0.4 | <0.9 ± 0.2 |

nd* - binding is not detected at 100 nM of target

TABLE 7
Determination of ka, kd and Kd by BIAcore assay

| Clone | Target | ka (1/M * s) × 10⁻⁴ | kd(1/s) × 10⁺⁵ | Kd (nM) |
|---|---|---|---|---|
| E6 | KDR | 89 | 6.7 | 0.08 |
| | Flk-1 | 67 | 136.0 | 2.02 |
| E18 | KDR | 26 | 12.1 | 0.46 |
| | Flk-1 | 60 | 19.5 | 0.33 |
| E19 | KDR | 30 | 1.7 | 0.06 |
| | Flk-1 | 66 | 22.3 | 0.34 |
| E25 | KDR | 25 | 5.2 | 0.21 |
| | Flk-1 | 50 | 37.8 | 0.76 |
| E26 | KDR | 11 | 5.8 | 0.51 |
| | Flk-1 | 22 | 47.7 | 2.14 |
| E29 | KDR | 36 | 7.0 | 0.19 |
| | Flk-1 | 79 | 28.8 | 0.37 |
| M5FL | KDR | 10 | 9.2 | 0.89 |
| | Flk-1 | 28 | 58.2 | 2.10 |
| VR28 | KDR | 3 | 34 | 13 |
| 159(Q8L) | KDR | 5 | 10 | 2 |

TABLE 8
Binding to KDR (CHO KDR) and Flk-1
(CHO Flk-1) expressing cells

| | CHO KDR | CHO Flk-1 |
|---|---|---|
| Clone | (EC50, nM) | (EC50, nM) |
| E18 | 4.2 ± 1.0 | 0.9 ± 0.4 |
| E19 | 7.6 ± 1.7 | 5.3 ± 2.5 |
| E26 | 2.6 ± 1.2 | 1.3 ± 0.7 |
| E29 | 2.3 ± 1.0 | 0.6 ± 0.1 |
| WT | no | no |

TABLE 9
Inhibition of VEGF-induced proliferation of
KDR (Ba/F3-KDR) and Flk-1 (Ba/F3-Flk)
expressing cells

| | Ba/F3-KDR | Ba/F3-Flk |
|---|---|---|
| Clone | (IC50, nM) | (IC50, nM) |
| E18 | 5.4 ± 1.2 | 2.4 ± 0.2 |
| E19 | 12.3 ± 2.6 | 5.8 ± 1.0 |
| E26 | 3.2 ± 0.5 | 5.3 ± 1.7 |
| E29 | 10.0 ± 2.1 | 4.7 ± 1.2 |
| M5FL | 3.9 ± 1.1 | 5.1 ± 0.2 |
| WT | no | no |
| Anti-KDR Ab | 17.3 ± 7.7 | ND |
| Anti-Flk-1 Ab | ND | 15.0 ± 3.2 |

TABLE 10
Inhibition of VEGF-induced proliferation
of HUVEC cells

| Clone | (IC50, nM) |
|---|---|
| E18 | 12.8 ± 4.6 |
| E19 | 11.8 ± 2.7 |
| E26 | 14.0 ± 5.9 |
| E29 | 8.4 ± 0.8 |
| M5FL | 8.5 ± 2.8 |
| WT | no |

TABLE 11

| | hKDR | | | Flk-1 | | |
|---|---|---|---|---|---|---|
| | kₐ | k_{d} | | kₐ | k_{d} | |
| | (1/Ms) × | (1/s) × | K_{D} | (1/Ms) × | (1/s) × | K_{D} |
| | 10⁻⁴ | 10⁵ | (nM) | 10⁻⁴ | 10⁵ | (nM) |
| M5FL | 7.4 | 6.7 | 0.9 | 14.6 | 30 | 2.1 |
| C100 | | | | | | |
| M5FL 20K | 0.9 | 5.4 | 5.9 | 2.4 | 55 | 22.8 |
| PEG | | | | | | |
| M5FL 40K | 0.5 | 5.9 | 1.3 | 1.0 | 54 | 57.1 |
| PEG | | | | | | |

All references cited herein are hereby incorporated by reference in their entirety

We claim:

1. A sustained-release intraocular drug delivery system comprising: a therapeutic component comprising an antiangiogenic polypeptide component; and a polymeric component associated with the therapeutic component to permit the therapeutic component to be released into the interior of an eye of an individual at a therapeutically effective dosage for a period of time after the drug delivery system is placed in the eye.

2. The system of claim 1 wherein said therapeutic component and said polymeric component are combined in a form selected from the group consisting of a) an implant device, or b) a plurality of particles.

3. The system of claim 2 wherein the antiangiogenic polypeptide component comprises an antibody, antibody fragment, or artificial antibody, and humanized versions of these polypeptides.

4. The system of claim 3 wherein the antiangiogenic component comprises an artificial antibody or a humanized version thereof.

5. The system of claim 4 wherein the artificial antibody comprises a scaffold region based upon a fibronectin.

6. The system of claim 5 wherein the artificial antibody comprises fibronectin based “addressable” therapeutic binding molecule (“FATBIM”).

7. The system of claim 6 wherein the FATBIM is selected from the group consisting of CT322, C7S100 and C7C100.

8. A sustained-release intraocular drug delivery system comprising: a therapeutic component comprising an antiangiogenic polypeptide component, wherein the therapeutic component is selected from the group consisting of C7S100 and C7C100; and a polymeric component associated with the therapeutic component to permit the therapeutic component to be released into the interior of an eye of an individual at a therapeutically effective dosage for a period of time after the drug delivery system is placed in the eye.

9. A method of treating a retinopathy, the method comprising administering, to a patient in need thereof, a therapeutically effective amount of a polypeptide that binds to human VEGFR-2, the polypeptide comprising between about 80 and about 150 amino acids that have a structural organization comprising:
i) at least five to seven beta strands or beta-like strands distributed among at least two beta sheets, and
ii) at least one loop portion connecting two strands that are beta strands or beta-like strands, which loop portion participates in binding to VEGFR-2,
wherein the polypeptide binds to an extracellular domain of the human VEGFR-2 protein with a dissociation constant (K_{D}) of less than 1×10⁻⁶ M and inhibits VEGFR-2 mediated angiogenesis.
